(19) United States
(12) Patent Application Publication
(10) Pub. No.: US 2024/0034723 A1
(43) Pub. Date: Feb. 1, 2024

(54) PIPERIDINYL SMALL MOLECULE DEGRADERS OF HELIOS AND METHODS OF USE

(71) Applicants: DANA-FARBER CANCER INSTITUTE, INC., Boston, MA (US); NEOMORPH, INC., San Diego, CA (US)

(72) Inventors: Hu Liu, Newton, MA (US); Tinghu Zhang, Brookline, MA (US); Lyn Jones, Winchester, MA (US); Jianwei Che, Sharon, MA (US); Brian Bear, San Diego, CA (US); Whitney Petrilli, San Diego, CA (US)

(73) Assignees: DANA-FARBER CANCER INSTITUTE, INC., Boston, MA (US); NEOMORPH, INC., San Diego, CA (US)

(21) Appl. No.: 18/031,077

(22) PCT Filed: Oct. 15, 2021
(86) PCT No.: PCT/US2021/055186

§ 371 Date: Apr. 10, 2023

Related U.S. Application Data
(60) Provisional application No. 63/153,599, filed on Feb. 25, 2021.
(60) Provisional application No. 63/092,610, filed on Oct. 16, 2020.

(51) Int. Cl. C07D 401/14; C07D 471/04; C07D 413/14; C07D 487/04; C07D 417/14; C07D 513/04

(52) U.S. Cl. CPC C07D 401/14; C07D 471/04; C07D 413/14; C07D 487/04; C07D 417/14; C07D 513/04

(57) ABSTRACT

Disclosed are compounds and pharmaceutically acceptable salts, hydrates, solvates, prodrugs, stereoisomers, or tautomers thereof that may cause degradation of various proteins e.g., IKZF2 (Helios). Also disclosed are pharmaceutical compositions containing same, and methods of making and using the compounds to treat diseases and disorders associated with Helios and which may benefit from Helios degradation.

### RELATED APPLICATIONS

This application claims the benefit of priority under 35 U.S.C. § 119(e) to U.S. Provisional Application No. 63/092,610, filed Oct. 16, 2020 and U.S. Provisional Application No. 63/153,599, filed Feb. 25, 2021, each of which are incorporated herein by reference in their entireties.

### BACKGROUND OF THE INVENTION

Imide molecules, such as thalidomide and its analogs, bind to Cereblon (CRBN), a substrate adaptor for the ubiquitously expressed cullin ring ligase 4 (CUL4)-RBX1-DDB1-CRBN (CUL4CRBN) E3 ligase (Kronke et al., Science 343:301-305 (2014); Ito et al., Science 327:1345-1350 (2010)). This results in the recruitment, ubiquitination, and the subsequent proteasomal degradation of neo-substrates, namely Ikaros (IKZF1) and Aiolos (IKZF3), but not any other members of the IKZF zinc finger transcription factor family. CC-885, an imide analog, is predicted to have some activity in inducing Helios degradation, but also induces degradation of GSPT1, a key translation termination factor (Matyskiela et al., Nature 535:252-257 (2016)).

Helios (IKZF2), a member of the IKZF family, is a critical regulator of T cell activity and function. Genetic deletion of Helios resulted in an enhanced anti-tumor immune response (Kim et al., Science 350:334-339 (2015)). Notably, Helios is highly expressed in regulatory T cells (Elkord et al., Expert Opin. Biol. Ther. 12:1423-1425 (2012)), a subpopulation of T cells that restricts the activity of effector T cells. Selective deletion of Helios in regulatory T cells resulted in both loss of suppressive activity and acquisition of effector T cell functions (Najagawa et al., Proc. Natl. Acad. Sci. USA 113:6248-6253 (2016); Yates et al., Proc. Natl. Acad. Sci. USA 115:2162-2167 (2018)). Thus, Helios is a critical factor in restricting T cell effector function in Tregs.

Helios expression has also been reported to be upregulated in ‘exhausted’ T cells, in the settings of both chronic viral infections (Crawford et al., Immunity 40:289-302 (2014), Doering et al., Immunity 371130-1144 (2012); Scott-Browne et al., Immunity 45:1327-1340 (2016)) and tumors (Martinez et al., Immunity 42:265-278 (2015); Mognol et al., Proc. Natl. Acad. Sci. USA 114:E2776-E2785 (2017); Pereira et al., J. Leukoc. Biol. 102:601-615 (2017); Singer et al., Cell 166:1500-1511 (2016); Schietinger et al., Immunity 45:389-401 (2016)), as well as in dysfunctional chimeric antigen receptor (CAR) T cells (Long et al., Nat. Med. 21:581-590 (2015)). Overexpression or aberrant expression of Helios and various splice isoforms have been reported in several hematological malignancies, including T cell leukemias and lymphomas (Nakase at al., Exp. Hematol. 30:313-317 (2002); Tabayashi et al., Cancer Sci. 98:182-188 (2007); Asanuma et al., Cancer Sci. 104:1097-1106 (2013)). Moreover, knockdown of Helios in a model of mixed lineage leukemia (MLL)-driven myeloid leukemia potently suppressed proliferation and increased cell death (Park et al., J. Clin. Invest. 125:1286-1298 (2015); Park et al., Cell Stem Cell 24:153-165 (2019)).

### SUMMARY OF THE INVENTION

A first aspect of the present invention is directed to a compound having a structure represented by formula (I):

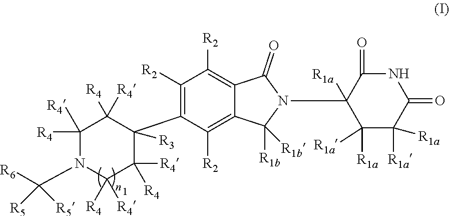

wherein R₁ₐ, R_{1b}, R₁ₐ′, R_{1b}′, R₂, R₃, R₄, R₄′, R₅, R₅′, R₆, and n₁ are as defined herein, or a pharmaceutically acceptable salt, hydrate, solvate, prodrug, stereoisomer, or tautomer thereof.

A second aspect of the present invention is directed to a compound having a structure represented by formula (II):

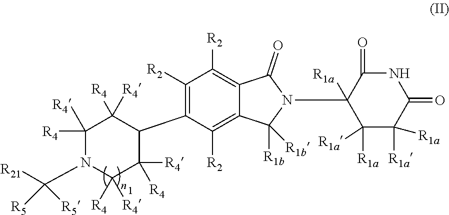

wherein R₁ₐ, R_{1b}, R₁ₐ′, R_{1b}′, R₂, R₄, R₄′, R₅, R₅′, R₂₁, and n₁ are as defined herein, or a pharmaceutically acceptable salt, hydrate, solvate, prodrug, stereoisomer, or tautomer thereof.

Another aspect of the present invention is directed to a pharmaceutical composition that includes a therapeutically effective amount of a compound of formula (I) or (II) or a pharmaceutically acceptable salt, hydrate, solvate, prodrug, stereoisomer, or tautomer thereof, and a pharmaceutically acceptable carrier. In some embodiments, the pharmaceutical composition comprises a co-crystal of a compound of formula (I) or (II).

A further aspect of the present invention is directed to methods of treating diseases or disorders that would benefit from IKZF2 (Helios) degradation.

In some embodiments, the disease or disorder is cancer. In some embodiments, the cancer is T cell leukemia, T cell lymphoma, Hodgkin's lymphoma, non-Hodgkin's lymphoma, myeloid leukemia, non-small cell lung cancer (NSCLC), melanoma, triple-negative breast cancer (TNBC), nasopharyngeal cancer (NPC), microsatellite stable colorectal cancer (mssCRC), thymoma, or carcinoid.

As demonstrated in the working examples, compounds of the present invention exhibit potent degradation of IKZF2 (Helios).

Although not intending to be bound by any particular theory of operation, it is believed that inventive compounds may enhance an anti-tumor immune response by converting regulatory T cells into effector T cells, and by rescuing effector T cell function in exhausted T cells or CAR-T cells.

### DETAILED DESCRIPTION OF THE INVENTION

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present invention.

As used in the description and the appended claims, the singular forms “a” “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2%, or 1%) of the particular value modified by the term “about.”

The transitional term “comprising,” which is synonymous with “including,” “containing,” or “characterized by,” is inclusive or open-ended and does not exclude additional, unrecited elements or method steps. When used in the context of the number of heteroatoms in a heterocyclic structure, it means that the heterocyclic group that that minimum number of heteroatoms. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

With respect to compounds of the present invention, and to the extent the following terms are used herein to further describe them, the following definitions apply.

As used herein, the term “alkyl” refers to a saturated linear or branched-chain monovalent hydrocarbon radical. In one embodiment, the alkyl radical is a C₁-C₁₈ group. In other embodiments, the alkyl radical is a C₀-C₆, C₀-C₅, C₀-C₃, C₁-C₁₂, C₁-C₈, C₁-C₆, C₁-C₅, C₁-C₄ or C₁-C₃ group (wherein C₀ alkyl refers to a bond). Examples of alkyl groups include methyl, ethyl, 1-propyl, 2-propyl, i-propyl, 1-butyl, 2-methyl-1-propyl, 2-butyl, 2-methyl-2-propyl, 1-pentyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, heptyl, octyl, nonyl, decyl, undecyl and dodecyl. In some embodiments, an alkyl group is a C₁-C₃ alkyl group. In some embodiments, an alkyl group is a C₁-C₂ alkyl group, or a methyl group.

As used herein, the term “alkylene” refers to a straight or branched divalent hydrocarbon chain linking the rest of the molecule to a radical group, consisting solely of carbon and hydrogen, containing no unsaturation and having from one to 12 carbon atoms, for example, methylene, ethylene, propylene, n-butylene, and the like. The alkylene chain may be attached to the rest of the molecule through a single bond and to the radical group through a single bond. In some embodiments, the alkylene group contains one to 8 carbon atoms (C₁-C₈ alkylene). In other embodiments, an alkylene group contains one to 5 carbon atoms (C₁-C₅ alkylene). In other embodiments, an alkylene group contains one to 4 carbon atoms (C₁-C₄ alkylene). In other embodiments, an alkylene contains one to three carbon atoms (C₁-C₃ alkylene). In other embodiments, an alkylene group contains one to two carbon atoms (C₁-C₂ alkylene). In other embodiments, an alkylene group contains one carbon atom (C₁ alkylene).

As used herein, the term “alkenyl” refers to a linear or branched-chain monovalent hydrocarbon radical with at least one carbon-carbon double bond. An alkenyl includes radicals having “cis” and “trans” orientations, or alternatively, “E” and “Z” orientations. In one example, the alkenyl radical is a C₂-C₁₈ group. In other embodiments, the alkenyl radical is a C₂-C₁₂, C₂-C₁₀, C₂-C₈, C₂-C₆ or C₂-C₃ group. Examples include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl.

As used herein, the term “alkynyl” refers to a linear or branched monovalent hydrocarbon radical with at least one carbon-carbon triple bond. In one example, the alkynyl radical is a C₂-C₁₈ group. In other examples, the alkynyl radical is C₂-C₁₂, C₂-C₁₀, C₂-C₈, C₂-C₆ or C₂-C₃. Examples include ethynyl prop-1-ynyl, prop-2-ynyl, but-1-ynyl, but-2-ynyl and but-3-ynyl.

The terms “alkoxyl” or “alkoxy” as used herein refer to an alkyl group, as defined above, having an oxygen radical attached thereto, and which is the point of attachment. Representative alkoxyl groups include methoxy, ethoxy, propyloxy, tert-butoxy and the like. An “ether” is two hydrocarbyl groups covalently linked by an oxygen. Accordingly, the substituent of an alkyl that renders that alkyl an ether is or resembles an alkoxyl, such as can be represented by one of —O-alkyl, —O-alkenyl, and —O-alkynyl.

As used herein, the term “halogen” (or “halo” or “halide”) refers to fluorine, chlorine, bromine, or iodine.

As used herein, the term “cyclic group” broadly refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. Cyclic groups may have one or more (e.g., fused) ring systems. Thus, for example, a cyclic group can contain one or more carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 20 carbon atoms, that is alone or part of a larger moiety (e.g., an alkcarbocyclic group). The term carbocyclyl includes mono-, bi-, tri-, fused, bridged, and spiro-ring systems, and combinations thereof. In one embodiment, carbocyclyl includes 3 to 15 carbon atoms (C₃-C₁₅). In one embodiment, carbocyclyl includes 3 to 12 carbon atoms (C₃-C₁₂). In another embodiment, carbocyclyl includes C₃-C₈, C₃-C₁₀ or C₅-C₁₀. In another embodiment, carbocyclyl, as a monocycle, includes C₃-C₈, C₃-C₆ or C₅-C₆. In some embodiments, carbocyclyl, as a bicycle, includes C₇-C₁₂. In another embodiment, carbocyclyl, as a spiro system, includes C₅-C₁₂. Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, phenyl, and cyclododecyl; bicyclic carbocyclyls having 7 to 12 ring atoms include [4,3], [4,4], [4,5], [5,5], [5,6] or [6,6] ring systems, such as for example bicyclo[2.2.1]heptane, bicyclo[2.2.2]octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocycyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

Thus, the term carbocyclic also embraces carbocyclylalkyl groups which as used herein refer to a group of the formula —R^{c}-carbocyclyl where R^{c} is an alkylene chain. The term carbocyclic also embraces carbocyclylalkoxy groups which as used herein refer to a group bonded through an oxygen atom of the formula —O—R^{c}-carbocyclyl where R^{c} is an alkylene chain.

As used herein, the term “aryl” used alone or as part of a larger moiety (e.g., “aralkyl”, wherein the terminal carbon atom on the alkyl group is the point of attachment, e.g., a benzyl group), “aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. In some embodiments, the aralkoxy group is a benzoxy group. The term “aryl” may be used interchangeably with the term “aryl ring”. In one embodiment, aryl includes groups having 6-18 carbon atoms. In another embodiment, aryl includes groups having 6-10 carbon atoms. Examples of aryl groups include phenyl, naphthyl, anthracyl, biphenyl, phenanthrenyl, naphthacenyl, 1,2,3,4-tetrahydronaphthalenyl, 1H-indenyl, 2,3-dihydro-1H-indenyl, naphthyridinyl, and the like, which may be substituted or independently substituted by one or more substituents described herein. A particular aryl is phenyl. In some embodiments, an aryl group includes an aryl ring fused to one or more (e.g., 1, 2 or 3) different cyclic groups (e.g., carbocyclic rings or heterocyclic rings), where the radical or point of attachment is on the aryl ring. The structure of any aryl group that is capable of having double bonds positioned differently is considered so as to embrace any and all such resonance structures.

Thus, the term aryl embraces aralkyl groups (e.g., benzyl) which as disclosed above refer to a group of the formula —R^{c}-aryl where R^{c} is an alkylene chain such as methylene or ethylene. In some embodiments, the aralkyl group is an optionally substituted benzyl group. The term aryl also embraces aralkoxy groups which as used herein refer to a group bonded through an oxygen atom of the formula —O—R^{c}-aryl where R^{c} is an alkylene chain such as methylene or ethylene.

As used herein, the term “heterocyclyl” refers to a “carbocyclyl” that used alone or as part of a larger moiety, contains a saturated, partially unsaturated or aromatic ring system, wherein one or more (e.g., 1, 2, 3, or 4) carbon atoms have been replaced with a heteroatom (e.g., O, N, N(O), S, S(O), or S(O)₂). The term heterocyclyl includes mono-, bi-, tri-, fused, bridged, and spiro-ring systems, and combinations thereof. In some embodiments, a heterocyclyl refers to a 3 to 15 membered heterocyclyl ring system. In some embodiments, a heterocyclyl refers to a 3 to 12 membered heterocyclyl ring system. In some embodiments, a heterocyclyl refers to a saturated ring system, such as a 3 to 12 membered saturated heterocyclyl ring system. In some embodiments, a heterocyclyl refers to a heteroaryl ring system, such as a 5 to 14 membered heteroaryl ring system. The term heterocyclyl also includes C₃-C₈ heterocycloalkyl, which is a saturated or partially unsaturated mono-, bi-, or spiro-ring system containing 3-8 carbons and one or more (1, 2, 3 or 4) heteroatoms.

In some embodiments, a heterocyclyl group includes 3-12 ring atoms and includes monocycles, bicycles, tricycles and spiro ring systems, wherein the ring atoms are carbon, and one to 5 ring atoms is a heteroatom such as nitrogen, sulfur or oxygen. In some embodiments, heterocyclyl includes 3- to 7-membered monocycles having one or more heteroatoms selected from nitrogen, sulfur and oxygen. In some embodiments, heterocyclyl includes 4- to 6-membered monocycles having one or more heteroatoms selected from nitrogen, sulfur and oxygen. In some embodiments, heterocyclyl includes 3-membered monocycles. In some embodiments, heterocyclyl includes 4-membered monocycles. In some embodiments, heterocyclyl includes 5-6 membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be quaternized (e.g., [NR₄]+Cl⁻, [NR₄]⁺OH⁻). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, thiophenyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl, including thiazol-2-yl and thiazol-2-yl N-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Example 5-membered ring heterocyclyls containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Representative examples of benzo-fused 5-membered heterocyclyls are benzoxazol-2-yl, benzthiazol-2-yl and benzimidazol-2-yl. Example 6-membered heterocyclyls contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl. The pyridine N-oxides and pyridazine N-oxides and the pyridyl, pyrimid-2-yl, pyrimid-4-yl, pyridazinyl and the 1,3,4-triazin-2-yl groups, are yet other examples of heterocyclyl groups. In some embodiments, a heterocyclic group includes a heterocyclic ring fused to one or more (e.g., 1, 2 or 3) different cyclic groups (e.g., carbocyclic rings or heterocyclic rings), where the radical or point of attachment is on the heterocyclic ring, and in some embodiments wherein the point of attachment is a heteroatom contained in the heterocyclic ring.

Thus, the term heterocyclic embraces N-heterocyclyl groups which as used herein refer to a heterocyclyl group containing at least one nitrogen and where the point of attachment of the heterocyclyl group to the rest of the molecule is through a nitrogen atom in the heterocyclyl group. Representative examples of N-heterocyclyl groups include 1-morpholinyl, 1-piperidinyl, 1-piperazinyl, 1-pyrrolidinyl, pyrazolidinyl, imidazolinyl and imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. Representative examples of C-heterocyclyl radicals include 2-morpholinyl, 2- or 3- or 4-piperidinyl, 2-piperazinyl, and 2- or 3-pyrrolidinyl. The term heterocyclic also embraces heterocyclylalkyl groups which as disclosed above refer to a group of the formula —R^{c}— heterocyclyl where R^{c} is an alkylene chain. The term heterocyclic also embraces heterocyclylalkoxy groups which as used herein refer to a radical bonded through an oxygen atom of the formula —O—R^{c}-heterocyclyl where R is an alkylene chain.

As used herein, the term “heteroaryl” used alone or as part of a larger moiety (e.g., “heteroarylalkyl” (also “heteroaralkyl”), or “heteroarylalkoxy” (also “heteroaralkoxy”), refers to a monocyclic, bicyclic or tricyclic ring system having 5 to 14 ring atoms, wherein at least one ring is aromatic and contains at least one heteroatom. In one embodiment, heteroaryl includes 5-6 membered monocyclic aromatic groups where one or more ring atoms is nitrogen, sulfur or oxygen. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridazinyl, triazinyl, tetrazinyl, tetrazolo[1,5-b]pyridazinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, 1,2,3-triazol-5-yl, and pyrid-2-yl N-oxide. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methylenedioxyphenyl, benzofuranyl, dibenzofuranyl, indazolyl, benzimidazolyl, benzodioxazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3(4H)-one. A heteroaryl group may be mono-, bi- or tri-cyclic. In some embodiments, a heteroaryl group includes a heteroaryl ring fused to one or more (e.g., 1, 2 or 3) different cyclic groups (e.g., carbocyclic rings or heterocyclic rings), where the radical or point of attachment is on the heteroaryl ring, and in some embodiments wherein the point of attachment is a heteroatom contained in the heterocyclic ring. The structure of any heteroaryl group that is capable of having double bonds positioned differently is considered so as to embrace any and all such resonance structures.

Thus, the term heteroaryl embraces N-heteroaryl groups which as used herein refer to a heteroaryl group as defined above containing at least one nitrogen and where the point of attachment of the heteroaryl group to the rest of the molecule is through a nitrogen atom in the heteroaryl group. The term heteroaryl also embraces C-heteroaryl groups which as used herein refer to a heteroaryl group as defined above and where the point of attachment of the heteroaryl group to the rest of the molecule is through a carbon atom in the heteroaryl group. The term heteroaryl also embraces heteroarylalkyl groups which as disclosed above refer to a group of the formula —R^{c}-heteroaryl, wherein R^{c} is an alkylene chain as defined above. The term heteroaryl also embraces heteroaralkoxy (or heteroarylalkoxy) groups which as used herein refer to a group bonded through an oxygen atom of the formula —O—R^{c}-heteroaryl, where R^{c} is an alkylene group as defined above.

Unless stated otherwise, and to the extent not further defined for any particular group(s), any of the groups described herein may be substituted or unsubstituted. As used herein, the term “substituted” broadly refers to all permissible substituents with the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc. Representative substituents include halogens, hydroxyl groups, and any other organic groupings containing any number of carbon atoms, e.g., 1-14 carbon atoms, and which may include one or more (e.g., 1, 2, 3, or 4) heteroatoms such as oxygen, sulfur, and nitrogen grouped in a linear, branched, or cyclic structural format.

To the extent not disclosed otherwise for any particular group(s), representative examples of substituents may include alkyl, substituted alkyl (e.g., C₁-C₆, C₁-C₅, C₁-C₄, C₁-C₃, C₁-C₂, C₁), alkoxy (e.g., C₁-C₆, C₁-C₅, C₁-C₄, C₁-C₃, C₁-C₂, C₁), substituted alkoxy (e.g., C₁-C₆, C₁-C₅, C₁-C₄, C₁-C₃, C₁-C₂, C₁), haloalkyl (e.g., CF₃), alkenyl (e.g., C₂-C₆, C₂-C₅, C₂-C₄, C₂-C₃, C₂), substituted alkenyl (e.g., C₂-C₆, C₂-C₅, C₂-C₄, C₂-C₃, C₂), alkynyl (e.g., C₂-C₆, C₂-C₅, C₂-C₄, C₂-C₃, C₂), substituted alkynyl (e.g., C₂-C₆, C₂-C₅, C₂-C₄, C₂-C₃, C₂), cyclic (e.g., C₃-C₁₂, C₅-C₆), substituted cyclic (e.g., C₃-C₁₂, C₅-C₆), carbocyclic (e.g., C₃-C₁₂, C₅-C₆), substituted carbocyclic (e.g., C₃-C₁₂, C₅-C₆), heterocyclic (e.g., C₃-C₁₂, C₅-C₆), substituted heterocyclic (e.g., C₃-C₁₂, C₅-C₆), aryl (e.g., benzyl and phenyl), substituted aryl (e.g., substituted benzyl or phenyl), heteroaryl (e.g., pyridyl or pyrimidyl), substituted heteroaryl (e.g., substituted pyridyl or pyrimidyl), aralkyl (e.g., benzyl), substituted aralkyl (e.g., substituted benzyl), halo, hydroxyl, aryloxy (e.g., C₆-C₁₂, C₆), substituted aryloxy (e.g., C₆-C₁₂, C₆), alkylthio (e.g., C₁-C₆), substituted alkylthio (e.g., C₁-C₆), arylthio (e.g., C₆-C₁₂, C₆), substituted arylthio (e.g., C₆-C₁₂, C₆), cyano, carbonyl, substituted carbonyl, carboxyl, substituted carboxyl, amino, substituted amino, amido, substituted amido, thio, substituted thio, sulfinyl, substituted sulfinyl, sulfonyl, substituted sulfonyl, sulfinamide, substituted sulfinamide, sulfonamide, substituted sulfonamide, urea, substituted urea, carbamate, substituted carbamate, amino acid, and peptide groups.

In one aspect, compounds of the invention are represented by formula (I):

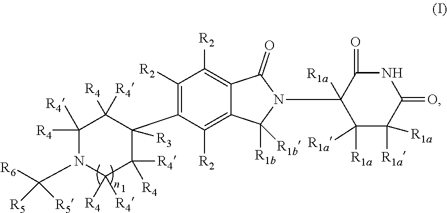

or a pharmaceutically acceptable salt, hydrate, solvate, prodrug, stereoisomer, or tautomer thereof,
wherein:
each R₁ₐ, R_{1b}, R₁ₐ′ and R_{1b}′ is independently hydrogen or (C₁-C₆)alkyl, or
R₁ₐ and R₁ₐ′, together with the same carbon atom to which they are attached, form a spiro (C₃-C₇)cycloalkyl group or a 4- to 7-membered heterocycloalkyl group, or
R₁ₐ and R₁ₐ′, when on different carbon atoms, together with the atoms to which they are attached, form a (C₃-C₇)cycloalkyl group or a 4- to 7-membered heterocycloalkyl group, or R_{1b} and R_{1b}′ form a spiro (C₃-C₇)cycloalkyl group or a 4- to 7-membered heterocycloalkyl group; wherein said alkyl, cycloalkyl, or heterocycloalkyl is further optionally and independently substituted by one or more identical or different R₁₅ groups;
each R₂ is independently selected from the group consisting of hydrogen, hydroxy, amino, cyano, halo, (C₁-C₆)alkyl, and (C₁-C₆)haloalkyl;
R₃ is selected from the group consisting of hydrogen, amino, hydroxyl, cyano, halogen, (C₁-C₆)alkyl, and (C₁-C₆)haloalkyl, wherein said alkyl, is further optionally and independently substituted by one or more identical or different R₁₅ groups, or
R₃ and R₄, together with the carbon atoms to which they are attached, form a (C₃-C₇)cycloalkyl group or a 4- to 7-membered heterocycloalkyl group, or
R₂ and R₃, together with the atoms to which they are attached, form a (C₃-C₇)cycloalkyl group or a 4- to 7-membered heterocycloalkyl group, wherein said cycloalkyl, heterocycloalkyl is further optionally and independently substituted by one or more identical or different R₁₅ groups;
each R₄ and R₄′ is independently selected from the group consisting of hydrogen, hydroxyl, amino, amido, carbonyl, cyano, halogen, (C₁-C₆)alkyl, (C₁-C₆)haloalkyl, (C₁-C₆)hydroxyalkyl, (C₃-C₇)cycloalkyl, 4- to 7-membered heterocycloalkyl, (C₆-C₁₀)aryl, monocyclic and bicyclic 5- to 10-membered heteroaryl, (C₂-C₆)alkenyl, and (C₂-C₆)alkynyl; wherein said alkyl, alkynyl, cycloalkyl, heterocycloalkyl, aryl, or heteroaryl is further optionally and independently substituted by one or more identical or different R₁₅ groups, or
R₄ and R₄′, together with the same carbon atom to which they are attached, form a spiro (C₃-C₇)cycloalkyl group or a 4- to 7-membered heterocycloalkyl group, or
R₄ and R₄′, together with the same carbon atom to which they are attached, form C═(O), or
R₄ and R₄′, when on different carbon atoms, together with the atoms to which they are attached, form a (C₃-C₇)cycloalkyl group or a 4- to 7-membered heterocycloalkyl group, or
R₄ and R₄′, together with the carbon atoms to which they are attached, form a (C₆-C₁₀)aryl or a 5- or 6-membered heteroaryl; wherein said cycloalkyl, heterocycloalkyl, aryl or heteroaryl is further optionally and independently substituted by one or more identical or different R₁₅ groups;
R₅ and R₅′ are independently selected from the group consisting of hydrogen, (C₁-C₆)alkyl, (C₂-C₆)alkenyl, (C₂-C₆)alkynyl, (C₁-C₆)haloalkyl, (C₁-C₆)hydroxyalkyl, (C₃-C₇)cycloalkyl, 4- to 7-membered heterocycloalkyl, (C₆-C₁₀)aryl, and monocyclic and bicyclic 5- to 10-membered heteroaryl; wherein said alkyl, cycloalkyl, heterocycloalkyl, aryl or heteroaryl is further optionally and independently substituted by one or more identical or different R₁₅ groups;
R₆ is an R₇-substituted aryl or a R₇-substituted heteroaryl; wherein said aryl or heteroaryl is further optionally and independently substituted by one or more identical or different R₁₅ groups, or
R₆ is:

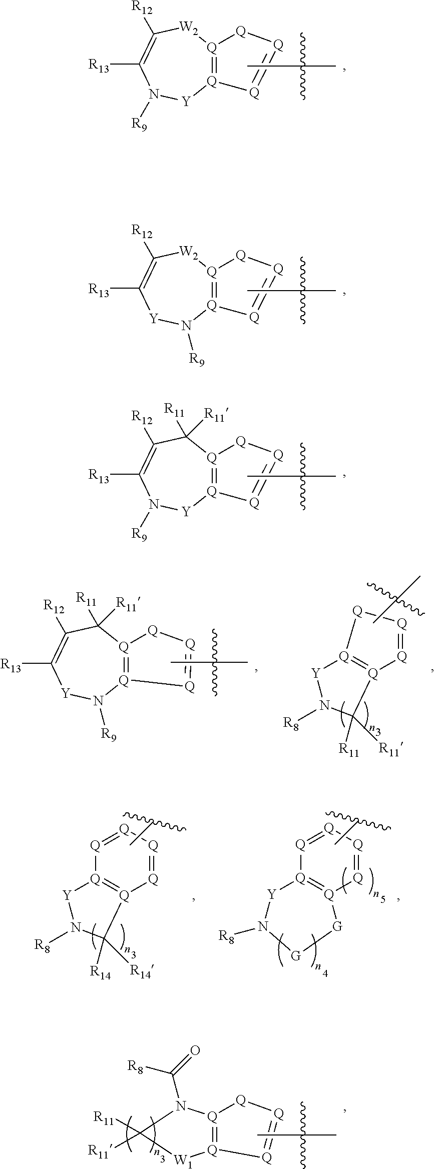

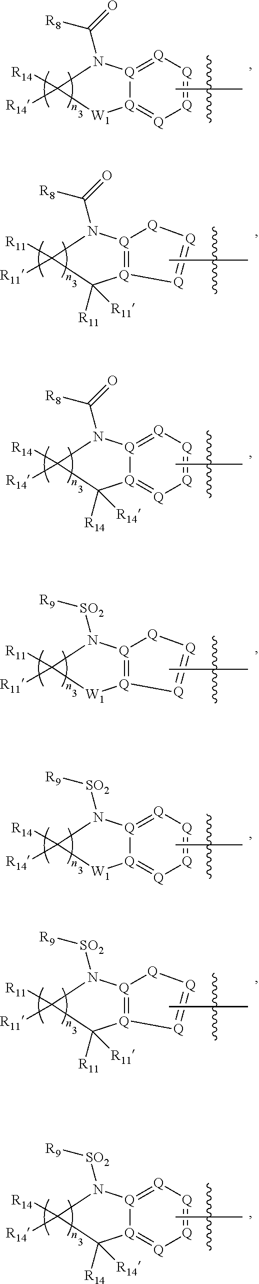

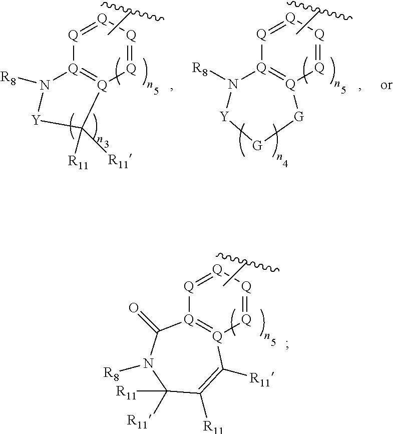

R₇ is selected from the group consisting of:

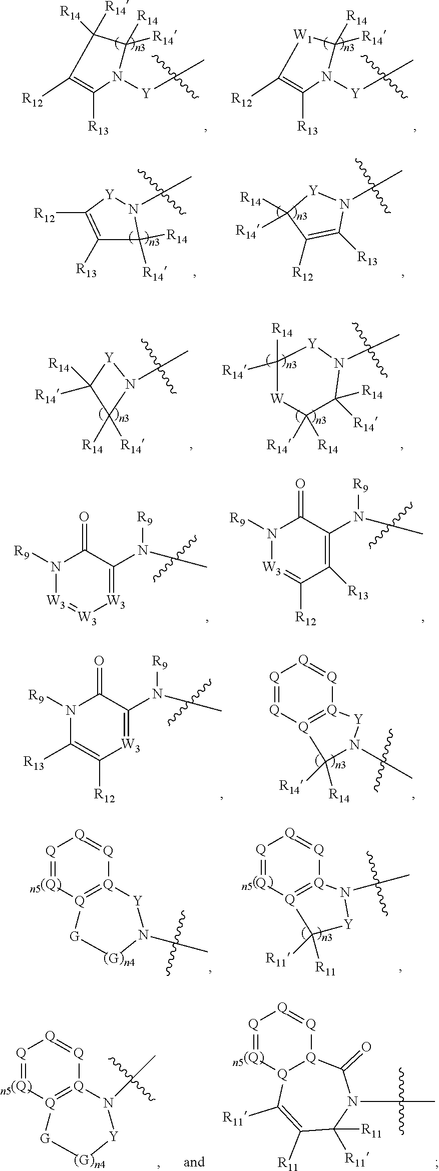

R₈ is selected from the group consisting of (C₆-C₁₀)aryl, and monocyclic and bicyclic 5- to 10-membered heteroaryl; wherein said aryl or heteroaryl is further optionally and independently substituted by one or more identical or different R₁₅ groups;
each R₉ is independently selected from the group consisting of hydrogen, (C₁-C₆)alkyl, (C₁-C₆)haloalkyl, (C₃-C₇)cycloalkyl, 4- to 7-membered heterocycloalkyl, (C₆-C₁₀)aryl, and monocyclic and bicyclic 5- to 10-membered heteroaryl; wherein said alkyl, cycloalkyl, heterocycloalkyl, aryl or heteroaryl is further optionally and independently substituted by one or more identical or different R₁₅ groups;
each R₁₁ and R₁₁′ is independently selected from the group consisting of hydrogen, (C₁-C₆)alkyl, (C₁-C₆)haloalkyl, (C₃-C₇)cycloalkyl, 4- to 7-membered heterocycloalkyl, (C₆-C₁₀)aryl, monocyclic and bicyclic 5- to 10-membered heteroaryl, halo, cyano, —N(R₉)₂, —OR₉, (C₁-C₆)alkoxy, (C₁-C₆)haloalkoxy, (C₂-C₆)alkenyl, and (C₂-C₆)alkynyl; wherein said alkyl, cycloalkyl, heterocycloalkyl, aryl, or heteroaryl is further optionally and independently substituted by one or more identical or different R₁₅ groups; or
R₁₁ and R₁₁′, together with the same carbon atom to which they are attached, form a spiro (C₃-C₇)cycloalkyl group or a 4- to 7-membered heterocycloalkyl group, or
R₁₁ and R₁₁′, together with the same carbon atom to which they are attached, form C═(O), or
R₁₁ and R₁₁′, when on different carbon atoms, together with the atoms to which they are attached, form a (C₃-C₇)cycloalkyl group or a 4- to 7-membered heterocycloalkyl group; wherein said cycloalkyl, or heterocycloalkyl is further optionally and independently substituted by one or more identical or different R₁₅ groups;
R₁₂ and R₁₃, together with the carbon atoms to which they are attached, form a (C₆-C₁₀)aryl, or a monocyclic or bicyclic 5- to 10-membered heteroaryl, wherein said aryl or heteroaryl is further optionally and independently substituted by one or more identical or different R₁₅ groups;
each R₁₄ and R₁₄′ is independently selected from the group consisting of hydrogen, (C₁-C₆)alkyl, (C₁-C₆)haloalkyl, (C₃-C₇)cycloalkyl, 4- to 7-membered heterocycloalkyl, (C₆-C₁₀)aryl, monocyclic and bicyclic 5- to 10-membered heteroaryl, halo, cyano, —N(R₉)₂, —OR₉, (C₁-C₆)alkoxy, (C₁-C₆)haloalkoxy, (C₂-C₆)alkenyl, and (C₂-C₆)alkynyl; wherein said alkyl, cycloalkyl, heterocycloalkyl, aryl, or heteroaryl is further optionally and independently substituted by one or more identical or different R₁₅ groups; or
R₁₄ and R₁₄′, together with the same carbon atom to which they are attached, form a spiro (C₃-C₇)cycloalkyl group or a 4- to 7-membered heterocycloalkyl group, or
R₁₄ and R₁₄′, together with the same carbon atom to which they are attached, form C═(O), or
R₁₄ and R₁₄′, when on different carbon atoms, together with the atoms to which they are attached, form a (C₃-C₇)cycloalkyl group or a 4- to 7-membered heterocycloalkyl group; wherein said cycloalkyl, or heterocycloalkyl is further optionally and independently substituted by one or more identical or different R₁₅ groups;
provided that at least one R₁₄ and at least one R₁₄′, together with the same carbon atom to which they are attached, form C═(O);
each R₁₅ is independently selected from the group consisting of alkyl, alkenyl, alkynyl, halo, haloalkyl, cycloalkyl, heterocycloalkyl, hydroxy, alkoxy, cycloalkoxy, heterocycloalkoxy, haloalkoxy, aryloxy, heteroaryloxy, aralkyloxy, alkyenyloxy, alkynyloxy, amino, alkylamino, cycloalkylamino, heterocycloalkylamino, arylamino, heteroarylamino, aralkylamino, N-alkyl-N-arylamino, N-alkyl-N-heteroarylamino, N-alkyl-N-aralkylamino, hydroxyalkyl, aminoalkyl, alkylthio, haloalkylthio, alkylsulfonyl, haloalkylsulfonyl, cycloalkylsulfonyl, heterocycloalkylsulfonyl, arylsulfonyl, heteroarylsulfonyl, aminosulfonyl, alkylaminosulfonyl, cycloalkylaminosulfonyl, heterocycloalkylaminosulfonyl, arylaminosulfonyl, heteroarylaminosulfonyl, N-alkyl-N-arylaminosulfonyl, N-alkyl-N-heteroarylaminosulfonyl, formyl, alkylcarbonyl, haloalkylcarbonyl, alkenylcarbonyl, alkynylcarbonyl, carboxy, alkoxycarbonyl, alkylcarbonyloxy, amino, alkylsulfonylamino, haloalkylsulfonylamino, cycloalkylsulfonylamino, heterocycloalkylsulfonylamino, arylsulfonylamino, heteroarylsulfonylamino, aralkylsulfonylamino, alkylcarbonylamino, haloalkylcarbonylamino, cycloalkylcarbonylamino, heterocycloalkylcarbonylamino, arylcarbonylamino, heteroarylcarbonylamino, aralkylsulfonylamino, aminocarbonyl, alkylaminocarbonyl, cycloalkylaminocarbonyl, heterocycloalkylaminocarbonyl, arylaminocarbonyl, heteroarylaminocarbonyl, N-alkyl-N-arylaminocarbonyl, N-alkyl-N-heteroarylaminocarbonyl, cyano, nitro, azido, phosphinyl, phosphoryl including phosphine oxide and phosphonate, cyclic acetal, 4- to 7-membered heterocycloalkyl which contains at least one nitrogen atom and is linked via the nitrogen atom, aryl, heteroaryl, and where two adjacent R₁₅, together with the respective atoms to which each is bonded, form aryl, heteroaryl, 5- to 8-membered cycloalkyl or 5- to 8-membered heterocycloalkyl;
R₁₆ is independently selected from the group consisting of hydrogen, (C₁-C₆)alkyl, (C₁-C₆)haloalkyl, (C₃-C₇)cycloalkyl, 4- to 7-membered heterocycloalkyl, (C₆-C₁₀)aryl, monocyclic and bicyclic 5- to 10-membered heteroaryl, halo, cyano, —N(R₉)₂, —OR₉, (C₁-C₆)alkoxy, (C₁-C₆)haloalkoxy, (C₂-C₆)alkenyl, (C₂-C₆)alkynyl, and a radical that participates in the formation of a single bond; wherein said alkyl, cycloalkyl, heterocycloalkyl, aryl, or heteroaryl is further optionally and independently substituted by one or more identical or different R₁₅ groups;
each G is independently selected from C(R₁₁)(R₁₁′), NR₁₁ and O, provided that at least one G is NR₁₁ or O;
W₁ is selected from the group consisting of —O—, —S— and —NR₉—;
W₂ is selected from the group consisting of —O—, —S—, —SO₂—, —C═(O)— and —NR₉—;
each W₃ is nitrogen or CR₁₆;
Y is —SO₂— or —C═(O)—;
each Q is independently selected from C, C(R₁₆), C═(O), O, S, N, and NR₁₆;
n₁ is 0, 1 or 2;
n₃ is independently 1, 2 or 3;
n₄ is independently 1 or 2; and
n₅ is independently 0 or 1.

In some embodiments, compounds are represented by formula I, wherein:
each R₁ₐ, R_{1b}, R₁ₐ′ and R_{1b}′ is hydrogen;
each R₂ is independently selected from the group consisting of hydrogen, halo, and (C₁-C₆)alkyl;
R₃ is selected from the group consisting of hydrogen, amino, hydroxyl, cyano, halogen, (C₁-C₆)alkyl, and (C₁-C₆)haloalkyl, wherein said alkyl, is further optionally and independently substituted by one or more identical or different R₁₅ groups;
each R₄ and R₄′ is independently selected from the group consisting of hydrogen, hydroxyl, halogen, (C₁-C₆)alkyl, (C₁-C₆)haloalkyl, and (C₁-C₆)hydroxyalkyl, wherein said alkyl is further optionally and independently substituted by one or more identical or different R₁₅ groups, or
R₄ and R₄′, together with the same carbon atom to which they are attached, form a spiro (C₃-C₇)cycloalkyl group or a 4- to 7-membered heterocycloalkyl group, or
R₄ and R₄′, when on different carbon atoms, together with the atoms to which they are attached, form a (C₃-C₇)cycloalkyl group or a 4- to 7-membered heterocycloalkyl group;
R₅ and R₅′ are independently hydrogen or (C₁-C₆)alkyl; wherein said alkyl is further optionally and independently substituted by one or more identical or different R₁₅ groups;
R₆ is an R₇-substituted aryl or a R₇-substituted heteroaryl; wherein said aryl or heteroaryl is further optionally and independently substituted by one or more identical or different R₁₅ groups, or
R₆ is:

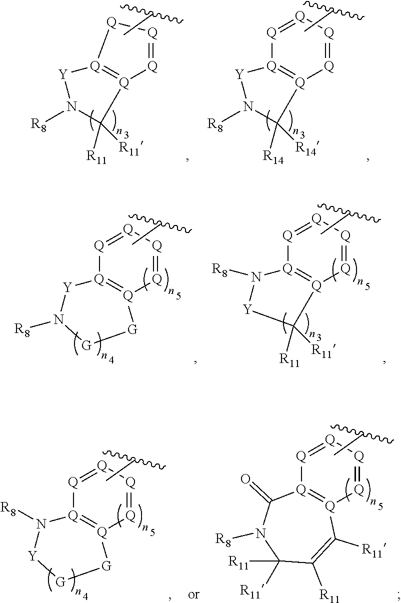

R₇ is selected from the group consisting of:

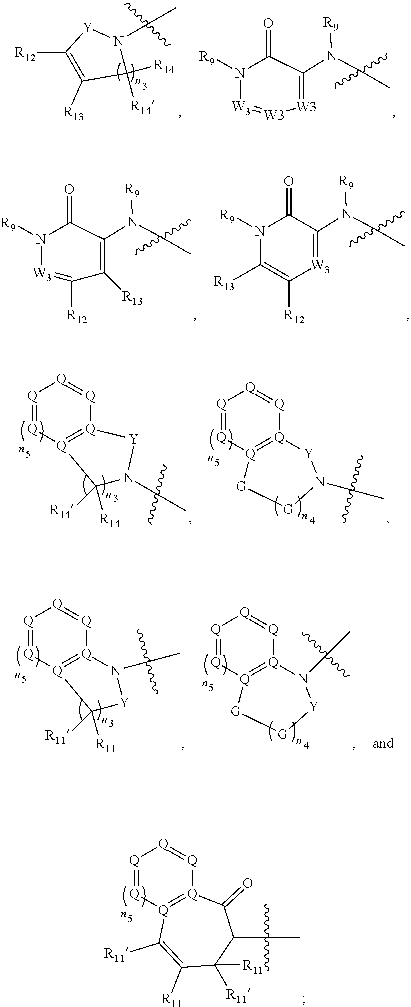

R₈ is selected from the group consisting of (C₆-C₁₀)aryl, and monocyclic and bicyclic 5- to 10-membered heteroaryl; wherein said aryl or heteroaryl is further optionally and independently substituted by one or more identical or different R₁₅ groups;
each R₉ is independently selected from the group consisting of hydrogen, (C₁-C₆)alkyl, (C₁-C₆)haloalkyl, (C₃-C₇)cycloalkyl, 4- to 7-membered heterocycloalkyl, (C₆-C₁₀)aryl, and monocyclic and bicyclic 5- to 10-membered heteroaryl; wherein said alkyl, cycloalkyl, heterocycloalkyl, aryl or heteroaryl is further optionally and independently substituted by one or more identical or different R₁₅ groups;
each R₁₁ and R₁₁′ is independently selected from the group consisting of hydrogen, (C₁-C₆)alkyl, (C₁-C₆)haloalkyl, (C₃-C₇)cycloalkyl, 4- to 7-membered heterocycloalkyl, (C₆-C₁₀)aryl, monocyclic and bicyclic 5- to 10-membered heteroaryl, halo, cyano, —N(R₉)₂, —OR₉, (C₁-C₆)alkoxy, (C₁-C₆)haloalkoxy, (C₂-C₆)alkenyl, and (C₂-C₆)alkynyl; wherein said alkyl, cycloalkyl, heterocycloalkyl, aryl, or heteroaryl is further optionally and independently substituted by one or more identical or different R₁₅ groups; or
R₁₁ and R₁₁′, together with the same carbon atom to which they are attached, form a spiro (C₃-C₇)cycloalkyl group or a 4- to 7-membered heterocycloalkyl group, or
R₁₁ and R₁₁′, together with the same carbon atom to which they are attached, form C═(O), or
R₁₁ and R₁₁′, when on different carbon atoms, together with the atoms to which they are attached, form a (C₃-C₇)cycloalkyl group or a 4- to 7-membered heterocycloalkyl group; wherein said cycloalkyl, or heterocycloalkyl is further optionally and independently substituted by one or more identical or different R₁₅ groups;
R₁₂ and R₁₃, together with the carbon atoms to which they are attached, form a (C₆-C₁₀)aryl, or a monocyclic or bicyclic 5- to 10-membered heteroaryl, wherein said aryl or heteroaryl is further optionally and independently substituted by one or more identical or different R₁₅ groups;
each R₁₄ and R₁₄′ is independently selected from the group consisting of hydrogen, (C₁-C₆)alkyl, (C₁-C₆)haloalkyl, (C₃-C₇)cycloalkyl, 4- to 7-membered heterocycloalkyl, (C₆-C₁₀)aryl, monocyclic and bicyclic 5- to 10-membered heteroaryl, halo, cyano, —N(R₉)₂, —OR₉, (C₁-C₆)alkoxy, (C₁-C₆)haloalkoxy, (C₂-C₆)alkenyl, and (C₂-C₆)alkynyl; wherein said alkyl, cycloalkyl, heterocycloalkyl, aryl, or heteroaryl is further optionally and independently substituted by one or more identical or different R₁₅ groups; or
R₁₄ and R₁₄′, together with the same carbon atom to which they are attached, form a spiro (C₃-C₇)cycloalkyl group or a 4- to 7-membered heterocycloalkyl group, or
R₁₄ and R₁₄′, together with the same carbon atom to which they are attached, form C═(O), or
R₁₄ and R₁₄′, when on different carbon atoms, together with the atoms to which they are attached, form a (C₃-C₇)cycloalkyl group or a 4- to 7-membered heterocycloalkyl group; wherein said cycloalkyl, or heterocycloalkyl is further optionally and independently substituted by one or more identical or different R₁₅ groups,
provided that at least one R₁₄ and at least one R₁₄′, together with the same carbon atom to which they are attached, form C═(O);
each R₁₅ is independently selected from the group consisting of alkyl, alkenyl, alkynyl, halo, haloalkyl, cycloalkyl, heterocycloalkyl, hydroxy, alkoxy, cycloalkoxy, heterocycloalkoxy, haloalkoxy, aryloxy, heteroaryloxy, aralkyloxy, alkyenyloxy, alkynyloxy, amino, alkylamino, cycloalkylamino, heterocycloalkylamino, arylamino, heteroarylamino, aralkylamino, N-alkyl-N-arylamino, N-alkyl-N-heteroarylamino, N-alkyl-N-aralkylamino, hydroxyalkyl, aminoalkyl, alkylthio, haloalkylthio, alkylsulfonyl, haloalkylsulfonyl, cycloalkylsulfonyl, heterocycloalkylsulfonyl, arylsulfonyl, heteroarylsulfonyl, aminosulfonyl, alkylaminosulfonyl, cycloalkylaminosulfonyl, heterocycloalkylaminosulfonyl, arylaminosulfonyl, heteroarylaminosulfonyl, N-alkyl-N-arylaminosulfonyl, N-alkyl-N-heteroarylaminosulfonyl, formyl, alkylcarbonyl, haloalkylcarbonyl, alkenylcarbonyl, alkynylcarbonyl, carboxy, alkoxycarbonyl, alkylcarbonyloxy, amino, alkylsulfonylamino, haloalkylsulfonylamino, cycloalkylsulfonylamino, heterocycloalkylsulfonylamino, arylsulfonylamino, heteroarylsulfonylamino, aralkylsulfonylamino, alkylcarbonylamino, haloalkylcarbonylamino, cycloalkylcarbonylamino, heterocycloalkylcarbonylamino, arylcarbonylamino, heteroarylcarbonylamino, aralkylsulfonylamino, aminocarbonyl, alkylaminocarbonyl, cycloalkylaminocarbonyl, heterocycloalkylaminocarbonyl, arylaminocarbonyl, heteroarylaminocarbonyl, N-alkyl-N-arylaminocarbonyl, N-alkyl-N-heteroarylaminocarbonyl, cyano, nitro, azido, phosphinyl, phosphoryl including phosphine oxide and phosphonate, cyclic acetal, 4- to 7-membered heterocycloalkyl which contains at least one nitrogen atom and is linked via the nitrogen atom, aryl, heteroaryl, and where two adjacent R₁₅ taken together with the respective atoms to which each is bonded form aryl, heteroaryl, 5- to 8-membered cycloalkyl or 5- to 8-membered heterocycloalkyl;
R₁₆ is independently selected from the group consisting of hydrogen, (C₁-C₆)alkyl, (C₁-C₆)haloalkyl, (C₃-C₇)cycloalkyl, 4- to 7-membered heterocycloalkyl, (C₆-C₁₀)aryl, monocyclic and bicyclic 5- to 10-membered heteroaryl, halo, cyano, —N(R₉)₂, —OR₉, (C₁-C₆)alkoxy, (C₁-C₆)haloalkoxy, (C₂-C₆)alkenyl, (C₂-C₆)alkynyl, and a radical that participates in the formation of a single bond; wherein said alkyl, cycloalkyl, heterocycloalkyl, aryl, or heteroaryl is further optionally and independently substituted by one or more identical or different R₁₅ groups;
each G is independently selected from C(R₁₁)(R₁₁′), NR₁₁ and O, provided that at least one G is NR₁₁ or O;
W₁ is selected from the group consisting of —O—, —S— and —NR₉—;
W₂ is selected from the group consisting of —O—, —S—, —SO₂—, —C═(O)— and —NR₉—;
each W₃ is nitrogen or CR₁₆;
Y is —SO₂— or —C═(O)—;
each Q is independently selected from C, C(R₁₆), C═(O), O, S, N, and NR₁₆,
n₁ is 0, 1 or 2;
n₃ is independently 1, 2 or 3;
n₄ is independently 1 or 2; and
n₅ is independently 0 or 1.

In some embodiments, compounds are represented by formula I, wherein:
each R₂ is hydrogen;
R₃ is hydrogen or hydroxyl;
each R₄ and R₄′ is independently hydrogen or (C₁-C₆)alkyl;
R₅ and R₅′ are independently selected from hydrogen or (C₁-C₆)alkyl;
R₆ is

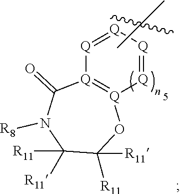

each R₁₁ and R₁₁′ is independently hydrogen or (C₁-C₆)alkyl, wherein said alkyl is further optionally and independently substituted by one or more identical or different R₁₅ groups; or R₁₁ and R₁₁′, together with the same carbon atom to which they are attached, form a spiro (C₃-C₇)cycloalkyl group or a 4- to 7-membered heterocycloalkyl group, or
R₁₁ and R₁₁′, together with the same carbon atom to which they are attached form, C═(O), or
R₁₁ and R₁₁′, when on different carbon atoms, together with the atoms to which they are attached, form a (C₃-C₇)cycloalkyl group or a 4- to 7-membered heterocycloalkyl group; wherein said cycloalkyl, or heterocycloalkyl is further optionally and independently substituted by one or more identical or different R₁₅ groups;
R₁₆ is independently selected from the group consisting of hydrogen, (C₁-C₆)alkyl, (C₁-C₆)haloalkyl, halo, cyano, —N(R₉)₂, —OR₉, (C₁-C₆)alkoxy, (C₁-C₆)haloalkoxy, and a radical that participates in the formation of a single bond; wherein said alkyl is further optionally and independently substituted by one or more identical or different R₁₅ groups; and n₁ is 1.

In some embodiments, compounds are represented by formula I, wherein R₆ is

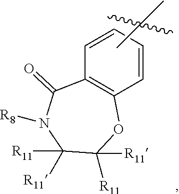

wherein R₆ is further optionally and independently substituted with one or more groups selected from (C₁-C₆)alkyl, halo, and cyano; and
each R₁₁ and R₁₁′ is independently hydrogen or (C₁-C₆)alkyl.

In some embodiments, compounds are represented by formula I, R₈ is selected from:

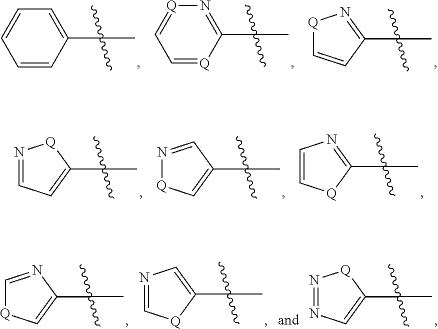

wherein R₈ is further optionally and independently substituted with one or more R₁₅.

In some embodiments, compounds are represented by formula I, R₈ is selected from:

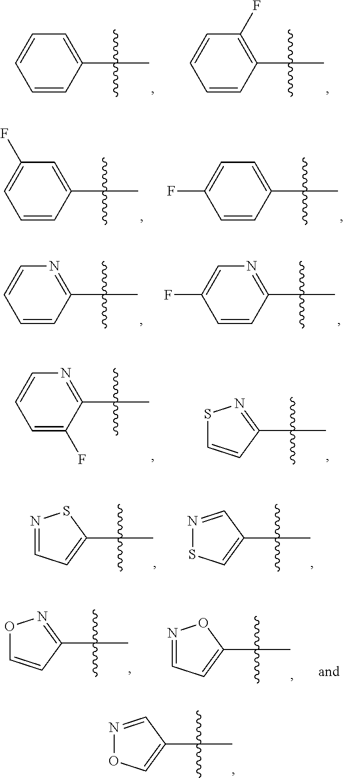

wherein R₈ is further optionally and independently substituted with one or more R₁₅.

A second aspect of the present invention is directed to a compound having a structure represented by formula (II):

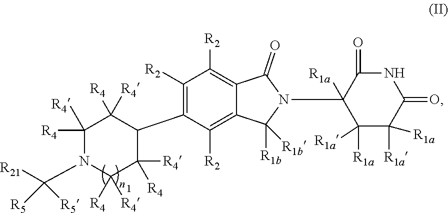

or a pharmaceutically acceptable salt, hydrate, solvate, prodrug, stereoisomer, or tautomer thereof,
wherein:
each R₁ₐ, R_{1b}, R₁ₐ′ and R_{1b}′ is independently hydrogen or (C₁-C₆)alkyl, or
R₁ₐ and R₁ₐ′, together with the same carbon atom to which they are attached, form a spiro (C₃-C₇)cycloalkyl group or a 4- to 7-membered heterocycloalkyl group, or
R₁ₐ and R₁ₐ′, when on different carbon atoms, together with the atoms to which they are attached, form a (C₃-C₇)cycloalkyl group or a 4- to 7-membered heterocycloalkyl group, or
R_{1b} and R_{1b}′ form a spiro (C₃-C₇)cycloalkyl group or a 4- to 7-membered heterocycloalkyl group; wherein said alkyl, cycloalkyl, or heterocycloalkyl is further optionally and independently substituted by one or more identical or different R₁₅ groups;
each R₂ is independently selected from the group consisting of hydrogen, hydroxy, amino, cyano, halo, (C₁-C₆)alkyl, and (C₁-C₆)haloalkyl;
each R₄ and R₄′ is independently selected from the group consisting of hydrogen, hydroxyl, amino, amido, carbonyl, cyano, halogen, (C₁-C₆)alkyl, (C₁-C₆)haloalkyl, (C₁-C₆)hydroxyalkyl, (C₃-C₇)cycloalkyl, 4- to 7-membered heterocycloalkyl, (C₆-C₁₀)aryl, monocyclic and bicyclic 5- to 10-membered heteroaryl, (C₂-C₆)alkenyl, and (C₂-C₆)alkynyl; wherein said alkyl, alkynyl, cycloalkyl, heterocycloalkyl, aryl, or heteroaryl is further optionally and independently substituted by one or more identical or different R₁₅ groups, or
R₄ and R₄′, together with the same carbon atom to which they are attached, form a spiro (C₃-C₇)cycloalkyl group or a 4- to 7-membered heterocycloalkyl group, or
R₄ and R₄′, together with the same carbon atom to which they are attached, form a C═(O), or
R₄ and R₄′, when on different carbon atoms, together with the atoms to which they are attached, form a (C₃-C₇)cycloalkyl group or a 4- to 7-membered heterocycloalkyl group, or
R₄ and R₄′, together with the carbon atoms to which they are attached, form a (C₆-C₁₀)aryl or a 5- or 6-membered heteroaryl; wherein said cycloalkyl, heterocycloalkyl, aryl or heteroaryl is further optionally and independently substituted by one or more identical or different R₁₅ groups;
R₅ and R₅′ are independently selected from the group consisting of hydrogen, (C₁-C₆)alkyl, (C₂-C₆)alkenyl, (C₂-C₆)alkynyl, (C₁-C₆)haloalkyl, (C₁-C₆)hydroxyalkyl, (C₃-C₇)cycloalkyl, 4- to 7-membered heterocycloalkyl, (C₆-C₁₀)aryl, and monocyclic and bicyclic 5- to 10-membered heteroaryl; wherein said alkyl, cycloalkyl, heterocycloalkyl, aryl or heteroaryl is further optionally and independently substituted by one or more identical or different R₁₅ groups;
each R₁₅ is independently selected from the group consisting of alkyl, alkenyl, alkynyl, halo, haloalkyl, cycloalkyl, heterocycloalkyl, hydroxy, alkoxy, cycloalkoxy, heterocycloalkoxy, haloalkoxy, aryloxy, heteroaryloxy, aralkyloxy, alkyenyloxy, alkynyloxy, amino, alkylamino, cycloalkylamino, heterocycloalkylamino, arylamino, heteroarylamino, aralkylamino, N-alkyl-N-arylamino, N-alkyl-N-heteroarylamino, N-alkyl-N-aralkylamino, hydroxyalkyl, aminoalkyl, alkylthio, haloalkylthio, alkylsulfonyl, haloalkylsulfonyl, cycloalkylsulfonyl, heterocycloalkylsulfonyl, arylsulfonyl, heteroarylsulfonyl, aminosulfonyl, alkylaminosulfonyl, cycloalkylaminosulfonyl, heterocycloalkylaminosulfonyl, arylaminosulfonyl, heteroarylaminosulfonyl, N-alkyl-N-arylaminosulfonyl, N-alkyl-N-heteroarylaminosulfonyl, formyl, alkylcarbonyl, haloalkylcarbonyl, alkenylcarbonyl, alkynylcarbonyl, carboxy, alkoxycarbonyl, alkylcarbonyloxy, amino, alkylsulfonylamino, haloalkylsulfonylamino, cycloalkylsulfonylamino, heterocycloalkylsulfonylamino, arylsulfonylamino, heteroarylsulfonylamino, aralkylsulfonylamino, alkylcarbonylamino, haloalkylcarbonylamino, cycloalkylcarbonylamino, heterocycloalkylcarbonylamino, arylcarbonylamino, heteroarylcarbonylamino, aralkylsulfonylamino, aminocarbonyl, alkylaminocarbonyl, cycloalkylaminocarbonyl, heterocycloalkylaminocarbonyl, arylaminocarbonyl, heteroarylaminocarbonyl, N-alkyl-N-arylaminocarbonyl, N-alkyl-N-heteroarylaminocarbonyl, cyano, nitro, azido, phosphinyl, phosphoryl including phosphine oxide and phosphonate, cyclic acetal, 4- to 7-membered heterocycloalkyl which contains at least one nitrogen atom and is linked via the nitrogen atom, aryl, heteroaryl, and where two adjacent R₁₅ taken together with the respective atoms to which each is bonded form aryl, heteroaryl, 5- to 8-membered cycloalkyl, or a 5- to 8-membered heterocycloalkyl;
R₂₁ is a substituted C₆-aryl, provided said aryl is substituted with at least two R₁₅, and provided two of the R₁₅, when on adjacent carbon atoms, form a 5- or 6-membered heteroaryl that is substituted with at least one (C₆-C₁₀)aryl, or monocyclic or bicyclic 5- to 10-membered heteroaryl; wherein said aryl and heteroaryl are further optionally and independently substituted with one or more R₁₅, or
R₂₁ is a substituted 5- or 6-membered heteroaryl, provided said heteroaryl is substituted with at least two R₁₅, and provided two of the R₁₅, when on adjacent atoms, form a C₆-aryl, or 5- or 6-membered heteroaryl that is substituted with at least one (C₆-C₁₀)aryl, or monocyclic or bicyclic 5- to 10-membered heteroaryl; wherein said aryl and heteroaryl are further optionally and independently substituted with one or more R₁₅, or
R₂₁ is

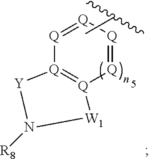

R₈ is selected from the group consisting of (C₆-C₁₀)aryl, and monocyclic and bicyclic 5- to 10-membered heteroaryl; wherein said aryl or heteroaryl is further optionally and independently substituted by one or more identical or different R₁₅ groups;
W₁ is selected from the group consisting of —O—, —S— and —NR₉—;
R₉ is independently selected from the group consisting of hydrogen, (C₁-C₆)alkyl, (C₁-C₆)haloalkyl, (C₃-C₇)cycloalkyl, 4- to 7-membered heterocycloalkyl, (C₆-C₁₀)aryl, and monocyclic and bicyclic 5- to 10-membered heteroaryl; wherein said alkyl, cycloalkyl, heterocycloalkyl, aryl or heteroaryl is further optionally and independently substituted by one or more identical or different R₁₅ groups;
Y is —SO₂— or —C═(O)—;
each Q is independently selected from C, C(R₁₆), C═(O), O, S, N, and NR₁₆;
R₁₆ is independently selected from the group consisting of hydrogen, (C₁-C₆)alkyl, (C₁-C₆)haloalkyl, (C₃-C₇)cycloalkyl, 4- one or more to 7-membered heterocycloalkyl, (C₆-C₁₀)aryl, monocyclic and bicyclic 5- to 10-membered heteroaryl, halo, cyano, —N(R₉)₂, —OR₉, (C₁-C₆)alkoxy, (C₁-C₆)haloalkoxy, (C₂-C₆)alkenyl, (C₂-C₆)alkynyl, and a radical that participates in the formation of a single bond; wherein said alkyl, cycloalkyl, heterocycloalkyl, aryl, or heteroaryl is further optionally and independently substituted by one or more identical or different R₁₅ groups;
n₁ is 0, 1 or 2; and
n₅ is independently 0 or 1.

In some embodiments, compounds are represented by formula II, wherein:
each R₁ₐ, R_{1b}, R₁ₐ′ and R_{1b}′ is hydrogen;
each R₂ is hydrogen;
each R₄ and R₄′ is independently hydrogen or (C₁-C₆)alkyl;
R₅ and R₅′ are each hydrogen or (C₁-C₆)alkyl;
R₂₁ is

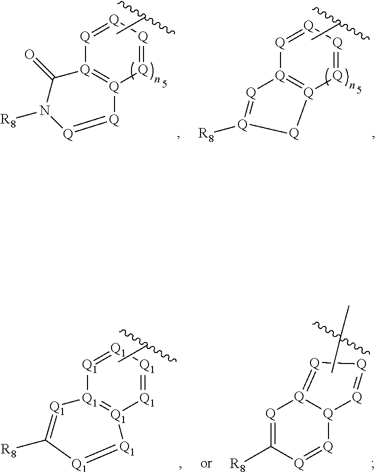

each Q₁ is independently selected from C, C(R₁₆), C═(O), O, S, N, and NR₁₆, provided that at least one Q₁ is N;
R₁₆ is independently selected from the group consisting of hydrogen, (C₁-C₆)alkyl, (C₁-C₆)haloalkyl, halo, cyano, —N(R₉)₂, —OR₉, (C₁-C₆)alkoxy, (C₁-C₆)haloalkoxy, and a radical that participates in the formation of a single bond; wherein said alkyl, cycloalkyl, heterocycloalkyl, aryl, or heteroaryl is further optionally and independently substituted by one or more identical or different R₁₅ groups; and
n₁ is 1.

In some embodiments, compounds are represented by formula II, wherein R₂₁ is selected from:

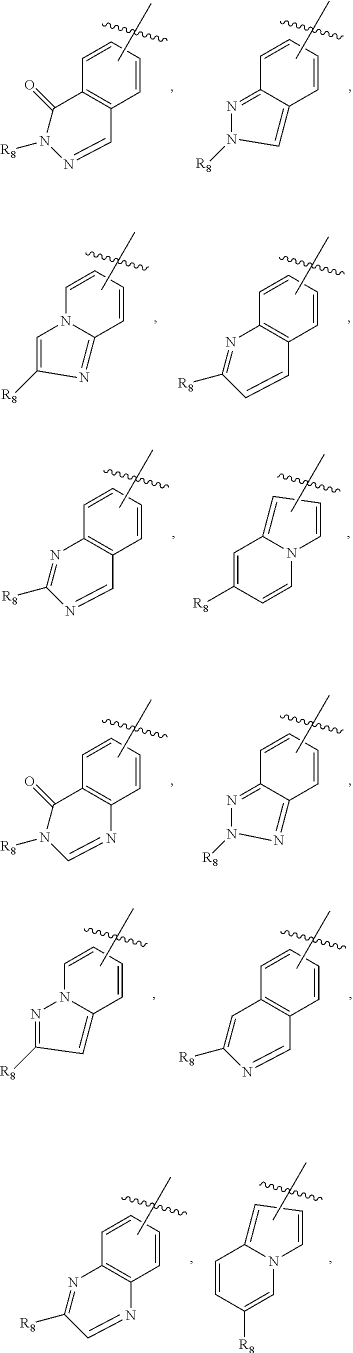

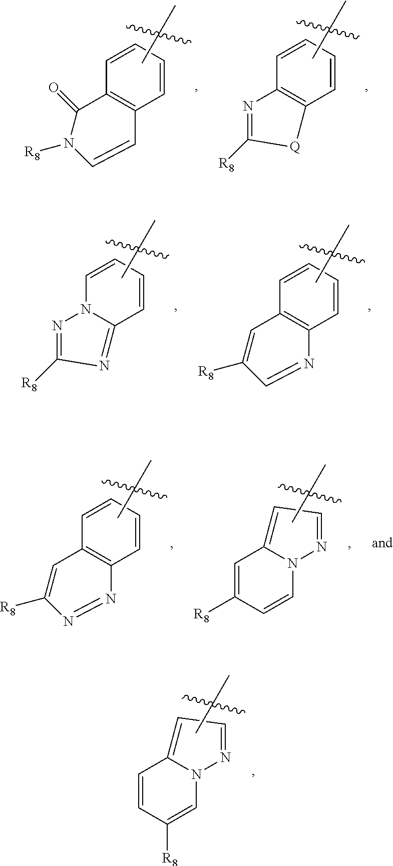

and wherein R₂₁ is optionally and independently substituted with one or more (C₁-C₆)alkyl, halo, and cyano.

In some embodiments, compounds are represented by formula II, R₈ is selected from:

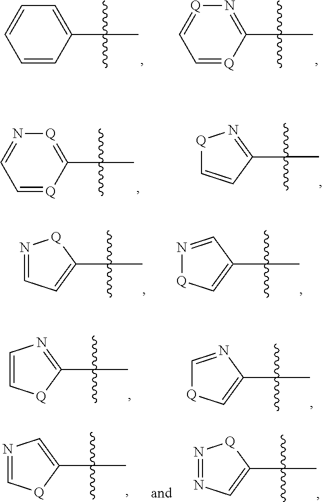

wherein R₈ is further optionally and independently substituted with one or more R₁₅.

In some embodiments, compounds are represented by formula II, R₈ is selected from:

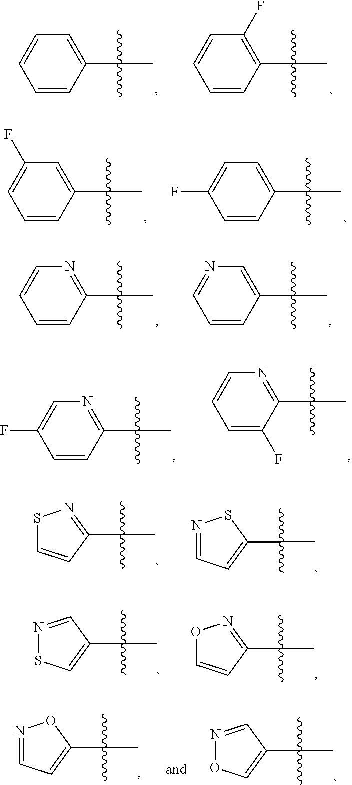

wherein R₈ is further optionally and independently substituted with one or more R₁₅.

In some embodiments, compounds of the invention are represented by formula IIa, IIb, IIc, IId, or IIe:

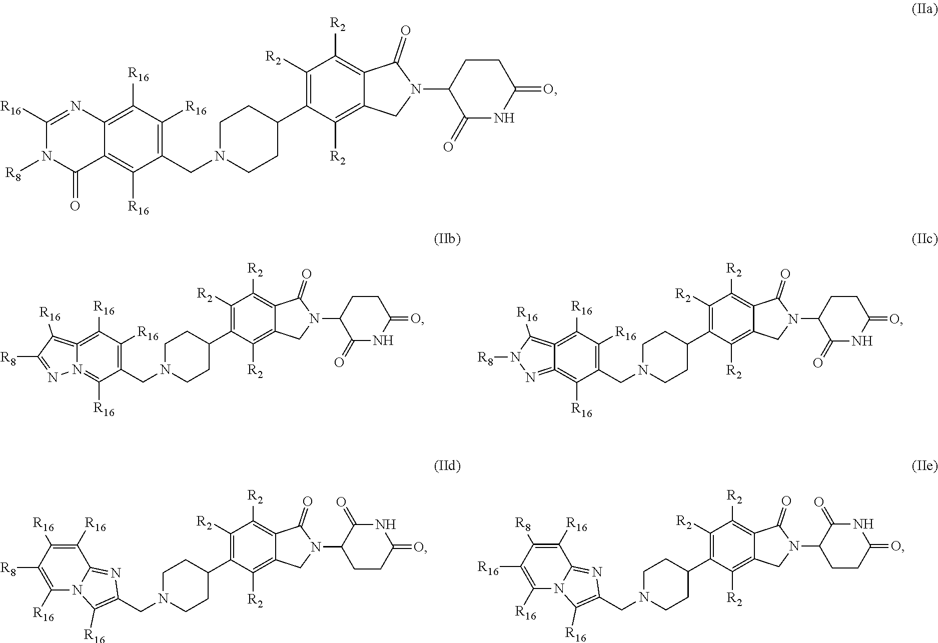

or a pharmaceutically acceptable salt, hydrate, solvate, prodrug, stereoisomer, or tautomer thereof,
wherein,
each R₂ is independently selected from the group consisting of hydrogen and halo;
R₈ is selected from the group consisting of (C₆-C₁₀)aryl, and monocyclic and bicyclic 5- to 10-membered heteroaryl; wherein said aryl or heteroaryl is further optionally and independently substituted by one or more identical or different R₁₅ groups;
   each R₁₅ is independently selected from the group consisting of alkyl, alkenyl, alkynyl, halo, haloalkyl, cycloalkyl, heterocycloalkyl, hydroxy, alkoxy, cycloalkoxy, heterocycloalkoxy, haloalkoxy, aryloxy, heteroaryloxy, aralkyloxy, alkyenyloxy, alkynyloxy, amino, alkylamino, cycloalkylamino, heterocycloalkylamino, arylamino, heteroarylamino, aralkylamino, N-alkyl-N-arylamino, N-alkyl-N-heteroarylamino, N-alkyl-N-aralkylamino, hydroxyalkyl, aminoalkyl, alkylthio, haloalkylthio, alkylsulfonyl, haloalkylsulfonyl, cycloalkylsulfonyl, heterocycloalkylsulfonyl, arylsulfonyl, heteroarylsulfonyl, aminosulfonyl, alkylaminosulfonyl, cycloalkylaminosulfonyl, heterocycloalkylaminosulfonyl, arylaminosulfonyl, heteroarylaminosulfonyl, N-alkyl-N-arylaminosulfonyl, N-alkyl-N-heteroarylaminosulfonyl, formyl, alkylcarbonyl, haloalkylcarbonyl, alkenylcarbonyl, alkynylcarbonyl, carboxy, alkoxycarbonyl, alkylcarbonyloxy, amino, alkylsulfonylamino, haloalkylsulfonylamino, cycloalkylsulfonylamino, heterocycloalkylsulfonylamino, arylsulfonylamino, heteroarylsulfonylamino, aralkylsulfonylamino, alkylcarbonylamino, haloalkylcarbonylamino, cycloalkylcarbonylamino, heterocycloalkylcarbonylamino, arylcarbonylamino, heteroarylcarbonylamino, aralkylsulfonylamino, aminocarbonyl, alkylaminocarbonyl, cycloalkylaminocarbonyl, heterocycloalkylaminocarbonyl, arylaminocarbonyl, heteroarylaminocarbonyl, N-alkyl-N-arylaminocarbonyl, N-alkyl-N-heteroarylaminocarbonyl, cyano, nitro, azido, phosphinyl, phosphoryl including phosphine oxide and phosphonate, cyclic acetal, 4- to 7-membered heterocycloalkyl which contains at least one nitrogen atom and is linked via the nitrogen atom, aryl, heteroaryl, and where two adjacent R₁₅ taken together with the respective atoms to which each is bonded form aryl, heteroaryl, 5- to 8-membered cycloalkyl, or a 5- to 8-membered heterocycloalkyl; and
each R₁₆ is independently selected from the group consisting of hydrogen, (C₁-C₆)alkyl, (C₁-C₆)haloalkyl, and halo.

In some embodiments, R₈ is selected from:

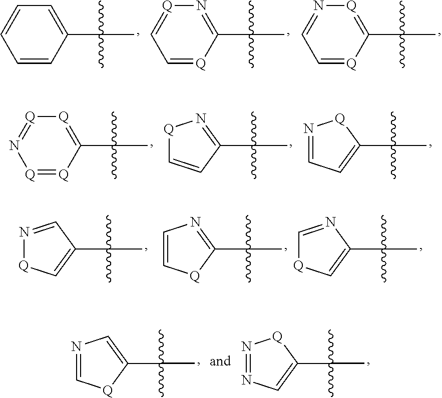

wherein R₈ is further optionally and independently substituted with one or more R₁₅.

In some embodiments, R₈ is selected from:

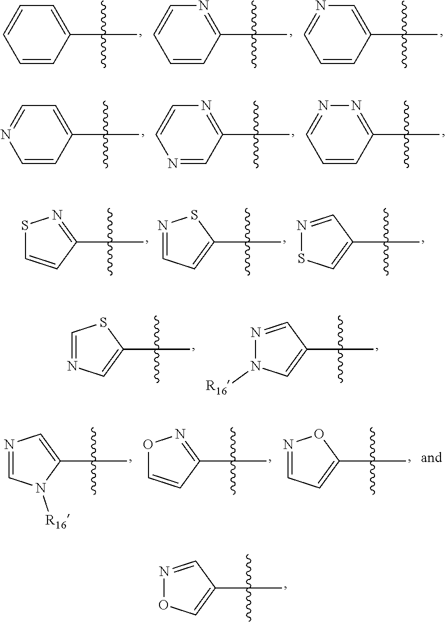

wherein
R₈ is further optionally and independently substituted with one or more (C₁-C₆)alkyl, (C₁-C₆)haloalkyl, (C₁-C₆)alkoxy, cyano, halo, and one or more groups selected from R₁₅; and
R₁₆′ is selected from the group consisting of hydrogen and (C₁-C₆)alkyl.

Representative compounds of the invention have the following structures:

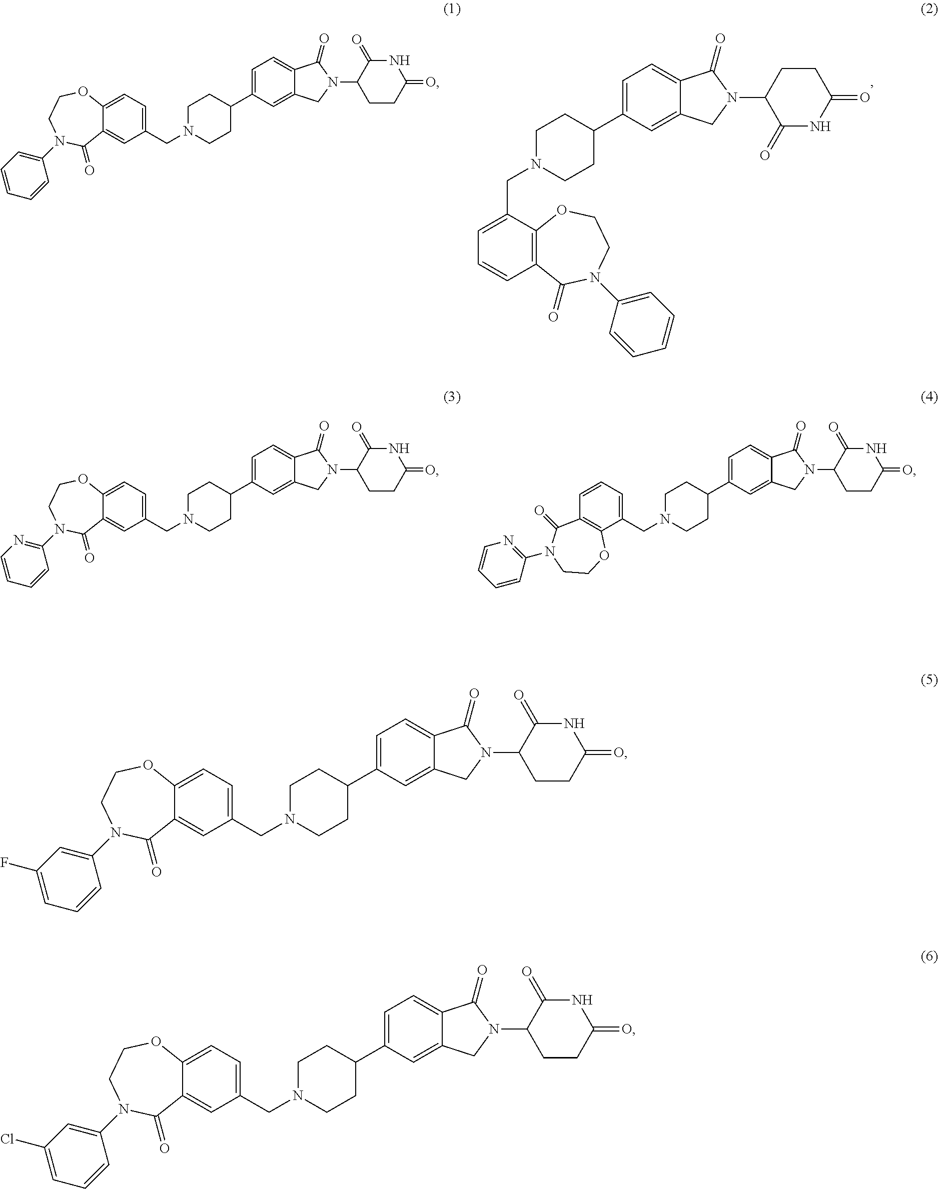 [SMILES: O=C1CCC(N2Cc3cc(C4CCN(Cc5ccc6c(c5)C(=O)N(c5ccccc5)CCO6)CC4)ccc3C2=O)C(=O)N1 ; O=C1CCC(N2Cc3cc(C4CCN(Cc5cccc6c5OCCN(c5ccccc5)C6=O)CC4)ccc3C2=O)C(=O)N1 ; O=C1CCC(N2Cc3cc(C4CCN(Cc5ccc6c(c5)C(=O)N(c5ccccn5)CCO6)CC4)ccc3C2=O)C(=O)N1 ; O=C1CCC(N2Cc3cc(C4CCN(Cc5cccc6c5OCCN(c5ccccn5)C6=O)CC4)ccc3C2=O)C(=O)N1 ; O=C1CCC(N2Cc3cc(C4CCN(Cc5ccc6c(c5)C(=O)N(c5cccc(F)c5)CCO6)CC4)ccc3C2=O)C(=O)N1 ; O=C1CCC(N2Cc3cc(C4CCN(Cc5ccc6c(c5)C(=O)N(c5cccc(Cl)c5)CCO6)CC4)ccc3C2=O)C(=O)N1]

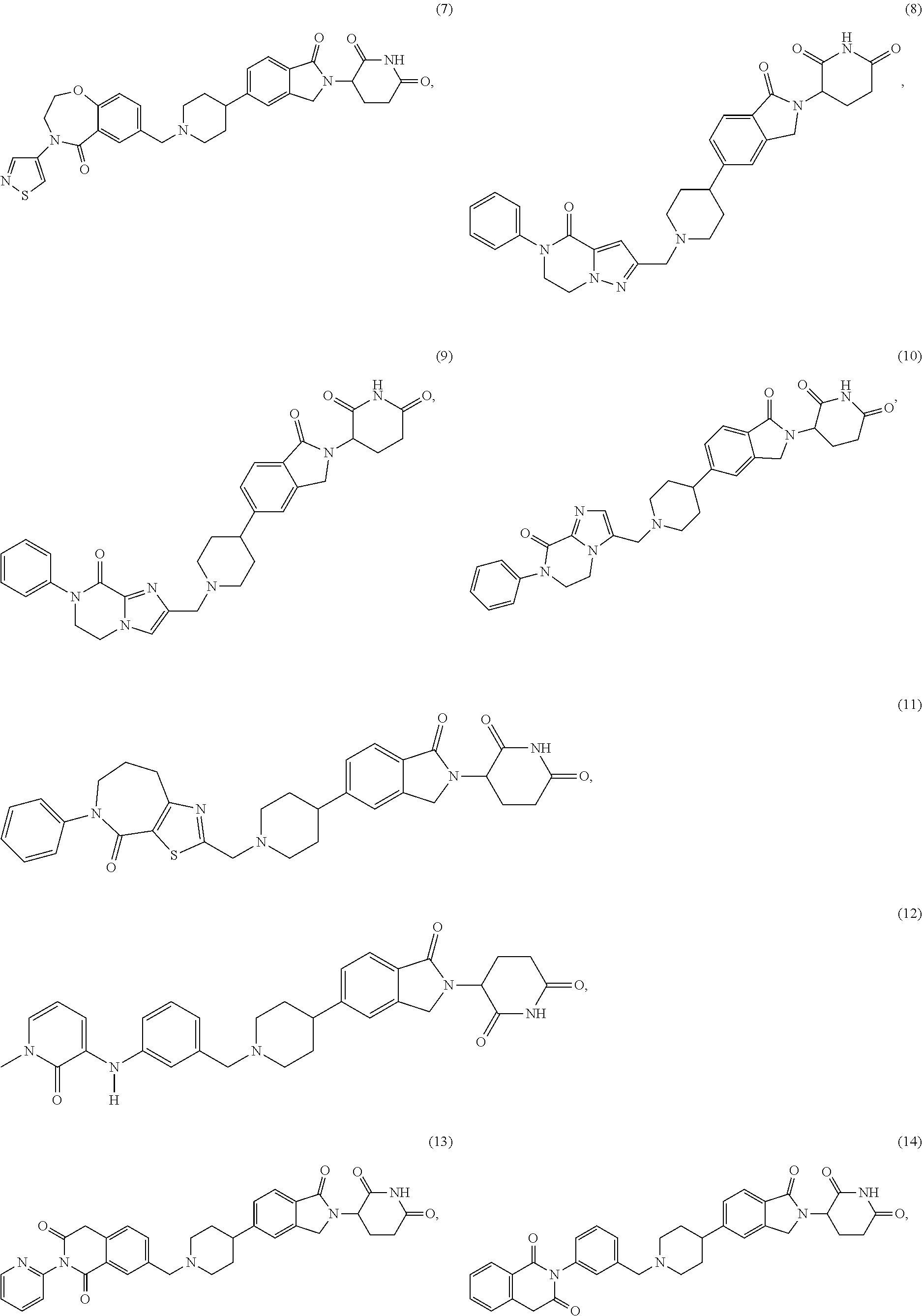

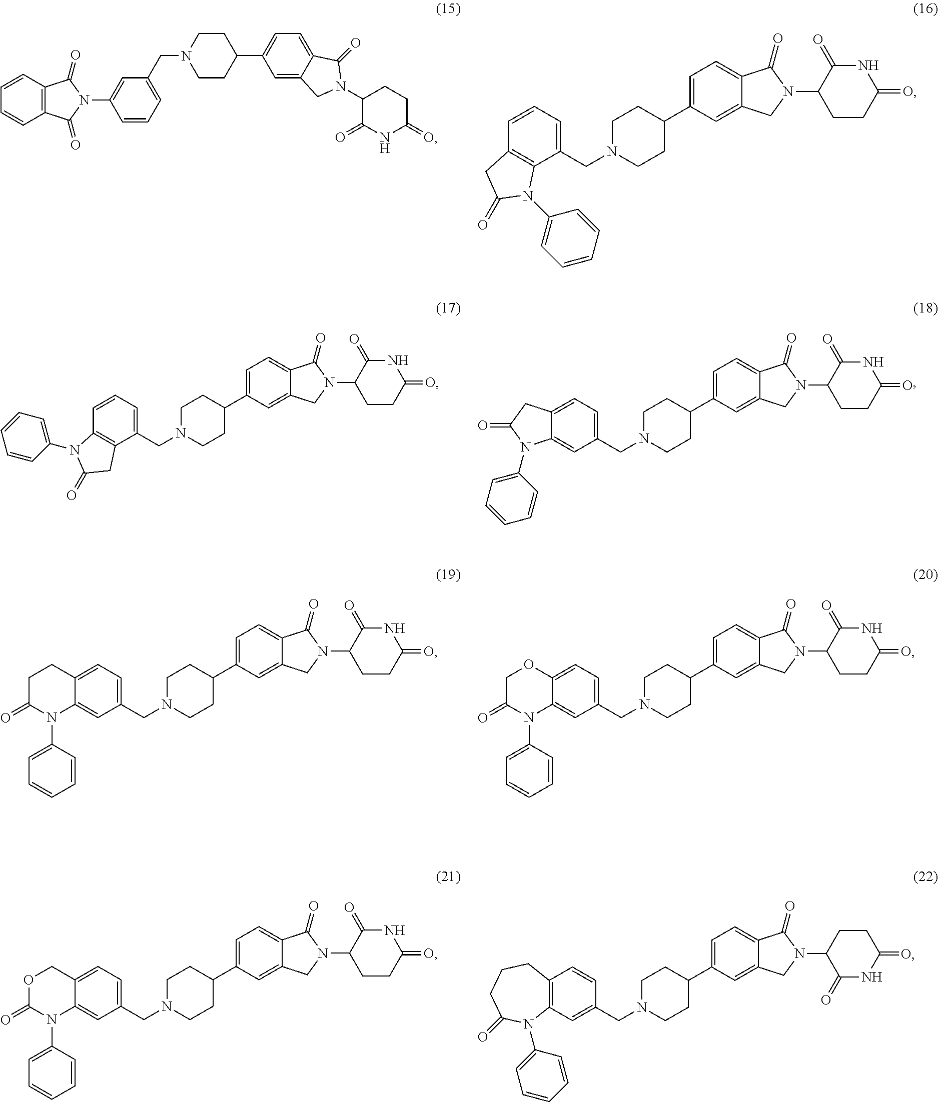

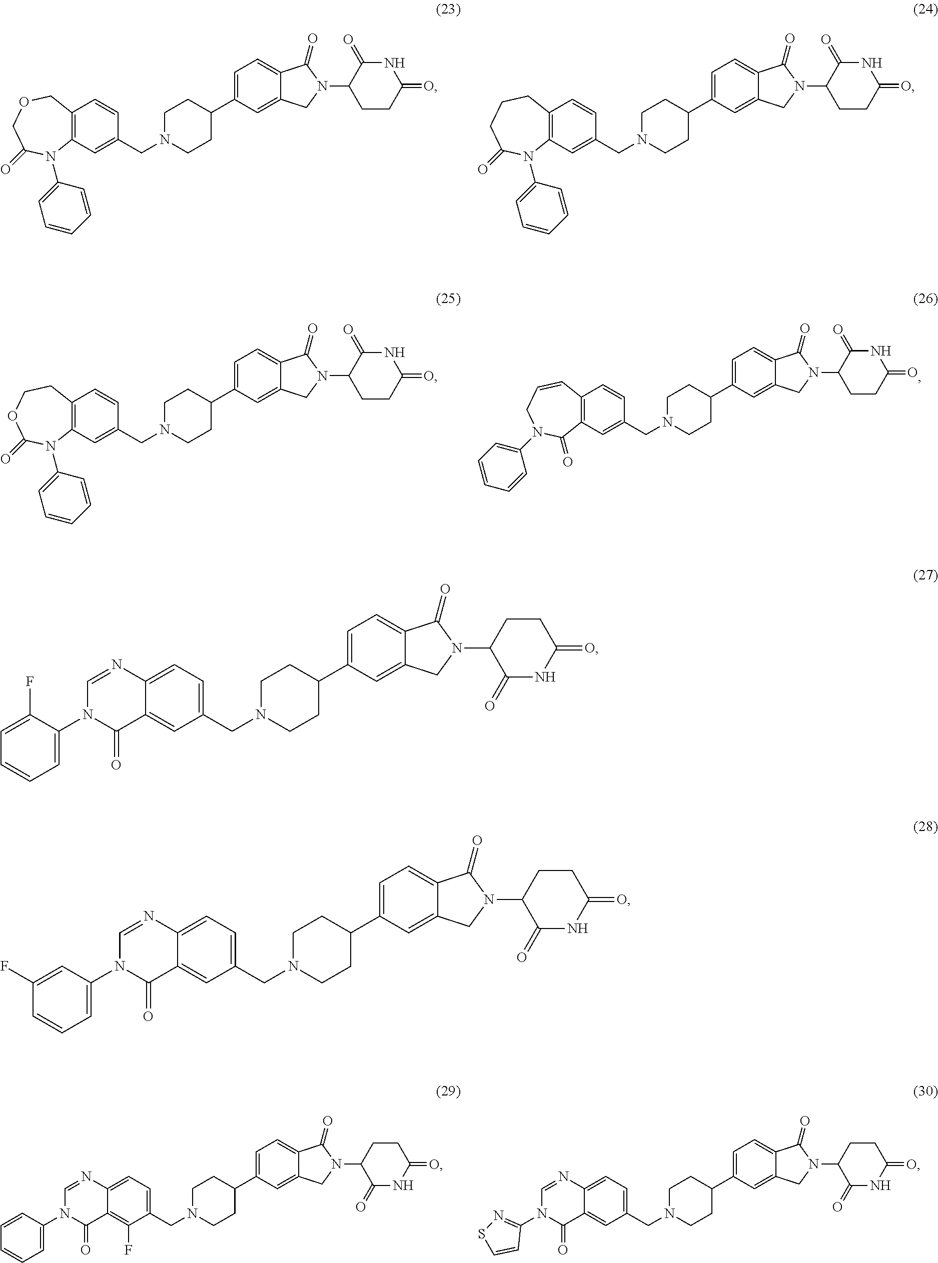

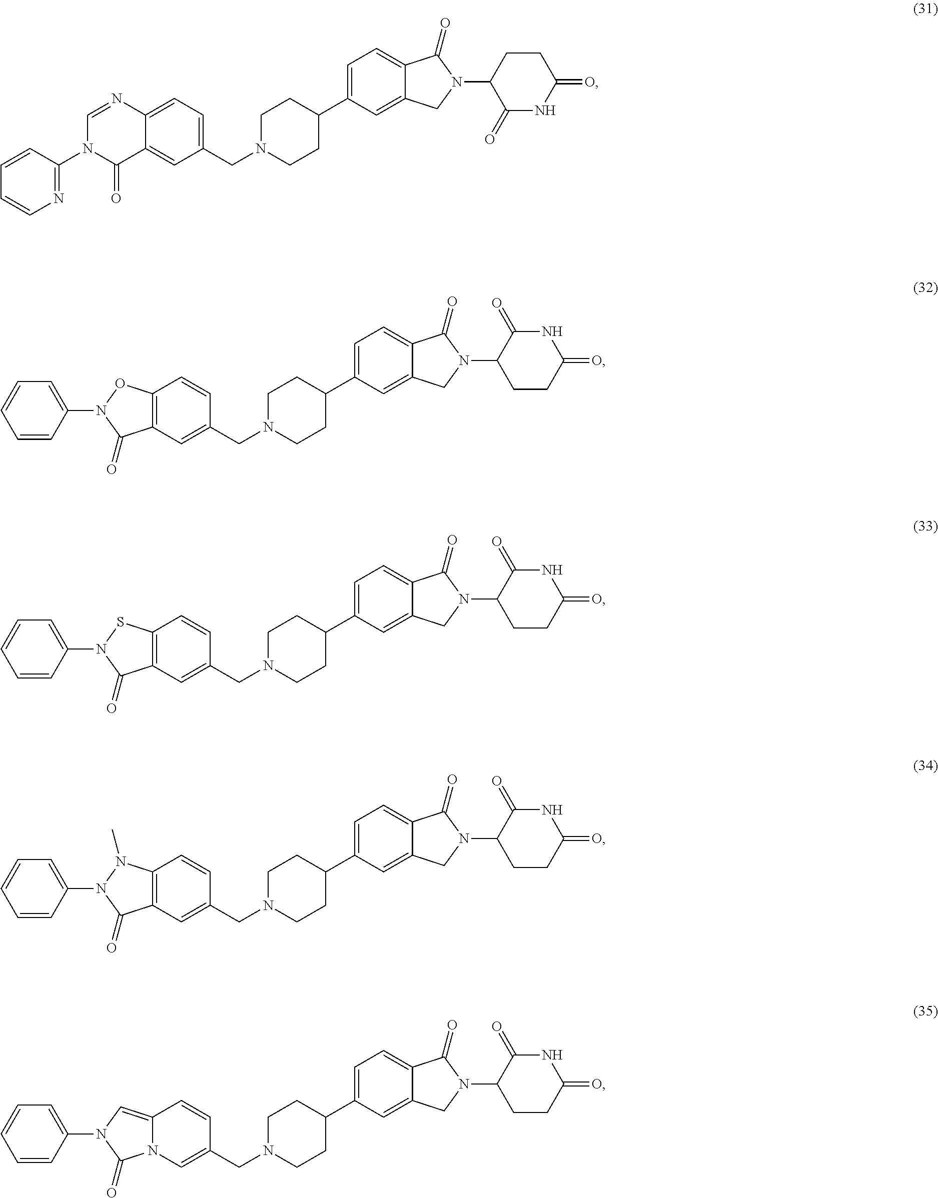 [SMILES: O=C1CCC(N2Cc3cc(C4CCN(Cc5ccc6ncn(-c7ccccn7)c(=O)c6c5)CC4)ccc3C2=O)C(=O)N1 ; O=C1CCC(N2Cc3cc(C4CCN(Cc5ccc6on(-c7ccccc7)c(=O)c6c5)CC4)ccc3C2=O)C(=O)N1 ; O=C1CCC(N2Cc3cc(C4CCN(Cc5ccc6sn(-c7ccccc7)c(=O)c6c5)CC4)ccc3C2=O)C(=O)N1 ; Cn1c2ccc(CN3CCC(c4ccc5c(c4)CN(C4CCC(=O)NC4=O)C5=O)CC3)cc2c(=O)n1-c1ccccc1 ; O=C1CCC(N2Cc3cc(C4CCN(Cc5ccc6cn(-c7ccccc7)c(=O)n6c5)CC4)ccc3C2=O)C(=O)N1]

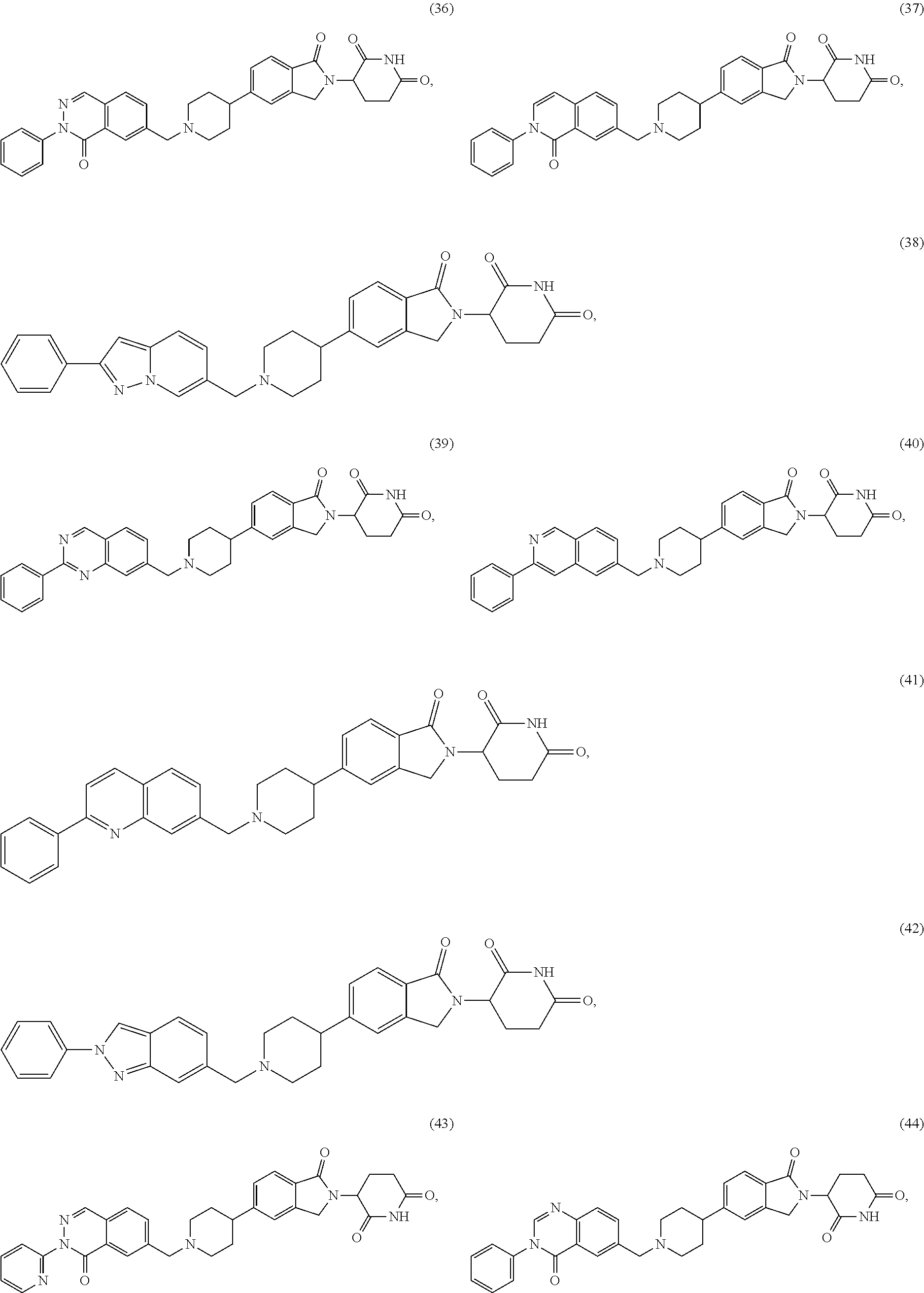

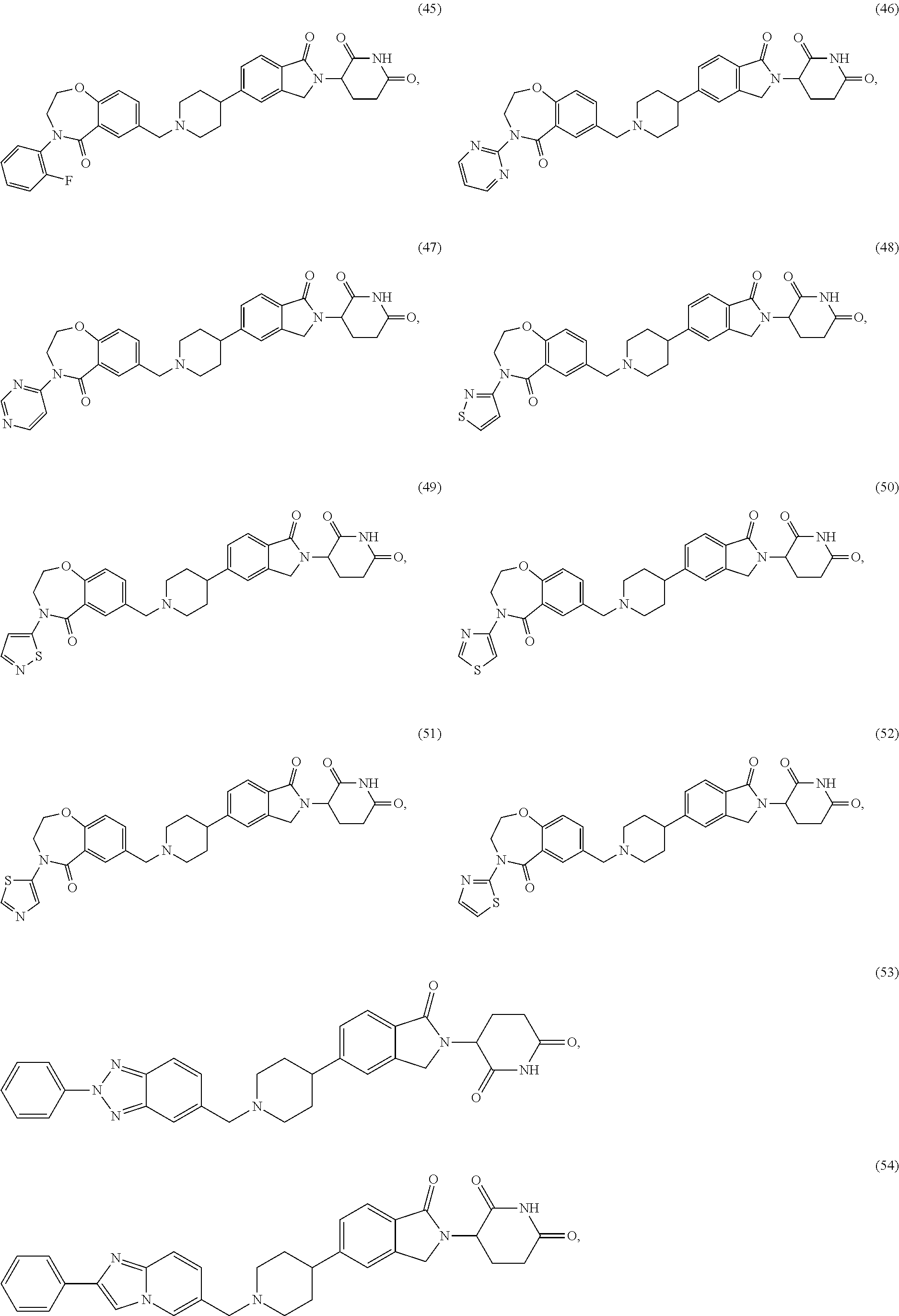

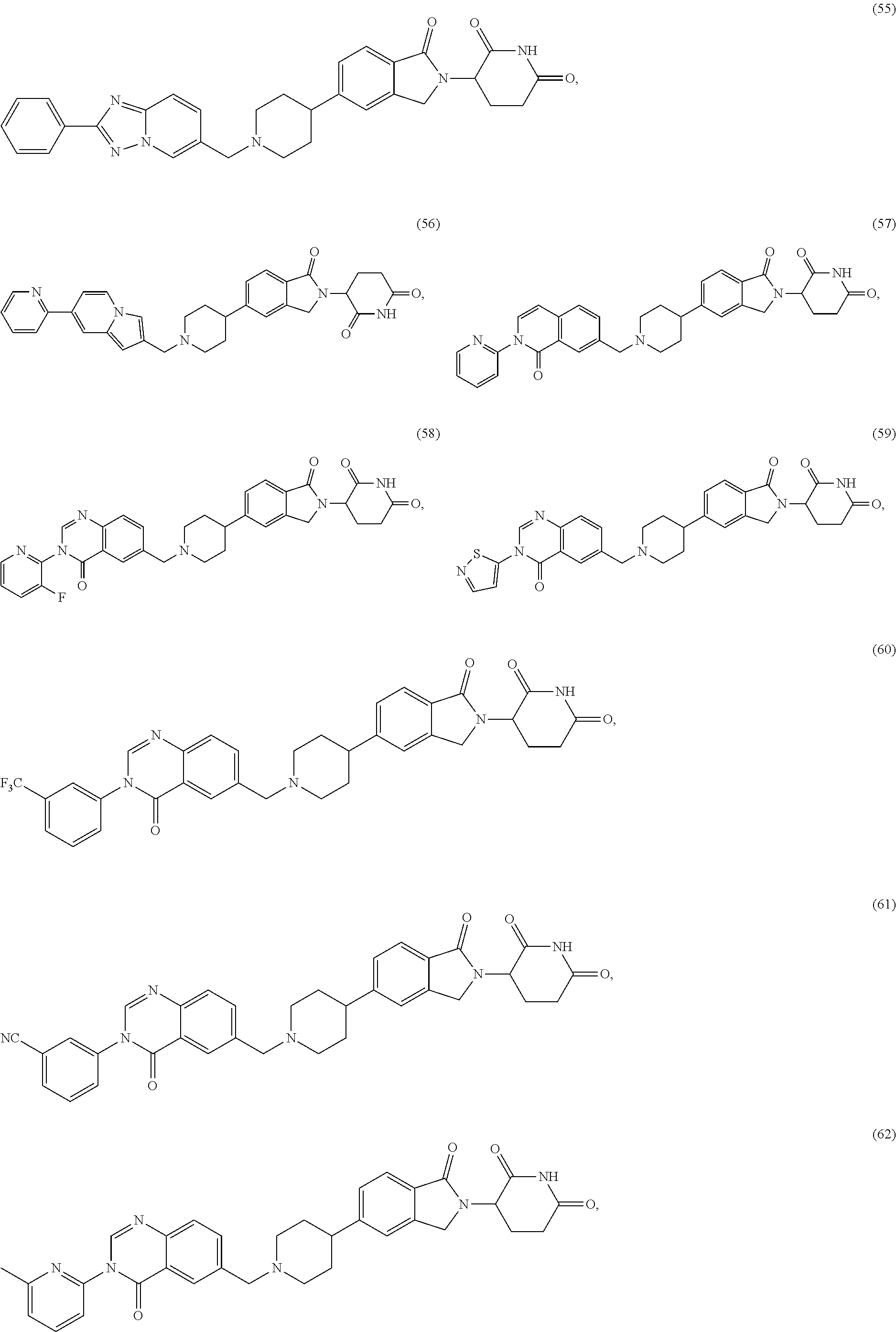

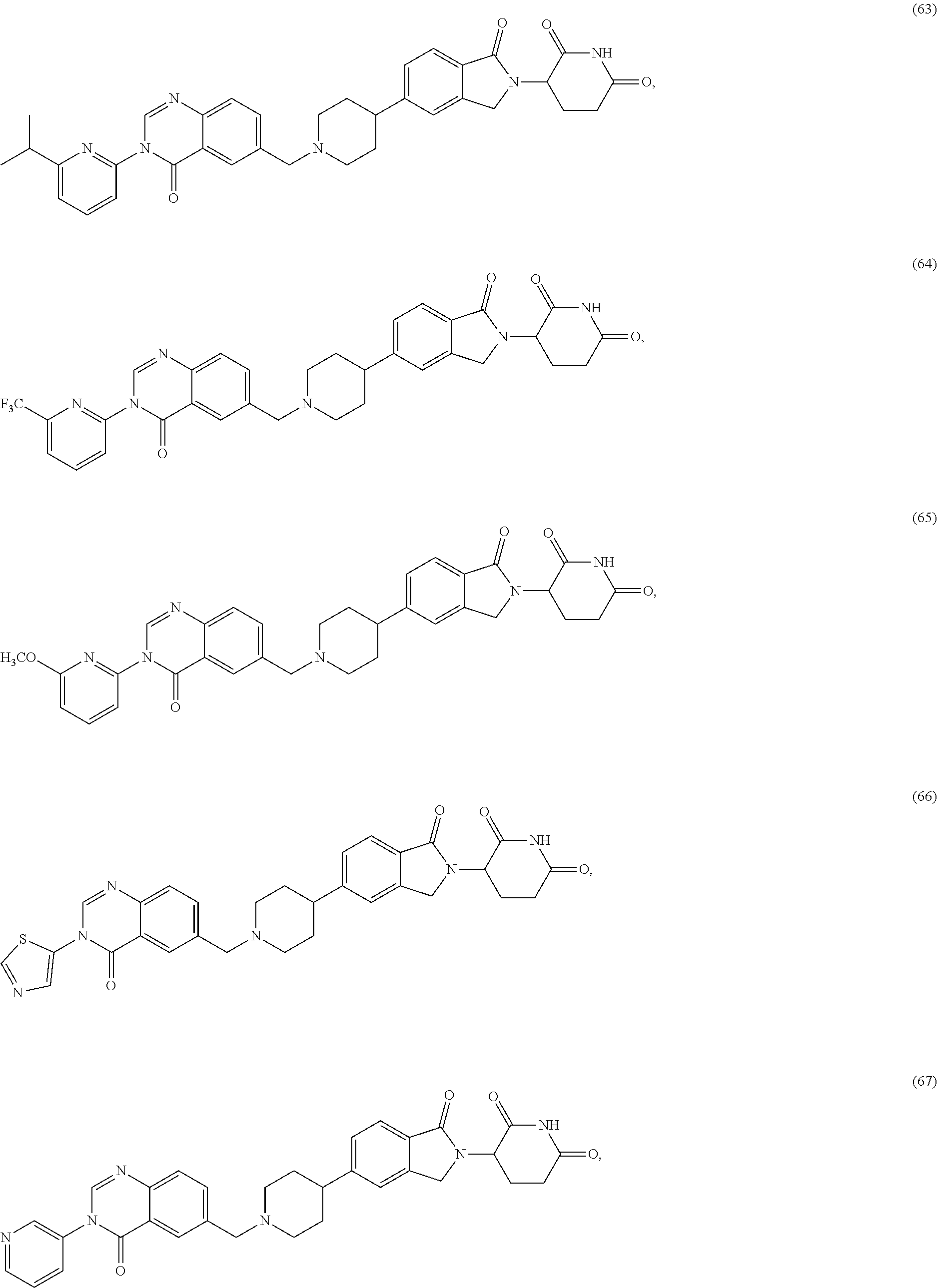 [SMILES: CC(C)c1cccc(-n2cnc3ccc(CN4CCC(c5ccc6c(c5)CN(C5CCC(=O)NC5=O)C6=O)CC4)cc3c2=O)n1 ; O=C1CCC(N2Cc3cc(C4CCN(Cc5ccc6ncn(-c7cccc(C(F)(F)F)n7)c(=O)c6c5)CC4)ccc3C2=O)C(=O)N1 ; COc1cccc(-n2cnc3ccc(CN4CCC(c5ccc6c(c5)CN(C5CCC(=O)NC5=O)C6=O)CC4)cc3c2=O)n1 ; O=C1CCC(N2Cc3cc(C4CCN(Cc5ccc6ncn(-c7cncs7)c(=O)c6c5)CC4)ccc3C2=O)C(=O)N1 ; O=C1CCC(N2Cc3cc(C4CCN(Cc5ccc6ncn(-c7cccnc7)c(=O)c6c5)CC4)ccc3C2=O)C(=O)N1]

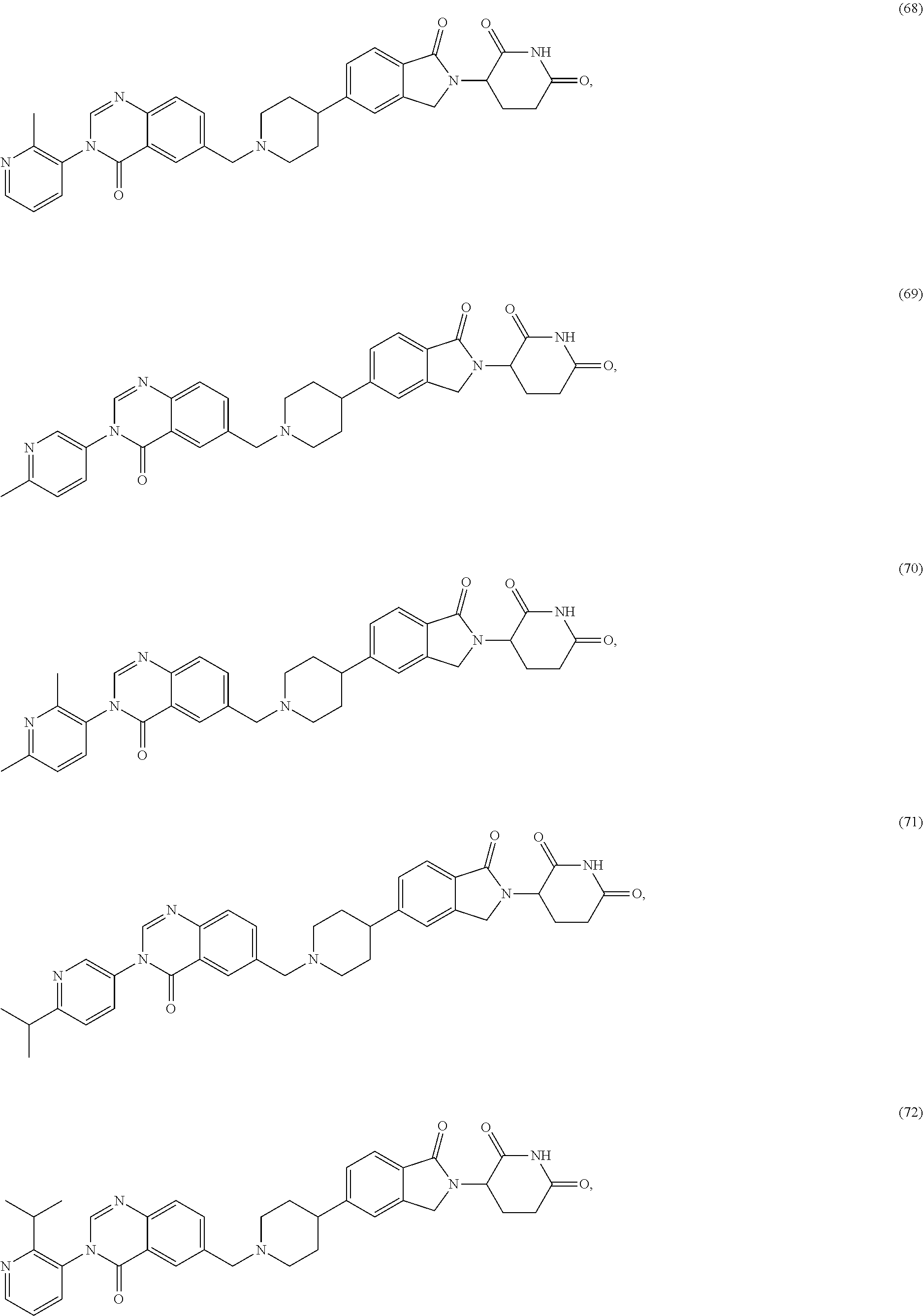 [SMILES: Cc1ncccc1-n1cnc2ccc(CN3CCC(c4ccc5c(c4)CN(C4CCC(=O)NC4=O)C5=O)CC3)cc2c1=O ; Cc1ccc(-n2cnc3ccc(CN4CCC(c5ccc6c(c5)CN(C5CCC(=O)NC5=O)C6=O)CC4)cc3c2=O)cn1 ; Cc1ccc(-n2cnc3ccc(CN4CCC(c5ccc6c(c5)CN(C5CCC(=O)NC5=O)C6=O)CC4)cc3c2=O)c(C)n1 ; CC(C)c1ccc(-n2cnc3ccc(CN4CCC(c5ccc6c(c5)CN(C5CCC(=O)NC5=O)C6=O)CC4)cc3c2=O)cn1 ; CC(C)c1ncccc1-n1cnc2ccc(CN3CCC(c4ccc5c(c4)CN(C4CCC(=O)NC4=O)C5=O)CC3)cc2c1=O]

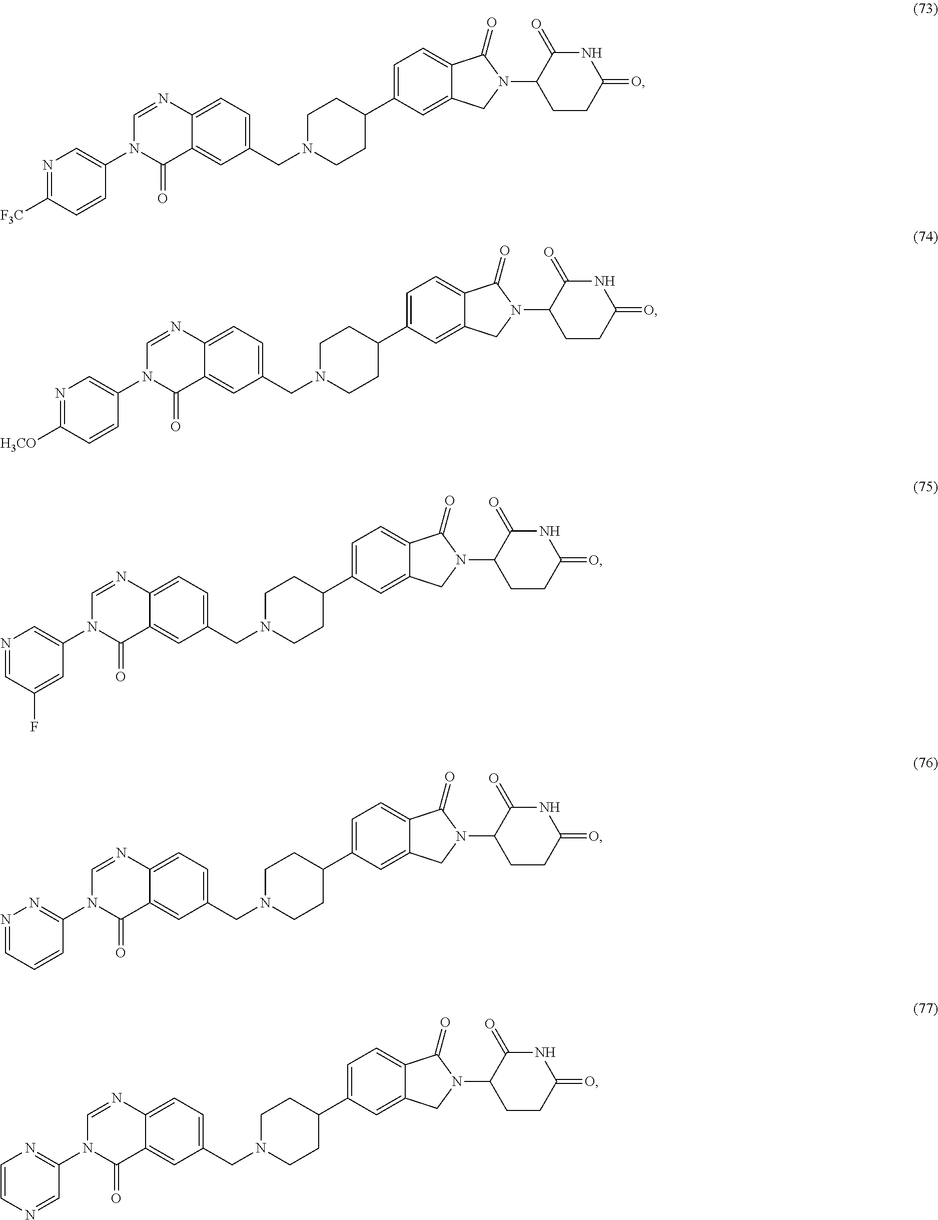 [SMILES: O=C1CCC(N2Cc3cc(C4CCN(Cc5ccc6ncn(-c7ccc(C(F)(F)F)nc7)c(=O)c6c5)CC4)ccc3C2=O)C(=O)N1 ; COc1ccc(-n2cnc3ccc(CN4CCC(c5ccc6c(c5)CN(C5CCC(=O)NC5=O)C6=O)CC4)cc3c2=O)cn1 ; O=C1CCC(N2Cc3cc(C4CCN(Cc5ccc6ncn(-c7cncc(F)c7)c(=O)c6c5)CC4)ccc3C2=O)C(=O)N1 ; O=C1CCC(N2Cc3cc(C4CCN(Cc5ccc6ncn(-c7cccnn7)c(=O)c6c5)CC4)ccc3C2=O)C(=O)N1 ; O=C1CCC(N2Cc3cc(C4CCN(Cc5ccc6ncn(-c7cnccn7)c(=O)c6c5)CC4)ccc3C2=O)C(=O)N1]

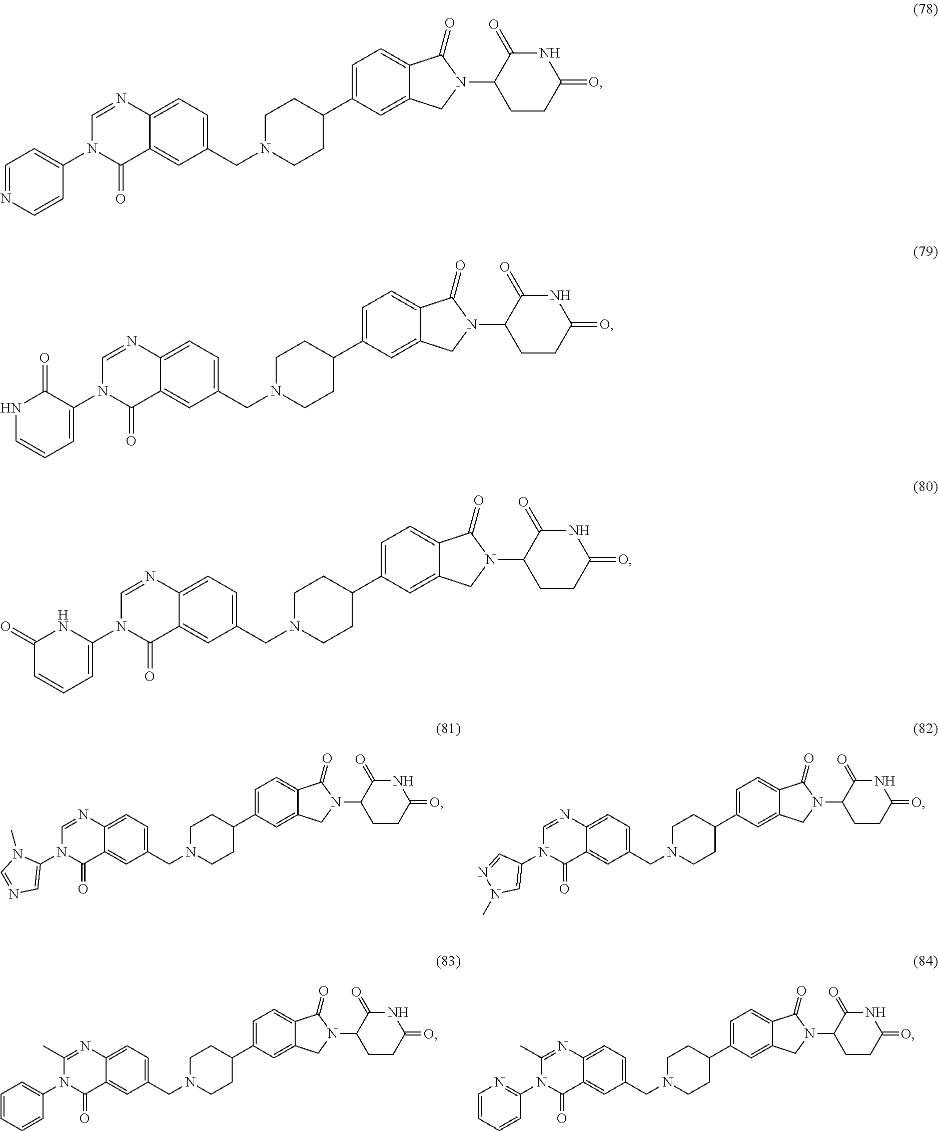

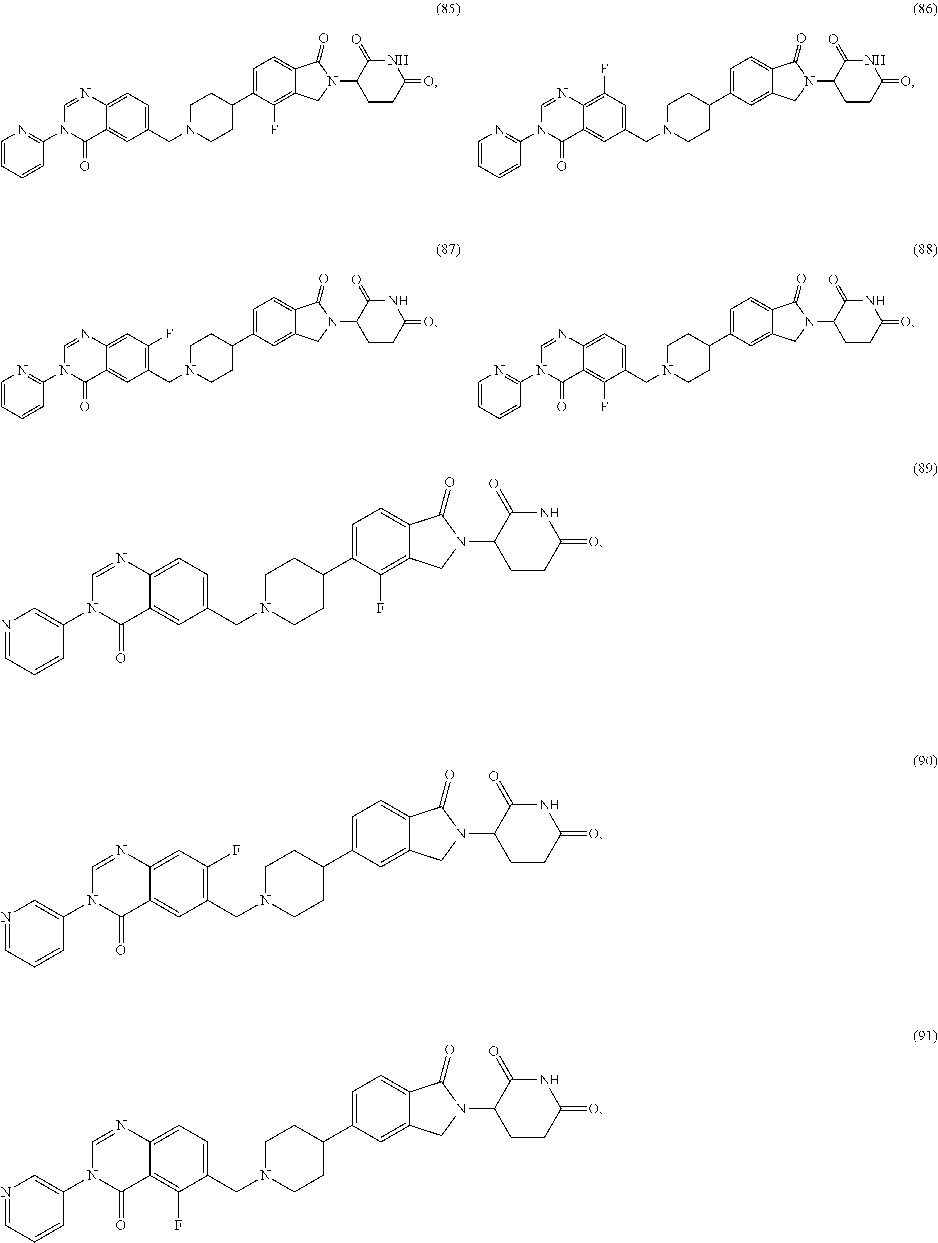

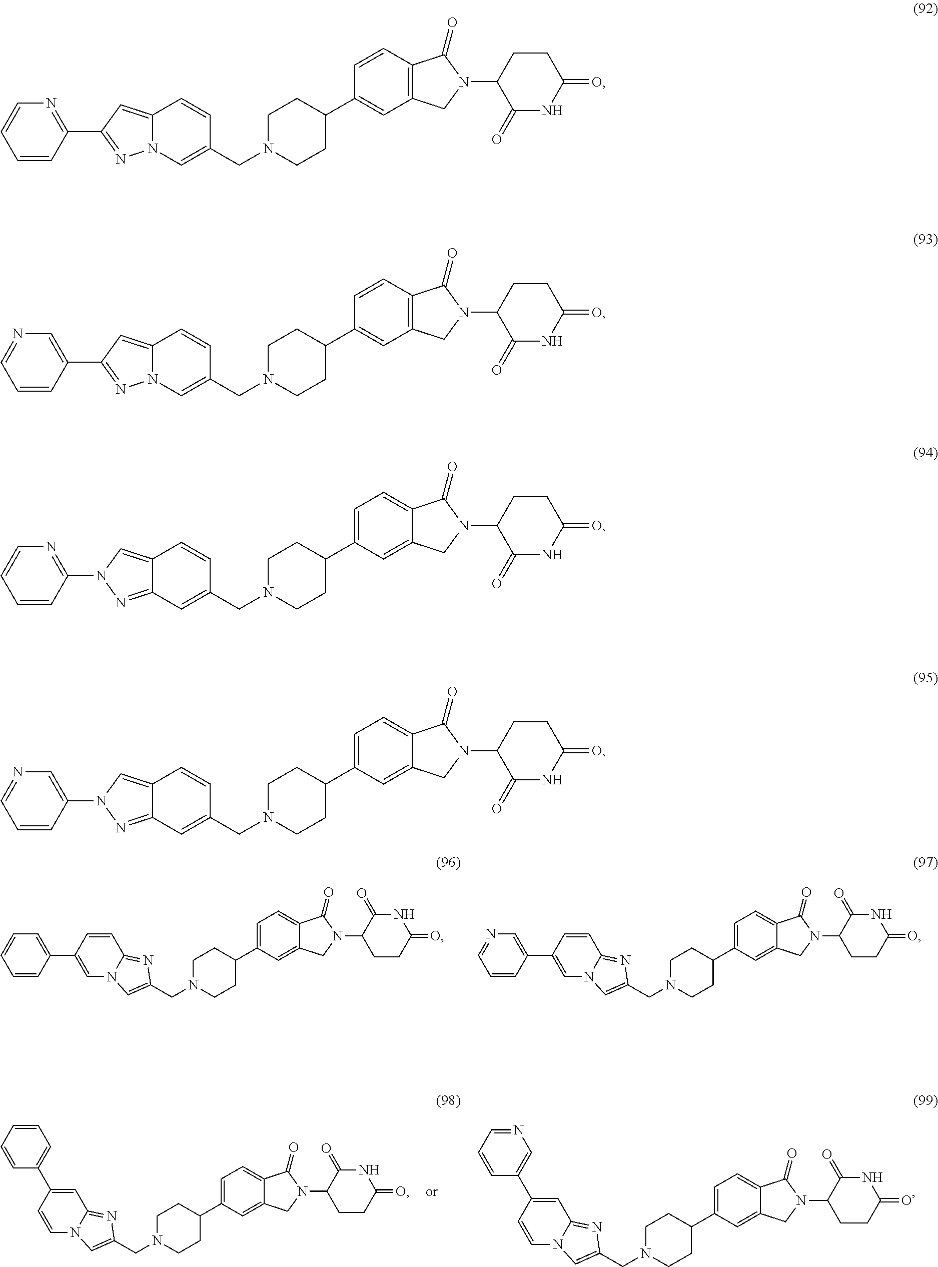

or a pharmaceutically acceptable salt, hydrate, solvate, prodrug, stereoisomer, or tautomer thereof.

Compounds 1-26, 32-34, and 45-52 are embraced by formula I. Compounds 27-31, 35-44 and 53-99 are embraced by formula II.

Compounds of the present invention (compounds of formulas (I) and (II)) may be in the form of a free acid or free base, or a pharmaceutically acceptable salt. As used herein, the term “pharmaceutically acceptable” in the context of a salt refers to a salt of the compound that does not abrogate the biological activity or properties of the compound, and is relatively nontoxic, i.e., the compound in salt form may be administered to a subject without causing undesirable biological effects (such as dizziness or gastric upset) or interacting in a deleterious manner with any of the other components of the composition in which it is contained. The term “pharmaceutically acceptable salt” refers to a product obtained by reaction of the compound of the present invention with a suitable acid or a base. Examples of pharmaceutically acceptable salts of the compounds of this invention include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the invention can form pharmaceutically acceptable salts with various organic bases such as lysine, arginine, guanidine, diethanolamine or metformin. Suitable base salts include aluminum, calcium, lithium, magnesium, potassium, sodium, or zinc salts.

Compounds of the present invention may have at least one chiral center and thus may be in the form of a stereoisomer, which as used herein, embraces all isomers of individual compounds that differ only in the orientation of their atoms in space. The term stereoisomer includes mirror image isomers (enantiomers which include the (R-) or (S-) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). The chiral centers of the compounds may undergo epimerization in vivo; thus, for these compounds, administration of the compound in its (R-) form is considered equivalent to administration of the compound in its (S-) form. Accordingly, the compounds of the present invention may be made and used in the form of individual isomers and substantially free of other isomers, or in the form of a mixture of various isomers, e.g., racemic mixtures of stereoisomers.

In some embodiments, the compound is an isotopic derivative in that it has at least one desired isotopic substitution of an atom, at an amount above the natural abundance of the isotope, i.e., enriched. In one embodiment, the compound includes deuterium or multiple deuterium atoms. Substitution with heavier isotopes such as deuterium, i.e. ²H, may afford certain therapeutic advantages resulting from greater metabolic stability, for example, increased in vivo half-life or reduced dosage requirements, and thus may be advantageous in some circumstances.

Compounds of the present invention may also be in the form of N-oxides, crystalline forms (also known as polymorphs), active metabolites of the compounds having the same type of activity, prodrugs, tautomers, and unsolvated as well as solvated (e.g., hydrated) forms with pharmaceutically acceptable solvents such as water, ethanol, and the like, of the compounds.

The compounds of the present invention may be prepared by crystallization under different conditions and may exist as one or a combination of polymorphs of the compound. For example, different polymorphs may be identified and/or prepared using different solvents, or different mixtures of solvents for recrystallization, by performing crystallizations at different temperatures, or by using various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffractogram and/or other known techniques.

In some embodiments, the pharmaceutical composition comprises a co-crystal of an inventive compound. The term “co-crystal”, as used herein, refers to a stoichiometric multi-component system comprising a compound of the invention and a co-crystal former wherein the compound of the invention and the co-crystal former are connected by non-covalent interactions. The term “co-crystal former”, as used herein, refers to compounds which can form intermolecular interactions with a compound of the invention and co-crystallize with it. Representative examples of co-crystal formers include benzoic acid, succinic acid, fumaric acid, glutaric acid, trans-cinnamic acid, 2,5-dihydroxybenzoic acid, glycolic acid, trans-2-hexanoic acid, 2-hydroxycaproic acid, lactic acid, sorbic acid, tartaric acid, ferulic acid, suberic acid, picolinic acid, salicyclic acid, maleic acid, saccharin, 4,4′-bipyridine p-aminosalicyclic acid, nicotinamide, urea, isonicotinamide, methyl-4-hydroxybenzoate, adipic acid, terephthalic acid, resorcinol, pyrogallol, phloroglucinol, hydroxyquinol, isoniazid, theophylline, adenine, theobromine, phenacetin, phenazone, etofylline, and phenobarbital.

### Methods of Synthesis

In another aspect, the present invention is directed to a method for making an inventive compound, or a pharmaceutically acceptable salt, hydrate, solvate, prodrug, stereoisomer, or tautomer thereof. Broadly, the inventive compounds or a pharmaceutically acceptable salt, hydrate, solvate, prodrug, stereoisomer, or tautomer thereof may be prepared by any process known to be applicable to the preparation of chemically related compounds. The compounds of the present invention will be better understood in connection with the synthetic schemes that described in various working examples and which illustrate nonlimiting methods by which the compounds of the invention may be prepared.

### Pharmaceutical Compositions

Another aspect of the present invention is directed to a pharmaceutical composition that includes a therapeutically effective amount of an inventive compound or a pharmaceutically acceptable salt, hydrate, solvate, prodrug, stereoisomer, or tautomer thereof, and a pharmaceutically acceptable carrier. The term “pharmaceutically acceptable carrier,” as known in the art, refers to a pharmaceutically acceptable material, composition or vehicle, suitable for administering compounds of the present invention to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may also include one or more pharmaceutically acceptable excipients.

Broadly, compounds of the invention and their pharmaceutically acceptable salts, hydrates, solvates, prodrugs, stereoisomers, or tautomers may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). The type of formulation depends on the mode of administration which may include enteral (e.g., oral, buccal, sublingual and rectal), parenteral (e.g., subcutaneous (s.c.), intravenous (i.v.), intramuscular (i.m.), and intrasternal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal). In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the compound may be administered relatively quickly such as in the case of a single-dose treatment and/or an acute condition.

In some embodiments, the compounds are formulated for oral or intravenous administration (e.g., systemic intravenous injection).

Accordingly, compounds of the invention may be formulated into solid compositions (e.g., powders, tablets, dispersible granules, capsules, cachets, and suppositories), liquid compositions (e.g., solutions in which the compound is dissolved, suspensions in which solid particles of the compound are dispersed, emulsions, and solutions containing liposomes, micelles, or nanoparticles, syrups and elixirs); semi-solid compositions (e.g., gels, suspensions and creams); and gases (e.g., propellants for aerosol compositions). Compounds may also be formulated for rapid, intermediate or extended release.

Solid dosage forms for oral administration include capsules, tablets, pills, powders, and granules. In such solid dosage forms, the active compound is mixed with a carrier such as sodium citrate or dicalcium phosphate and an additional carrier or excipient such as a) fillers or extenders such as starches, lactose, sucrose, glucose, mannitol, and silicic acid, b) binders such as, for example, methylcellulose, microcrystalline cellulose, hydroxypropylmethylcellulose, carboxymethylcellulose, sodium carboxymethylcellulose, alginates, gelatin, polyvinylpyrrolidinone, sucrose, and acacia, c) humectants such as glycerol, d) disintegrating agents such as crosslinked polymers (e.g., crosslinked polyvinylpyrrolidone (crospovidone), crosslinked sodium carboxymethyl cellulose (croscarmellose sodium), sodium starch glycolate, agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings. They may further contain an opacifying agent.

In some embodiments, compounds of the invention may be formulated in a hard or soft gelatin capsule. Representative excipients that may be used include pregelatinized starch, magnesium stearate, mannitol, sodium stearyl fumarate, lactose anhydrous, microcrystalline cellulose and croscarmellose sodium. Gelatin shells may include gelatin, titanium dioxide, iron oxides and colorants.

Liquid dosage forms for oral administration include solutions, suspensions, emulsions, micro-emulsions, syrups and elixirs. In addition to the compound, the liquid dosage forms may contain an aqueous or non-aqueous carrier (depending upon the solubility of the compounds) commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Oral compositions may also include an excipients such as wetting agents, suspending agents, coloring, sweetening, flavoring, and perfuming agents.

Injectable preparations for parenteral administration may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished by the use of a liquid suspension or crystalline or amorphous material with poor water solubility. Prolonged absorption of the compound from a parenterally administered formulation may also be accomplished by suspending the compound in an oily vehicle.

In certain embodiments, compounds of the invention may be administered in a local rather than systemic manner, for example, via injection of the conjugate directly into an organ, often in a depot preparation or sustained release formulation. In specific embodiments, long acting formulations are administered by implantation (for example subcutaneously or intramuscularly) or by intramuscular injection. Injectable depot forms are made by forming microencapsule matrices of the compound in a biodegradable polymer, e.g., polylactide-polyglycolides, poly(orthoesters) and poly(anhydrides). The rate of release of the compound may be controlled by varying the ratio of compound to polymer and the nature of the particular polymer employed. Depot injectable formulations are also prepared by entrapping the compound in liposomes or microemulsions that are compatible with body tissues. Furthermore, in other embodiments, the compound is delivered in a targeted drug delivery system, for example, in a liposome coated with organ-specific antibody. In such embodiments, the liposomes are targeted to and taken up selectively by the organ.

The compositions may be formulated for buccal or sublingual administration, examples of which include tablets, lozenges and gels.

The compounds of the invention may be formulated for administration by inhalation. Various forms suitable for administration by inhalation include aerosols, mists or powders. Pharmaceutical compositions may be delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In some embodiments, the dosage unit of a pressurized aerosol may be determined by providing a valve to deliver a metered amount. In some embodiments, capsules and cartridges including gelatin, for example, for use in an inhaler or insufflator, may be formulated containing a powder mix of the compound and a suitable powder base such as lactose or starch.

Compounds of the invention may be formulated for topical administration which as used herein, refers to administration intradermally by invention of the formulation to the epidermis. These types of compositions are typically in the form of ointments, pastes, creams, lotions, gels, solutions and sprays.

Representative examples of carriers useful in formulating compounds for topical application include solvents (e.g., alcohols, poly alcohols, water), creams, lotions, ointments, oils, plasters, liposomes, powders, emulsions, microemulsions, and buffered solutions (e.g., hypotonic or buffered saline). Creams, for example, may be formulated using saturated or unsaturated fatty acids such as stearic acid, palmitic acid, oleic acid, palmito-oleic acid, cetyl, or oleyl alcohols. Creams may also contain a non-ionic surfactant such as polyoxy-40-stearate.

In some embodiments, the topical formulations may also include an excipient, an example of which is a penetration enhancing agent. These agents are capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). Representative examples of penetration enhancing agents include triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Representative examples of yet other excipients that may be included in topical as well as in other types of formulations (to the extent they are compatible), include preservatives, antioxidants, moisturizers, emollients, buffering agents, solubilizing agents, skin protectants, and surfactants. Suitable preservatives include alcohols, quaternary amines, organic acids, parabens, and phenols. Suitable antioxidants include ascorbic acid and its esters, sodium bisulfite, butylated hydroxytoluene, butylated hydroxyanisole, tocopherols, and chelating agents like EDTA and citric acid. Suitable moisturizers include glycerin, sorbitol, polyethylene glycols, urea, and propylene glycol. Suitable buffering agents include citric, hydrochloric, and lactic acid buffers. Suitable solubilizing agents include quaternary ammonium chlorides, cyclodextrins, benzyl benzoate, lecithin, and polysorbates. Suitable skin protectants include vitamin E oil, allatoin, dimethicone, glycerin, petrolatum, and zinc oxide.

Transdermal formulations typically employ transdermal delivery devices and transdermal delivery patches wherein the compound is formulated in lipophilic emulsions or buffered, aqueous solutions, dissolved and/or dispersed in a polymer or an adhesive. Patches may be constructed for continuous, pulsatile, or on demand delivery of pharmaceutical agents. Transdermal delivery of the compounds may be accomplished by means of an iontophoretic patch. Transdermal patches may provide controlled delivery of the compounds wherein the rate of absorption is slowed by using rate-controlling membranes or by trapping the compound within a polymer matrix or gel. Absorption enhancers may be used to increase absorption, examples of which include absorbable pharmaceutically acceptable solvents that assist passage through the skin.

Ophthalmic formulations include eye drops.

Formulations for rectal administration include enemas, rectal gels, rectal foams, rectal aerosols, and retention enemas, which may contain conventional suppository bases such as cocoa butter or other glycerides, as well as synthetic polymers such as polyvinylpyrrolidone, PEG, and the like. Compositions for rectal or vaginal administration may also be formulated as suppositories which can be prepared by mixing the compound with suitable non-irritating carriers and excipients such as cocoa butter, mixtures of fatty acid glycerides, polyethylene glycol, suppository waxes, and combinations thereof, all of which are solid at ambient temperature but liquid at body temperature and therefore melt in the rectum or vaginal cavity and release the compound.

### Dosage Amounts

As used herein, the term, “therapeutically effective amount” refers to an amount of an inventive compound or a pharmaceutically acceptable salt, hydrate, solvate, prodrug, stereoisomer, or tautomer thereof that is effective in producing the desired therapeutic response in a patient suffering from a disease or disorder involving IKZF2 (Helios) and would benefit from IKZF2 degradation. The term “therapeutically effective amount” thus includes the amount of the inventive compound or a pharmaceutically acceptable salt, hydrate, solvate, prodrug, stereoisomer, or tautomer thereof, that when administered, induces a positive modification in the disease or disorder to be treated, or is sufficient to prevent development or progression of the disease or disorder, or alleviate to some extent, one or more of the symptoms of the disease or disorder being treated in a subject, or which simply kills or inhibits the growth of diseased cells, or reduces the amounts of IKZF2 in diseased cells.

The total daily dosage of the compounds and usage thereof may be decided in accordance with standard medical practice, e.g., by the attending physician using sound medical judgment. The specific therapeutically effective dose for any particular subject will depend upon a variety of factors, including the following: the disease or disorder being treated and the severity thereof (e.g., its present status); the activity of the compound employed; the specific composition employed; the age, body weight, general health, sex and diet of the subject; the time of administration, route of administration, and rate of excretion of the compound employed; the duration of the treatment; drugs used in combination or coincidental with the specific compound employed; and like factors well known in the medical arts (see, for example, Hardman et al., eds., Goodman and Gilman's The Pharmacological Basis of Therapeutics, 10th Edition, McGraw-Hill Press, 155-173, 2001).

Compounds of the invention may be effective over a wide dosage range. In some embodiments, the total daily dosage (e.g., for adult humans) may range from about 0.001 to about 1600 mg, from 0.01 to about 1000 mg, from 0.01 to about 500 mg, from about 0.01 to about 100 mg, from about 0.5 to about 100 mg, from 1 to about 100-400 mg per day, from about 1 to about 50 mg per day, from about 5 to about 40 mg per day, and in yet other embodiments from about 10 to about 30 mg per day. Individual dosages may be formulated to contain the desired dosage amount depending upon the number of times the compound is administered per day. By way of example, capsules may be formulated with from about 1 to about 200 mg of compound (e.g., 1, 2, 2.5, 3, 4, 5, 10, 15, 20, 25, 50, 100, 150, and 200 mg). In some embodiments, the compound may be administered at a dose in range from about 0.01 mg to about 200 mg/kg of body weight per day. In some embodiments, a dose of from 0.1 to 100, e.g., from 1 to 30 mg/kg per day in one or more dosages per day may be effective. By way of example, a suitable dose for oral administration may be in the range of 1-30 mg/kg of body weight per day, and a suitable dose for intravenous administration may be in the range of 1-10 mg/kg of body weight per day.

### Methods of Use

In some aspects, the present invention is directed to methods of treating diseases or disorders involving IKZF2, that entails administration of a therapeutically effective amount of a compound of formula (I) and/or (II), or a pharmaceutically acceptable salt, hydrate, solvate, prodrug, stereoisomer, or tautomer thereof, to a subject in need thereof.

Broadly, the diseases or disorders that may be amenable to treatment with compounds of the present invention involve IKZF2 or otherwise functionally abnormal IKZF2 activity relative to a non-pathological state. A “disease” is generally regarded as a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health. In some embodiments, compounds of formula (I) and (II) may be useful in the treatment of cell proliferative diseases and disorders (e.g., cancer or benign neoplasms). As used herein, the term “cell proliferative disease or disorder” refers to the conditions characterized by deregulated or abnormal cell growth, or both, including noncancerous conditions such as neoplasms, precancerous conditions, benign tumors, and cancer.

The term “subject” (or “patient”) as used herein includes all members of the animal kingdom prone to or suffering from the indicated disease or disorder. In some embodiments, the subject is a mammal, e.g., a human or a non-human mammal. The methods are also applicable to companion animals such as dogs and cats as well as livestock such as cows, horses, sheep, goats, pigs, and other domesticated and wild animals. A subject “in need of” treatment according to the present invention may be “suffering from or suspected of suffering from” a specific disease or disorder may have been positively diagnosed or otherwise presents with a sufficient number of risk factors or a sufficient number or combination of signs or symptoms such that a medical professional could diagnose or suspect that the subject was suffering from the disease or disorder. Thus, subjects suffering from, and suspected of suffering from, a specific disease or disorder are not necessarily two distinct groups.

Exemplary types of non-cancerous (e.g., cell proliferative) diseases or disorders that may be amenable to treatment with the compounds of the present invention include inflammatory diseases and conditions, autoimmune diseases, neurodegenerative diseases, heart diseases, viral diseases, chronic and acute kidney diseases or injuries, metabolic diseases, and allergic and genetic diseases.

Representative examples of specific non-cancerous diseases and disorders include rheumatoid arthritis, alopecia areata, lymphoproliferative conditions, autoimmune hematological disorders (e.g. hemolytic anemia, aplastic anemia, anhidrotic ectodermal dysplasia, pure red cell anemia and idiopathic thrombocytopenia), cholecystitis, acromegaly, rheumatoid spondylitis, osteoarthritis, gout, scleroderma, sepsis, septic shock, dacryoadenitis, cryopyrin associated periodic syndrome (CAPS), endotoxic shock, endometritis, gram-negative sepsis, keratoconjunctivitis sicca, toxic shock syndrome, asthma, adult respiratory distress syndrome, chronic obstructive pulmonary disease, chronic pulmonary inflammation, chronic graft rejection, hidradenitis suppurativa, inflammatory bowel disease, Crohn's disease, Behcet's syndrome, systemic lupus erythematosus, glomerulonephritis, multiple sclerosis, juvenile-onset diabetes, autoimmune uveoretinitis, autoimmune vasculitis, thyroiditis, Addison's disease, lichen planus, appendicitis, bullous pemphigus, pemphigus vulgaris, pemphigus foliaceus, paraneoplastic pemphigus, myasthenia gravis, immunoglobulin A nephropathy, Hashimoto's disease, Sjogren's syndrome, vitiligo, Wegener granulomatosis, granulomatous orchitis, autoimmune oophoritis, sarcoidosis, rheumatic carditis, ankylosing spondylitis, Grave's disease, autoimmune thrombocytopenic purpura, psoriasis, psoriatic arthritis, eczema, dermatitis herpetiformis, ulcerative colitis, pancreatic fibrosis, hepatitis, hepatic fibrosis, CD14 mediated sepsis, non-CD14 mediated sepsis, acute and chronic renal disease, irritable bowel syndrome, pyresis, restenosis, cervicitis, stroke and ischemic injury, neural trauma, acute and chronic pain, allergic rhinitis, allergic conjunctivitis, chronic heart failure, congestive heart failure, acute coronary syndrome, cachexia, malaria, leprosy, leishmaniasis, Lyme disease, Reiter's syndrome, acute synovitis, muscle degeneration, bursitis, tendonitis, tenosynovitis, herniated, ruptured, or prolapsed intervertebral disk syndrome, osteopetrosis, rhinosinusitis, thrombosis, silicosis, pulmonary sarcoidosis, bone resorption diseases, such as osteoporosis, fibromyalgia, AIDS and other viral diseases such as Herpes Zoster, Herpes Simplex I or II, influenza virus and cytomegalovirus, diabetes Type I and II, obesity, insulin resistance and diabetic retinopathy, 22q11.2 deletion syndrome, Angelman syndrome, Canavan disease, celiac disease, Charcot-Marie-Tooth disease, color blindness, Cri du chat, Down syndrome, cystic fibrosis, Duchenne muscular dystrophy, haemophilia, Klinefleter's syndrome, neurofibromatosis, phenylketonuria, Prader-Willi syndrome, sickle cell disease, Tay-Sachs disease, Turner syndrome, urea cycle disorders, thalassemia, otitis, pancreatitis, parotitis, pericarditis, peritonitis, pharyngitis, pleuritis, phlebitis, pneumonitis, uveitis, polymyositis, proctitis, interstitial lung fibrosis, dermatomyositis, atherosclerosis, arteriosclerosis, amyotrophic lateral sclerosis, asociality, varicosis, vaginitis, depression, and Sudden Infant Death Syndrome.

In other embodiments, the methods are directed to treating subjects having cancer. Broadly, the compounds of the present invention may be effective in the treatment of carcinomas (solid tumors including both primary and metastatic tumors), sarcomas, melanomas, and hematological cancers (cancers affecting blood including lymphocytes, bone marrow and/or lymph nodes) such as leukemia, lymphoma and multiple myeloma. Adult tumors/cancers and pediatric tumors/cancers are included. The cancers may be vascularized, or not yet substantially vascularized, or non-vascularized tumors.

Representative examples of cancers includes adrenocortical carcinoma, AIDS-related cancers (e.g., Kaposi's and AIDS-related lymphoma), appendix cancer, childhood cancers (e.g., childhood cerebellar astrocytoma, childhood cerebral astrocytoma), basal cell carcinoma, skin cancer (non-melanoma), biliary cancer, extrahepatic bile duct cancer, intrahepatic bile duct cancer, bladder cancer, urinary bladder cancer, brain cancer (e.g., gliomas and glioblastomas such as brain stem glioma, gestational trophoblastic tumor glioma, cerebellar astrocytoma, cerebral astrocytoma/malignant glioma, ependymoma, medulloblastoma, supratentorial primitive neuroectodermal tumors, visual pathway and hypothalamic glioma), breast cancer, bronchial adenomas/carcinoids, carcinoid tumor, nervous system cancer (e.g., central nervous system cancer, central nervous system lymphoma), cervical cancer, chronic myeloproliferative disorders, colorectal cancer (e.g., colon cancer, rectal cancer), polycythemia vera, lymphoid neoplasm, mycosis fungoids, Sezary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastrointestinal cancer (e.g., stomach cancer, small intestine cancer, gastrointestinal carcinoid tumor, gastrointestinal stromal tumor (GIST)), germ cell tumor, ovarian germ cell tumor, head and neck cancer, Hodgkin's lymphoma, leukemia, lymphoma, multiple myeloma, hepatocellular carcinoma, hypopharyngeal cancer, intraocular melanoma, ocular cancer, islet cell tumors (endocrine pancreas), renal cancer (e.g., Wilm's Tumor, clear cell renal cell carcinoma), liver cancer, lung cancer (e.g., non-small cell lung cancer and small cell lung cancer), Waldenstrom's macroglobulinema, melanoma, intraocular (eye) melanoma, merkel cell carcinoma, mesothelioma, metastatic squamous neck cancer with occult primary, multiple endocrine neoplasia (MEN), myelodysplastic syndromes, essential thrombocythemia, myelodysplastic/myeloproliferative diseases, nasopharyngeal cancer, neuroblastoma, oral cancer (e.g., mouth cancer, lip cancer, oral cavity cancer, tongue cancer, oropharyngeal cancer, throat cancer, laryngeal cancer), ovarian cancer (e.g., ovarian epithelial cancer, ovarian germ cell tumor, ovarian low malignant potential tumor), pancreatic cancer, islet cell pancreatic cancer, paranasal sinus and nasal cavity cancer, parathyroid cancer, penile cancer, pharyngeal cancer, pheochromocytoma, pineoblastoma, pituitary tumor, plasma cell neoplasm, pleuropulmonary blastoma, prostate cancer, retinoblastoma rhabdomyosarcoma, salivary gland cancer, uterine cancer (e.g., endometrial uterine cancer, uterine sarcoma, uterine corpus cancer), squamous cell carcinoma, testicular cancer, thymoma, thymic carcinoma, thyroid cancer, transitional cell cancer of the renal pelvis and ureter and other urinary organs, urethral cancer, gestational trophoblastic tumor, vaginal cancer and vulvar cancer.

Sarcomas that may be treatable with compounds of the present invention include both soft tissue and bone cancers alike, representative examples of which include osteosarcoma or osteogenic sarcoma (bone) (e.g., Ewing's sarcoma), chondrosarcoma (cartilage), leiomyosarcoma (smooth muscle), rhabdomyosarcoma (skeletal muscle), mesothelial sarcoma or mesothelioma (membranous lining of body cavities), fibrosarcoma (fibrous tissue), angiosarcoma or hemangioendothelioma (blood vessels), liposarcoma (adipose tissue), glioma or astrocytoma (neurogenic connective tissue found in the brain), myxosarcoma (primitive embryonic connective tissue) and mesenchymous or mixed mesodermal tumor (mixed connective tissue types).

In some embodiments, methods of the present invention entail treatment of subjects having cell proliferative diseases or disorders of the hematological system, liver, brain, lung, colon, pancreas, prostate, ovary, breast, skin, and endometrium.

As used herein, “cell proliferative diseases or disorders of the hematologic system” include lymphoma, leukemia, myeloid neoplasms, mast cell neoplasms, myelodysplasia, benign monoclonal gammopathy, lymphomatoid papulosis, polycythemia vera, chronic myelocytic leukemia, agnogenic myeloid metaplasia, and essential thrombocythemia. Representative examples of hematologic cancers may thus include multiple myeloma, lymphoma (including T-cell lymphoma, Hodgkin's lymphoma, non-Hodgkin's lymphoma (diffuse large B-cell lymphoma (DLBCL), follicular lymphoma (FL), mantle cell lymphoma (MCL) and ALK+ anaplastic large cell lymphoma (e.g., B-cell non-Hodgkin's lymphoma selected from diffuse large B-cell lymphoma (e.g., germinal center B-cell-like diffuse large B-cell lymphoma or activated B-cell-like diffuse large B-cell lymphoma), Burkitt's lymphoma/leukemia, mantle cell lymphoma, mediastinal (thymic) large B-cell lymphoma, follicular lymphoma, marginal zone lymphoma, lymphoplasmacytic lymphoma/Waldenstrom macroglobulinemia, metastatic pancreatic adenocarcinoma, refractory B-cell non-Hodgkin's lymphoma, and relapsed B-cell non-Hodgkin's lymphoma, childhood lymphomas, and lymphomas of lymphocytic and cutaneous origin, e.g., small lymphocytic lymphoma, leukemia, including childhood leukemia, hairy-cell leukemia, acute lymphocytic leukemia, acute myelocytic leukemia, acute myeloid leukemia (e.g., acute monocytic leukemia), chronic lymphocytic leukemia, small lymphocytic leukemia, chronic myelocytic leukemia, chronic myelogenous leukemia, and mast cell leukemia, myeloid neoplasms and mast cell neoplasms.

As used herein, “cell proliferative diseases or disorders of the liver” include all forms of cell proliferative disorders affecting the liver. Cell proliferative disorders of the liver may include liver cancer (e.g., hepatocellular carcinoma, intrahepatic cholangiocarcinoma and hepatoblastoma), a precancer or precancerous condition of the liver, benign growths or lesions of the liver, and malignant growths or lesions of the liver, and metastatic lesions in tissue and organs in the body other than the liver. Cell proliferative disorders of the brain may include hyperplasia, metaplasia, dysplasia of the liver, hepatocellular carcinoma, intrahepatic cholangiocarcinoma (bile duct cancer), angiosarcoma, hemangiosarcoma, hepatoblastoma, and secondary liver cancer (metastatic liver cancer).

As used herein, “cell proliferative diseases or disorders of the brain” include all forms of cell proliferative disorders affecting the brain. Cell proliferative disorders of the brain may include brain cancer (e.g., gliomas, glioblastomas, meningiomas, pituitary adenomas, vestibular schwannomas, and primitive neuroectodermal tumors (medulloblastomas)), a precancer or precancerous condition of the brain, benign growths or lesions of the brain, and malignant growths or lesions of the brain, and metastatic lesions in tissue and organs in the body other than the brain. Cell proliferative disorders of the brain may include hyperplasia, metaplasia, and dysplasia of the brain.

As used herein, “cell proliferative diseases or disorders of the lung” include all forms of cell proliferative disorders affecting lung cells. Cell proliferative disorders of the lung include lung cancer, precancer and precancerous conditions of the lung, benign growths or lesions of the lung, hyperplasia, metaplasia, and dysplasia of the lung, and metastatic lesions in the tissue and organs in the body other than the lung. Lung cancer includes all forms of cancer of the lung, e.g., malignant lung neoplasms, carcinoma in situ, typical carcinoid tumors, and atypical carcinoid tumors. Lung cancer includes small cell lung cancer (“SLCL”), non-small cell lung cancer (“NSCLC”), squamous cell carcinoma, adenocarcinoma, small cell carcinoma, large cell carcinoma, squamous cell carcinoma, and mesothelioma. Lung cancer can include “scar carcinoma”, bronchoalveolar carcinoma, giant cell carcinoma, spindle cell carcinoma, and large cell neuroendocrine carcinoma. Lung cancer also includes lung neoplasms having histologic and ultrastructural heterogeneity (e.g., mixed cell types). In some embodiments, a compound of the present invention may be used to treat non-metastatic or metastatic lung cancer (e.g., NSCLC, ALK-positive NSCLC, NSCLC harboring ROS1 Rearrangement, Lung Adenocarcinoma, and Squamous Cell Lung Carcinoma).

As used herein, “cell proliferative diseases or disorders of the colon” include all forms of cell proliferative disorders affecting colon cells, including colon cancer, a precancer or precancerous conditions of the colon, adenomatous polyps of the colon and metachronous lesions of the colon. Colon cancer includes sporadic and hereditary colon cancer, malignant colon neoplasms, carcinoma in situ, typical carcinoid tumors, and atypical carcinoid tumors, adenocarcinoma, squamous cell carcinoma, and squamous cell carcinoma. Colon cancer can be associated with a hereditary syndrome such as hereditary nonpolyposis colorectal cancer, familiar adenomatous polyposis, MYH associated polyposis, Gardner's syndrome, Peutz-Jeghers syndrome, Turcot's syndrome and juvenile polyposis. Cell proliferative disorders of the colon may also be characterized by hyperplasia, metaplasia, or dysplasia of the colon.

As used herein, “cell proliferative diseases or disorders of the pancreas” include all forms of cell proliferative disorders affecting pancreatic cells. Cell proliferative disorders of the pancreas may include pancreatic cancer, a precancer or precancerous condition of the pancreas, hyperplasia of the pancreas, dysplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas, including ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma, and pancreatic neoplasms having histologic and ultrastructural heterogeneity (e.g., mixed cell types).

As used herein, “cell proliferative diseases or disorders of the prostate” include all forms of cell proliferative disorders affecting the prostate. Cell proliferative disorders of the prostate may include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate may include hyperplasia, metaplasia, and dysplasia of the prostate.

As used herein, “cell proliferative diseases or disorders of the ovary” include all forms of cell proliferative disorders affecting cells of the ovary. Cell proliferative disorders of the ovary may include a precancer or precancerous condition of the ovary, benign growths or lesions of the ovary, ovarian cancer, and metastatic lesions in tissue and organs in the body other than the ovary. Cell proliferative disorders of the ovary may include hyperplasia, metaplasia, and dysplasia of the ovary.

As used herein, “cell proliferative diseases or disorders of the breast” include all forms of cell proliferative disorders affecting breast cells. Cell proliferative disorders of the breast may include breast cancer, a precancer or precancerous condition of the breast, benign growths or lesions of the breast, and metastatic lesions in tissue and organs in the body other than the breast. Cell proliferative disorders of the breast may include hyperplasia, metaplasia, and dysplasia of the breast.

As used herein, “cell proliferative diseases or disorders of the skin” include all forms of cell proliferative disorders affecting skin cells. Cell proliferative disorders of the skin may include a precancer or precancerous condition of the skin, benign growths or lesions of the skin, melanoma, malignant melanoma or other malignant growths or lesions of the skin, and metastatic lesions in tissue and organs in the body other than the skin. Cell proliferative disorders of the skin may include hyperplasia, metaplasia, and dysplasia of the skin.

As used herein, “cell proliferative diseases or disorders of the endometrium” include all forms of cell proliferative disorders affecting cells of the endometrium. Cell proliferative disorders of the endometrium may include a precancer or precancerous condition of the endometrium, benign growths or lesions of the endometrium, endometrial cancer, and metastatic lesions in tissue and organs in the body other than the endometrium. Cell proliferative disorders of the endometrium may include hyperplasia, metaplasia, and dysplasia of the endometrium.

In some embodiments, a compound of the present invention may be used to treat T cell leukemia or T cell lymphoma.

In some embodiments, a compound of the present invention may be used to treat Hodgkin's lymphoma or non-Hodgkin's lymphoma.

In some embodiments, a compound of the present invention may be used to treat myeloid leukemia.

In some embodiments, a compound of the present invention may be used to treat non-small cell lung cancer (NSCLC).

In some embodiments, a compound of the present invention may be used to treat melanoma.

In some embodiments, a compound of the present invention may be used to treat triple-negative breast cancer (TNBC).

In some embodiments, a compound of the present invention may be used to treat nasopharyngeal cancer (NPC).

In some embodiments, a compound of the present invention may be used to treat microsatellite stable colorectal cancer (mssCRC).

In some embodiments, a compound of the present invention may be used to treat thymoma.

In some embodiments, a compound of the present invention may be used to treat carcinoid.

In some embodiments, a compound of the present invention may be used to treat gastrointestinal stromal tumor (GIST).

The compounds of the present invention and their pharmaceutically acceptable salts and stereoisomers may be administered to a patient, e.g., a cancer patient, as a monotherapy or by way of combination therapy. Therapy may be “front/first-line”, i.e., as an initial treatment in patients who have undergone no prior anti-cancer treatment regimens, either alone or in combination with other treatments; or “second-line” as a treatment in patients who have undergone a prior anti-cancer treatment regimen, either alone or in combination with other treatments; or as “third-line”, “fourth-line”, etc. treatments, either alone or in combination with other treatments. Therapy may also be given to patients who have had previous treatments which have been unsuccessful, or partially successful but who became non-responsive or intolerant to the particular treatment. Therapy may also be given as an adjuvant treatment, i.e., to prevent reoccurrence of cancer in patients with no currently detectable disease or after surgical removal of a tumor. Thus, in some embodiments, the compound may be administered to a patient who has received prior therapy, such as chemotherapy, radioimmunotherapy, surgical therapy, immunotherapy, radiation therapy, targeted therapy or any combination thereof.

The methods of the present invention may entail administration of an inventive compound or a pharmaceutical composition thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5, or 6 weeks, and in other embodiments entails at least one 28-day cycle which includes daily administration for 3 weeks (21 days) followed by a 7-day off period. In other embodiments, the compound may be dosed twice a day (BID) over the course of two and a half days (for a total of 5 doses) or once a day (QD) over the course of two days (for a total of 2 doses). In other embodiments, the compound may be dosed once a day (QD) over the course of five days.

### Combination Therapy

The compounds of the present invention and their pharmaceutically acceptable salts, hydrates, solvates, prodrugs, stereoisomers, or tautomers may be used in combination or concurrently with at least one other active agent e.g., anti-cancer agent or regimen, in treating diseases and disorders. The terms “in combination” and “concurrently” in this context mean that the agents are co-administered, which includes substantially contemporaneous administration, by way of the same or separate dosage forms, and by the same or different modes of administration, or sequentially, e.g., as part of the same treatment regimen, or by way of successive treatment regimens. Thus, if given sequentially, at the onset of administration of the second agent, the first of the two agents is in some cases still detectable at effective concentrations at the site of treatment. The sequence and time interval may be determined such that they can act together (e.g., synergistically to provide an increased benefit than if they were administered otherwise). For example, the agents may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents at exactly the same time.

In some embodiments, the treatment regimen may include administration of a compound of the present invention or a pharmaceutically acceptable salt, hydrate, solvate, prodrug, stereoisomer, or tautomer thereof in combination with one or more additional therapeutic agents known for use in treating the disease or disorder (e.g., cancer). The dosage of the additional anticancer therapeutic may be the same or even lower than known or recommended doses. See, Hardman et al., eds., Goodman & Gilman's The Pharmacological Basis Of Basis Of Therapeutics, 10th ed., McGraw-Hill, New York, 2001; Physician's Desk Reference 60th ed., 2006. For example, anti-cancer agents that may be used in combination with the inventive compounds are known in the art. See, e.g., U.S. Pat. No. 9,101,622 (Section 5.2 thereof) and U.S. Pat. No. 9,345,705 B2 (Columns 12-18 thereof). Representative examples of additional anti-cancer agents and treatment regimens include radiation therapy, chemotherapeutics (e.g., mitotic inhibitors, angiogenesis inhibitors, anti-hormones, autophagy inhibitors, alkylating agents, intercalating antibiotics, growth factor inhibitors, anti-androgens, signal transduction pathway inhibitors, anti-microtubule agents, platinum coordination complexes, HDAC inhibitors, proteasome inhibitors, and topoisomerase inhibitors), immune-modulators, therapeutic antibodies (e.g., mono-specific and bispecific antibodies) and CAR-T therapy.

In some embodiments, the compound of the invention and the additional anticancer therapeutic agent may be administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. The two or more anticancer therapeutics may be administered within the same patient visit.

In some embodiments, the compound of the present invention and the additional therapeutic agent (e.g., an anti-cancer therapeutic) are cyclically administered. By way of example in the context of cancer treatment, cycling therapy involves the administration of one anticancer therapeutic for a period of time, followed by the administration of a second anti-cancer therapeutic for a period of time and repeating this sequential administration, i.e., the cycle, in order to reduce the development of resistance to one or both of the anticancer therapeutics, to avoid or reduce the side effects of one or both of the anticancer therapeutics, and/or to improve the efficacy of the therapies. In one example, cycling therapy involves the administration of a first anticancer therapeutic for a period of time, followed by the administration of a second anticancer therapeutic for a period of time, optionally, followed by the administration of a third anticancer therapeutic for a period of time and so forth, and repeating this sequential administration, i.e., the cycle in order to reduce the development of resistance to one of the anticancer therapeutics, to avoid or reduce the side effects of one of the anticancer therapeutics, and/or to improve the efficacy of the anticancer therapeutics.

In some embodiments, and depending on the particular cancer being treated, the compound of the present invention may be used in combination with at least one other anti-cancer agents such as Paclitaxel (e.g., ovarian cancer, breast cancer, lung cancer, Kaposi sarcoma, cervical cancer, and pancreatic cancer), Topotecan (e.g., ovarian cancer and lung cancer), Irinotecan (e.g., colon cancer, and small cell lung cancer), Etoposide (e.g., testicular cancer, lung cancer, lymphomas, and non-lymphocytic leukemia), Vincristine (e.g., leukemia), Leucovorin (e.g., colon cancer), Altretamine (e.g., ovarian cancer), Daunorubicin (e.g., acute myeloid leukemia (AML), acute lymphocytic leukemia (ALL), chronic myelogenous leukemia (CML), and Kaposi's sarcoma), Trastuzumab (e.g., breast cancer, stomach cancer, and esophageal cancer), Rituximab (e.g., non-Hodgkin's lymphoma), Cetuximab (e.g., colorectal cancer, metastatic non-small cell lung cancer and head and neck cancer), Pertuzumab (e.g., metastatic HER2-positive breast cancer), Alemtuzumab (e.g., chronic lymphocytic leukemia (CLL), cutaneous T-cell lymphoma (CTCL) and T-cell lymphoma), Panitumumab (e.g., colon and rectum cancer), Tamoxifen (e.g., breast cancer), Fulvestrant (e.g., breast cancer), Letrazole (e.g., breast cancer), Exemestane (e.g., breast cancer), Azacytidine (e.g., myelodysplastic syndromes), Mitomycin C (e.g., gastro-intestinal cancers, anal cancers, and breast cancers), Dactinomycin (e.g., Wilms tumor, rhabdomyosarcoma, Ewing's sarcoma, trophoblastic neoplasm, testicular cancer, and ovarian cancer), Erlotinib (e.g., non-small cell lung cancer and pancreatic cancer), Sorafenib (e.g., kidney cancer and liver cancer), Temsirolimus (e.g., kidney cancer), Bortezomib (e.g., multiple myeloma and mantle cell lymphoma), Pegaspargase (e.g., acute lymphoblastic leukemia), Cabometyx (e.g., hepatocellular carcinoma, medullary thyroid cancer, and renal cell carcinoma), Pembrolizumab (e.g., cervical cancer, gastric cancer, hepatocellular carcinoma, Hodgkin's lymphoma, melanoma, Merkel cell carcinoma, non-small cell lung cancer, urothelial carcinoma, and squamous cell carcinoma of the head and neck), Nivolumab (e.g., colorectal cancer, hepatocellular carcinoma, melanoma, non-small cell lung cancer, renal cell carcinoma, small cell lung cancer, and urothelial carcinoma), Regorafenib (e.g., colorectal cancer, gastrointestinal stromal tumor, and hepatocellular carcinoma), Cemiplimab (e.g., cutaneous squamous cell carcinoma (CSCC)), Avelumab (e.g., Merkel cell carcinoma, urothelial carcinoma, and renal cell carcinoma), Durvalumab (e.g., bladder and lung cancer), Atezolizumab (e.g., urothelial carcinoma, non-small cell lung cancer (NSCLC), triple-negative breast cancer (TNBC), small cell lung cancer (SCLC), and heptatocellular carcinoma (HCC)), and Ipilimuimab (e.g., melanoma, non-small cell lung carcinoma (NSCLC), small cell lung cancer (SCLC), bladder cancer, and prostate cancer).

### Pharmaceutical Kits

The present compositions may be assembled into kits or pharmaceutical systems. Kits or pharmaceutical systems according to this aspect of the invention include a carrier or package such as a box, carton, tube or the like, having in close confinement therein one or more containers, such as vials, tubes, ampoules, or bottles, which contain a compound of the present invention or a pharmaceutical composition which contains the compound and a pharmaceutically acceptable carrier wherein the compound and the carrier may be disposed in the same or separate containers. The kits or pharmaceutical systems of the invention may also include printed instructions for using the compounds and compositions.

These and other aspects of the present invention will be further appreciated upon consideration of the following Examples, which are intended to illustrate certain particular embodiments of the invention but are not intended to limit its scope, as defined by the claims.

### EXAMPLES

These and other aspects of the present invention will be further appreciated upon consideration of the following Examples, which are intended to illustrate certain particular embodiments of the invention but are not intended to limit its scope, as defined by the claims.

### Example 1: Synthesis of 3-(1-oxo-5-(piperidin-4-yl)isoindolin-2-yl)piperidine-2,6-dione

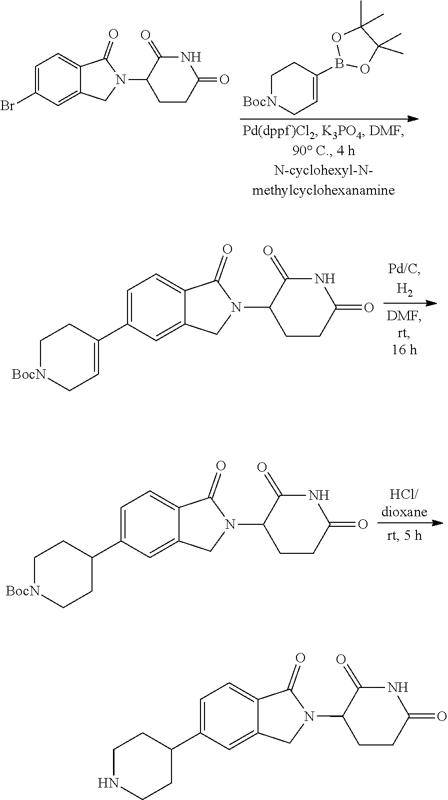 [SMILES: O=C1CCC(N2Cc3cc(Br)ccc3C2=O)C(=O)N1 ; CC(C)(C)OC(=O)N1CC=C(B2OC(C)(C)C(C)(C)O2)CC1 ; CC(C)(C)OC(=O)N1CC=C(c2ccc3c(c2)CN(C2CCC(=O)NC2=O)C3=O)CC1 ; CC(C)(C)OC(=O)N1CCC(c2ccc3c(c2)CN(C2CCC(=O)NC2=O)C3=O)CC1 ; O=C1CCC(N2Cc3cc(C4CCNCC4)ccc3C2=O)C(=O)N1]

### tert-Butyl-4-(2-(2,6-dioxopiperidin-3-yl)-1-oxoisoindolin-5-yl)-3,6-dihydropyridine-1(2H)-carboxylate

A mixture of 3-(6-bromo-3-oxo-1H-isoindol-2-yl)piperidine-2,6-dione (3.0 g, 9.3 mmol), tert-butyl 4-(4,4,5,5-tetramethyl-1,3,2-dioxaborolan-2-yl)-3,6-dihydropyridine-1(2H)-carboxylate (11.5 g, 37.3 mmol), potassium phosphate (2.0 g, 9.3 mmol), [1,1″-bis(diphenylphosphino)ferrocene]dichloropalladium(II) (0.7 g, 1.9 mmol) in N,N-dimethylformamide (20 mL) was stirred at 90° C. for 4 hours. The reaction mixture was then concentrated to afford a residue, which was dissolved in ethyl acetate (500 mL). Water (500 mL) was added and the layers were separated. Solid NaCl was added to the aqueous layer under vigorous stirring until saturation of NaCl was reached. The undissolved NaCl was removed by filtration, and the aqueous phase was further extracted with tetrahydrofuran (500 mL×2). The combined organic layers were dried and concentrated to afford a crude product, which was purified using silica gel column chromatography (petroleum ether/ethyl acetate=1:1 to 100% ethyl acetate) to afford the title compound as a yellow solid (1.1 g, 36%). MS [M+H]⁺=426.3.

### tert-Butyl 4-(2-(2,6-dioxopiperidin-3-yl)-1-oxoisoindolin-5-yl)piperidine-1-carboxylate

To a mixture of tert-butyl-4-(2-(2,6-dioxopiperidin-3-yl)-1-oxoisoindolin-5-yl)-3,6-dihydropyridine-1(2H)-carboxylate (1.0 g, 2.4 mmol) and 10% Pd/C (400 mg) was added N,N-dimethylformamide (10 mL). The suspension was stirred at room temperature (rt) for 16 hours under hydrogen atmosphere. The reaction mixture was then diluted with dichloromethane, filtered and concentrated to afford the title compound as a yellow solid (1.0 g, 91%), which was used without further purification. MS [M+H]⁺=428.3.

### 3-(1-oxo-5-(piperidin-4-yl)isoindolin-2-yl)piperidine-2,6-dione

To tert-butyl 4-(2-(2,6-dioxopiperidin-3-yl)-1-oxoisoindolin-5-yl)piperidine-1-carboxylate (1.0 g, 2.3 mmol) was added 4.0 M HCl/dioxane (6 mL), and the reaction vessel was closed and the reaction was stirred at rt for 5 hours. The reaction mixture was concentrated under vacuum to afford the title compound as a yellow solid (1.0 g, 100%), which was used without further purification. MS [M+H]⁺=328.3.

### Example 2: Synthesis of 5-oxo-4-phenyl-2,3,4,5-tetrahydrobenzo[f][1,4]oxazepine-7-carbaldehyde

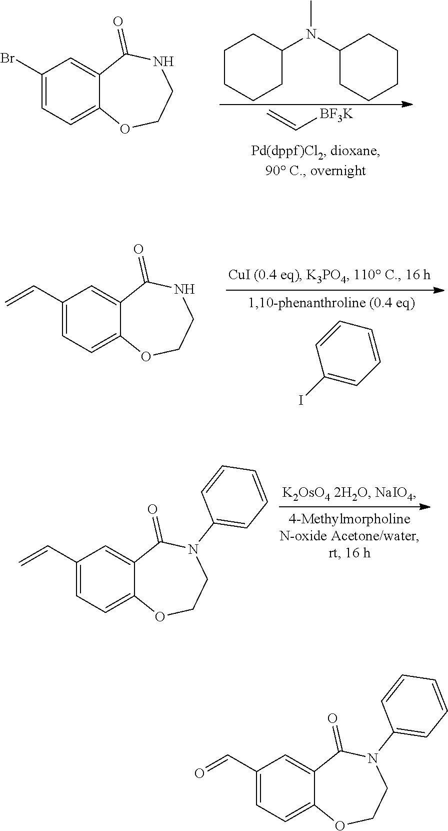

### 7-Vinyl-3,4-dihydrobenzo[f][1,4]oxazepin-5(2H)-one

A mixture of 7-bromo-3,4-dihydrobenzo[f][1,4]oxazepin-5(2H)-one (4.5 g, 18.7 mmol), trifluoro(vinyl)-borane potassium salt (5.0 g, 37.3 mmol), N-cyclohexyl-N-methylcyclohexanamine (7.3 g, 37.3 mmol), [1,1″-bis(diphenylphosphino)ferrocene]dichloropalladium(II) (1.4 g, 1.9 mmol) in 1,4-dioxane (30 mL) was stirred at 90° C. for 16 hours. The reaction mixture was concentrated, and then diluted with ethyl acetate (500 mL). The organic layer was washed with water (500 mL). The layers were separated and NaCl was added to the aqueous layer until a saturated solution was formed. The aqueous layer was then extracted with tetrahydrofuran (500 mL×2). The organic layers were combined and dried with MgSO₄ and then filtered. The filtrate was concentrated to afford a crude product which was purified by silica gel column (eluting of petroleum ether/ethyl acetate=10:1 to 5:1) to afford the title compound as a yellow solid (2.5 g, 71%). MS [M+H]⁺=190.2.

### 4-Phenyl-7-vinyl-3,4-dihydrobenzo[f][1,4]oxazepin-5(2H)-one

A mixture of the 7-vinyl-3,4-dihydrobenzo[f][1,4]oxazepin-5(2H)-one (2.4 g, 12.7 mmol), iodobenzene (2.6 g, 12.7 mmol), potassium phosphate (0.7 g, 3.2 mmol), 1,10-phenanthroline (0.9 g, 5.1 mmol), and copper (I) iodide (1.0 g, 5.1 mmol) in toluene (20 mL) was stirred at 110° C. for 16 hours. The reaction mixture was concentrated and then diluted with ethyl acetate (500 mL). The organic phase was washed with water (500 mL). NaCl was added to the aqueous layer until a saturated solution formed, and extracted with tetrahydrofuran (500 mL×2). The combined organic layers were dried and concentrated. The crude product was purified by silica gel column (eluting of petroleum ether/ethyl acetate=10:1 to 5:1) to afford the title compound as a yellow solid (1.9 g, 56%). MS [M+H]⁺=266.2.

### 5-Oxo-4-phenyl-2,3,4,5-tetrahydrobenzo[f][1,4]oxazepine-7-carbaldehyde

To a solution of 4-phenyl-7-vinyl-3,4-dihydrobenzo[f][1,4]oxazepin-5(2H)-one (600 mg, 2.3 mmol), 4-methylmorpholine N-oxide (796 mg, 6.8 mmol), and sodium periodate (963 mg, 4.5 mmol) in acetone/water (15 mL, 3:2) at rt was added potassium osmate (VI) dihydrate (40 mg, 0.1 mmol). The reaction mixture stirred at rt for 16 hours. Ethyl acetate (500 mL) was then added and the organic layer was washed with water (500 mL). NaCl was added to the aqueous layer until a saturated solution formed. The aqueous layer was extracted with THF (500 mL×2). The combined organic layers were dried and concentrated. The crude product was purified by silica gel column (petroleum ether/ethyl acetate=5:1 to 3:1) to afford the title compound (300 mg, 50%) as a yellow solid. MS [M+H]⁺=268.2.

### Example 3: Synthesis of 3-(1-oxo-5-(1-((5-oxo-4-phenyl-2,3,4,5-tetrahydrobenzo[f][1,4]oxazepin-7-yl)methyl)piperidin-4-yl)isoindolin-2-yl)piperidine-2,6-dione (1)

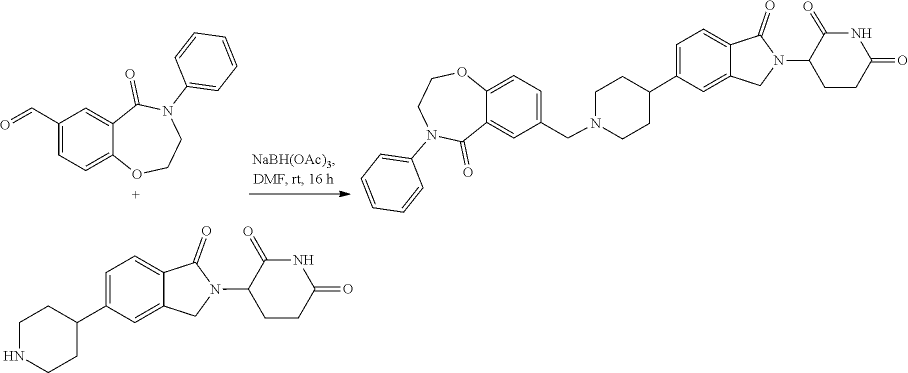 [SMILES: O=Cc1ccc2c(c1)C(=O)N(c1ccccc1)CCO2 ; O=C1CCC(N2Cc3cc(C4CCN(Cc5ccc6c(c5)C(=O)N(c5ccccc5)CCO6)CC4)ccc3C2=O)C(=O)N1 ; O=C1CCC(N2Cc3cc(C4CCNCC4)ccc3C2=O)C(=O)N1]

A mixture 5-oxo-4-phenyl-2,3,4,5-tetrahydrobenzo[f][1,4]oxazepine-7-carbaldehyde (80 mg, 0.3 mmol), 3-(1-oxo-5-(piperidin-4-yl)isoindolin-2-yl)piperidine-2,6-dione (98 mg, 0.3 mmol), and sodium triacetoxyborohydride (191 mg, 0.9 mmol) in DMF (5 mL) was stirred at rt for 16 hours. The suspension was concentrated and purified with prep-HPLC to afford the title compound (29.9 mg, 17%) as a white solid. ¹H NMR (400 MHz, DMSO-d₆) δ 10.98 (s, 1H), 7.69-7.62 (m, 2H), 7.55-7.39 (m, 8H), 7.37-7.28 (m, 1H), 7.09 (d, J=8.4 Hz, 1H), 5.11 (dd, J=13.3, 5.2 Hz, 1H), 4.49-4.39 (m, 3H), 4.30 (d, J=17.2 Hz, 1H), 3.92 (t, J=5.2 Hz, 2H), 3.56 (s, 2H), 2.97 (dd, J=8.4, 5.5 Hz, 2H), 2.94-2.87 (m, 1H), 2.73-2.57 (m, 2H), 2.48-2.34 (m, 1H), 2.13 (t, J=11.0 Hz, 2H), 2.05-1.95 (m, 1H), 1.84-1.67 (m, 4H). MS [M+H]⁺=579.8.

### Example 4: Synthesis of 4-oxo-5-phenyl-4,5,6,7-tetrahydropyrazolo[1,5-a]pyrazine-2-carbaldehyde

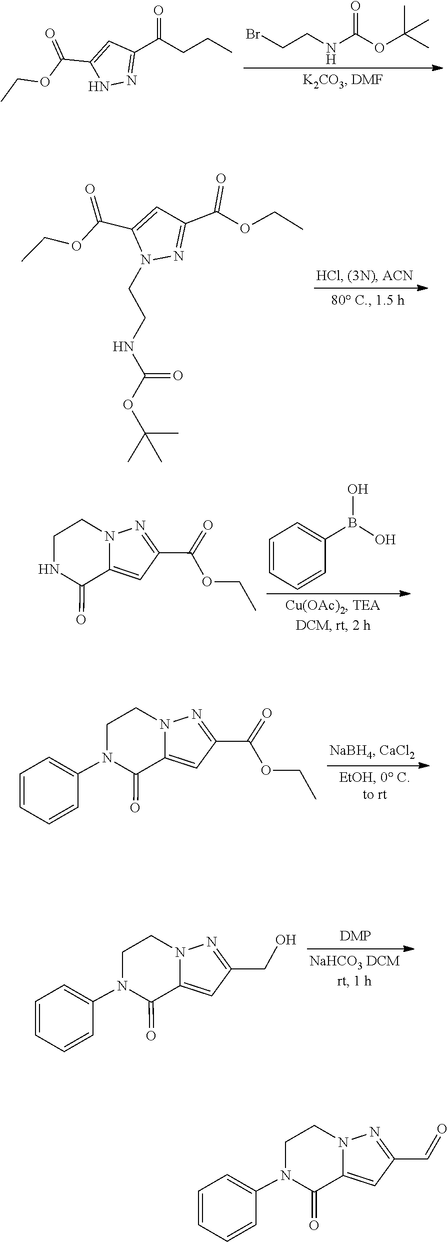

### Diethyl 1-(2-((tert-butoxycarbonyl)amino)ethyl)-1H-pyrazole-3,5-dicarboxylate

To a stirred solution of diethyl 1H-pyrazole-3,5-dicarboxylate (2.0 g, 9.43 mmol) and tert-butyl (2-bromoethyl) carbamate (2.7 g, 12.2 mmol) in DMF (20 mL), was added K₂CO₃ (2.6 g, 18.8 mmol). The reaction mixture stirred at rt overnight. The reaction mixture was diluted with ethyl acetate (50 mL) and water (50 mL). The organic layer was washed with water (50 mL×3). The combined organic layers were concentrated under reduced pressure. The residue was purified by silica gel column chromatography (dichloromethane/methanol=20/1) to afford the title compound (2.6 g, 78%) as a white solid. MS [M+H]⁺=356.17.

### Ethyl 4-oxo-4,5,6,7-tetrahydropyrazolo[1,5-a]pyrazine-2-carboxylate

To a solution of diethyl 1-(2-((tert-butoxycarbonyl)amino)ethyl)-1H-pyrazole-3,5-dicarboxylate (2.6 g, 7.3 mmol) in CH₃CN (20 mL) was added aq. HCl (3 N, 5 mL) and the reaction was stirred at 80° C. for 1.5 hours. The reaction was then neutralized to pH=7 with aq. NaHCO₃ and diluted with water (50 mL). The aq. layer was extracted with dichloromethane (50 mL×3). The organic layers were combined and concentrated under reduced pressure to afford a residue which was purified by silica gel column chromatography (dichloromethane/methanol=20/1) to afford the title compound (1.15 g, 62%) as a white solid. MS [M+H]⁺=210.08.

### Ethyl 4-oxo-5-phenyl-4,5,6,7-tetrahydropyrazolo[1,5-a]pyrazine-2-carboxylate

Under nitrogen, a solution of ethyl 4-oxo-4,5,6,7-tetrahydropyrazolo[1,5-a]pyrazine-2-carboxylate (1.15 g, 5.5 mmol), phenylboronic acid (1.05 g, 8.25 mmol), Cu(OAc)₂ (995 mg, 5.5 mmol), and triethylamine (1.1 g, 11.0 mmol) in dichloromethane (DCM) (10 mL) was stirred at rt for 2 hours. The reaction mixture was diluted with ethyl acetate (50 mL) and the organic layer was washed with water (50 mL×3). The organic layer was concentrated under reduced pressure to afford a residue which was purified by silica gel column chromatography (dichloromethane/methanol=20/1) to afford the title compound (700 mg, 44.6%) as a white solid. MS [M+H]⁺=286.10.

### 2-(Hydroxymethyl)-5-phenyl-6,7-dihydropyrazolo[1,5-a]pyrazin-4(5H)-one

A solution of ethyl 4-oxo-5-phenyl-4,5,6,7-tetrahydropyrazolo[1,5-a]pyrazine-2-carboxylate (700 mg, 2.45 mmol) and CaCl₂ (430 mg, 3.8 mmol) in EtOH (10 mL) was stirred at 0° C. for 10 minutes. Solid NaBH₄ (280 mg, 7.4 mmol) was added and the reaction mixture was stirred at rt for 16 hours. The reaction mixture was diluted with water (20 mL) and extracted with ethyl acetate (20 mL×3). The organic layers were combined, dried over Na₂SO₄ and concentrated under reduced pressure. The residue was purified by silica gel column to afford the title compound (250 mg, 84%) as a white solid. MS [M+H]⁺=244.10.

### 4-Oxo-5-phenyl-4,5,6,7-tetrahydropyrazolo[1,5-a]pyrazine-2-carbaldehyde

A suspension of 2-(hydroxymethyl)-5-phenyl-6,7-dihydropyrazolo[1,5-a]pyrazin-4(5H)-one (250 mg, 1.0 mmol), NaHCO₃ (864 mg, 10 mmol), and Dess-Martin periodinane (DMP, 875 mg, 2.0 mmol) in DCM (25 mL) was stirred at rt for 1 hour. Water (20 mL) was then added and the aq. phase was extracted with ethyl acetate (20 mL×3). The organic layers were combined, dried over Na₂SO₄ and concentrated to afford a residue which was purified by silica gel column (dichloromethane/methanol=15:1) to afford the title compound (150 mg, 62%) as a white solid. MS [M+H]⁺=242.10.

### Example 5: Synthesis of 3-(1-oxo-5-(1-((4-oxo-5-phenyl-4,5,6,7-tetrahydropyrazolo[1,5a]pyrazin-2-yl)methyl)piperidin-4-yl)isoindolin-2-yl)piperidine-2,6-dione (8)

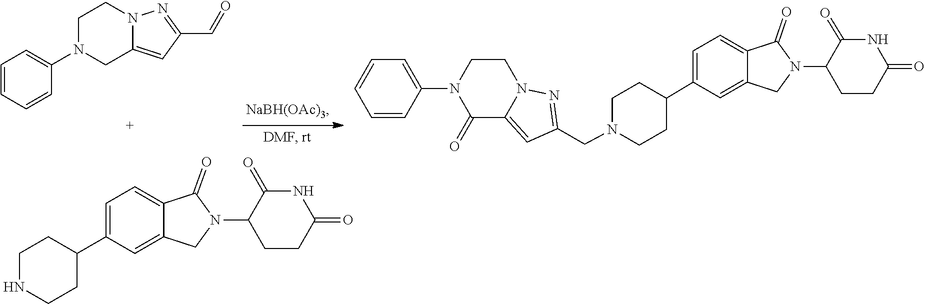 [SMILES: O=Cc1cc2n(n1)CCN(c1ccccc1)C2 ; O=C1CCC(N2Cc3cc(C4CCN(Cc5cc6n(n5)CCN(c5ccccc5)C6=O)CC4)ccc3C2=O)C(=O)N1 ; O=C1CCC(N2Cc3cc(C4CCNCC4)ccc3C2=O)C(=O)N1]

A solution of 4-oxo-5-phenyl-4,5,6,7-tetrahydropyrazolo[1,5-a]pyrazine-2-carbaldehyde (150 mg, 0.62 mmol), 3-(1-oxo-5-(piperidin-4-yl)isoindolin-2-yl)piperidine-2,6-dione (203 mg, 0.62 mmol), and NaBH(OAc)₃ (254 mg, 1.2 mmol) in DMF (5 mL) was stirred at rt for 16 hours. The reaction was diluted with water (20 mL), and extracted with ethyl acetate (20 mL×3). The organic layers were combined and dried over Na₂SO₄. The solvent was removed under reduced pressure, and the residue purified by prep-HPLC to afford the title compound (12 mg, 3.5%) as a white solid. ¹H NMR (400 MHz, DMSO-d₆) δ 10.97 (s, 1H), 8.17 (s, 1H), 7.64 (s, 1H), 7.51-7.37 (m, 5H), 7.29 (d, J=7.2 Hz, 1H), 6.75 (s, 1H), 5.09 (s, 1H), 4.54-4.14 (m, 7H), 3.09-2.85 (m, 3H), 2.65 (d, J=15.0 Hz, 1H), 2.44-2.33 (m, 1H), 2.23-1.94 (m, 3H), 1.91-1.59 (m, 4H). MS [M+H]⁺=553.20.

### Example 6: Synthesis of 3-(5-(1-(3-((1-Methyl-2-oxo-1,2-dihydropyridin-3-yl)amino)benzyl)piperidin-4-yl)-1-oxoisoindolin-2-yl)piperidine-2,6-dione (12)

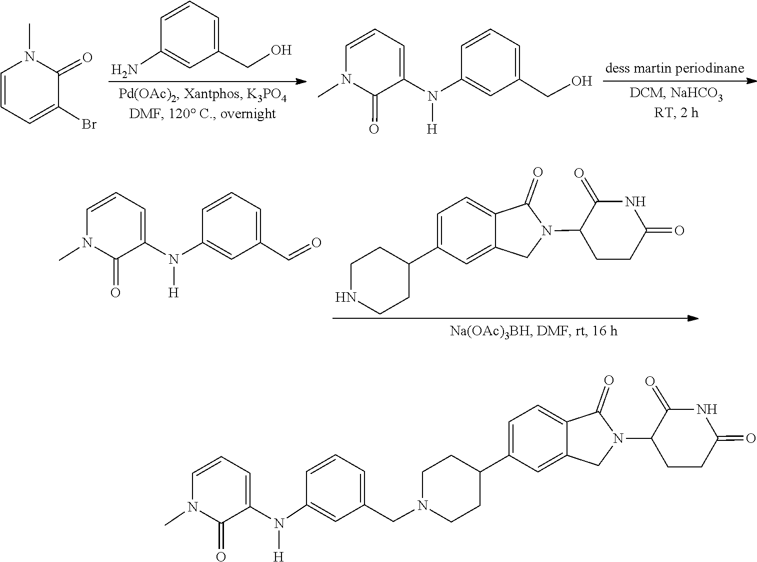

### 3-((3-(Hydroxymethyl)phenyl)amino)-1-methylpyridin-2(1H)-one

A solution of 3-bromo-1-methylpyridin-2(1H)-one (400 mg, 2.2 mmol) in 10 mL of anhydrous DMF under nitrogen atmosphere was treated with (3-aminophenyl)methanol (320 mg, 2.6 mmol), Pd(OAc)₂ (24 mg, 0.11 mmol), Xantphos (64 mg, 0.11 mmol) and K₃PO₄ (552 mg, 2.6 mmol), and the reaction mixture stirred at 120° C. for 16 hours. After completion, the reaction mixture was extracted with ethyl acetate (EtOAc) (50 mL) and the layers were separated. The organic phase was concentrated to afford a crude product that was purified by prep-high-performance liquid chromatography (HPLC) to give the title compound (30 mg, 6%) as a yellow solid. MS [M+H]⁺=231.0.

### 3-((1-Methyl-2-oxo-1,2-dihydropyridin-3-yl)amino)benzaldehyde

To a solution of 3-((3-(hydroxymethyl)phenyl)amino)-1-methylpyridin-2(1H)-one (20 mg, 0.09 mmol) in DCM (2 mL) was added Dess-Martin periodinane (74 mg, 0.17 mmol) and NaHCO₃ (34 mg, 0.40 mmol). The mixture stirred at rt for 2 hours. The suspension was then filtered and the filtrate was concentration to afford the crude title compound (10 mg, 49%) as a yellow solid, which was used without further purification. ¹H NMR (400 MHz, CDCl₃) δ 9.98 (s, 1H), 7.71 (dt, J=2.3, 1.0 Hz, 1H), 7.48-7.45 (m, 2H), 7.39-7.36 (m, 1H), 7.27 (s, 1H), 7.14 (dd, J=7.4, 1.8 Hz, 1H), 6.83 (dd, J=6.8, 1.6 Hz, 1H), 6.18 (t, J=7.2 Hz, 1H), 3.63 (s, 3H).

### 3-(5-(1-(3-((1-Methyl-2-oxo-1,2-dihydropyridin-3-yl)amino)benzyl)piperidin-4-yl)-1-oxoisoindolin-2-yl)piperidine-2,6-dione (12)

To a solution of 3-((1-methyl-2-oxo-1,2-dihydropyridin-3-yl)amino)benzaldehyde (10 mg, 0.04 mmol) and 3-(1-oxo-5-(piperidin-4-yl)isoindolin-2-yl)piperidine-2,6-dione (14 mg, 0.04 mmol) in DMF (1 mL) was added Na(OAc)₃BH (17 mg, 0.08 mmol). The mixture stirred at rt for 2 hours. The mixture was then filtered and the filtrate was concentration to give a crude residue, which was purified by prep-HPLC to afford the compound 12 (3.3 mg, 15%) as a light yellow solid. ¹H NMR (400 MHz, DMSO-d₆) δ 10.96 (s, 1H), 7.67-7.58 (m, 2H), 7.49 (s, 1H), 7.40 (d, J=8.0 Hz, 1H), 7.26-7.19 (m, 2H), 7.13-7.06 (m, 3H), 6.88 (d, J=7.6 Hz, 1H), 6.17 (t, J=7.2 Hz, 1H), 5.09 (dd, J=13.4, 5.0 Hz, 1H), 4.42 (dd, J=17.2, 2.6 Hz, 1H), 4.29 (dd, J=17.4, 2.8 Hz, 1H), 3.80 (d, J=13.0 Hz, 1H), 3.52 (s, 3H), 3.48 (s, 2H), 3.21-3.11 (m, 1H), 2.99-2.87 (m, 3H), 2.67-2.56 (m, 2H), 2.44-2.33 (m, 1H), 2.13-2.04 (m, 2H), 2.01-1.96 (m, 1H), 1.87-1.68 (m, 4H). MS [M+H]⁺=540.3.

### Example 7: Synthesis of 3-[1-oxo-5-[1-[(2-oxo-1-phenyl-indolin-6-yl)methyl]-4-piperidyl]isoindolin-2-yl]piperidine-2,6-dione (18)

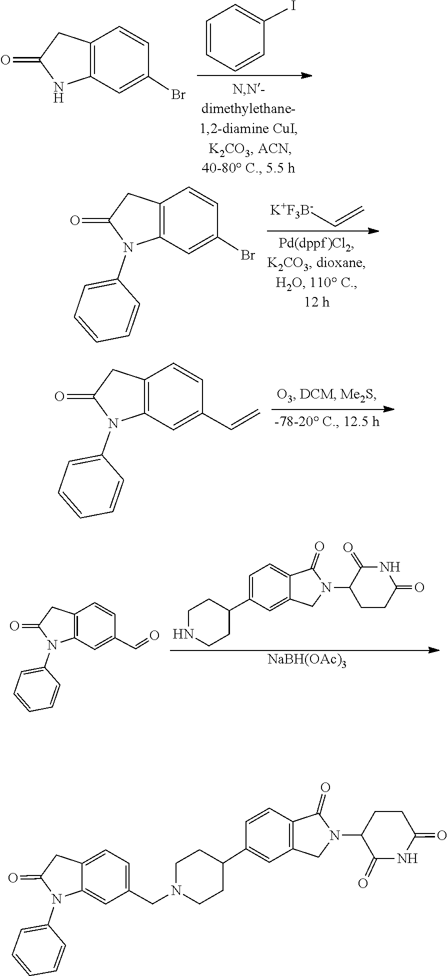

### 6-Bromo-1-phenyl-indolin-2-one

Iodobenzene (5.53 g, 27.12 mmol) was added to a suspension of 6-bromoindolin-2-one (5 g, 23.58 mmol) in acetonitrile (ACN, 75 mL) under a nitrogen atmosphere. A steady stream of nitrogen was bubbled though the suspension as it was heated to 40° C. and stirred for 30 minutes. K₂CO₃ (7.17 g, 51.88 mmol), CuI (449.08 mg, 2.36 mmol), and N,N′-dimethylethane-1,2-diamine (415.72 mg, 4.72 mmol) were added and the reaction mixture was heated to 80° C. for 5 hours under a nitrogen atmosphere. After completion of the reaction, it was allowed to cool to rt, 1 M HCl (100 mL) was added, and the solution was extracted with EtOAc (100 mL×3). The combined organic extracts were dried over Na₂SO₄, and solvent was removed in vacuo. The residue was purified by silica gel chromatography (eluent: 0-50% ethyl acetate/petroleum ether) to afford the title compound (3.3 g, 49% yield) as an orange solid. MS [M+H]⁺=288.0.

### Phenyl-6-vinyl-indolin-2-one

To a solution of 6-bromo-1-phenyl-indolin-2-one (2.8 g, 9.72 mmol) in dioxane (32 mL) and H₂O (4 mL) was added potassium vinyl trifluoroborate (2.60 g, 19.44 mmol), Pd(dppf)Cl₂ (711.04 mg, 0.972 mmol), and K₂CO₃ (4.03 g, 29.15 mmol). The mixture stirred at 110° C. for 12 hours. After completion of the reaction, it was cooled to the rt, and poured into H₂O (40 mL) and extracted with EtOAc (40 mL×3). The combined organic layers were washed with brine (40 mL×2), dried over Na₂SO₄, filtered and concentrated under reduced pressure to give a crude residue. The residue was purified by MPLC (SiO₂, petroleum ether:EtOAc=10/1 to 1/1) to afford the title compound (1.5 g, 66% yield) as a red solid. ¹H NMR (400 MHz, CDCl₃) δ 7.54-7.42 (m, 2H), 7.40-7.28 (m, 3H), 7.23-7.13 (m, 1H), 7.08-6.97 (m, 1H), 6.79-6.69 (m, 1H), 6.63-6.48 (m, 1H), 5.63-5.49 (m, 1H), 5.14 (d, J=10.9 Hz, 1H), 3.63 (s, 2H).

### 2-Oxo-1-phenyl-indoline-6-carbaldehyde

Ozone was bubbled into a solution of 1-phenyl-6-vinyl-indolin-2-one (1.5 g, 6.38 mmol) in DCM (30 mL) at −78° C. for 30 minutes. After excess O₃ was purged by N₂, Me₂S (7.92 g, 127.51 mmol) was added at −78° C. The reaction was stirred at 20° C. for 12 hours. The reaction mixture was concentrated in vacuo to give the crude product. The residue was purified by MPLC (SiO₂, petroleum ether:EtOAc=10/1 to 1/1) to afford the title compound (370 mg, 25% yield) as a red solid. MS [M+H]⁺=238.1.

### 3-[1-Oxo-5-[1-[(2-oxo-1-phenyl-indolin-6-yl)methyl]-4-piperidyl]isoindolin-2-yl]piperidine-2,6-dione (18)

Compound 18 was prepared similarly as compound 12 in Example 6. ¹H NMR: (400 MHz, DMSO-d₆) δ 11.07-10.93 (m, 1H), 8.23 (s, 0.5H), 7.64-7.53 (m, 3H), 7.50-7.22 (m, 6H), 7.05-6.99 (m, 1H), 6.71-6.65 (m, 1H), 5.14-5.04 (m, 1H), 4.47-4.20 (m, 2H), 3.77-3.70 (m, 2H), 3.44 (br s, 2H), 2.89 (br d, J=11.9 Hz, 2H), 2.66-2.55 (m, 2H), 2.46-2.34 (m, 2H), 2.07-1.93 (m, 3H), 1.78-1.69 (m, 2H), 1.68-1.56 (m, 2H). MS [M+H]⁺=549.1.

### Example 8: Synthesis of 3-(1-oxo-5-(1-((4-oxo-3-(pyridin-2-yl)-3,4-dihydroquinazolin-6-yl)methyl)piperidin-4-yl)isoindolin-2-yl)piperidine-2,6-dione (31)

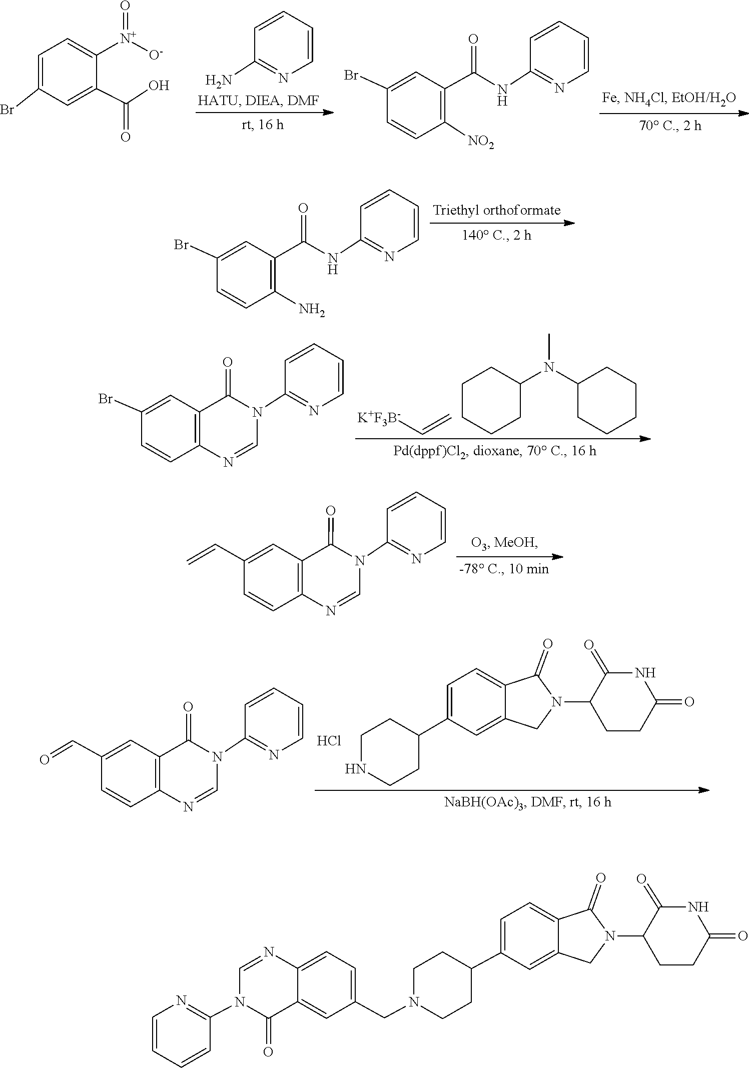

### 5-Bromo-2-nitro-N-(pyridin-2-yl)benzamide

To a solution of 5-bromo-2-nitrobenzoic acid (5 g, 20.32 mmol), pyridin-2-amine (1.91 g, 20.32 mmol), and N,N-diisopropylethylamine (7.88 g, 60.97 mmol) in 50 mL of DMF was added HATU (9.3 g, 24.38 mmol). The mixture stirred at rt for 16 hours. H₂O (200 mL) was added and the aqueous phase was extracted with DCM (2×200 mL). The organic phases were combined, concentrated and purified by silica gel column chromatography (methanol (MeOH):DCM=1/100 to 3/100) to give the title compound (3.6 g, 55%).

### 2-Amino-5-bromo-N-(pyridin-2-yl)benzamide

A solution of 5-bromo-2-nitro-N-(pyridin-2-yl)benzamide (2.60 g, 8.07 mmol), NH₄Cl (2.16 g, 40.36 mmol) in ethanol (EtOH)/H₂O (30 mL, v:v=7/3) was stirred at 50° C. under a N₂ atmosphere for 30 minutes. Fe (2.25 g, 40.36 mmol) was added and the reaction stirred for another 2 hours. The suspension was filtered to give a clear organic phase, which was concentrated and purified by silica gel column chromatography (MeOH:DCM=1/100 to 5/100) to give the title compound (0.8 g, 33.9%).

### 6-Bromo-3-(pyridin-2-yl)quinazolin-4(3H)-one

A solution of 2-amino-5-bromo-N-(pyridin-2-yl)benzamide (1.1 g, 3.77 mmol) in triethyl orthoformate (22 mL) stirred at 140° C. for 2 hours. The mixture was then concentrated to give a crude product that was purified by silica gel column chromatography (MeOH:DCM=1/100 to 3/100) to give the title compound (800 mg, 70%). ¹H NMR (400 MHz, DMSO-d₆) δ 8.69 (ddd, J=4.9, 1.9, 0.8 Hz, 1H), 8.64 (s, 1H), 8.33 (d, J=2.3 Hz, 1H), 8.11 (dd, J=8.0, 1.9 Hz, OH), 8.10-8.06 (m, 1H), 7.86 (dt, J=8.1, 0.9 Hz, 1H), 7.75 (d, J=8.7 Hz, 1H), 7.60 (ddd, J=7.5, 4.9, 1.0 Hz, 1H).

### 3-(Pyridin-2-yl)-6-vinylquinazolin-4(3H)-one

To a solution of 6-bromo-3-(pyridin-2-yl)quinazolin-4(3H)-one (300 mg, 1.33 mmol), potassium vinyl trifluoroborate (357.13 mg, 2.67 mmol), and N-cyclohexyl-N-methylcyclohexanamine (520.83 mg, 2.67 mmol) in 1,4-dioxane (6 mL) was added [1,1′-bis(diphenylphosphino)ferrocene]dichloropalladium (II) (97.1 mg, 0.13 mmol). The mixture stirred at 70° C. under a N₂ atmosphere for 16 hours. The mixture was then concentrated and purified by silica gel column chromatography (MeOH:DCM=1/100 to 3/100) to give the title compound (180 mg, 73%).

### 4-Oxo-3-(pyridin-2-yl)-3,4-dihydroquinazoline-6-carbaldehyde

A solution of 3-(pyridin-2-yl)-6-vinylquinazolin-4(3H)-one (150 mg, 0.6 mmol) in 6 mL of MeOH stirred at −78° C. for 5 minutes, then O₃ was bubbled through for 10 minutes. The mixture was then concentrated to give a crude product which was purified by prep-HPLC to give the title compound (60 mg, 40%). ¹H NMR (400 MHz, DMSO-d₆) δ 10.19 (s, 1H), 8.79 (d, J=1.8 Hz, 1H), 8.73 (s, 1H), 8.70-8.66 (m, 2H), 8.32 (dd, J=8.4, 1.9 Hz, 1H), 8.10 (td, J=7.8, 1.9 Hz, 1H), 7.92 (d, J=8.4 Hz, 1H), 7.87 (d, J=8.1 Hz, 1H), 7.60 (dd, J=7.0, 5.3 Hz, 1H).

### 3-(1-Oxo-5-(1-((4-oxo-3-(pyridin-2-yl)-3,4-dihydroquinazolin-6-yl)methyl)piperidin-4-yl)isoindolin-2-yl)piperidine-2,6-dione (31)

Compound 31 was prepared similarly as compound 12 in Example 6 with a yield of 13%. ¹H NMR (400 MHz, DMSO-d₆) δ 10.97 (s, 1H), 8.67 (dd, J=4.9, 1.1 Hz, 1H), 8.55 (s, 1H), 8.18 (d, J=1.7 Hz, 1H), 8.08 (td, J=7.8, 1.9 Hz, 1H), 7.88 (dd, J=8.3, 1.9 Hz, 1H), 7.84 (d, J=8.1 Hz, 1H), 7.75 (d, J=8.3 Hz, 1H), 7.64 (d, J=7.8 Hz, 1H), 7.57 (ddd, J=7.5, 4.9, 1.0 Hz, 1H), 7.51 (s, 1H), 7.41 (d, J=7.9 Hz, 1H), 5.09 (dd, J=13.3, 5.1 Hz, 1H), 4.42 (d, J=17.3 Hz, 1H), 4.28 (d, J=17.4 Hz, 1H), 3.70 (s, 2H), 2.97 (d, J=11.3 Hz, 3H), 2.94-2.82 (m, 1H), 2.70-2.54 (m, 1H), 2.46-2.29 (m, 1H), 2.16 (t, J=10.0 Hz, 2H), 2.05-1.93 (m, 1H), 1.82-1.69 (m, 3H). MS [M+H]⁺=563.0.

### Example 9: Synthesis of 3-[1-oxo-5-[1-[(2-phenylpyrazolo[1,5-a]pyridin-6-yl)methyl]-4-piperidyl]isoindolin-2-yl]piperidine-2,6-dione (38)

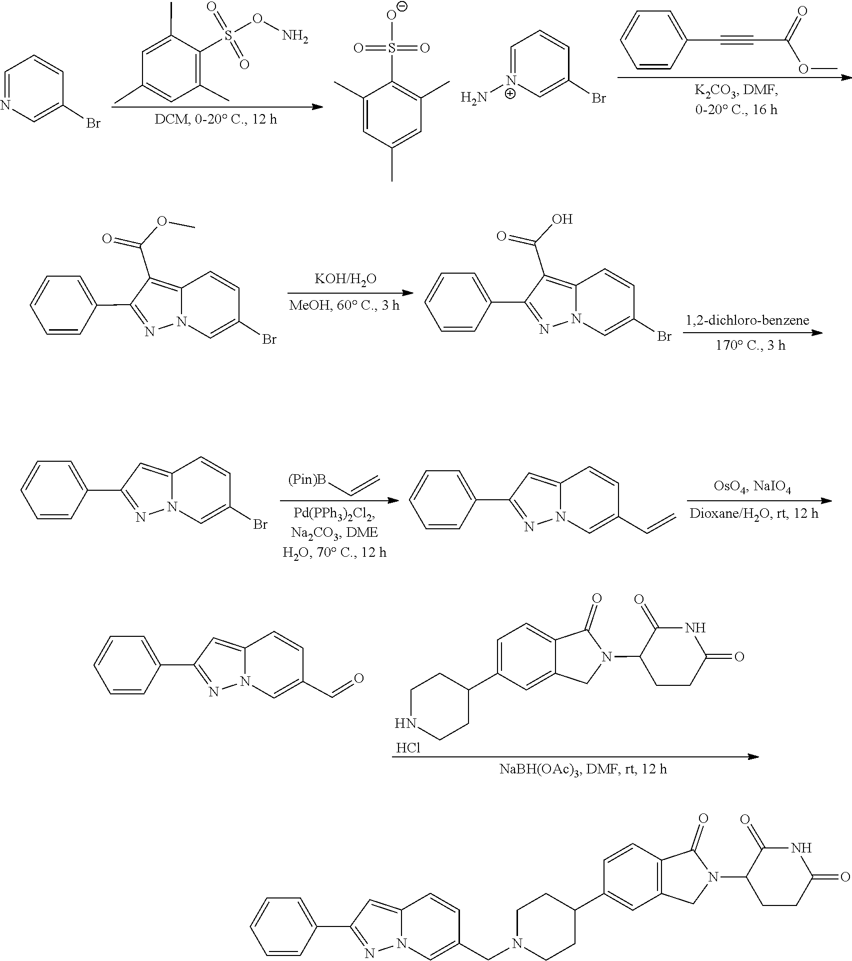

### 1-Amino pyridinium salt

To a mixture of amino 2,4,6-trimethylbenzenesulfonate (17.06 g, 79.27 mmol) in DCM (200 mL) at 0° C. was added dropwise a solution of 3-bromopyridine (12 g, 75.95 mmol) in DCM (150 mL). The mixture stirred at 20° C. for 12 hours. The white solid was collected by filtration and washed with EtOAc (100 mL). The filter cake was dried in vacuo to afford the title compound (23.3 g, 78.7%) as a white solid, which was used without further purification.

### Methyl 6-bromo-2-phenyl-pyrazolo[1,5-a]pyridine-3-carboxylate

To a solution of 1-amino pyridinium salt (23.30 g, 62.43 mmol) in dimethylformamide (DMF) (100 mL) was added K₂CO₃ (21.57 g, 156.09 mmol) and methyl 3-phenylprop-2-ynoate (6.25 g, 39.02 mmol) at 0° C. The mixture then stirred at 20° C. for 16 hours. To the reaction was added water (300 mL), and the aqueous phase was extracted with ethyl acetate (3×100 mL). The combined organic phases were dried over Na₂SO₄, filtered and concentrated in vacuo. The residue was purified by silica gel column chromatography (petroleum ether/ethyl acetate=15/1 to 4/1) to afford the title compound (5.2 g, 32%) as a light yellow solid.

### 6-Bromo-2-phenyl-pyrazolo[1,5-a]pyridine-3-carboxylic acid

To a solution of methyl 6-bromo-2-phenyl-pyrazolo[1,5-a]pyridine-3-carboxylate (5.2 g, 15.70 mmol) in H₂O (26 mL) and MeOH (50 mL) was added KOH (4.41 g, 78.51 mmol) in one portion. The mixture stirred at 60° C. for 3 hours. The mixture was concentrated in vacuo. The aqueous phase was acidified with 1N HCl to pH=3, and the white solid that formed was filtered and washed with water (20 mL). The filter cake was dried in vacuo to afford the title compound (4.6 g) as a white solid, which was used without further purification.

### 6-Bromo-2-phenyl-pyrazolo[1,5-a]pyridine

A suspension of 6-bromo-2-phenyl-pyrazolo[1,5-a]pyridine-3-carboxylic acid (4.6 g, 14.50 mmol) in 1,2-dichlorobenzene (30 mL) was degassed and purged with N₂ three times, and then the mixture stirred at 170° C. for 3 hours under N₂. The reaction was then cooled to rt, and purified by silica gel column chromatography (petroleum ether/ethyl acetate=1/0 to 10/1) to afford the title compound (2.1 g, 53%) as a white solid. ¹H NMR: (400 MHz, CDCl₃) δ 8.63 (d, J=0.4 Hz, 1H), 7.95 (d, J=7.2 Hz, 2H), 7.37-7.49 (m, 4H), 7.15-7.18 (m, 1H), 6.82 (s, 1H).

### 2-Phenyl-6-vinyl-pyrazolo[1,5-a]pyridine

To a solution of 6-bromo-2-phenyl-pyrazolo[1,5-a]pyridine (200 mg, 0.73 mmol) in dimethoxyethane (1.6 mL) and H₂O (0.8 mL) was added 4,4,5,5-tetramethyl-2-vinyl-1,3,2-dioxaborolane (169.17 mg, 1.10 mmol), Na₂CO₃ (256.12 mg, 2.42 mmol) and Pd(PPh₃)₂Cl₂ (51.40 mg, 73 μmol). The mixture stirred at 70° C. for 12 hours under N₂. The mixture was then diluted with water (15 mL), and the aqueous phase extracted with ethyl acetate (3×10 mL). The combined organic phases were dried over Na₂SO₄, filtered and concentrated in vacuo. The crude material was purified by silica gel column chromatography (hexane:EtOAc=4:1) to afford the title compound (130 mg, 81%) as a brown solid. ¹H NMR: (400 MHz, CDCl₃) δ 8.42 (s, 1H), 7.97 (d, J=7.2 Hz, 2H), 7.44-7.51 (m, 3H), 7.36-7.40 (m, 1H), 7.33-7.34 (m, 1H), 6.79 (s, 1H), 6.64-6.72 (m, 1H), 5.76 (d, J=17.6 Hz, 1H), 5.33 (d, J=11.2 Hz, 1H).

### 2-Phenylpyrazolo[1,5-a]pyridine-6-carbaldehyde

A solution of 2-phenyl-6-vinyl-pyrazolo[1,5-a]pyridine (730 mg, 3.31 mmol) in dioxane (20 mL) and H₂O (10 mL) was treated with NaIO₄ (1.77 g, 8.29 mmol) and OsO₄ (42.13 mg, 166 μmol) at 20° C. for 12 hours. The mixture was quenched with sat. Na₂SO₃ (20 mL) and the aqueous phase was extracted with EtOAc (2×10 mL). The combined organic phases were dried with Na₂SO₄, filtered and concentrated. The residue was purified by silica gel column chromatography (petroleum ether/ethyl acetate=I/O to 0/1) to afford the title compound (200 mg, 27%) as a yellow solid.

### 3-[1-Oxo-5-[1-[(2-phenylpyrazolo[1,5-a]pyridin-6-yl)methyl]-4-piperidyl]isoindolin-2-yl]piperidine-2,6-dione (38)

Compound 38 was prepared similarly as compound 12 in Example 6 with a yield of 18% as a white solid. ¹H NMR (400 MHz, DMSO-d₆) δ 11.00 (s, 1H), 10.71 (s, 1H), 8.67 (s, 1H), 9.00 (s, 1H), 8.01 (d, J=7.2 Hz, 2H), 7.79 (d, J=8.8 Hz, 1H), 7.70 (d, J=8.0 Hz, 1H), 7.45-7.52 (m, 4H), 7.37-7.43 (m, 2H), 7.16 (s, 1H), 5.08-5.13 (m, 1H), 4.47 (s, 1H), 4.40-4.43 (m, 2H), 4.28-4.33 (m, 1H), 3.06-3.15 (m, 3H), 2.86-3.01 (m, 3H), 2.35-2.40 (m, 1H), 1.69-2.12 (m, 6H). MS [M+H]⁺=534.3.

### Example 10: Synthesis of 3-[1-oxo-5-[1-[[4-oxo-3-(2-pyridyl)phthalazin-6-yl]methyl]-4-piperidyl]isoindolin-2-yl]piperidine-2,6-dione (43)

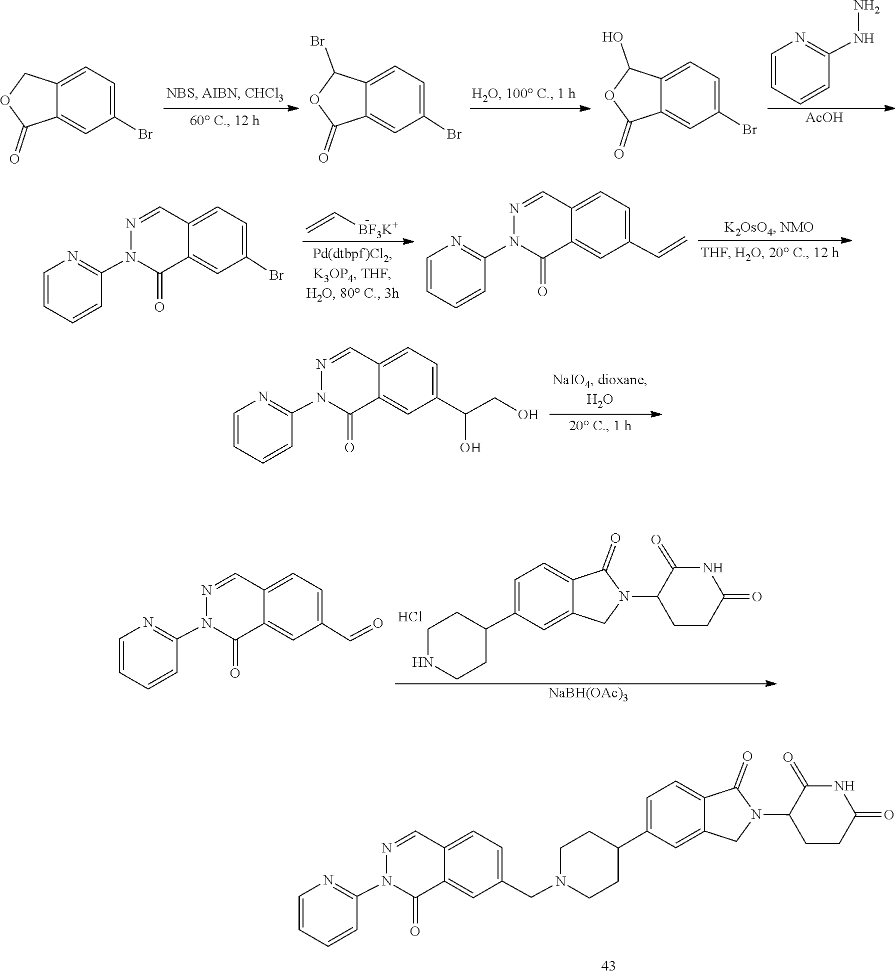

### 3,6-Dibromo-3H-isobenzofuran-1-one

To a solution of 6-bromo-3H-isobenzofuran-1-one (5.7 g, 26.76 mmol) in CHCl₃ (60 mL) was added NBS (5.24 g, 29.46 mmol) and AIBN (439.37 mg, 2.68 mmol). The mixture stirred at 80° C. for 2 hours. The reaction was then filtered and the filtrate was concentrated in vacuo to afford the title compound (10 g) as a white solid, which was used without further purification. ¹H NMR (400 MHz, DMSO-d₆) δ 7.92-7.90 (m, 1H), 7.56 (d, J=7.9 Hz, 1H), 6.58 (s, 1H).

### 6-Bromo-3-hydroxy-3H-isobenzofuran-1-one

A suspension of 3,6-dibromo-3H-isobenzofuran-1-one (5.7 g, 19.53 mmol) in H₂O (30 mL) was degassed and further purged with N₂ three times, and then the mixture stirred at 100° C. for 1 hour under N₂ atmosphere. The reaction mixture was poured into H₂O (20 mL), and the aqueous phase was extracted with EtOAc (3×50 mL). The combined organic layers were washed with brine (2×50 mL), dried over Na₂SO₄, filtered and concentrated in vacuo to afford the title compound (6.87 g) as a white solid, which was used without further purification. ¹H NMR (400 MHz, DMSO-d₆) δ 8.32 (br s, 1H), 8.11-7.98 (m, 3H), 7.70 (d, J=8.0 Hz, 1H), 6.71 (br s, 1H).

### 7-Bromo-2-(2-pyridyl)phthalazin-1-one

To a solution of 6-bromo-3-hydroxy-3H-isobenzofuran-1-one (1 g, 4.37 mmol) in AcOH (30 mL) was added 2-pyridylhydrazine (476.49 mg, 4.37 mmol). The reaction stirred at 100° C. for 12 hours, then concentrated in vacuo to give a crude residue which was suspended in H₂O (50 mL) and EtOAc (50 mL). The aqueous phase was extracted with EtOAc (2×50 mL). The combined organic layers were washed with brine (2×50 mL), dried over Na₂SO₄, filtered and concentrated in vacuo to afford the title compound (0.94 g) as a yellow solid, which was used without further purification. ¹H NMR (400 MHz, DMSO-d₆) δ 8.62-8.58 (m, 1H), 8.57 (s, 1H), 8.38 (d, J=2.0 Hz, 1H), 8.19 (dd, J=2.0, 8.3 Hz, 1H), 8.05-7.95 (m, 2H), 7.67-7.60 (m, 1H), 7.55-7.48 (m, 1H).

### 2-(2-Pyridyl)-7-vinyl-phthalazin-1-one

A mixture of 7-bromo-2-(2-pyridyl)phthalazin-1-one (0.84 g, 2.78 mmol), potassium vinyltrifluoroborate (558.63 mg, 4.17 mmol), di-tert-butyl(cyclopentyl)phosphane dichloropalladium iron (181.20 mg, 278 μmol) and K₃PO₄ (1.18 g, 5.56 mmol) in THE (16 mL) and H₂O (4 mL) was degassed and purged with N₂ three times. The reaction stirred at 80° C. for 1 hour under N₂ atmosphere. The reaction mixture was poured into H₂O (50 mL), and extracted with EtOAc (3×50 mL). The combined organic layers were washed with brine (2×50 mL), dried over Na₂SO₄, filtered and concentrated in vacuo to give a crude residue. The residue was purified by silica gel column chromatography (petroleum ether/ethyl acetate=10/1 to 1/1) to afford the title compound (0.5 g, 72%) as a yellow solid. ¹H NMR (400 MHz, DMSO-d₆) δ 8.63 (dd, J=1.1, 4.8 Hz, 1H), 8.54 (s, 1H), 8.31 (s, 1H), 8.19 (dd, J=1.6, 8.2 Hz, 1H), 8.07-7.98 (m, 2H), 7.65 (d, J=8.0 Hz, 1H), 7.53 (dd, J=4.9, 7.3 Hz, 1H), 7.02 (dd, J=11.0, 17.6 Hz, 1H), 6.15 (d, J=17.6 Hz, 1H), 5.53 (d, J=11.0 Hz, 1H). MS [M+H]⁺=250.2.

### 7-(1,2-Dihydroxyethyl)-2-(2-pyridyl)phthalazin-1-one

To a solution of 2-(2-pyridyl)-7-vinyl-phthalazin-1-one (0.3 g, 1.20 mmol) in THE (3 mL) and H₂O (0.3 mL) was added K₂OsO₄·2H₂O (44.35 mg, 120 μmol) and NMO (422.98 mg, 3.61 mmol). The mixture stirred at 20° C. for 12 hours. The reaction was quenched with aqueous saturated Na₂SO₃ (20 mL) and the aqueous phase was extracted with EtOAc (2×10 mL). The combined organic phases were dried with Na₂SO₄, filtered and concentrated to afford the title compound (0.27 g) as yellow liquid, which was used without further purification.

### 4-Oxo-3-(2-pyridyl)phthalazine-6-carbaldehyde

To a solution of 7-(1,2-dihydroxyethyl)-2-(2-pyridyl)phthalazin-1-one (0.27 g, 0.95 mmol) in dioxane (4 mL) and H₂O (0.4 mL) was added NaIO₄ (407.72 mg, 1.91 mmol). The mixture stirred at 20° C. for 2 hours. The reaction mixture was poured into H₂O (10 mL) and extracted with EtOAc (3×10 mL). The combined organic layers were washed with brine (2×10 mL), dried over Na₂SO₄, filtered and concentrated in vacuo to give a crude residue. The residue was purified by silica gel column chromatography (petroleum ether/ethyl acetate=10/1 to 1/1) to afford the title compound (0.05 g, 21%) as a yellow solid. ¹H NMR (400 MHz, DMSO-d₆) δ 10.25 (s, 1H), 8.83 (s, 1H), 8.68 (s, 1H), 8.64 (dd, J=1.1, 4.7 Hz, 1H), 8.66-8.60 (m, 1H), 8.41 (dd, J=1.4, 8.1 Hz, 1H), 8.21 (d, J=8.1 Hz, 1H), 8.06 (dt, J=1.9, 7.8 Hz, 1H), 7.69 (d, J=7.9 Hz, 1H), 7.55 (dd, J=5.0, 6.8 Hz, 1H). MS [M+H]⁺=252.1.

### 3-[1-Oxo-5-[1-[[4-oxo-3-(2-pyridyl)phthalazin-6-yl]methyl]-4-piperidyl]isoindolin-2-yl]piperidine-2,6-dione (43)

Compound 43 was prepared similarly as compound 12 in Example 6 with a yield of 27%. ¹H NMR (400 MHz, DMSO-d₆) δ 11.18 (br s, 1H), 10.98 (s, 1H), 8.66-8.59 (m, 2H), 8.57 (s, 1H), 8.36 (dd, J=1.5, 8.1 Hz, 1H), 8.15 (d, J=8.1 Hz, 1H), 8.06 (dt, J=1.9, 7.8 Hz, 1H), 7.68 (dd, J=7.9, 13.1 Hz, 2H), 7.58-7.52 (m, 1H), 7.45 (s, 1H), 7.39 (d, J=7.9 Hz, 1H), 5.10 (dd, J=5.0, 13.3 Hz, 1H), 4.61 (br d, J=4.8 Hz, 2H), 4.49-4.27 (m, 2H), 3.48 (br d, J=11.4 Hz, 2H), 3.21-3.06 (m, 2H), 3.00-2.85 (m, 2H), 2.59 (br d, J=16.9 Hz, 1H), 2.44-2.34 (m, 1H), 2.15 (br d, J=12.9 Hz, 2H), 2.00 (br d, J=11.1 Hz, 3H). MS [M+H]⁺=563.1.

### Example 11: Synthesis of 3-(4-fluoro-1-oxo-5-(piperidin-4-yl)isoindolin-2-yl)piperidine-2,6-dione hydrochloride

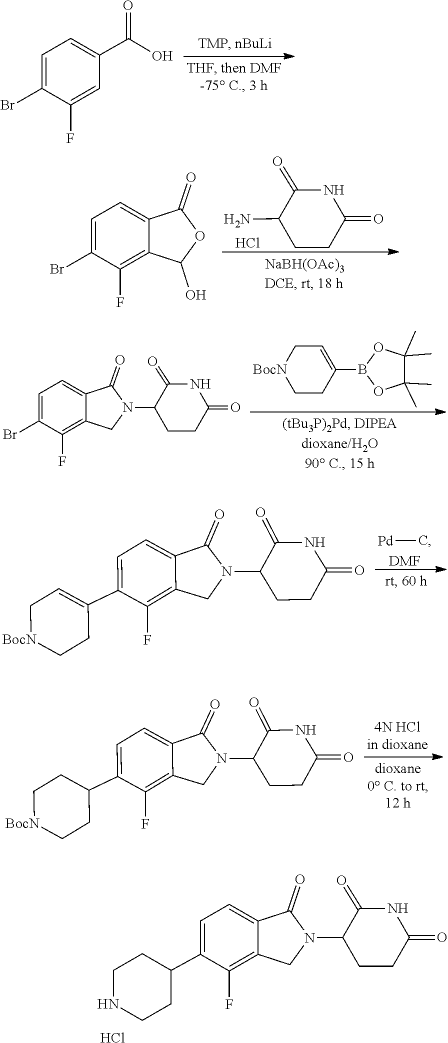

### 5-Bromo-4-fluoro-3-hydroxyisobenzofuran-1(3H)-one

To a solution of 2,2,6,6-tetramethylpiperidine (48.4 g, 342 mmol) in THF (150 mL) was added n-BuLi (2 M in cyclohexane, 137 mL, 274 mmol) dropwise at −75° C. and the mixture was warmed to 0° C. where it stirred for 20 min. The reaction mixture was then cooled to −75° C. and a solution of 4-bromo-3-fluorobenzoic acid (15.0 g, 68.5 mmol) in THF (30 mL) was added dropwise. The reaction mixture was stirred for 40 min, then DMF (10.0 g, 137 mmol) in THF (15 mL) was added dropwise at −75° C. and the resulting mixture was stirred for an additional 2 h. The reaction mixture was quenched with 5 M HCl (10 mL) and was diluted with brine solution (50 mL). The resulting mixture was extracted with DCM (2×100 mL) and the combined organic extracts were dried over anhydrous Na₂SO₄ and filtered. The solvent was removed under reduced pressure and the crude material was purified by silica gel column chromatography using EtOAc in hexanes (60-80%) as an eluent to afford the title compound as an off white solid (14.0 g, 71%). MS [M−H]⁺=244.8.

### 3-(5-Bromo-4-fluoro-1-oxoisoindolin-2-yl) piperidine-2,6-dione

To a solution of 5-bromo-4-fluoro-3-hydroxyisobenzofuran-1(3H)-one (2.00 g, 8.10 mmol) in 1,2-dichloroethane (DCE, 20 mL) was added 3-aminopiperidine-2,6-dione hydrochloride (2.00 g, 12.1 mmol) at 25° C. under N₂ atmosphere. The reaction mixture was stirred for 30 min, then sodium triacetoxyborohydride (5.15 g, 24.3 mmol) was added and the resulting mixture was stirred for 18 h at rt. The reaction mixture was quenched with brine (15 mL) and the aqueous layer was extracted with EtOAc (2×100 mL). The combined organic extracts were dried over anhydrous Na₂SO₄ and filtered. The solvent was removed under reduced pressure to afford a crude residue that was dissolved in ACN (5 mL)/methyl tert-butyl ether (MTBE, 5 mL) and stirred for 10 min to afford the title compound as a pale blue solid which was isolated by filtration, washed with excess MTBE, and dried by vacuum (1.32 g, 48%). ¹H NMR (400 MHz, DMSO-d₆) δ 11.20-10.76 (m, 1H), 7.88 (dd, J=6.1, 7.9 Hz, 1H), 7.55 (d, J=8.0 Hz, 1H), 5.13 (dd, J=5.1, 13.4 Hz, 1H), 4.67-4.59 (m, 1H), 4.50-4.43 (m, 1H), 2.98-2.86 (m, 1H), 2.66-2.56 (m, 1H), 2.44 (dd, J=4.4, 12.9 Hz, 1H), 2.01 (dtd, J=2.3, 5.2, 12.7 Hz, 1H). MS [M+H]⁺=340.9.

### tert-Butyl 4-(2-(2,6-dioxopiperidin-3-yl)-4-fluoro-1-oxoisoindolin-5-yl)-3,6-dihydropyridine-1(2H)-carboxylate

To a stirred solution of 3-(5-bromo-4-fluoro-1-oxoisoindolin-2-yl)piperidine-2,6-dione (1.32 g, 3.87 mmol) in dioxane (20 mL)/water (2.2 mL) at rt were added tert-butyl 4-(4,4,5,5-tetramethyl-1,3,2-dioxaborolan-2-yl)-3,6-dihydropyridine-1(2H)-carboxylate (2.39 g, 7.74 mmol), N,N-diisopropylethylamine (DIPEA, 1.00 g, 7.74 mmol) and Pd(tBu₃P)₂ (0.198 g, 0.387 mmol) under continuous N₂ bubbling for 20 min. The reaction mixture was stirred at 100° C. for 15 h. Excess solvent was removed under reduced pressure to afford a crude residue that was dissolved in ACN (5 mL)/MTBE (5 mL) and stirred for 10 min to afford the title compound as a white solid which was isolated by filtration, washed with excess MTBE, and dried by vacuum (1.30 g, 72%). ¹H NMR (400 MHz, DMSO-d₆) δ 11.01 (s, 1H), 7.80-7.35 (m, 2H), 6.11 (brs, 1H), 5.12 (dd, J=5.1, 13.3 Hz, 1H), 4.62-4.49 (m, 1H), 4.46-4.25 (m, 1H), 4.03 (brs, 2H), 3.56 (t, J=5.5 Hz, 2H), 2.98-2.81 (m, 1H), 2.73-2.56 (m, 1H), 2.49-2.41 (m, 2H), 2.4 (m, 1H), 2.08-1.89 (m, 1H), 1.44 (s, 9H). MS [M+H]⁺=444.2.

### tert-Butyl 4-(2-(2,6-dioxopiperidin-3-yl)-4-fluoro-1-oxoisoindolin-5-yl)piperidine-1-carboxylate

To a solution of tert-butyl 4-(2-(2,6-dioxopiperidin-3-yl)-4-fluoro-1-oxoisoindolin-5-yl)-3,6-dihydropyridine-1(2H)-carboxylate (0.500 g, 1.13 mmol) in DMF (10 mL) was added Pd/C (10-50% wet, 0.120 g, 0.113 mmol) at rt under N₂ atmosphere and the mixture was stirred for 60 h under a hydrogen atmosphere. The reaction mixture was filtered through Celite® and was washed with THE (50 mL×2). The solvent was removed under reduced pressure to afford a crude residue that was dissolved in ACN (5 mL)/MTBE (5 mL) and stirred for 10 min to afford the title compound as a white solid which was isolated by filtration, washed with excess MTBE, and dried by vacuum (0.380 g, 73%). ¹H NMR (400 MHz, DMSO-d₆) δ 11.00 (s, 1H), 7.55-7.50 (m, 2H), 5.14-5.08 (m, 1H), 4.55 (d, J=17.4 Hz, 1H), 4.42-4.34 (m, 1H), 4.10 (d, J=11.0 Hz, 2H), 3.16-3.06 (m, 1H), 2.98-2.78 (m, 3H), 2.66-2.55 (m, 1H), 2.44 (dd, J=4.6, 13.1 Hz, 1H), 2.05-1.95 (m, 1H), 1.81-1.69 (m, 2H), 1.67-1.54 (m, 2H), 1.43 (s, 9H). MS [M−H]⁺=444.

### 3-(4-Fluoro-1-oxo-5-(piperidin-4-yl) isoindolin-2-yl) piperidine-2,6-dione hydrochloride

To a solution of tert-butyl 4-(2-(2,6-dioxopiperidin-3-yl)-4-fluoro-1-oxoisoindolin-5-yl)piperidine-1-carboxylate (0.45 g, 1.0 mmol) in 1,4-dioxane (2 mL) was added HCl (4M in 1,4-dioxane, 5.0 mL, 20 mmol) at 0° C. and the mixture was stirred for 10 min, then warmed to rt for 12 h. The reaction mixture was concentrated under reduced pressure to afford a crude residue that was dissolved in ACN (5 mL)/MTBE (5 mL) and stirred for 10 min to afford the title compound as an off-white solid which was isolated by filtration, washed with excess MTBE, and dried by vacuum (0.32 g, 91%). ¹H NMR (400 MHz, DMSO-d₆) δ 11.35-10.61 (m, 1H), 8.37 (s, 1H), 7.59 (d, J=7.9 Hz, 1H), 7.52-7.45 (m, 1H), 5.16-5.07 (m, 1H), 4.61-4.51 (m, 1H), 4.43-4.33 (m, 1H), 3.19 (brs, 3H), 2.83 (brs, 3H), 2.64-2.58 (m, 1H), 2.47-2.37 (m, 2H), 2.05-1.94 (m, 1H), 1.84-1.74 (m, 3H). MS [M+H]⁺=346.

### Example 12: Synthesis of 6-methyl-3-(6-(trifluoromethyl)pyridin-2-yl)quinazolin-4(3H)-one

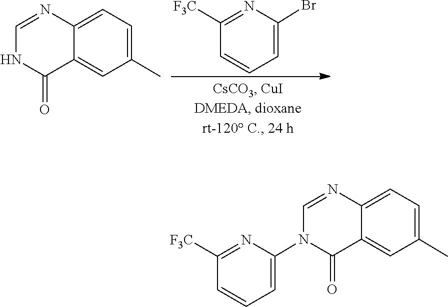 [SMILES: Cc1ccc2nc[nH]c(=O)c2c1 ; FC(F)(F)c1cccc(Br)n1 ; Cc1ccc2ncn(-c3cccc(C(F)(F)F)n3)c(=O)c2c1]

To a stirred solution of 6-methylquinazolin-4(3H)-one (0.500 g, 3.12 mmol) in 1,4-dioxane (10 mL) at 25° C. was added 2-bromo-6-(trifluoromethyl)pyridine (1.13 g, 4.99 mmol), cesium carbonate (3.05 g, 9.36 mmol) and 1,2-dimethylethylenediamine (DMEDA, 0.549 g, 6.24 mmol). The reaction mixture was degassed with N₂ for 10 min before copper(I) iodide (0.297 g, 1.56 mmol) was added at rt and the reaction mixture was heated at 120° C. for 24 h. The reaction mixture was cooled to rt and the volatiles were evaporated under reduced pressure. The crude material was purified by reverse phase chromatography purification using 10 mM ammonium acetate in water and ACN followed by lyophilization to afford the title compound as an off-white solid (130 mg, 33%). ¹H NMR (400 MHz, DMSO-d₆) δ 8.53 (s, 1H), 8.38 (t, J=7.9 Hz, 1H), 8.23-7.99 (m, 3H), 7.77-7.58 (m, 2H), 2.51 (br s, 3H). MS [M+H]⁺=306.

### Example 13: Synthesis of 3-(6-methyl-4-oxoquinazolin-3(4H)-yl)benzonitrile

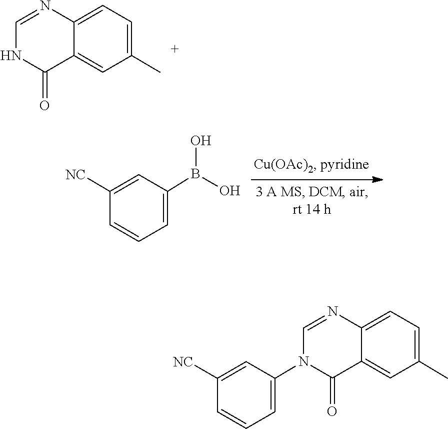 [SMILES: Cc1ccc2nc[nH]c(=O)c2c1 ; N#Cc1cccc(B(O)O)c1 ; Cc1ccc2ncn(-c3cccc(C#N)c3)c(=O)c2c1]

To a solution of 6-methylquinazolin-4(3H)-one (1.00 g, 6.24 mmol) in DCM (20 mL) were added 3 Å molecular sieves (2.00 g, 6.24 mmol), (3-cyanophenyl)boronic acid (1.84 g, 12.5 mmol), pyridine (1.01 ml, 12.5 mmol) and copper (II) acetate (1.13 g, 6.24 mmol) at 25° C. The reaction mixture was stirred at rt under a balloon filled with air for 14 h. The reaction mixture was filtered through a pad of Celite® and was washed with ethyl acetate (50 mL). The filtrate was evaporated under reduced pressure to give a solid (1.5 g) which was purified by silica gel column chromatography using ethyl acetate-hexanes to afford the title compound as an off-white solid (0.300 g, 17%). ¹H NMR (400 MHz, DMSO-d₆) δ 8.44-8.30 (m, 1H), 8.15 (t, J=1.7 Hz, 1H), 8.05-7.99 (m, 2H), 7.95 (ddd, J=1.1, 2.1, 8.1 Hz, 1H), 7.85-7.71 (m, 2H), 7.70-7.62 (m, 1H), 2.53 (d, J=2.0 Hz, 3H). MS [M+H]⁺=262.1.

### Example 14: Synthesis of 5-fluoro-6-methyl-3-(pyridin-2-yl)quinazolin-4(3H)-one

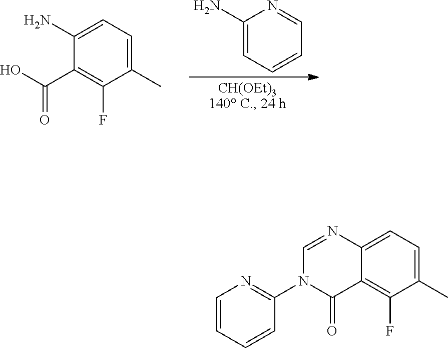

To the solution of 6-amino-2-fluoro-3-methylbenzoic acid (0.500 g, 2.96 mmol) in triethyl orthoformate (5 mL) was added pyridin-2-amine (0.278 g, 2.96 mmol) at rt. The resulting reaction mixture was stirred at 140° C. for 24 h. The reaction mixture was concentrated under reduced pressure to give a crude material which was purified by silica gel column chromatography using EtOAc in hexane (30%) as an eluent to afford the title compound as an off-white solid (0.150 g, 19%). MS [M+H]⁺=256.4.

### Example 15: Synthesis of 3-(1-oxo-5-(1-((4-oxo-3-(6-(trifluoromethyl)pyridin-2-yl)-3,4-dihydroquinazolin-6-yl)methyl)piperidin-4-yl)isoindolin-2-yl)piperidine-2,6-dione (64)

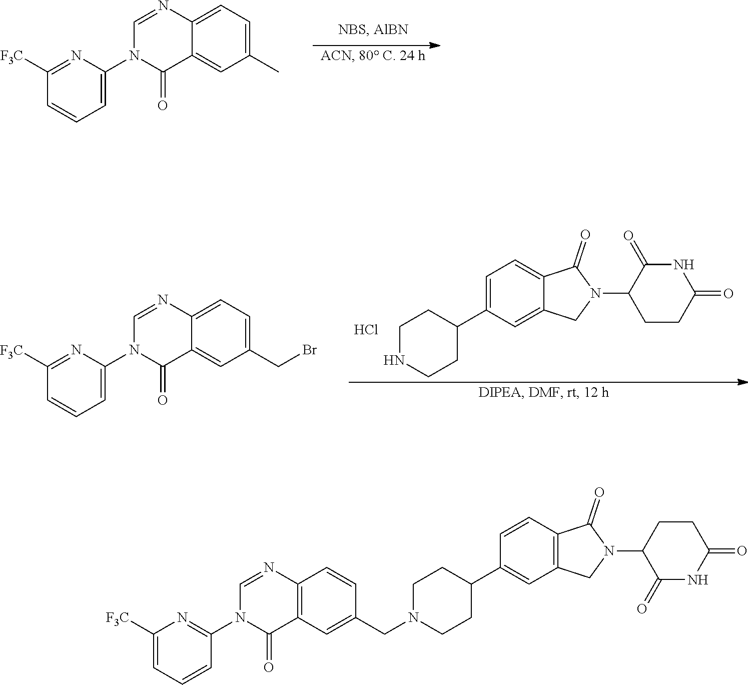

### 6-(Bromomethyl)-3-(6-(trifluoromethyl) pyridin-2-yl) quinazolin-4(3H)-one

To a stirred solution of 6-methyl-3-(6-(trifluoromethyl)pyridin-2-yl)quinazolin-4(3H)-one (0.250 g, 0.819 mmol) in ACN (10 mL) were added N-bromosuccinimide (NBS, 0.292 g, 1.64 mmol) and azobisisobutyronitrile (AIBN, 0.067 g, 0.41 mmol) at 25° C. The reaction mixture was heated at reflux for 24 h. The reaction mixture was evaporated under reduced pressure to give crude material which was purified by silica gel column chromatography using 15% ethyl acetate in hexanes to afford the title compound as a pale yellow solid (100 mg, 25%). MS [M+H]⁺=384.2.

### 3-(1-Oxo-5-(1-((4-oxo-3-(6-(trifluoromethyl)pyridin-2-yl)-3,4-dihydroquinazolin-6-yl)methyl)piperidin-4-yl)isoindolin-2-yl)piperidine-2,6-dione

To a solution of 3-(1-oxo-5-(piperidin-4-yl)isoindolin-2-yl)piperidine-2,6-dione hydrochloride (90.0 mg, 0.246 mmol) in DMF (2 mL) was added DIPEA (0.209 mL, 1.23 mmol) at 0° C. and the mixture was stirred for 10 min. 6-(Bromomethyl)-3-(6-(trifluoromethyl) pyridin-2-yl) quinazolin-4(3H)-one (95.0 mg, 0.246 mmol) was added, and reaction mixture was stirred at rt for 12 h. The reaction mixture was evaporated under reduced pressure to give a crude solid that was purified by prep-HPLC [Method info: column: X select (150 mm*19) 5 μm, 0.1% HCOOH H₂O:ACN, Flow rate: 15 mL/min]. The pure fractions were lyophilized to afford the title compound as an off-white solid (5.8 mg, 4%). ¹H NMR (400 MHz, DMSO-d₆) δ 10.98 (s, 1H), 8.59 (brs, 1H), 8.46-8.34 (m, 1H), 8.19 (d, J=8.1 Hz, 1H), 8.15-8.09 (m, 1H), 8.01-7.74 (m, 2H), 7.66 (d, J=7.8 Hz, 1H), 7.56-7.36 (m, 2H), 6.53 (s, 1H), 5.11 (dd, J=5.1, 13.3 Hz, 1H), 4.49-4.38 (m, 1H), 4.33-4.24 (m, 1H), 3.92-3.64 (m, 1H), 3.17-2.84 (m, 3H), 2.71 (m, 1H), 2.66-2.56 (m, 3H), 2.39 (dd, J=4.3, 12.9 Hz, 2H), 2.14 (m, 1H), 2.06-1.95 (m, 1H), 1.91-1.74 (m, 3H). MS [M+H]⁺=631.1.

### Example 16: Synthesis of 6-bromo-3-(pyridin-3-yl)quinazolin-4(3H)-one

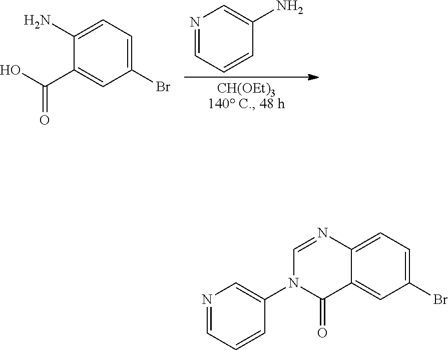

A solution of 2-amino-5-bromobenzoic acid (10.0 g, 46.3 mmol) and pyridin-3-amine (4.36 g, 46.3 mmol) in triethyl orthoformate (100 mL, 600 mmol) was heated at 140° C. for 48 h. The reaction mixture was concentrated under reduced pressure to give a crude solid. The crude solid was washed with 2-propanol and the solids were filtered to afford the title compound as a pale brown solid (9.00 g, 52%). ¹H NMR (400 MHz, DMSO-d₆) δ 8.78 (d, J=2.0 Hz, 1H), 8.72 (dd, J=1.6, 3.6 Hz, 1H), 8.48 (s, 1H), 8.30 (d, J=2.4 Hz, 1H), 8.08-8.04 (m, 2H), 7.74 (d, J=8.8 Hz, 1H), 8.66-8.63 (m, 1H). MS [M+H, M+2H]⁺=302.0, 304.0.

### Example 17: Synthesis of 6-bromo-2-methyl-3-(pyridin-2-yl)quinazolin-4(3H)-one

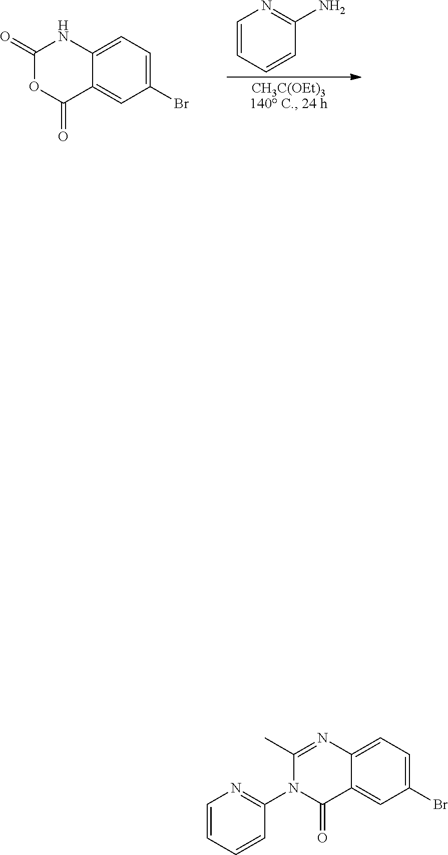

A mixture of 6-bromo-2H-benzo[d][1,3]oxazine-2,4(1H)-dione (3.00 g, 12.4 mmol), 2-aminopyridine (1.28 g, 13.6 mmol) and triethyl orthoacetate (3.02 g, 18.6 mmol) under N₂ was stirred at 140° C. for 24 h. The reaction mixture was cooled to rt before it was concentrated under reduced pressure to give a crude residue. The crude residue was purified by silica gel column chromatography using 50-90% EtOAc in hexanes as an eluent to afford the title compound as a light yellow solid (1.80 g, 46%). ¹H NMR (400 MHz, DMSO-d₆) δ 8.76-8.64 (m, 1H), 8.20 (d, J=2.3 Hz, 1H), 8.12 (dt, J=1.9, 7.7 Hz, 1H), 8.03 (dd, J=2.4, 8.6 Hz, 1H), 7.71-7.60 (m, 3H), 2.11 (s, 3H). MS [M+H, M+2H]⁺=316.2, 318.2.

### Example 18: Synthesis of 3-(1-oxo-5-(1-((4-oxo-3-(pyridin-3-yl)-3,4-dihydroquinazolin-6-yl)methyl)piperidin-4-yl)isoindolin-2-yl)piperidine-2,6-dione (67)

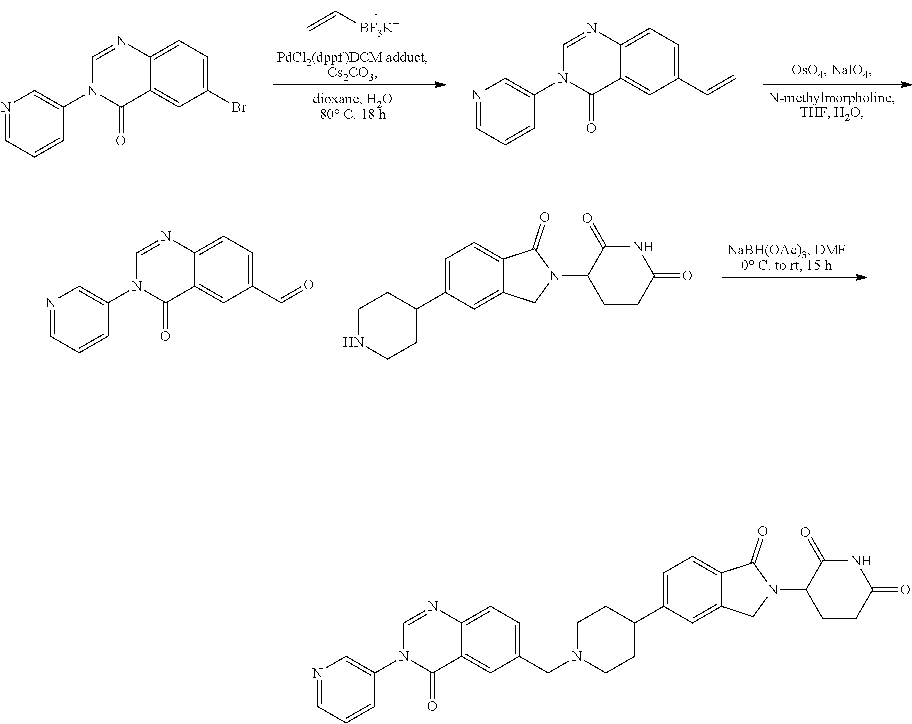

### 3-(Pyridin-3-yl)-6-vinylquinazolin-4(3H)-one

To a solution of 6-bromo-3-(pyridin-3-yl)quinazolin-4(3H)-one (700 mg, 2.32 mmol) in 1,4-dioxane (8 mL) were added potassium vinyltrifluoroborate (310 mg, 2.32 mmol) followed by a solution of cesium carbonate (2 M in water, 1.16 mL, 2.32 mmol) and PdCl₂(dppf)₂-CH₂Cl₂ adduct (1.89 g, 2.32 mmol) at rt under continuous N₂ bubbling. The reaction mixture was stirred at 85° C. for 18 h. Ethyl acetate (50 mL) was added to reaction mixture and the mixture was filtered through a pad of Celite®. The filtrate was concentrated under reduced pressure to obtain a crude product that was purified by silica gel column chromatography using ethyl acetate and hexane as eluents to afford the title compound as a pale yellow solid (475 mg, 71%). ¹H NMR (400 MHz, DMSO-d₆) δ 8.79 (d, J=2.0 Hz, 1H), 8.71 (dd, J=1.2, 4.8 Hz, 1H), 8.41 (s, 1H), 8.16 (d, J=2.0 Hz, 1H), 8.11-8.05 (m, 2H), 7.75 (d, J=8.4 Hz, 1H), 8.66-8.63 (m, 1H), 6.96 (dd, J=11.0, 17.6 Hz, 1H), 6.02 (d, J=17.6 Hz, 1H), 5.42 (d, J=11.0 Hz, 1H). MS [M+H]⁺=250.3.

### 4-Oxo-3-(pyridin-3-yl)-3,4-dihydroquinazoline-6-carbaldehyde

To a solution of 3-(pyridin-3-yl)-6-vinylquinazolin-4(3H)-one (475 mg, 1.91 mmol) in 1,4-dioxane (7 mL) and water (0.2 mL) at 0° C. were added sodium periodate (815 mg, 3.81 mmol) and 4-methylmorpholine (0.105 mL, 0.953 mmol) followed by a dropwise addition of osmium(VIII) oxide (4 wt. % in water, 1.50 mL, 0.191 mmol) at 0° C. The reaction mixture was warmed to 25° C. and stirred for 3 h. During progress of the reaction, a large amount of solid formation was observed. The reaction mixture was filtered and the solid residue was washed with ethyl acetate. The filtrate was concentrated under reduced pressure to give the crude residue that was purified by silica gel column chromatography using ethyl acetate in hexane (45-95%) as an eluent to afford the title compound as a light yellow solid (225 mg, 45%). ¹H NMR (400 MHz, DMSO-d₆) δ 10.19 (s, 1H), 8.82 (d, J=2.0 Hz, 1H), 8.78 (d, J=2.0 Hz, 1H), 8.73 (dd, J=1.6, 4.8 Hz, 1H), 8.59 (s, 1H), 8.32 (dd, J=2.0, 4.4 Hz, 1H), 8.11-8.07 (m, 1H), 7.92 (d, J=8.4 Hz, 1H), 6.68-6.65 (m, 1H). MS [M+H]⁺=252.1.

### 3-(1-Oxo-5-(1-((4-oxo-3-(pyridin-3-yl)-3,4-dihydroquinazolin-6-yl)methyl)piperidin-4-yl)isoindolin-2-yl)piperidine-2,6-dione

A solution 4-oxo-3-(pyridin-3-yl)-3,4-dihydroquinazoline-6-carbaldehyde (121 mg, 0.481 mmol) and 3-(1-oxo-5-(piperidin-4-yl)isoindolin-2-yl)piperidine-2,6-dione, HCl (175 mg, 0.481 mmol) in DMF (3 mL) was stirred for 15 min at rt. To this reaction mixture was added sodium triacetoxyhydroborate (255 mg, 1.20 mmol) at rt under N₂ and the reaction mixture stirred for 15 h. The reaction mixture was concentrated under reduced pressure to give a crude solid which was dissolved in ACN:water (1:1) and purified by reverse phase column chromatography by using C-18 column eluting with 10-50% acetonitrile in water with 0.1% formic acid. The fractions were lyophilized to afford the title compound as an off-white solid (40 mg, 14%). A portion of the solid (6.3 mg, 11 μmol) was take up in acetonitrile (0.50 mL) and water (0.50 mL). To the suspension was added formic acid (5.0 μL, 0.13 mmol) at room temperature. The resulting solution stirred for 10 min at rt and was then lyophilized to afford 3-(1-oxo-5-(1-((4-oxo-3-(pyridin-3-yl)-3,4-dihydroquinazolin-6-yl)methyl)piperidin-4-yl)isoindolin-2-yl)piperidine-2,6-dione formate (6.0 mg, 9.9 μmol) as an off white solid. ¹H NMR (500 MHz, DMSO-d₆) δ 10.97 (s, 1H) 8.78 (br s, 1H) 8.70 (br d, J=4.4 Hz, 1H) 8.39 (s, 1H) 8.16 (br s, 1H) 8.05 (br d, J=7.7 Hz, 1H) 7.87 (br d, J=8.2 Hz, 1H) 7.75 (br d, J=8.2 Hz, 1H) 7.63 (br d, J=7.1 Hz, 2H) 7.51 (br s, 1H) 7.41 (br d, J=7.7 Hz, 1H) 5.09 (br s, 1H) 4.24-4.51 (m, 2H) 3.70 (br s, 1H) 3.32 (br s, 3H) 2.84-3.04 (m, 3H) 2.56-2.72 (m, 2H) 2.33-2.45 (m, 1H) 2.15 (br t, J=10.4 Hz, 2H) 1.93-2.06 (m, 1H) 1.67-1.85 (m, 3H). MS [M+H]⁺=563.2.

### Example 19: Synthesis of 3-(1-oxo-5-(1-((2-phenylimidazo[1,2-a]pyridin-6-yl)methyl)piperidin-4-yl)isoindolin-2-yl)piperidine-2,6-dione (54)

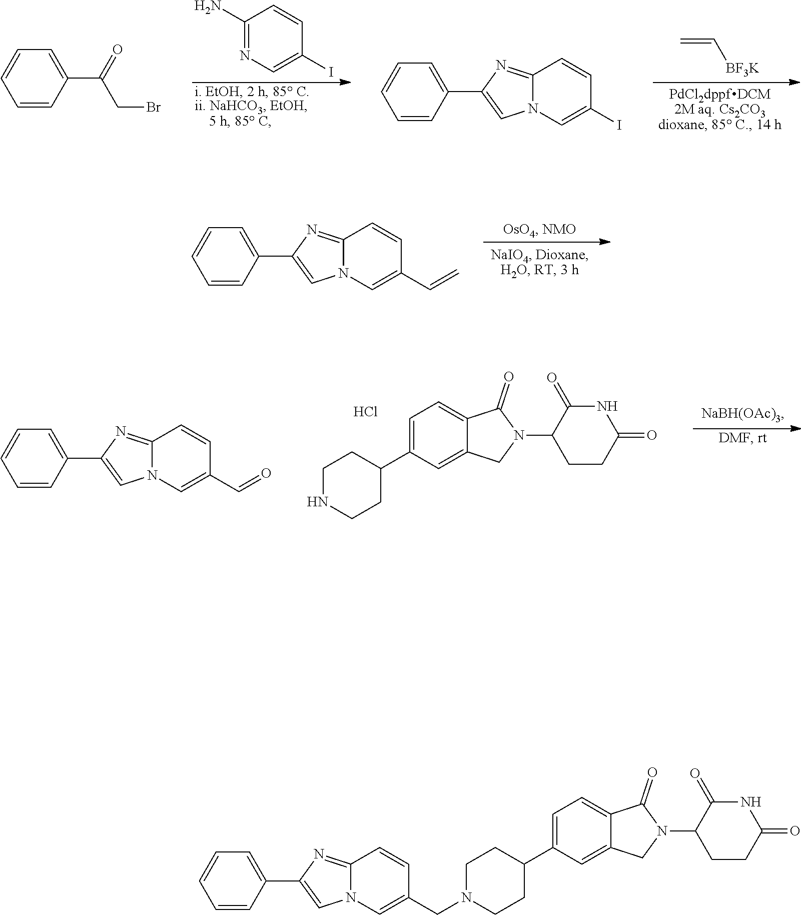

### 6-Iodo-2-phenylimidazo[1,2-a]pyridine

A mixture of 2-bromo-1-phenylethan-1-one (5.00 g, 25.1 mmol) and 5-iodopyridin-2-amine (5.53 g, 25.1 mmol) in ethanol (100 mL) was stirred at reflux for 2 h. Sodium bicarbonate (4.64 g, 55.3 mmol) was added to the reaction mixture at rt and the mixture was heated at reflux for 5 h. The reaction mixture was diluted with EtOAc (300 mL) and the mixture was washed with water (2×150 mL). The organic layer was dried over sodium sulfate and concentrated under reduced pressure to afford the crude compound. The crude compound was triturated with hexane, filtered and dried under vacuum to afford the title compound as a pale brown solid (5.70 g, 67%). ¹H NMR (400 MHz, CDCl₃) δ 8.42 (dd, J=0.9, 1.7 Hz, 1H), 7.96 (dd, J=1.3, 8.3 Hz, 2H), 7.84 (d, J=0.6 Hz, 1H), 7.52-7.42 (m, 3H), 7.41-7.33 (m, 2H). MS [M+H]⁺=321.

### 2-Phenyl-6-vinylimidazo[1,2-a]pyridine

To a stirred solution of 6-iodo-2-phenylimidazo[1,2-a]pyridine (5.70 g, 17.8 mmol) in 1,4-dioxane (57 mL) was added potassium vinyltrifluoroborate (2.39 g, 17.8 mmol), Cs₂CO₃ (17.4 g, 53.4 mmol) in water (26.7 mL), followed by the addition of PdCl₂(dppf)-CH₂Cl₂ adduct (1.45 g, 1.78 mmol) at rt under continuous N₂ bubbling. The reaction mixture was stirred at 85° C. for 14 h. The reaction mixture was filtered through a pad of Celite® and washed with ethyl acetate. The combined organic layers were washed with water, dried over anhydrous Na₂SO₄ and filtered. The solvent was removed under reduced pressure to afford the title compound as a pale brown solid (3.50 g, 79%). ¹H NMR (400 MHz, CDCl₃) δ 8.09 (s, 1H), 8.02-7.95 (m, 1H), 7.86 (s, 1H), 7.68 (s, 1H), 7.53-7.41 (m, 5H), 6.68 (dd, J=11.0, 17.4 Hz, 1H), 5.79 (d, J=17.4 Hz, 1H), 5.38 (d, J=11.0 Hz, 1H). MS [M+H]⁺=221.

### 2-Phenylimidazo[1,2-a]pyridine-6-carbaldehyde

A solution of 2-phenyl-6-vinylimidazo[1,2-a]pyridine (1.00 g, 4.54 mmol) in 1,4-dioxane (12.5 mL)/water (1.3 mL) was cooled to 0° C. before sodium periodate (1.94 g, 9.08 mmol) and N-methylmorpholine (0.230 g, 2.27 mmol) were added followed by dropwise addition of osmium tetroxide (4 wt. % in water, 2.89 g, 0.454 mmol) at the same temperature. The reaction mixture was stirred at 25° C. for 3 h. The reaction mixture was quenched with ice-cold brine (100 mL) before it was extracted with EtOAc (3×200 mL). The combined organic extracts were dried over anhydrous Na₂SO₄ and filtered. The solvent was removed under reduced pressure to afford a crude residue that was purified by silica gel column chromatography using 40-60% ethyl acetate in hexane as an eluent to afford the title compound as a yellow solid (250 mg, 25%). ¹H NMR (400 MHz, CDCl₃) δ 9.98 (s, 1H), 8.70 (s, 1H), 8.04-7.94 (m, 3H), 7.77-7.64 (m, 2H), 7.54-7.46 (m, 2H), 7.45-7.35 (m, 1H). MS [M+H]⁺=223.

### 3-(1-Oxo-5-(1-((2-phenylimidazo[1,2-a]pyridin-6-yl)methyl)piperidin-4-yl)isoindolin-2-yl)piperidine-2,6-dione

To a stirred solution of 2-phenylimidazo[1,2-a]pyridine-6-carbaldehyde (100 mg, 0.450 mmol) in DMF (2 mL) was added 3-(1-oxo-5-(piperidin-4-yl)isoindolin-2-yl)piperidine-2,6-dione hydrochloride (180 mg, 0.495 mmol) at rt. The reaction mixture was stirred at rt for 30 min before sodium triacetoxyhydroborate (238 mg, 1.13 mmol) was added at rt and the mixture was stirred for 15 h. The reaction mixture was diluted with cold water (2×30 mL) and was extracted with 10% MeOH/DCM (2×100 mL). The combined organic layers were washed with brine, dried over sodium sulfate, and concentrated to afford a crude residue that was purified by preparative HPLC [(Column: X select (150 mm*19) 5 μm, Mobile phase A: 0.1% HCOOH in H₂O, Mobile phase B: acetonitrile, flow rate: 15 mL/min)]. ACN/water was removed by lyophilization to afford the title compound as a white solid (51 mg, 21%). ¹H NMR (400 MHz, DMSO-d₆) δ 10.98 (s, 1H), 8.46 (s, 1H), 8.37 (s, 1H), 8.00-7.92 (m, 2H), 7.65 (d, J=7.9 Hz, 1H), 7.59-7.48 (m, 2H), 7.47-7.39 (m, 3H), 7.35-7.29 (m, 1H), 7.26 (dd, J=1.6, 9.3 Hz, 1H), 5.10 (dd, J=5.1, 13.3 Hz, 1H), 4.47-4.38 (m, 1H), 4.33-4.25 (m, 1H), 3.54 (s, 2H), 3.06-2.84 (m, 3H), 2.66-2.55 (m, 2H), 2.48-2.36 (m, 1H), 2.20-2.08 (m, 2H), 2.04-1.95 (m, 1H), 1.86-1.68 (m, 4H). MS [M+H]⁺=534.2.

### Example 20: Synthesis of 3-(1-oxo-5-(1-((2-phenyl-[1,2,4]triazolo[1,5-a]pyridin-6-yl)methyl)piperidin-4-yl)isoindolin-2-yl)piperidine-2,6-dione (55)

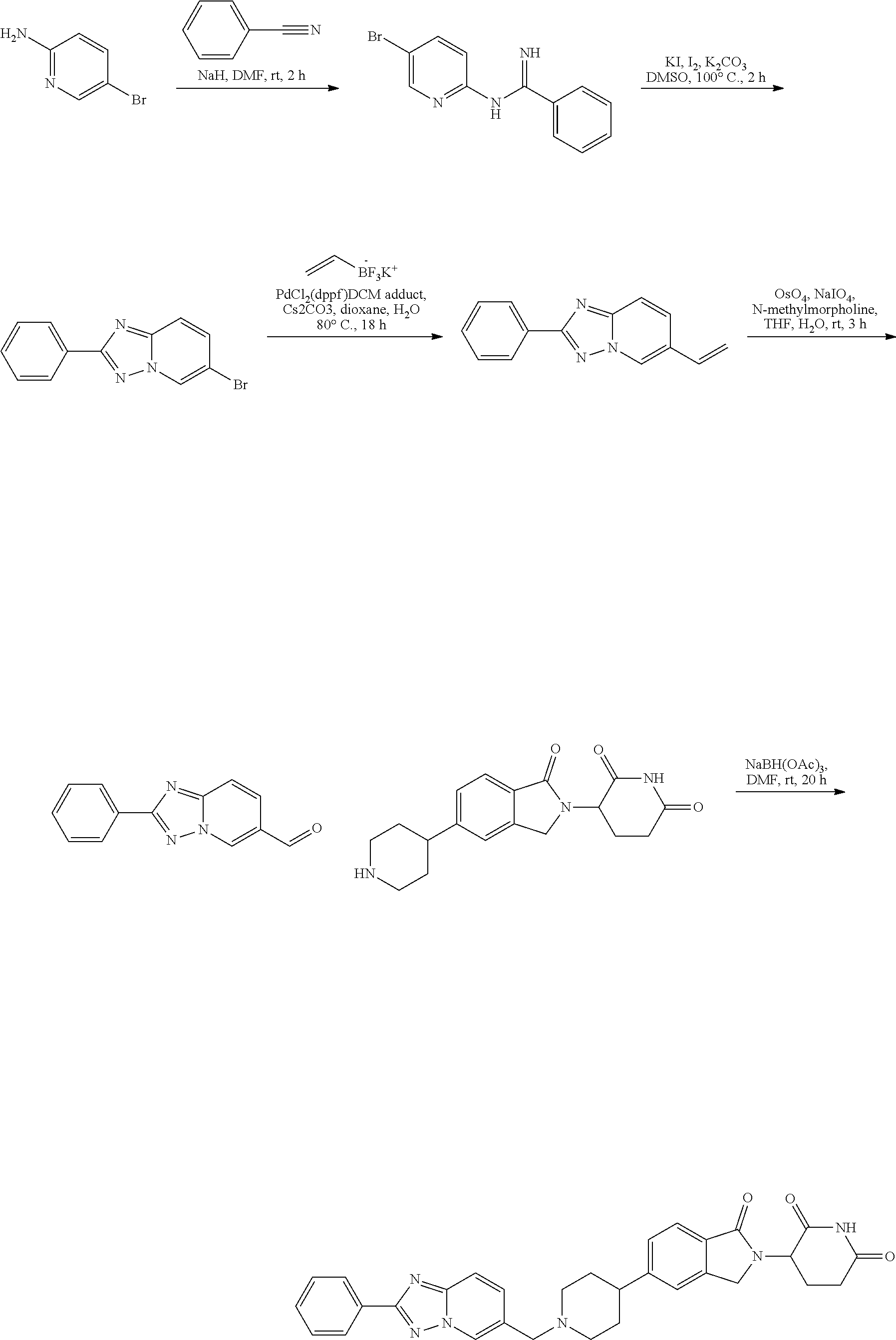

### N-(5-bromopyridin-2-yl)benzimidamide

To a stirred solution of 5-bromopyridin-2-amine (2.00 g, 11.6 mmol) in DMF (6 mL) was added sodium hydride (0.555 g, 13.9 mmol) at 0° C. The resulting reaction mixture was allowed to stir at 0° C. for 30 min before benzonitrile (1.43 g, 13.9 mmol) was added. The reaction mixture was allowed to stir under nitrogen atmosphere at rt for 2 h. An aqueous solution of sodium bicarbonate (5%, 20.0 mL) was added and the mixture was extracted with ethyl acetate (2×30 mL). The organic layers were dried over Na₂SO₄ and evaporated under reduced pressure to afford a crude residue that was purified by silica gel column chromatography using ethyl acetate in hexane (8-10%) as an eluent to afford the title compound as a white solid (1.00 g, 23%). ¹H NMR (400 MHz, DMSO-d₆) δ 8.44 (dd, J=0.4, 2.8 Hz, 1H), 8.04 (dd, J=1.6, 8.4 Hz, 2H), 7.88 (dd, J=2.4, 8.8 Hz, 1H), 7.53-7.47 (m, 3H), 7.06 (d, J=8.8 Hz, 1H). MS [M+H, M+2H]⁺=276.0, 278.0.

### 6-Bromo-2-phenyl-[1,2,4]triazolo[1,5-a]pyridine

A mixture of potassium iodide (0.812 g, 4.89 mmol) and iodine (0.993 g, 3.91 mmol) in DMSO (15 mL) was stirred at rt for 10 min. N-(5-Bromopyridin-2-yl)benzimidamide (0.900 g, 3.26 mmol) and K₂CO₃ (1.35 g, 9.78 mmol) were added to the mixture at rt. The mixture was heated at 100° C. under nitrogen atmosphere for 2 h. To the reaction mixture were added 5% aqueous Na₂S₂O₃ (5 mL) and brine (50 mL). The mixture was extracted with ethyl acetate (2×40 mL). The combined organic layers were dried over anhydrous Na₂SO₄ and concentrated under reduced pressure to afford a crude residue which was purified by silica gel column chromatography using ethyl acetate in hexane (4-6%) as eluents to afford the title compound as a white solid (0.580 g, 61%). ¹H NMR (400 MHz, DMSO-d₆) δ 8.77 (dd, J=0.8, 1.6 Hz, 1H), 8.29-8.27 (m, 2H), 7.68 (d, J=9.2 Hz, 1H), 7.61 (dd, J=2.0, 9.2 Hz, 1H), 7.54-7.50 (m, 3H). MS [M+H, M+2H]⁺=273.8, 275.8.

### 2-Phenyl-6-vinyl-[1,2,4]triazolo[1,5-a]pyridine

To a stirred solution of 6-bromo-2-phenyl-[1,2,4]triazolo[1,5-a]pyridine (0.580 g, 2.12 mmol) in 1,4-dioxane (8 mL) were added potassium vinyltrifluoroborate (0.850 g, 6.35 mmol), cesium carbonate (2M, 1.06 ml, 2.12 mmol) and PdCl₂(dppf)-CH₂Cl₂ adduct (0.173 g, 0.212 mmol) at rt under continuous N₂ bubbling. The resulting mixture was allowed to stir at 85° C. for 18 h. The mixture was concentrated under reduced pressure to afford a crude residue that was purified by silica gel column chromatography using ethyl acetate in hexane (9-10%) as eluents to afford the title compound as a white solid (0.340 g, 72%). ¹H NMR (400 MHz, DMSO-d₆) δ 8.56 (s, 1H), 8.31-8.28 (m, 2H), 7.74-7.71 (m, 2H), 7.55-7.48 (m, 3H), 6.75 (dd, J=11.2, 17.6 Hz, 1H), 5.85 (d, J=17.6 Hz, 1H), 5.46 (d, J=11.2 Hz, 1H). MS [M+H]⁺=222.3.

### 2-Phenyl-[1,2,4]triazolo[1,5-a]pyridine-6-carbaldehyde

To a stirred solution of 2-phenyl-6-vinyl-[1,2,4]triazolo[1,5-a]pyridine (0.340 g, 1.54 mmol) in 1,4-dioxane (6 mL):water (0.2 mL) were added sodium periodate (0.657 g, 3.07 mmol) and N-methylmorpholine (0.084 mL, 0.768 mmol) followed by dropwise addition of osmium tetroxide (4 wt % water, 1.21 ml, 0.154 mmol) at 0° C. The resulting reaction mixture was allowed to stir at rt for 3 h. The reaction mixture was quenched with ice-cold brine (20 mL) and was extracted with EtOAc (3×30 mL). The combined organic extracts were dried over anhydrous Na₂SO₄ and filtered. The solvent was removed under reduced pressure to afford a crude residue that was purified by silica gel column chromatography using ethyl acetate in hexane (25-28%) as an eluent to afford the title compound as a white solid (0.120 g, 32%). ¹H NMR (400 MHz, DMSO-d₆) δ 10.07 (s, 1H), 9.77 (dd, J=0.8, 1.6 Hz, 1H), 8.27-8.25 (m, 2H), 8.04-7.96 (m, 2H), 7.61-7.56 (m, 3H). MS [M+H]⁺=224.2.

### 3-(1-Oxo-5-(1-((2-phenyl-[1,2,4]triazolo[1,5-a]pyridin-6-yl)methyl)piperidin-4-yl)isoindolin-2-yl)piperidine-2,6-dione

To a stirred solution of 3-(1-oxo-5-(piperidin-4-yl)isoindolin-2-yl)piperidine-2,6-dione hydrochloride (0.160 g, 0.440 mmol) in DMF (4 mL) was added 2-phenyl-[1,2,4]triazolo[1,5-a]pyridine-6-carbaldehyde (0.118 g, 0.528 mmol) at rt. The reaction mixture was stirred at rt for 30 min before it was cooled to 0° C. and sodium triacetoxyborohydride (0.242 g, 1.143 mmol) was added portion-wise. The resulting reaction mixture was allowed to stir under nitrogen atmosphere at rt for 20 h. The reaction mixture was concentrated under reduced pressure to afford a crude residue that was purified using reverse phase column chromatography, eluting with 0.1% HCOOH in water:acetonitrile. The acetonitrile/water was removed by lyophilization to afford the title compound as a white solid (0.074 g, 30%). ¹H NMR (400 MHz, DMSO-d₆) δ 10.98 (br s, 1H), 8.92 (s, 1H), 8.20 (dd, J=1.6, 4.0 Hz, 2H), 7.84 (d, J=9.2 Hz, 1H), 7.71-7.63 (m, 2H), 7.57-7.51 (m, 4H), 7.43-7.41 (m, 1H), 5.10 (dd, J=5.2, 13.2 Hz, 2H), 4.43 (d, J=17.2 Hz, 2H), 4.29 (d, J=17.2 Hz, 2H), 3.65 (s, 2H), 3.02-2.87 (m, 3H), 2.68-2.62 (m, 1H), 2.428-2.37 (m, 1H), 2.20-2.15 (m, 2H), 2.00-1.98 (m, 1H), 1.79-1.74 (m, 2H). MS [M+H]⁺=535.2.

### Example 21: Synthesis of 3-(5-(1-((6-bromoimidazo[1,2-a]pyridin-2-yl)methyl)piperidin-4-yl)-1-oxoisoindolin-2-yl)piperidine-2,6-dione

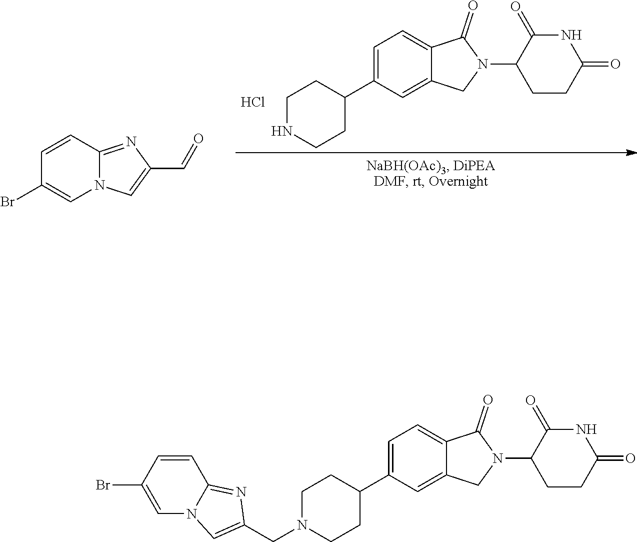 [SMILES: O=Cc1cn2cc(Br)ccc2n1 ; Cl ; O=C1CCC(N2Cc3cc(C4CCNCC4)ccc3C2=O)C(=O)N1 ; "NaBH(OAc)3, DiPEADMF, rt, Overnight" ; O=C1CCC(N2Cc3cc(C4CCN(Cc5cn6cc(Br)ccc6n5)CC4)ccc3C2=O)C(=O)N1]

To a stirred solution of 3-(1-oxo-5-(piperidin-4-yl)isoindolin-2-yl)piperidine-2,6-dione hydrochloride (0.628 g, 1.73 mmol) in DMF (5 mL) were added 6-bromoindolizine-2-carbaldehyde (0.300 g, 1.15 mmol) and DIPEA (0.603 ml, 3.45 mmol) at 25° C. The mixture was stirred for 10 min. Sodium triacetoxyborohydride (1.22 g, 5.76 mmol) was added portion-wise at 0° C. The reaction mixture was stirred under nitrogen atmosphere at 25° C. for 18 h. The reaction mixture was then concentrated under reduced pressure to afford a crude residue that was purified by normal phase column chromatography with a gradient of 12% IPA in DCM as an eluent to afford the title compound as a pale yellow solid (0.240 g). A portion of the isolated compound (70 mg, 0.124 mmol) was further purified by prep-HPLC [Method info: column: X select (150 mm×19) 5 μm, 0.1% HCl in H₂O and ACN, Flow rate: 15 mL/min]. The collected fraction was lyophilized to afford the title compound as an off-white solid (17 mg, 11%). ¹H NMR (400 MHz, DMSO-d₆) δ 11.0 (s, 1H), 10.65 (brs, 1H), 9.10 (s, 1H), 8.20 (s, 1H), 7.80-7.60 (m, 2H), 7.55-7.50 (m, 1H), 7.5-7.4 (m, 1H), 7.40-7.30 (m, 1H), 5.15-5.05 (m, 1H), 4.55-4.35 (m, 3H), 4.32 (d, J=17.6 Hz, 1H), 3.60-3.50 (m, 2H), 3.25-3.10 (m, 2H), 3.00-2.85 (m, 2H), 2.65-2.50 (m, 1H), 2.34-2.20 (m, 1H), 2.15-1.90 (m, 5H). MS [M+2H]⁺=538.1.

### Example 22: Synthesis of 3-(1-oxo-5-(1-((6-phenylimidazo[1,2-a]pyridin-2-yl)methyl)piperidin-4-yl)isoindolin-2-yl)piperidine-2,6-dione (96)

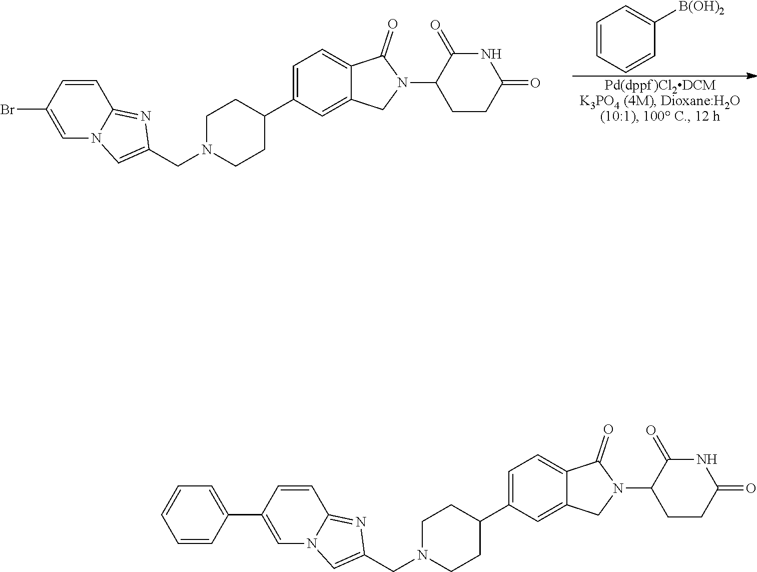

To a stirred solution of 3-(5-(1-((6-bromoimidazo[1,2-a]pyridin-2-yl)methyl)piperidin-4-yl)-1-oxoisoindolin-2-yl)piperidine-2,6-dione (100 mg, 0.186 mmol) in 1,4-dioxane (3 mL)/water (0.15 mL) were added phenylboronic acid (34.1 mg, 0.280 mmol) and K₃PO₄ tribasic in H₂O (4M, 0.140 mL, 0.559 mmol) followed by PdCl₂(dppf)-CH₂Cl₂ adduct (15 mg, 0.019 mmol) under continuous bubbling of N₂ for 10 min. The reaction mixture was stirred under N₂ atmosphere at 100° C. for 12 h. The reaction mixture was concentrated under reduced pressure to afford a crude residue which was purified by normal phase column chromatography with a gradient of 16% IPA in DCM as an eluent to afford the title compound as a pale brown solid (80 mg). This material was further purified by prep-HPLC [Method info: column: X select (150 mm×19) 5 μm, 0.1% HCl in H₂O and ACN, Flow rate: 15 mL/min]. The collected fraction was lyophilized to afford 3-(1-oxo-5-(1-((6-phenylimidazo[1,2-a]pyridin-2-yl)methyl)piperidin-4-yl)isoindolin-2-yl)piperidine-2,6-dione hydrochloride as an off-white solid (6.5 mg, 6.4%). ¹H NMR (400 MHz, DMSO-d₆) δ 10.99 (s, 1H), 10.55 (brs, 1H), 9.12 (s, 1H), 8.26 (s, 1H), 7.90-7.65 (m, 5H), 7.60-7.50 (m, 2H), 7.50-7.32 (m, 3H), 5.20-5.05 (m, 1H), 4.60-4.51 (m, 2H), 4.45 (d, J=17.2 Hz, 1H), 4.32 (d, J=17.6 Hz, 1H), 3.64-3.61 (m, 2H), 3.31-3.11 (m, 2H), 3.05-2.85 (m, 2H), 2.65-2.55 (m, 1H), 2.44-2.35 (m, 1H), 2.10-1.90 (m, 5H). MS [M+H]⁺=534.2.

### Example 23: Synthesis of 3-(1-oxo-5-(1-((6-(pyridin-3-yl)imidazo[1,2-a]pyridin-2-yl)methyl)piperidin-4-yl)isoindolin-2-yl)piperidine-2,6-dione (97)

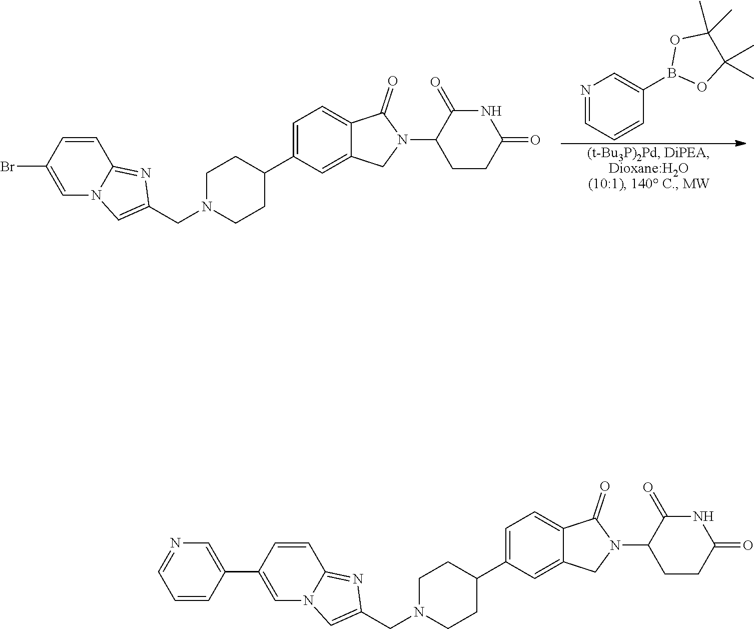

To a stirred solution of 3-(5-(1-((6-bromoimidazo[1,2-a]pyridin-2-yl)methyl)piperidin-4-yl)-1-oxoisoindolin-2-yl)piperidine-2,6-dione (0.100 g, 0.186 mmol) in 1,4-dioxane (3 mL)/water (0.15 mL) were added 3-(4,4,5,5-tetramethyl-1,3,2-dioxaborolan-2-yl)pyridine (0.057 g, 0.28 mmol) and DIPEA (0.072 g, 0.56 mmol) followed by bis(tri-t-butylphosphine)palladium(0) (9.50 mg, 0.019 mmol) under continuous bubbling of N₂ for 10 min. The reaction mixture was irradiated under microwave at 140° C. for 4 h. The reaction mixture was concentrated under reduced pressure to afford a crude residue that was purified by reverse phase column chromatography 4% ACN in water as an eluent to afford the title compound (30 mg). The isolated compound was further purified by prep-HPLC [Method info: column: X select (150 mm×19) 5 μm, 0.1% HCl in H₂O and ACN, flow rate: 15 mL/min]. The collected fraction was lyophilized to afford 3-(1-oxo-5-(1-((6-(pyridin-3-yl)imidazo[1,2-a]pyridin-2-yl)methyl)piperidin-4-yl)isoindolin-2-yl)piperidine-2,6-dione hydrochloride as a pale yellow solid (6.1 mg, 6%). ¹H NMR (400 MHz, DMSO-d₆) δ 11.28 (brs, 1H), 11.0 (s, 1H), 9.42 (s, 1H), 9.21 (s, 1H), 8.84 (s, 1H), 8.69-8.58 (m, 1H), 8.12-8.01 (m, 1H), 7.95-7.87 (m, 2H), 7.79-7.67 (m, 1H), 7.48 (s, 1H), 7.41-7.33 (m, 1H), 5.19-5.06 (m, 1H), 4.65-4.50 (m, 2H), 4.46 (d, J=17.6 Hz, 1H), 4.32 (d, J=17.6 Hz, 1H), 3.68-3.55 (m, 2H), 3.30-3.11 (m, 2H), 3.05-2.85 (m, 2H), 2.65-2.55 (m, 1H), 2.45-2.35 (m, 1H), 2.23-1.90 (m, 5H). MS [M+H]⁺=535.3.

### Example 24: Synthesis of 3-(1-oxo-5-(1-((2-(pyridin-3-yl)-2H-indazol-6-yl)methyl)piperidin-4-yl)isoindolin-2-yl)piperidine-2,6-dione (95)

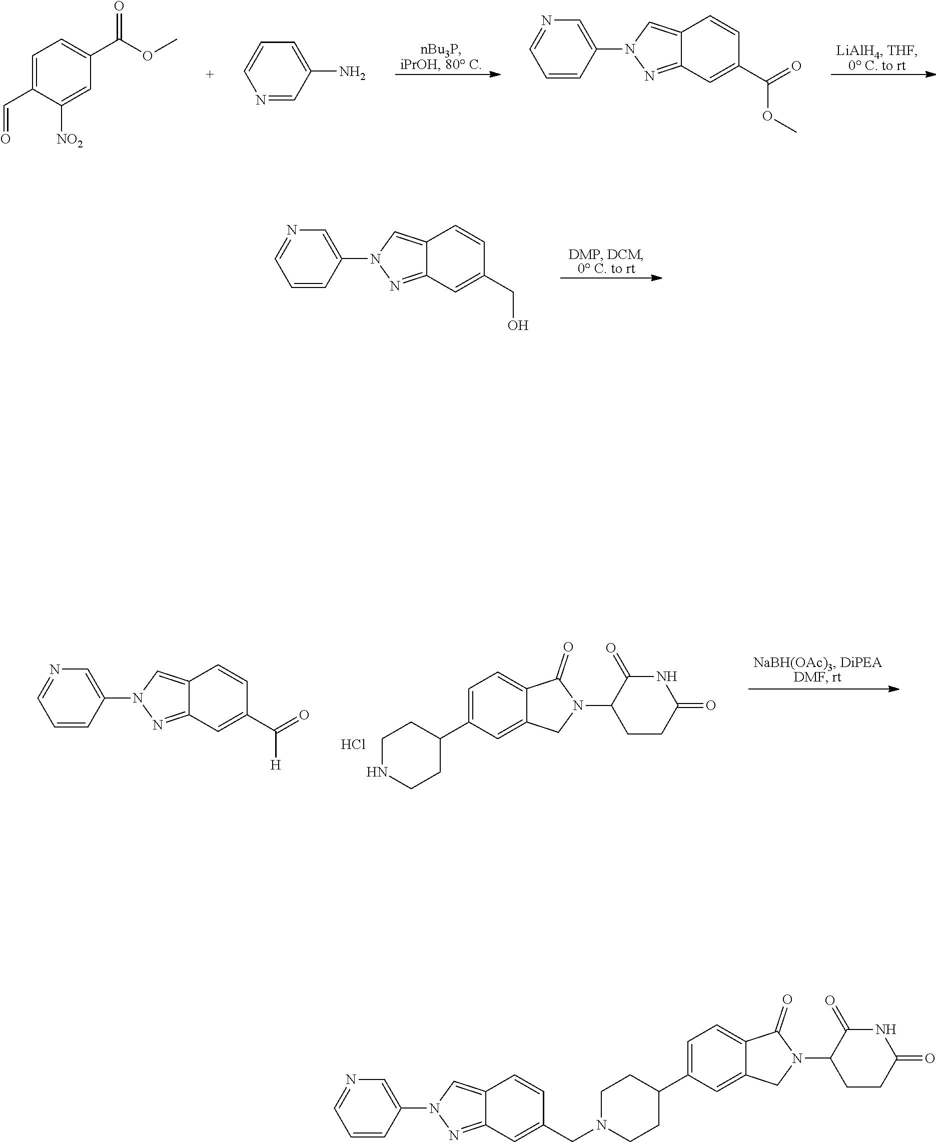

### Methyl 2-(pyridin-3-yl)-2H-indazole-6-carboxylate

To a vial with 4-(methoxycarbonyl)-2-nitrobenzaldehyde (500 mg, 2.39 mmol) was added isopropanol (6.0 mL) followed by 3-aminopyridine (247 mg, 2.63 mmol). The vial was flushed with nitrogen and then heated at 80° C. After 4 hours, the mixture was cooled to rt before tri-n-butylphosphine (1.77 mL, 7.17 mmol) was added. The resulting mixture was heated at 80° C. for 16 hr overnight. The mixture was cooled to rt, diluted with ethyl acetate (4 mL) and washed with sat. aq. ammonium chloride solution and brine. The organic layer was dried over sodium sulfate, filtered and concentrated. The crude residue was purified by column chromatography on a C18 reverse-phase column, eluting with 20-100% acetonitrile in water containing 0.1% formic acid. The fractions containing product were combined and lyophilized to afford methyl 2-(pyridin-3-yl)-2H-indazole-6-carboxylate formate as a tan solid (255 mg, 42%). ¹H NMR (500 MHz, CDCl₃) δ 9.14-9.30 (m, 1H) 8.70 (dd, J=1.0, 4.8 Hz, 1H) 8.58 (s, 1H) 8.52 (s, 1H) 8.31-8.43 (m, 1H) 8.11 (s, 1H) 7.77 (s, 2H) 7.57 (dd, J=4.8, 8.4 Hz, 1H) 3.98 (s, 3H). MS [M+H]⁺=254.1.

### (2-(Pyridin-3-yl)-2H-indazol-6-yl)methanol

A solution of methyl 2-(pyridin-3-yl)-2H-indazole-6-carboxylate (255 mg, 1.01 mmol) in THE (10 mL) was cooled to 0° C. To the solution was added LiAlH₄ (1M in THF, 3.00 mL, 3.00 mmol) and the mixture was slowly warmed to rt. The mixture was quenched with MeOH (drop-wise addition) and the solution was stirred for 30 min at rt. The mixture was diluted with ethyl acetate and sat. sodium bicarbonate solution. The layers were separated and the aq. layer was extracted with ethyl acetate×2. The combined organics were washed with water and brine, dried over sodium sulfated, filtered and concentrated to afford a crude residue that was purified by column chromatography on silica gel eluting with 40-100% ethyl acetate in hexanes, then up to 10% methanol in ethyl acetate to afford the title compound as a yellow solid (78 mg, 34%). ¹H NMR (500 MHz, methanol-d₄) δ 9.18-9.29 (m, 1H) 8.86 (s, 1H) 8.61 (d, J=4.9 Hz, 1H) 8.45 (dd, J=1.4, 8.5 Hz, 1H) 7.75 (d, J=8.8 Hz, 1H) 7.68 (s, 1H) 7.65 (dd, J=4.9, 8.2 Hz, 1H) 7.14 (d, J=8.8 Hz, 1H) 4.72 (s, 2H). MS [M+H]⁺=226.1.

### 2-(Pyridin-3-yl)-2H-indazole-6-carbaldehyde

To a solution of (2-(pyridin-3-yl)-2H-indazol-6-yl)methanol (75 mg, 0.33 mmol) in DCM (1.7 mL) at rt was added DMP (0.17 g, 0.40 mmol). The mixture became an orange homogeneous solution upon addition of the DMP. After stirring for 30 min the solution was passed through a syringe filter and was purified by column chromatography on silica gel, eluting with 30-100% ethyl acetate in hexanes. The fractions containing product were combined and concentrated to afford the title compound (43 mg, 58%). ¹H NMR (500 MHz, CDCl₃) δ 10.07-10.17 (m, 1H) 9.22 (d, J=2.2 Hz, 1H) 8.72 (d, J=3.8 Hz, 1H) 8.54 (s, 1H) 8.27-8.36 (m, 2H) 7.83 (d, J=8.8 Hz, 1H) 7.68 (dd, J=1.1, 8.8 Hz, 1H) 7.54 (dd, J=4.7, 8.2 Hz, 1H). MS [M+H]⁺=224.1.

### 3-(1-Oxo-5-(1-((2-(pyridin-3-yl)-2H-indazol-6-yl)methyl)piperidin-4-yl)isoindolin-2-yl)piperidine-2,6-dione

To a solution of 3-(1-oxo-5-(piperidin-4-yl)isoindolin-2-yl)piperidine-2,6-dione (40 mg, 0.12 mmol) and 2-(pyridin-3-yl)-2H-indazole-6-carbaldehyde (40 mg, 0.18 mmol) in dimethylacetamide (DMA, 4 mL) at rt was added sodium triacetoxyborohydride (78 mg, 0.37 mmol) followed by DIPEA (64 μL, 0.37 mmol). After stirring for 18 h, the mixture was diluted with water (20 mL) and was then extracted with ethyl acetate (3×10 mL). The combined organics were washed with brine (2×30 mL), dried over sodium sulfate, filtered and concentrated. The crude residue was purified by column chromatography on a C18 column, eluting with 10-100% acetonitrile in water containing 0.1% formic acid to afford the title compound as a white solid. MS [M+H]⁺=535.3.

### Example 25: Synthesis of 3-(5-(1-((3-(3-fluorophenyl)-4-oxo-3,4-dihydroquinazolin-6-yl)methyl)piperidin-4-yl)-1-oxoisoindolin-2-yl)piperidine-2,6-dione (28)

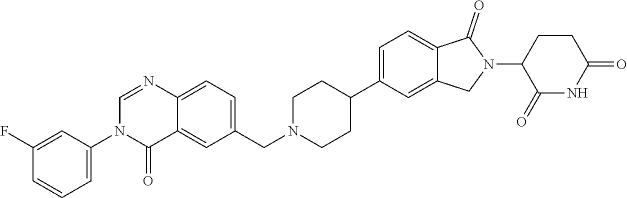 [SMILES: O=C1CCC(N2Cc3cc(C4CCN(Cc5ccc6ncn(-c7cccc(F)c7)c(=O)c6c5)CC4)ccc3C2=O)C(=O)N1]

Compound 28 was prepared according to similar procedures as described in Examples 16 and 18. MS [M+H]⁺=580.2.

### Example 26: Synthesis of 3-(1-oxo-5-(1-((4-oxo-3-(3-(trifluoromethyl)phenyl)-3,4-dihydroquinazolin-6-yl)methyl)piperidin-4-yl)isoindolin-2-yl)piperidine-2,6-dione (60)

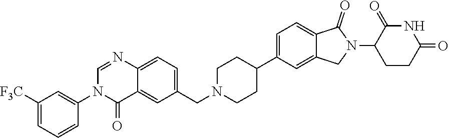 [SMILES: O=C1CCC(N2Cc3cc(C4CCN(Cc5ccc6ncn(-c7cccc(C(F)(F)F)c7)c(=O)c6c5)CC4)ccc3C2=O)C(=O)N1]

Compound 60 was prepared according to similar procedures as described in Examples 16 and 18. MS [M+H]⁺=630.1.

### Example 27: Synthesis of 3-(6-((4-(2-(2,6-dioxopiperidin-3-yl)-1-oxoisoindolin-5-yl)piperidin-1-yl)methyl)-4-oxoquinazolin-3(4H)-yl)benzonitrile (61)

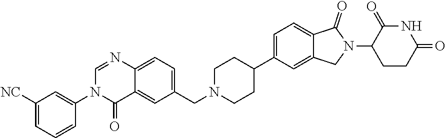 [SMILES: N#Cc1cccc(-n2cnc3ccc(CN4CCC(c5ccc6c(c5)CN(C5CCC(=O)NC5=O)C6=O)CC4)cc3c2=O)c1]

Compound 61 was prepared according to similar procedures as described in Examples 13 and 15. MS [M+H]⁺=587.2.

### Example 28: Synthesis of 3-(5-(1-((3-(6-methylpyridin-2-yl)-4-oxo-3,4-dihydroquinazolin-6-yl)methyl)piperidin-4-yl)-1-oxoisoindolin-2-yl)piperidine-2,6-dione (62)

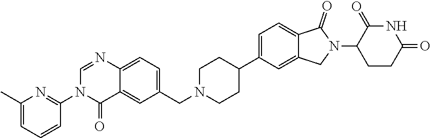 [SMILES: Cc1cccc(-n2cnc3ccc(CN4CCC(c5ccc6c(c5)CN(C5CCC(=O)NC5=O)C6=O)CC4)cc3c2=O)n1]

Compound 62 was prepared according to similar procedures as described in Examples 16 and 18. MS [M+H]⁺=577.1.

### Example 29: Synthesis of 3-(5-(1-((3-(6-isopropylpyridin-2-yl)-4-oxo-3,4-dihydroquinazolin-6-yl)methyl)piperidin-4-yl)-1-oxoisoindolin-2-yl)piperidine-2,6-dione (63)

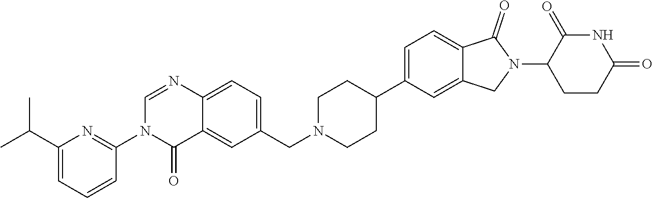 [SMILES: CC(C)c1cccc(-n2cnc3ccc(CN4CCC(c5ccc6c(c5)CN(C5CCC(=O)NC5=O)C6=O)CC4)cc3c2=O)n1]

Compound 63 was prepared according to similar procedures as described in Examples 16 and 18. MS [M+H]⁺=605.1.

### Example 30: Synthesis of 3-(5-(1-((3-(6-methoxypyridin-2-yl)-4-oxo-3,4-dihydroquinazolin-6-yl)methyl)piperidin-4-yl)-1-oxoisoindolin-2-yl)piperidine-2,6-dione (65)

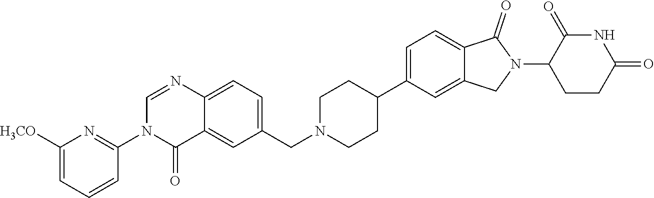 [SMILES: COc1cccc(-n2cnc3ccc(CN4CCC(c5ccc6c(c5)CN(C5CCC(=O)NC5=O)C6=O)CC4)cc3c2=O)n1]

Compound 65 was prepared according to similar procedures as described in Examples 16 and 18. MS [M+H]⁺=593.1.

### Example 31: Synthesis of 3-(1-oxo-5-(1-((4-oxo-3-(thiazol-5-yl)-3,4-dihydroquinazolin-6-yl)methyl)piperidin-4-yl)isoindolin-2-yl)piperidine-2,6-dione (66)

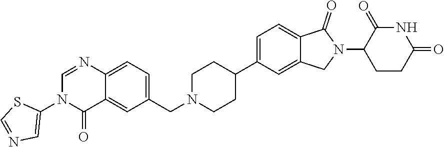 [SMILES: O=C1CCC(N2Cc3cc(C4CCN(Cc5ccc6ncn(-c7cncs7)c(=O)c6c5)CC4)ccc3C2=O)C(=O)N1]

Compound 66 is prepared according to similar procedures as described in Examples 16 and 18.

### Example 32: Synthesis of 3-(5-(1-((3-(2-methylpyridin-3-yl)-4-oxo-3,4-dihydroquinazolin-6-yl)methyl)piperidin-4-yl)-1-oxoisoindolin-2-yl)piperidine-2,6-dione (68)

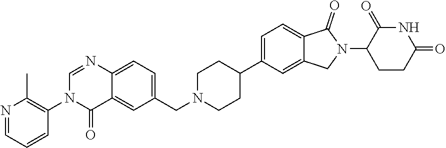 [SMILES: Cc1ncccc1-n1cnc2ccc(CN3CCC(c4ccc5c(c4)CN(C4CCC(=O)NC4=O)C5=O)CC3)cc2c1=O]

Compound 68 was prepared according to similar procedures as described in Examples 16 and 18. MS [M+H]⁺=577.4.

### Example 33: Synthesis of 3-(5-(1-((3-(6-methylpyridin-3-yl)-4-oxo-3,4-dihydroquinazolin-6-yl)methyl)piperidin-4-yl)-1-oxoisoindolin-2-yl)piperidine-2,6-dione (69)

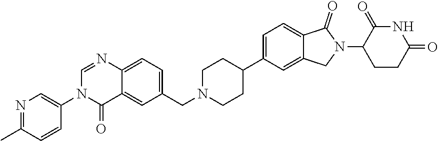 [SMILES: Cc1ccc(-n2cnc3ccc(CN4CCC(c5ccc6c(c5)CN(C5CCC(=O)NC5=O)C6=O)CC4)cc3c2=O)cn1]

Compound 69 was prepared according to similar procedures as described in Examples 16 and 18. MS [M+H]⁺=577.3.

### Example 34: Synthesis of 3-(5-(1-((3-(2,6-dimethylpyridin-3-yl)-4-oxo-3,4-dihydroquinazolin-6-yl)methyl)piperidin-4-yl)-1-oxoisoindolin-2-yl)piperidine-2,6-dione (70)

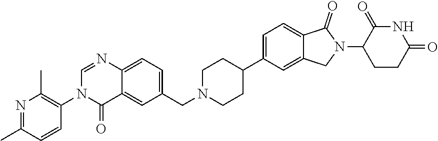 [SMILES: Cc1ccc(-n2cnc3ccc(CN4CCC(c5ccc6c(c5)CN(C5CCC(=O)NC5=O)C6=O)CC4)cc3c2=O)c(C)n1]

Compound 70 is prepared according to similar procedures as described in Examples 16 and 18.

### Example 35: Synthesis of 3-(5-(1-((3-(6-isopropylpyridin-3-yl)-4-oxo-3,4-dihydroquinazolin-6-yl)methyl)piperidin-4-yl)-1-oxoisoindolin-2-yl)piperidine-2,6-dione (71)

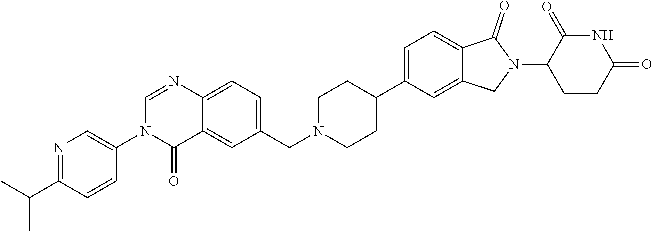 [SMILES: CC(C)c1ccc(-n2cnc3ccc(CN4CCC(c5ccc6c(c5)CN(C5CCC(=O)NC5=O)C6=O)CC4)cc3c2=O)cn1]

Compound 71 is prepared according to similar procedures as described in Examples 16 and 18.

### Example 36: Synthesis of 3-(5-(1-((3-(2-isopropylpyridin-3-yl)-4-oxo-3,4-dihydroquinazolin-6-yl)methyl)piperidin-4-yl)-1-oxoisoindolin-2-yl)piperidine-2,6-dione (72)

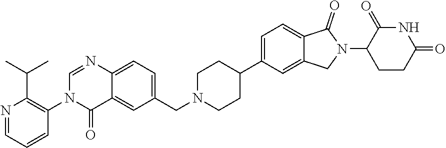 [SMILES: CC(C)c1ncccc1-n1cnc2ccc(CN3CCC(c4ccc5c(c4)CN(C4CCC(=O)NC4=O)C5=O)CC3)cc2c1=O]

Compound 72 is prepared according to similar procedures as described in Examples 16 and 18.

### Example 37: Synthesis of 3-(1-oxo-5-(1-((4-oxo-3-(6-(trifluoromethyl)pyridin-3-yl)-3,4-dihydroquinazolin-6-yl)methyl)piperidin-4-yl)isoindolin-2-yl)piperidine-2,6-dione (73)

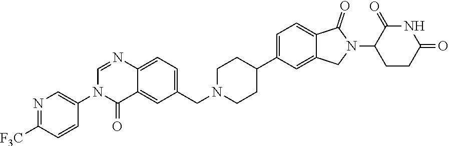 [SMILES: O=C1CCC(N2Cc3cc(C4CCN(Cc5ccc6ncn(-c7ccc(C(F)(F)F)nc7)c(=O)c6c5)CC4)ccc3C2=O)C(=O)N1]

Compound 73 was prepared according to similar procedures as described in Examples 16 and 18. MS [M+H]⁺=631.1.

### Example 38: Synthesis of 3-(5-(1-((3-(6-methoxypyridin-3-yl)-4-oxo-3,4-dihydroquinazolin-6-yl)methyl)piperidin-4-yl)-1-oxoisoindolin-2-yl)piperidine-2,6-dione (74)

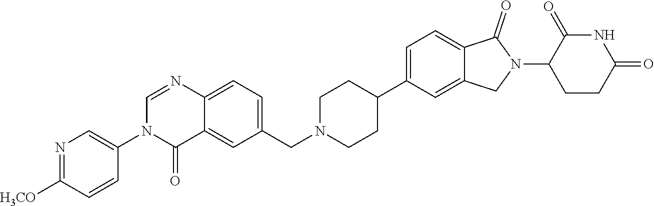 [SMILES: COc1ccc(-n2cnc3ccc(CN4CCC(c5ccc6c(c5)CN(C5CCC(=O)NC5=O)C6=O)CC4)cc3c2=O)cn1]

Compound 74 is prepared according to similar procedures as described in Examples 16 and 18.

### Example 39: Synthesis of 3-(5-(1-((3-(5-fluoropyridin-3-yl)-4-oxo-3,4-dihydroquinazolin-6-yl)methyl)piperidin-4-yl)-1-oxoisoindolin-2-yl)piperidine-2,6-dione (75)

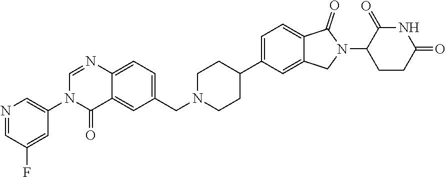 [SMILES: O=C1CCC(N2Cc3cc(C4CCN(Cc5ccc6ncn(-c7cncc(F)c7)c(=O)c6c5)CC4)ccc3C2=O)C(=O)N1]

Compound 75 was prepared according to similar procedures as described in Examples 16 and 18. MS [M+H]⁺=581.3.

### Example 40: Synthesis of 3-(1-oxo-5-(1-((4-oxo-3-(pyridazin-3-yl)-3,4-dihydroquinazolin-6-yl)methyl)piperidin-4-yl)isoindolin-2-yl)piperidine-2,6-dione (76)

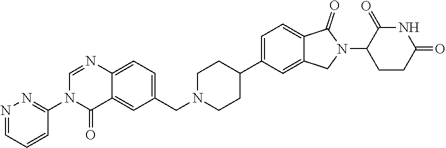 [SMILES: O=C1CCC(N2Cc3cc(C4CCN(Cc5ccc6ncn(-c7cccnn7)c(=O)c6c5)CC4)ccc3C2=O)C(=O)N1]

Compound 76 is prepared according to similar procedures as described in Examples 16 and 18.

### Example 41: Synthesis of 3-(1-oxo-5-(1-((4-oxo-3-(pyrazin-2-yl)-3,4-dihydroquinazolin-6-yl)methyl)piperidin-4-yl)isoindolin-2-yl)piperidine-2,6-dione (77)

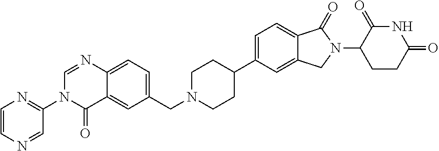 [SMILES: O=C1CCC(N2Cc3cc(C4CCN(Cc5ccc6ncn(-c7cnccn7)c(=O)c6c5)CC4)ccc3C2=O)C(=O)N1]

Compound 77 is prepared according to similar procedures as described in Examples 16 and 18.

### Example 42: Synthesis of 3-(1-oxo-5-(1-((4-oxo-3-(pyridin-4-yl)-3,4-dihydroquinazolin-6-yl)methyl)piperidin-4-yl)isoindolin-2-yl)piperidine-2,6-dione (78)

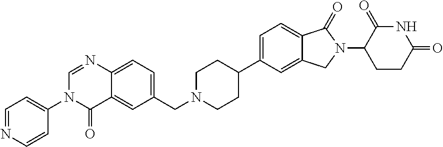 [SMILES: O=C1CCC(N2Cc3cc(C4CCN(Cc5ccc6ncn(-c7ccncc7)c(=O)c6c5)CC4)ccc3C2=O)C(=O)N1]

Compound 78 is prepared according to similar procedures as described in Examples 16 and 18.

### Example 43: Synthesis of 3-(1-oxo-5-(1-((4-oxo-3-(2-oxo-1,2-dihydropyridin-3-yl)-3,4-dihydroquinazolin-6-yl)methyl)piperidin-4-yl)isoindolin-2-yl)piperidine-2,6-dione (79)

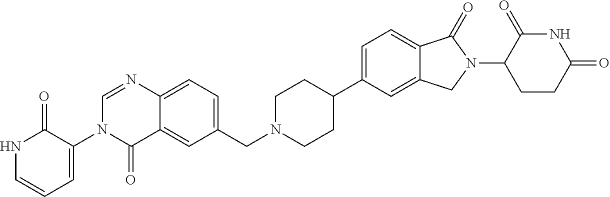 [SMILES: O=C1CCC(N2Cc3cc(C4CCN(Cc5ccc6ncn(-c7ccc[nH]c7=O)c(=O)c6c5)CC4)ccc3C2=O)C(=O)N1]

Compound 79 is prepared according to similar procedures as described in Examples 16 and 18.

### Example 44: Synthesis of 3-(1-oxo-5-(1-((4-oxo-3-(6-oxo-1,6-dihydropyridin-2-yl)-3,4-dihydroquinazolin-6-yl)methyl)piperidin-4-yl)isoindolin-2-yl)piperidine-2,6-dione (80)

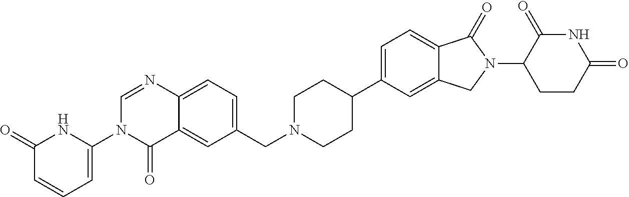 [SMILES: O=C1CCC(N2Cc3cc(C4CCN(Cc5ccc6ncn(-c7cccc(=O)[nH]7)c(=O)c6c5)CC4)ccc3C2=O)C(=O)N1]

Compound 80 is prepared according to similar procedures as described in Examples 16 and 18.

### Example 45: Synthesis of 3-(5-(1-((3-(1-methyl-1H-imidazol-5-yl)-4-oxo-3,4-dihydroquinazolin-6-yl)methyl)piperidin-4-yl)-1-oxoisoindolin-2-yl)piperidine-2,6-dione (81)

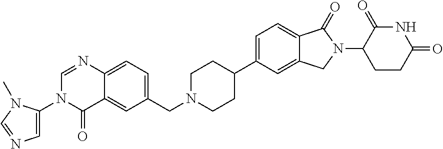 [SMILES: Cn1cncc1-n1cnc2ccc(CN3CCC(c4ccc5c(c4)CN(C4CCC(=O)NC4=O)C5=O)CC3)cc2c1=O]

Compound 81 is prepared according to similar procedures as described in Example 8.

### Example 46: Synthesis of 3-(5-(1-((3-(1-methyl-1H-pyrazol-4-yl)-4-oxo-3,4-dihydroquinazolin-6-yl)methyl)piperidin-4-yl)-1-oxoisoindolin-2-yl)piperidine-2,6-dione (82)

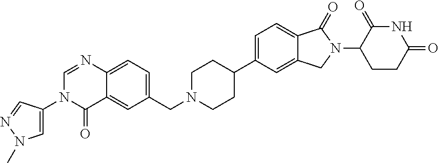

Compound 82 was prepare according to similar procedures as described in Examples 16 and 18. MS [M+H]⁺=566.2.

### Example 47: Synthesis of 3-(5-(1-((2-methyl-4-oxo-3-phenyl-3,4-dihydroquinazolin-6-yl)methyl)piperidin-4-yl)-1-oxoisoindolin-2-yl)piperidine-2,6-dione (83)

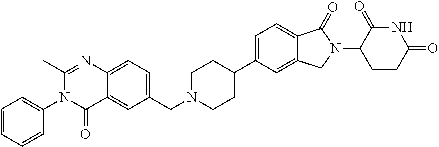 [SMILES: Cc1nc2ccc(CN3CCC(c4ccc5c(c4)CN(C4CCC(=O)NC4=O)C5=O)CC3)cc2c(=O)n1-c1ccccc1]

Compound 83 was prepared according to similar procedures as described in Examples 17 and 18. MS [M+H]⁺=576.8.

### Example 48: Synthesis of 3-(5-(1-((2-methyl-4-oxo-3-(pyridin-2-yl)-3,4-dihydroquinazolin-6-yl)methyl)piperidin-4-yl)-1-oxoisoindolin-2-yl)piperidine-2,6-dione (84)

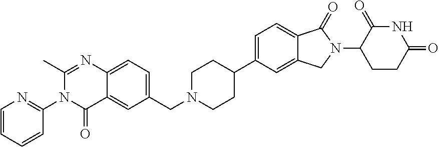 [SMILES: Cc1nc2ccc(CN3CCC(c4ccc5c(c4)CN(C4CCC(=O)NC4=O)C5=O)CC3)cc2c(=O)n1-c1ccccn1]

Compound 84 was prepared according to similar procedures as described in Examples 17 and 18. MS [M+H]⁺=577.3.

### Example 49: Synthesis of 3-(4-fluoro-1-oxo-5-(1-((4-oxo-3-(pyridin-2-yl)-3,4-dihydroquinazolin-6-yl)methyl)piperidin-4-yl)isoindolin-2-yl)piperidine-2,6-dione (85)

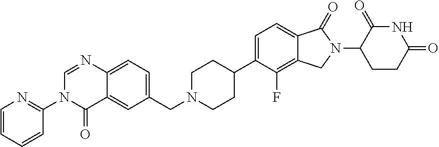 [SMILES: O=C1CCC(N2Cc3c(ccc(C4CCN(Cc5ccc6ncn(-c7ccccn7)c(=O)c6c5)CC4)c3F)C2=O)C(=O)N1]

Compound 85 was prepared according to similar procedures as described in Examples 11, 16, and 18. MS [M+H]⁺=581.7.

### Example 50: Synthesis of 3-(5-(1-((8-fluoro-4-oxo-3-(pyridin-2-yl)-3,4-dihydroquinazolin-6-yl)methyl)piperidin-4-yl)-1-oxoisoindolin-2-yl)piperidine-2,6-dione (86)

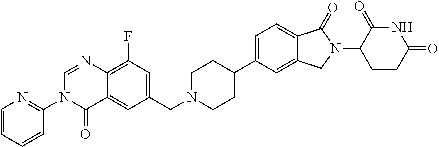 [SMILES: O=C1CCC(N2Cc3cc(C4CCN(Cc5cc(F)c6ncn(-c7ccccn7)c(=O)c6c5)CC4)ccc3C2=O)C(=O)N1]

Compound 86 was prepared according to similar procedures as described in Examples 16 and 18. MS [M+H]⁺=581.0.

### Example 51: Synthesis of 3-(5-(1-((7-fluoro-4-oxo-3-(pyridin-2-yl)-3,4-dihydroquinazolin-6-yl)methyl)piperidin-4-yl)-1-oxoisoindolin-2-yl)piperidine-2,6-dione (87)

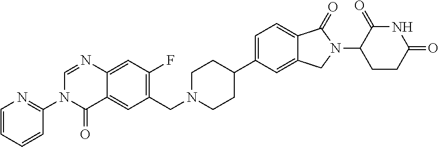 [SMILES: O=C1CCC(N2Cc3cc(C4CCN(Cc5cc6c(=O)n(-c7ccccn7)cnc6cc5F)CC4)ccc3C2=O)C(=O)N1]

Compound 87 was prepared according to similar procedures as described in Examples 16 and 18. MS [M+H]⁺=581.4.

### Example 52: Synthesis of 3-(5-(1-((5-fluoro-4-oxo-3-(pyridin-2-yl)-3,4-dihydroquinazolin-6-yl)methyl)piperidin-4-yl)-1-oxoisoindolin-2-yl)piperidine-2,6-dione (88)

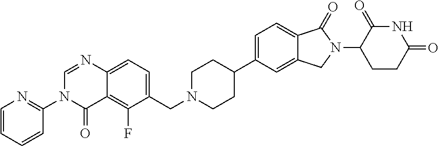 [SMILES: O=C1CCC(N2Cc3cc(C4CCN(Cc5ccc6ncn(-c7ccccn7)c(=O)c6c5F)CC4)ccc3C2=O)C(=O)N1]

Compound 88 was prepared according to similar procedures as described in Examples 14 and 15. MS [M+H]⁺=581.2.

### Example 53: Synthesis of 3-(4-fluoro-1-oxo-5-(1-((4-oxo-3-(pyridin-3-yl)-3,4-dihydroquinazolin-6-yl)methyl)piperidin-4-yl)isoindolin-2-yl)piperidine-2,6-dione (89)

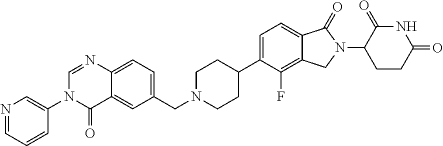 [SMILES: O=C1CCC(N2Cc3c(ccc(C4CCN(Cc5ccc6ncn(-c7cccnc7)c(=O)c6c5)CC4)c3F)C2=O)C(=O)N1]

Compound 89 was prepared according to similar procedures as described in Examples 11, 16, and 18. MS [M+H]⁺=581.2.

### Example 54: Synthesis of 3-(5-(1-((7-fluoro-4-oxo-3-(pyridin-3-yl)-3,4-dihydroquinazolin-6-yl)methyl)piperidin-4-yl)-1-oxoisoindolin-2-yl)piperidine-2,6-dione (90)

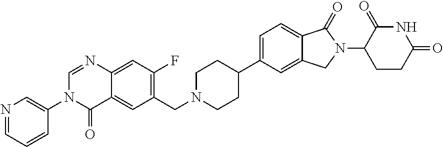 [SMILES: O=C1CCC(N2Cc3cc(C4CCN(Cc5cc6c(=O)n(-c7cccnc7)cnc6cc5F)CC4)ccc3C2=O)C(=O)N1]

Compound 90 is prepared according to similar procedures as described in Examples 16 and 18.

### Example 55: Synthesis of 3-(5-(1-((5-fluoro-4-oxo-3-(pyridin-3-yl)-3,4-dihydroquinazolin-6-yl)methyl)piperidin-4-yl)-1-oxoisoindolin-2-yl)piperidine-2,6-dione (91)

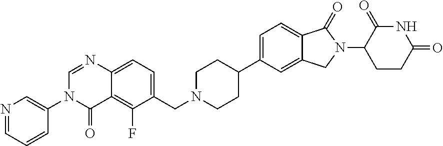 [SMILES: O=C1CCC(N2Cc3cc(C4CCN(Cc5ccc6ncn(-c7cccnc7)c(=O)c6c5F)CC4)ccc3C2=O)C(=O)N1]

Compound 91 is prepared according to similar procedures as described in Examples 16 and 18.

### Example 56: Synthesis of 3-(1-oxo-5-(1-((2-(pyridin-2-yl)pyrazolo[1,5-a]pyridin-6-yl)methyl)piperidin-4-yl)isoindolin-2-yl)piperidine-2,6-dione (92)

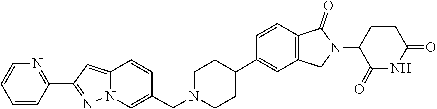 [SMILES: O=C1CCC(N2Cc3cc(C4CCN(Cc5ccc6cc(-c7ccccn7)nn6c5)CC4)ccc3C2=O)C(=O)N1]

Compound 92 was prepared according to similar procedures as described in Example 9. MS [M+H]⁺=535.4.

### Example 57: Synthesis of 3-(1-oxo-5-(1-((2-(pyridin-3-yl)pyrazolo[1,5-a]pyridin-6-yl)methyl)piperidin-4-yl)isoindolin-2-yl)piperidine-2,6-dione (93)

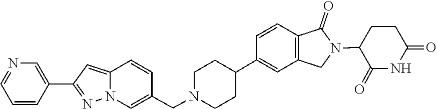 [SMILES: O=C1CCC(N2Cc3cc(C4CCN(Cc5ccc6cc(-c7cccnc7)nn6c5)CC4)ccc3C2=O)C(=O)N1]

Compound 93 is prepared according to similar procedures as described in Example 9.

### Example 58: Synthesis of 3-(1-oxo-5-(1-((2-(pyridin-2-yl)-2H-indazol-6-yl)methyl)piperidin-4-yl)isoindolin-2-yl)piperidine-2,6-dione (94)

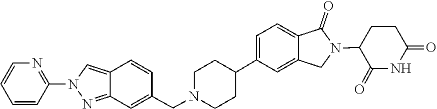 [SMILES: O=C1CCC(N2Cc3cc(C4CCN(Cc5ccc6cn(-c7ccccn7)nc6c5)CC4)ccc3C2=O)C(=O)N1]

Compound 94 is prepared according to similar procedures as described in Example 24.

### Example 59: Synthesis of 3-(1-oxo-5-(1-((7-phenylimidazo[1,2-a]pyridin-2-yl)methyl)piperidin-4-yl)isoindolin-2-yl)piperidine-2,6-dione (98)

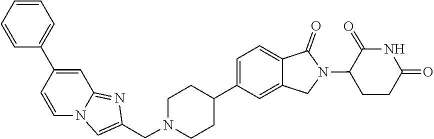 [SMILES: O=C1CCC(N2Cc3cc(C4CCN(Cc5cn6ccc(-c7ccccc7)cc6n5)CC4)ccc3C2=O)C(=O)N1]

Compound 98 was prepared according to similar procedures as described in Examples 21 and 22. MS [M+H]⁺=534.2.

### Example 60: Synthesis of 3-(1-oxo-5-(1-((7-(pyridin-3-yl)imidazo[1,2-a]pyridin-2-yl)methyl)piperidin-4-yl)isoindolin-2-yl)piperidine-2,6-dione (99)

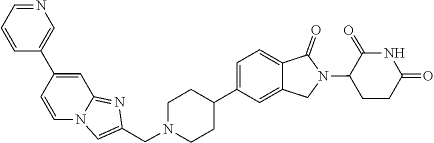 [SMILES: O=C1CCC(N2Cc3cc(C4CCN(Cc5cn6ccc(-c7cccnc7)cc6n5)CC4)ccc3C2=O)C(=O)N1]

Compound 99 was prepared according to similar procedures as described in Examples 21 and 23. MS [M+H]⁺=535.3.

### Example 61: Liquid Chromatography Mass Spectrometry (LCMS) Data

Reaction monitoring and final compound characterization were done using Shimadzu LC-20AD series (binary pump and diode array detector) with Luna®-C18 column (3 μm, 2.0×30 mm). Mobile phase: A: 0.04% Trifluoroacetic acid in water (v/v), B: 0.02% Trifluoroacetic acid in MeCN (v/v). Flow Rate: 1 mL/min (0.00-1.80 min) and 1.2 mL/min (1.81-2.00 min) at 25° C. MS: 2020, Quadrupole LC/MS, Ion Source: API-ESI, TIC: 100-1000 m/z; Drying gas flow: 15 L/min; Nebulizer pressure: 1.5 L/min; Drying gas temperature: 250° C., Vcap: 1400V.

TABLE 1
LCMS data of inventive compounds

| | Calculated | Observed |
|---|---|---|
| Compound # | Mass | Mass (M + H) |
| 2 | 578.25 | 579.42 |
| 3 | 579.25 | 580.23 |
| 5 | 596.24 | 597.35 |
| 6 | 612.21 | 613.35 |
| 15 | 562.22 | 563.34 |
| 22 | 576.27 | 577.35 |
| 27 | 579.23 | 580.10 |
| 41 | 544.25 | 545.40 |
| 42 | 533.24 | 534.35 |
| 44 | 561.24 | 562.40 |
| 45 | 596.24 | 597.45 |
| 46 | 580.24 | 581.35 |
| 47 | 580.24 | 581.35 |
| 48 | 585.20 | 586.35 |
| 49 | 585.20 | 586.35 |
| 57 | 561.24 | 562.10 |
| 58 | 580.22 | 581.20 |
| 59 | 568.19 | 569.10 |

### Example 62: Additional LCMS Data

Reaction monitoring and final compound characterization were collected using the Shimadzu N-Series UPLC-MS system (LCMS-2020). All masses reported are the m/z of the protonated parent ions unless recorded otherwise. The sample was dissolved in a suitable solvent such as methanol, acetonitrile, or DMSO and was directly injected into the column using an automated sample handler. The analysis was performed on, Waters® Acquity UPLC CSH C18 1.7 μm, 2.1×30 mm: flow rate: 1.0 mL/min; 40° C. (Column temperature) Solvent A: 0.1% formic acid in water, solvent B: 0.1% formic acid in Acetonitrile, gradient: Solvent A:0.01 min-3.0%, 1.5-1.9 min-97.0%, 2.0 min-3.0%. Agilent Zorbax eclipse plus C18 2.1×50 mm 1.8 μm: flow rate: 0.8 mL/Min: 40° C. (Column temperature) Solvent A: 0.1% formic acid in water, solvent B: 0.1% formic acid in Acetonitrile, gradient: Solvent A: 0.01-0.25 min-5.0%, 2.5-3.0 min-100.0%, 3.1-4.0 min-5.0%. Waters X-Bridge C18 (50×4.6) mm, 3.5 μm: flow rate: 0.8 mL/min; 40° C. (Column temperature): Solvent A: 10 mM Ammonium bicarbonate in water, solvent B: Acetonitrile, gradient: Solvent A: 0.01 min-5.0%, 1.5-3.0 min-95.0%, 3.5-4.0 min-5.0%. Waters X-Bridge C18 (50×4.6) mm, 3.5 μm: flow rate: 0.8 mL/min; 40° C. (Column temperature): Solvent A: 10 mm Ammonium acetate in water, solvent B: Acetonitrile, gradient: Solvent A: 0.01 min-5.0%, 1.5-3.0 min-95.0%, 3.5-4.0 min-5.0%.

TABLE 2
LCMS data of inventive compounds

| | | Observed |
|---|---|---|
| Compound | Calculated | Mass |
| # | Mass | (M + H) |
| 28 | 579.2 | 580.2 |
| 54 | 533.2 | 534.2 |
| 55 | 534.2 | 535.2 |
| 60 | 629.2 | 630.1 |
| 61 | 586.2 | 587.2 |
| 62 | 576.2 | 577.1 |
| 63 | 604.3 | 605.1 |
| 64 | 630.2 | 631.1 |
| 65 | 592.2 | 593.1 |
| 67 | 562.2 | 563.2 |
| 68 | 576.2 | 577.4 |
| 69 | 576.2 | 577.3 |
| 73 | 630.2 | 631.1 |
| 75 | 580.2 | 581.3 |
| 82 | 565.2 | 566.2 |
| 83 | 575.3 | 576.8 |
| 84 | 576.2 | 577.3 |
| 85 | 580.2 | 581.7 |
| 86 | 580.2 | 581.0 |
| 87 | 580.2 | 581.4 |
| 88 | 580.2 | 581.2 |
| 89 | 580.2 | 581.2 |
| 92 | 534.2 | 535.4 |
| 95 | 534.2 | 535.3 |
| 96 | 533.2 | 534.2 |
| 97 | 534.2 | 535.3 |
| 98 | 533.2 | 534.2 |
| 99 | 534.2 | 535.3 |

### Example 63: HiBiT Protocol

The HiBiT protein tagging system was applied to MOLT4 cells via a CRISPR/Cas-mediated insertion of the HiBiT peptide tag (Promega™) to the N-terminus of the IKZF2 gene locus (Neon™ Transfection System). The resulting HiBiT-Helios stable cell line was treated with the following inventive compounds in triplicates following a 13-point concentration scheme ranging from 10 μM to 0.00026 μM. At the indicated timepoints, the Nano-Glo® HiBiT Lytic Detection system (Promega™) was utilized for detecting bioluminescence of the HiBiT tag in treated cells: the abundance of the tag is proportionate to the level of luminescence. Following normalization to DMSO, dose-response curves were plotted (GraphPad Prism) to determine the concentration points at which 50% of HiBiT-Helios degradation was achieved by each compound. The extent of degradation (range of luminescence) from the highest to lowest concentration points was calculated to determine Dmax. The results are shown in Table 3.

TABLE 3
Degradation Activity (HiBiT-IKZF2 assay) of inventive
compounds after 6 hours of treatment

| Compound Number | IKZF2 DC₅₀ (M) |
|---|---|
| 1 | +++ |
| 2 | ++ |
| 3 | ++ |
| 5 | +++ |
| 6 | +++ |
| 8 | ++ |
| 12 | ++ |
| 18 | +++ |
| 22 | ++ |
| 27 | ++ |
| 31 | ++ |
| 38 | ++ |
| 41 | + |
| 42 | +++ |
| 43 | ++ |
| 44 | ++ |
| 45 | +++ |
| 46 | ++ |
| 47 | ++ |
| 48 | ++ |
| 49 | +++ |
| 57 | +++ |
| 58 | +++ |
| 59 | ++ |

“+” is from greater than 25 to less than 100 × 10⁻⁹M;
“++” is from greater than 5 to less than 25 × 10⁻⁹M;
“+++” is less than 5 × 10⁻⁹M

### Example 64: MOLT4 IKZF2 HiBit Assay Protocol

This protocol uses MOLT4 cells that were engineered using CRISPR/Cas9-mediated genomic insertion of HiBit, tagged to the N terminus of the IKZF2 coding sequence. Day 1. Created a 10-point dose response starting from 30 μM down to 1 nM using a Tecan D300e. Next added 8,000 cells/well of the MOLT4 IKZF2 HiBit cells to the compound plate. Incubated for 24 hours. Day 2. Added Nano-Glo® HiBit Lytic Buffer/Nano-Glo® HiBit Lytic substrate/LgBit Protein mix to each well and incubated for 15 minutes. Finally, read luminescence signal using a BMG Labtech PHERAstar® FSX. Data was normalized to DMSO and graphed using GraphPad Prism to determine the concentration points at which 50% of HiBiT-Helios degradation was achieved by each compound. The extent of degradation (range of luminescence) from the highest to lowest concentration points was calculated to determine Dmax. Data for selected compounds is provided in Table 4.

TABLE 4
Degradation Activity (HiBiT-IKZF2 assay) of inventive
compounds after 6 hours of treatment

| Compound Number | IKZF2 DC₅₀ (M) |
|---|---|
| 28 | ++ |
| 54 | +++ |
| 55 | + |
| 60 | ++ |
| 61 | ++ |
| 62 | ++ |
| 63 | ++ |
| 64 | ++ |
| 65 | ++ |
| 67 | ++ |
| 68 | +++ |
| 69 | + + |
| 75 | + |
| 82 | ++ |
| 83 | +++ |
| 84 | +++ |
| 85 | + |
| 87 | +++ |
| 89 | ++ |
| 92 | ++ |
| 96 | + |
| 97 | + |
| 98 | + |
| 99 | + |

“+” is from greater than 25 to less than 100 × 10⁻⁹M;
“++” is from greater than 5 to less than 25 × 10⁻⁹M;
“+++” is less than 5 × 10⁻⁹M

All patent publications and non-patent publications are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A compound having a structure represented by formula (I): 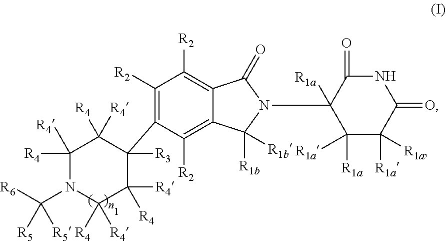
or a pharmaceutically acceptable salt, hydrate, solvate, prodrug, stereoisomer, or tautomer thereof,
wherein:
each R₁ₐ, R_{1b}, R₁ₐ′ and R_{1b}′ are independently hydrogen or (C₁-C₆)alkyl, or
R₁ₐ and R₁ₐ′, together with the same carbon atom to which they are attached, form a spiro (C₃-C₇)cycloalkyl group or a 4- to 7-membered heterocycloalkyl group, or
R₁ₐ and R₁ₐ′, when on different carbon atoms, together with the atoms to which they are attached, form a (C₃-C₇)cycloalkyl group or a 4- to 7-membered heterocycloalkyl group, or
R_{1b} and R_{1b}′ form a spiro (C₃-C₇)cycloalkyl group or a 4- to 7-membered heterocycloalkyl group; wherein said alkyl, cycloalkyl, or heterocycloalkyl is further optionally and independently substituted by one or more identical or different R₁₅ groups;
each R₂ is independently selected from the group consisting of hydrogen, hydroxy, amino, cyano, halo, (C₁-C₆)alkyl, and (C₁-C₆)haloalkyl;
R₃ is selected from the group consisting of hydrogen, amino, hydroxyl, cyano, halogen, (C₁-C₆)alkyl, and (C₁-C₆)haloalkyl, wherein said alkyl, is further optionally and independently substituted by one or more identical or different R₁₅ groups, or
R₃ and R₄, together with the carbon atoms to which they are attached, form a (C₃-C₇)cycloalkyl group or a 4- to 7-membered heterocycloalkyl group, or
R₂ and R₃, together with the atoms to which they are attached, form a (C₃-C₇)cycloalkyl group or a 4- to 7-membered heterocycloalkyl group, wherein said cycloalkyl, heterocycloalkyl is further optionally and independently substituted by one or more identical or different R₁₅ groups;
each R₄ and R₄′ is independently selected from the group consisting of hydrogen, hydroxyl, amino, amido, carbonyl, cyano, halogen, (C₁-C₆)alkyl, (C₁-C₆)haloalkyl, (C₁-C₆)hydroxyalkyl, (C₃-C₇)cycloalkyl, 4- to 7-membered heterocycloalkyl, (C₆-C₁₀)aryl, monocyclic and bicyclic 5- to 10-membered heteroaryl, (C₂-C₆)alkenyl, and (C₂-C₆)alkynyl; wherein said alkyl, alkynyl, cycloalkyl, heterocycloalkyl, aryl, or heteroaryl is further optionally and independently substituted by one or more identical or different R₁₅ groups, or
R₄ and R₄′, together with the same carbon atom to which they are attached, form a spiro (C₃-C₇)cycloalkyl group or a 4- to 7-membered heterocycloalkyl group, or
R₄ and R₄′, together with the same carbon atom to which they are attached, form C═(O), or
R₄ and R₄′, when on different carbon atoms, together with the atoms to which they are attached, form a (C₃-C₇)cycloalkyl group or a 4- to 7-membered heterocycloalkyl group, or
R₄ and R₄′, together with the carbon atoms to which they are attached, form a (C₆-C₁₀)aryl or a 5- or 6-membered heteroaryl; wherein said cycloalkyl, heterocycloalkyl, aryl or heteroaryl is further optionally and independently substituted by one or more identical or different R₁₅ groups;
R₅ and R₅′ are independently selected from the group consisting of hydrogen, (C₁-C₆)alkyl, (C₂-C₆)alkenyl, (C₂-C₆)alkynyl, (C₁-C₆)haloalkyl, (C₁-C₆)hydroxyalkyl, (C₃-C₇)cycloalkyl, 4- to 7-membered heterocycloalkyl, (C₆-C₁₀)aryl, and monocyclic and bicyclic 5- to 10-membered heteroaryl; wherein said alkyl, cycloalkyl, heterocycloalkyl, aryl or heteroaryl is further optionally and independently substituted by one or more identical or different R₁₅ groups;
R₆ is an R₇-substituted aryl or a R₇-substituted heteroaryl; wherein said aryl or heteroaryl is further optionally and independently substituted by one or more identical or different R₁₅ groups, or
R₆ is: 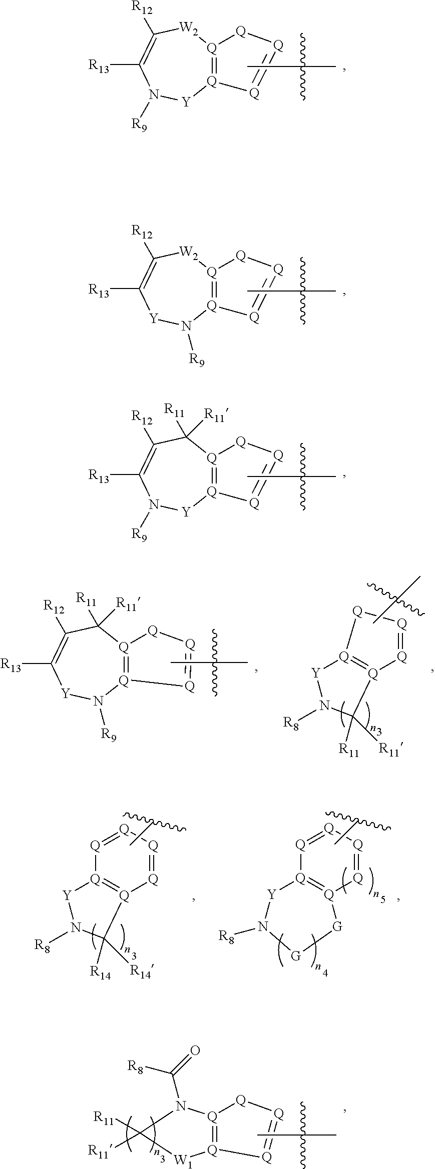 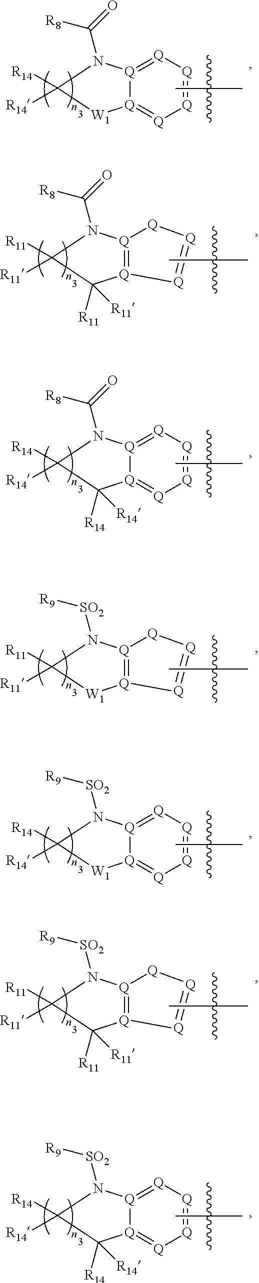 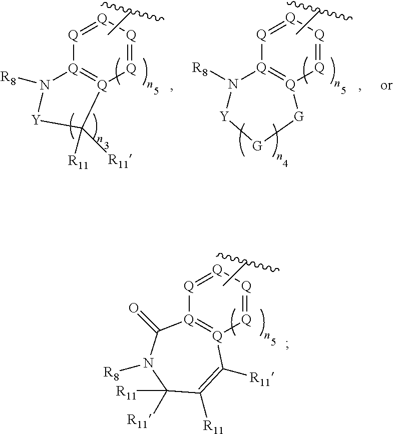
R₇ is selected from the group consisting of 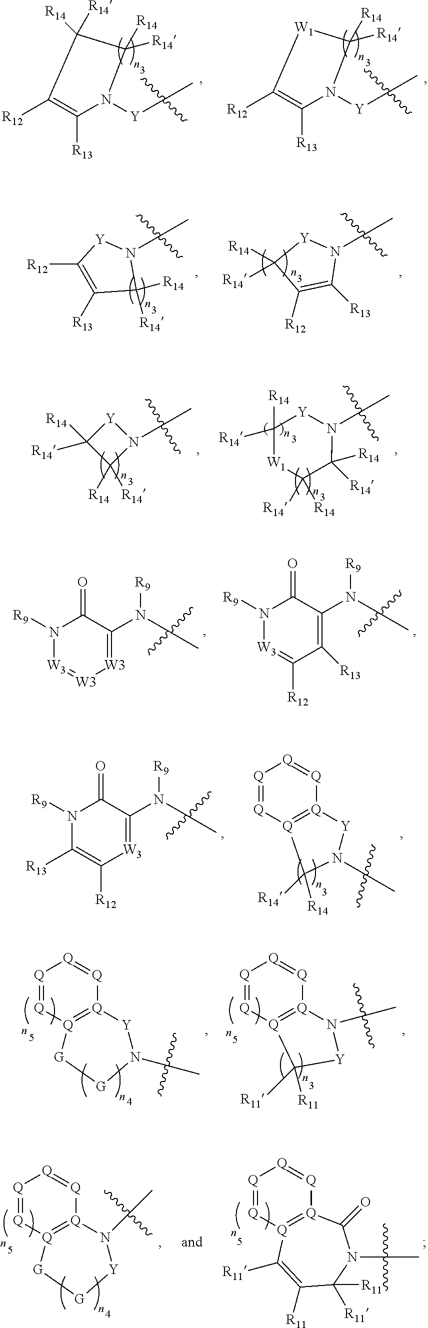
R₈ is selected from the group consisting of (C₆-C₀)aryl, and monocyclic and bicyclic 5- to 10-membered heteroaryl; wherein said aryl or heteroaryl is further optionally and independently substituted by one or more identical or different R₁₅ groups;
each R₉ is independently selected from the group consisting of hydrogen, (C₁-C₆)alkyl, (C₁-C₆)haloalkyl, (C₃-C₇)cycloalkyl, 4- to 7-membered heterocycloalkyl, (C₆-C₁₀)aryl, and monocyclic and bicyclic 5- to 10-membered heteroaryl; wherein said alkyl, cycloalkyl, heterocycloalkyl, aryl or heteroaryl is further optionally and independently substituted by one or more identical or different R₁₅ groups;
each R₁₁ and R₁₁′ is independently selected from the group consisting of hydrogen, (C₁-C₆)alkyl, (C₁-C₆)haloalkyl, (C₃-C₇)cycloalkyl, 4- to 7-membered heterocycloalkyl, (C₆-C₁₀)aryl, monocyclic and bicyclic 5- to 10-membered heteroaryl, halo, cyano, —N(R₉)₂, —OR₉, (C₁-C₆)alkoxy, (C₁-C₆)haloalkoxy, (C₂-C₆)alkenyl, and (C₂-C₆)alkynyl; wherein said alkyl, cycloalkyl, heterocycloalkyl, aryl, or heteroaryl is further optionally and independently substituted by one or more identical or different R₁₅ groups; or
R₁₁ and R₁₁′, together with the same carbon atom to which they are attached, form a spiro (C₃-C₇)cycloalkyl group or a 4- to 7-membered heterocycloalkyl group, or
R₁₁ and R₁₁′, together with the same carbon atom to which they are attached, form C═(O), or
R₁₁ and R₁₁′, when on different carbon atoms, together with the atoms to which they are attached, form a (C₃-C₇)cycloalkyl group or a 4- to 7-membered heterocycloalkyl group; wherein said cycloalkyl or heterocycloalkyl is further optionally and independently substituted by one or more identical or different R₁₅ groups;
R₁₂ and R₁₃, together with the carbon atoms to which they are attached, form a (C₆-C₁₀)aryl, or a monocyclic or bicyclic 5- to 10-membered heteroaryl, wherein said aryl or heteroaryl is further optionally and independently substituted by one or more identical or different R₁₅ groups;
each R₁₄ and R₁₄′ is independently selected from the group consisting of hydrogen, (C₁-C₆)alkyl, (C₁-C₆)haloalkyl, (C₃-C₇)cycloalkyl, 4- to 7-membered heterocycloalkyl, (C₆-C₁₀)aryl, monocyclic and bicyclic 5- to 10-membered heteroaryl, halo, cyano, —N(R₉)₂, —OR₉, (C₁-C₆)alkoxy, (C₁-C₆)haloalkoxy, (C₂-C₆)alkenyl, and (C₂-C₆)alkynyl; wherein said alkyl, cycloalkyl, heterocycloalkyl, aryl, or heteroaryl is further optionally and independently substituted by one or more identical or different R₁₅ groups; or
R₁₄ and R₁₄′, together with the same carbon atom to which they are attached, form a spiro (C₃-C₇)cycloalkyl group or a 4- to 7-membered heterocycloalkyl group, or
R₁₄ and R₁₄′, together with the same carbon atom to which they are attached, form C═(O), or
R₁₄ and R₁₄′, when on different carbon atoms, together with the atoms to which they are attached, form a (C₃-C₇)cycloalkyl group or a 4- to 7-membered heterocycloalkyl group; wherein said cycloalkyl, or heterocycloalkyl is further optionally and independently substituted by one or more identical or different R₁₅ groups,
provided that at least one R₁₄ and at least one R₁₄′, together with the same carbon atom to which they are attached, form C═(O);
each R₁₅ is independently selected from the group consisting of alkyl, alkenyl, alkynyl, halo, haloalkyl, cycloalkyl, heterocycloalkyl, hydroxy, alkoxy, cycloalkoxy, heterocycloalkoxy, haloalkoxy, aryloxy, heteroaryloxy, aralkyloxy, alkyenyloxy, alkynyloxy, amino, alkylamino, cycloalkylamino, heterocycloalkylamino, arylamino, heteroarylamino, aralkylamino, N-alkyl-N-arylamino, N-alkyl-N-heteroarylamino, N-alkyl-N-aralkylamino, hydroxyalkyl, aminoalkyl, alkylthio, haloalkylthio, alkylsulfonyl, haloalkylsulfonyl, cycloalkylsulfonyl, heterocycloalkylsulfonyl, arylsulfonyl, heteroarylsulfonyl, aminosulfonyl, alkylaminosulfonyl, cycloalkylaminosulfonyl, heterocycloalkylaminosulfonyl, arylaminosulfonyl, heteroarylaminosulfonyl, N-alkyl-N-arylaminosulfonyl, N-alkyl-N-heteroarylaminosulfonyl, formyl, alkylcarbonyl, haloalkylcarbonyl, alkenylcarbonyl, alkynylcarbonyl, carboxy, alkoxycarbonyl, alkylcarbonyloxy, amino, alkylsulfonylamino, haloalkylsulfonylamino, cycloalkylsulfonylamino, heterocycloalkylsulfonylamino, arylsulfonylamino, heteroarylsulfonylamino, aralkylsulfonylamino, alkylcarbonylamino, haloalkylcarbonylamino, cycloalkylcarbonylamino, heterocycloalkylcarbonylamino, arylcarbonylamino, heteroarylcarbonylamino, aralkylsulfonylamino, aminocarbonyl, alkylaminocarbonyl, cycloalkylaminocarbonyl, heterocycloalkylaminocarbonyl, arylaminocarbonyl, heteroarylaminocarbonyl, N-alkyl-N-arylaminocarbonyl, N-alkyl-N-heteroarylaminocarbonyl, cyano, nitro, azido, phosphinyl, phosphoryl including phosphine oxide and phosphonate, cyclic acetal, 4- to 7-membered heterocycloalkyl which contains at least one nitrogen atom and is linked via the nitrogen atom, aryl, heteroaryl, and where two adjacent R₁₅ taken together with the respective atoms to which each is bonded form aryl, heteroaryl, 5- to 8-membered cycloalkyl, or 5- to 8-membered heterocycloalkyl;
R₁₆ is independently selected from the group consisting of hydrogen, (C₁-C₆)alkyl, (C₁-C₆)haloalkyl, (C₃-C₇)cycloalkyl, 4- to 7-membered heterocycloalkyl, (C₆-C₁₀)aryl, monocyclic and bicyclic 5- to 10-membered heteroaryl, halo, cyano, —N(R₉)₂, —OR₉, (C₁-C₆)alkoxy, (C₁-C₆)haloalkoxy, (C₂-C₆)alkenyl, (C₂-C₆)alkynyl, and a radical that participates in the formation of a single bond; wherein said alkyl, cycloalkyl, heterocycloalkyl, aryl, or heteroaryl is further optionally and independently substituted by one or more identical or different R₁₅ groups;
each G is independently selected from C(R₁₁)(R₁₁′), NR₁₁ and O, provided that at least one G is NR₁₁ or O;
W₁ is selected from the group consisting of —O—, —S— and —NR₉—;
W₂ is selected from the group consisting of —O—, —S—, —SO₂—, —C═(O)— and —NR₉—;
each W₃ is nitrogen or CR₁₆;
Y is —SO₂— or —C═(O)—;
each Q is independently selected from C, C(R₁₆), C═(O), O, S, N, and NR₁₆;
n₁ is 0, 1 or 2;
n₃ is independently 1, 2 or 3;
n₄ is independently 1 or 2; and
n₅ is independently 0 or 1.

2. The compound of claim 1, wherein:
each R₁ₐ, R_{1b}, R₁ₐ′ and R_{1b}′ is hydrogen;
each R₂ is independently selected from the group consisting of hydrogen, halo, and (C₁-C₆)alkyl;
R₃ is selected from the group consisting of hydrogen, amino, hydroxyl, cyano, halogen, (C₁-C₆)alkyl, and (C₁-C₆)haloalkyl, wherein said alkyl is further optionally and independently substituted by one or more identical or different R₁₅ groups;
each R₄ and R₄′ is independently selected from the group consisting of hydrogen, hydroxyl, halogen, (C₁-C₆)alkyl, (C₁-C₆)haloalkyl, and (C₁-C₆)hydroxyalkyl; wherein said alkyl is further optionally and independently substituted by one or more identical or different R₁₅ groups, or
R₄ and R₄′, together with the same carbon atom to which they are attached, form a spiro (C₃-C₇)cycloalkyl group or a 4- to 7-membered heterocycloalkyl group, or
R₄ and R₄′, when on different carbon atoms, together with the atoms to which they are attached, form a (C₃-C₇)cycloalkyl group or a 4- to 7-membered heterocycloalkyl group;
R₅ and R₅′ are independently hydrogen or (C₁-C₆)alkyl; wherein said alkyl is further optionally and independently substituted by one or more identical or different R₁₅ groups;
R₆ is an R₇-substituted aryl or a R₇-substituted heteroaryl; wherein said aryl or heteroaryl is further optionally and independently substituted by one or more identical or different R₁₅ groups, or
R₆ is: 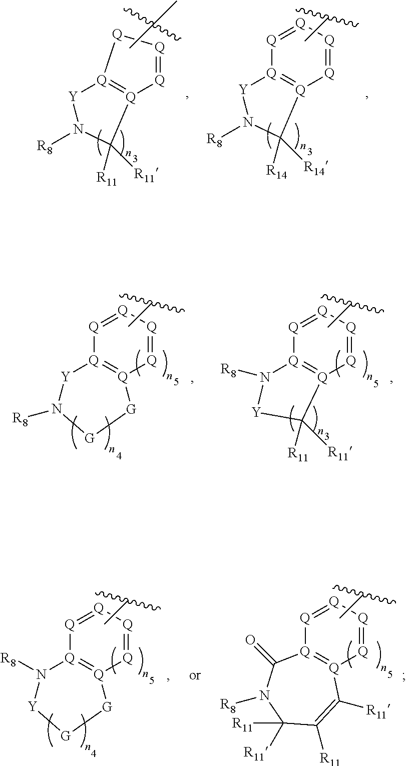
R₇ is selected from the group consisting of: 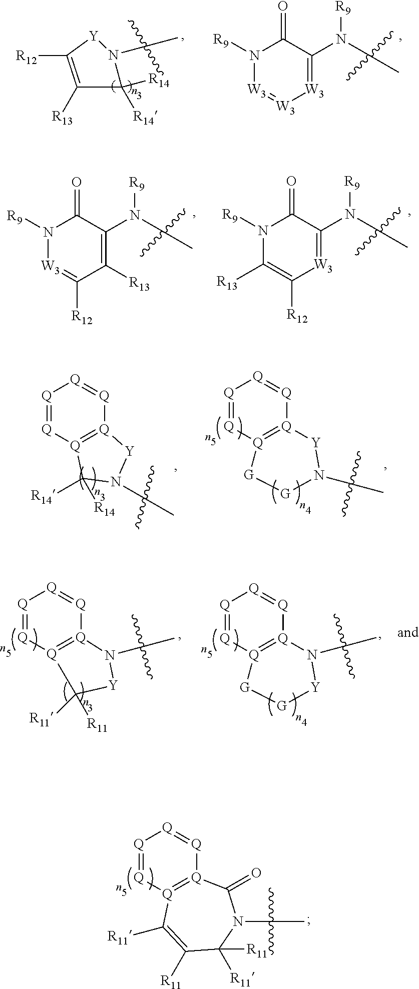
R₈ is selected from the group consisting of (C₆-C₁₀)aryl, and monocyclic and bicyclic 5- to 10-membered heteroaryl; wherein said aryl or heteroaryl is further optionally and independently substituted by one or more identical or different R₁₅ groups;
each R₉ is independently selected from the group consisting of hydrogen, (C₁-C₆)alkyl, (C₁-C₆)haloalkyl, (C₃-C₇)cycloalkyl, 4- to 7-membered heterocycloalkyl, (C₆-C₁₀)aryl, and monocyclic and bicyclic 5- to 10-membered heteroaryl; wherein said alkyl, cycloalkyl, heterocycloalkyl, aryl or heteroaryl is further optionally and independently substituted by one or more identical or different R₁₅ groups;
each R₁₁ and R₁₁′ is independently selected from the group consisting of hydrogen, (C₁-C₆)alkyl, (C₁-C₆)haloalkyl, (C₃-C₇)cycloalkyl, 4- to 7-membered heterocycloalkyl, (C₆-C₁₀)aryl, monocyclic and bicyclic 5- to 10-membered heteroaryl, halo, cyano, —N(R₉)₂, —OR₉, (C₁-C₆)alkoxy, (C₁-C₆)haloalkoxy, (C₂-C₆)alkenyl, and (C₂-C₆)alkynyl; wherein said alkyl, cycloalkyl, heterocycloalkyl, aryl, or heteroaryl is further optionally and independently substituted by one or more identical or different R₁₅ groups; or
R₁₁ and R₁₁′, together with the same carbon atom to which they are attached, form a spiro (C₃-C₇)cycloalkyl group or a 4- to 7-membered heterocycloalkyl group, or
R₁₁ and R₁₁′, together with the same carbon atom to which they are attached form, C═(O), or
R₁₁ and R₁₁′, when on different carbon atoms, together with the atoms to which they are attached, form a (C₃-C₇)cycloalkyl group or a 4- to 7-membered heterocycloalkyl group; wherein said cycloalkyl, or heterocycloalkyl is further optionally and independently substituted by one or more identical or different R₁₅ groups;
R₁₂ and R₁₃, together with the carbon atoms to which they are attached form, a (C₆-C₁₀)aryl, or a monocyclic or bicyclic 5- to 10-membered heteroaryl, wherein said aryl or heteroaryl is further optionally and independently substituted by one or more identical or different R₁₅ groups;
each R₁₄ and R₁₄′ is independently selected from the group consisting of hydrogen, (C₁-C₆)alkyl, (C₁-C₆)haloalkyl, (C₃-C₇)cycloalkyl, 4- to 7-membered heterocycloalkyl, (C₆-C₁₀)aryl, monocyclic and bicyclic 5- to 10-membered heteroaryl, halo, cyano, —N(R₉)₂, —OR₉, (C₁-C₆)alkoxy, (C₁-C₆)haloalkoxy, (C₂-C₆)alkenyl, and (C₂-C₆)alkynyl; wherein said alkyl, cycloalkyl, heterocycloalkyl, aryl, or heteroaryl is further optionally and independently substituted by one or more identical or different R₁₅ groups; or
R₁₄ and R₁₄′, together with the same carbon atom to which they are attached, form a spiro (C₃-C₇)cycloalkyl group or a 4- to 7-membered heterocycloalkyl group, or
R₁₄ and R₁₄′, together with the same carbon atom to which they are attached, form C═(O), or
R₁₄ and R₁₄′, when on different carbon atoms, together with the atoms to which they are attached, form a (C₃-C₇)cycloalkyl group or a 4- to 7-membered heterocycloalkyl group; wherein said cycloalkyl, or heterocycloalkyl is further optionally and independently substituted by one or more identical or different R₁₅ groups, provided that at least one R₁₄ and at least one R₁₄′, together with the same carbon atom to which they are attached, form C═(O);
each R₁₅ is independently selected from the group consisting of alkyl, alkenyl, alkynyl, halo, haloalkyl, cycloalkyl, heterocycloalkyl, hydroxy, alkoxy, cycloalkoxy, heterocycloalkoxy, haloalkoxy, aryloxy, heteroaryloxy, aralkyloxy, alkyenyloxy, alkynyloxy, amino, alkylamino, cycloalkylamino, heterocycloalkylamino, arylamino, heteroarylamino, aralkylamino, N-alkyl-N-arylamino, N-alkyl-N-heteroarylamino, N-alkyl-N-aralkylamino, hydroxyalkyl, aminoalkyl, alkylthio, haloalkylthio, alkylsulfonyl, haloalkylsulfonyl, cycloalkylsulfonyl, heterocycloalkylsulfonyl, arylsulfonyl, heteroarylsulfonyl, aminosulfonyl, alkylaminosulfonyl, cycloalkylaminosulfonyl, heterocycloalkylaminosulfonyl, arylaminosulfonyl, heteroarylaminosulfonyl, N-alkyl-N-arylaminosulfonyl, N-alkyl-N-heteroarylaminosulfonyl, formyl, alkylcarbonyl, haloalkylcarbonyl, alkenylcarbonyl, alkynylcarbonyl, carboxy, alkoxycarbonyl, alkylcarbonyloxy, amino, alkylsulfonylamino, haloalkylsulfonylamino, cycloalkylsulfonylamino, heterocycloalkylsulfonylamino, arylsulfonylamino, heteroarylsulfonylamino, aralkylsulfonylamino, alkylcarbonylamino, haloalkylcarbonylamino, cycloalkylcarbonylamino, heterocycloalkylcarbonylamino, arylcarbonylamino, heteroarylcarbonylamino, aralkylsulfonylamino, aminocarbonyl, alkylaminocarbonyl, cycloalkylaminocarbonyl, heterocycloalkylaminocarbonyl, arylaminocarbonyl, heteroarylaminocarbonyl, N-alkyl-N-arylaminocarbonyl, N-alkyl-N-heteroarylaminocarbonyl, cyano, nitro, azido, phosphinyl, phosphoryl including phosphine oxide and phosphonate, cyclic acetal, 4- to 7-membered heterocycloalkyl which contains at least one nitrogen atom and is linked via the nitrogen atom, aryl, heteroaryl, and where two adjacent R₁₅ taken together with the respective atoms to which each is bonded form aryl, heteroaryl, 5- to 8-membered cycloalkyl, or 5- to 8-membered heterocycloalkyl;
R₁₆ is independently selected from the group consisting of hydrogen, (C₁-C₆)alkyl, (C₁-C₆)haloalkyl, (C₃-C₇)cycloalkyl, 4- to 7-membered heterocycloalkyl, (C₆-C₁₀)aryl, monocyclic and bicyclic 5- to 10-membered heteroaryl, halo, cyano, —N(R₉)₂, —OR₉, (C₁-C₆)alkoxy, (C₁-C₆)haloalkoxy, (C₂-C₆)alkenyl, (C₂-C₆)alkynyl, and a radical that participates in the formation of a single bond; wherein said alkyl, cycloalkyl, heterocycloalkyl, aryl, or heteroaryl is further optionally and independently substituted by one or more identical or different R₁₅ groups;
each G is independently selected from C(R₁₁)(R₁₁′), NR₁₁ and O, provided that at least one G is NR₁₁ or O,
W₁ is selected from the group consisting of —O—, —S— and —NR₉—;
W₂ is selected from the group consisting of —O—, —S—, —SO₂—, —C═(O)— and —NR₉—;
each W₃ is nitrogen or CR₁₆;
Y is —SO₂— or —C═(O)—;
each Q is independently selected from C, C(R₁₆), C═(O), O, S, N, and NR₁₆,
n₁ is 0, 1 or 2;
n₃ is independently 1, 2 or 3;
n₄ is independently 1 or 2; and
n₅ is independently 0 or 1.

3. The compound of claim 2, wherein:
each R₂ is hydrogen;
R₃ is hydrogen or hydroxyl;
each R₄ and R₄′ is independently hydrogen or (C₁-C₆)alkyl;
R₅ and R₅′ are independently selected from hydrogen or (C₁-C₆)alkyl;
R₆ is 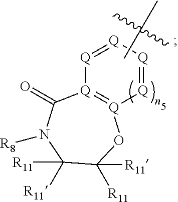
each R₁₁ and R₁₁′ is independently hydrogen or (C₁-C₆)alkyl, wherein said alkyl is further optionally and independently substituted by one or more identical or different R₁₅ groups; or
R₁₁ and R₁₁′, together with the same carbon atom to which they are attached, form a spiro (C₃-C₇)cycloalkyl group or a 4- to 7-membered heterocycloalkyl group, or
R₁₁ and R₁₁′, together with the same carbon atom to which they are attached form, C═(O), or
R₁₁ and R₁₁′, when on different carbon atoms, together with the atoms to which they are attached, form a (C₃-C₇)cycloalkyl group or a 4- to 7-membered heterocycloalkyl group; wherein said cycloalkyl, or heterocycloalkyl is further optionally and independently substituted by one or more identical or different R₁₅ groups;
R₁₆ is independently selected from the group consisting of hydrogen, (C₁-C₆)alkyl, (C₁-C₆)haloalkyl, halo, cyano, —N(R₉)₂, —OR₉, (C₁-C₆)alkoxy, (C₁-C₆)haloalkoxy, and a radical that participates in the formation of a single bond; wherein said alkyl is further optionally and independently substituted by one or more identical or different R₁₅ groups; and
n₁ is 1.

4. The compound of claim 3, wherein R₆ is 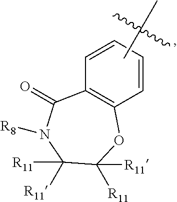
wherein R₆ is further optionally and independently substituted with one or more groups selected from (C₁-C₆)alkyl, halo, and cyano; and
each R₁₁ and R₁₁′ is independently hydrogen or (C₁-C₆)alkyl.

5. The compound of claim 3, wherein R₈ is selected from: 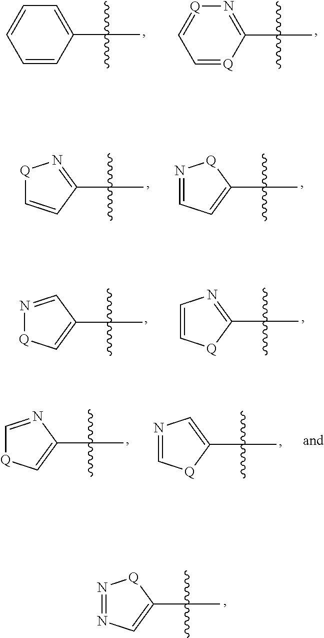
wherein R₈ is further optionally and independently substituted with one or more R₁₅.

6. The compound of claim 5, wherein R₈ is selected from: 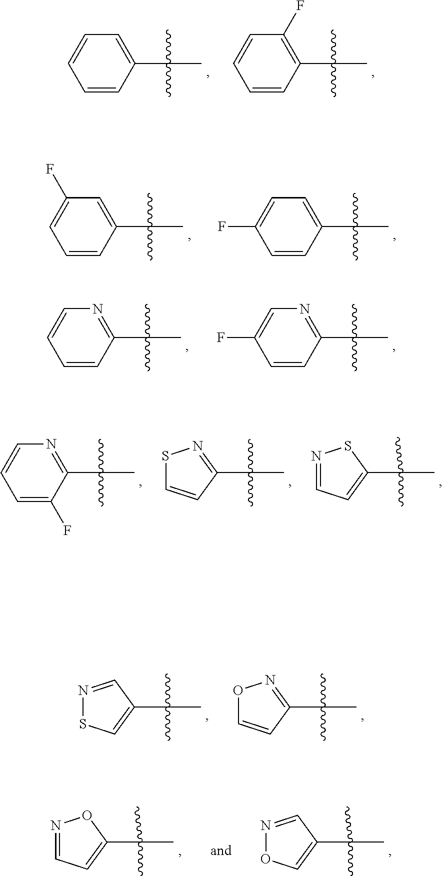
wherein R₈ is further optionally and independently substituted with one or more R₁₅.

7. A compound having a structure represented by formula (II): 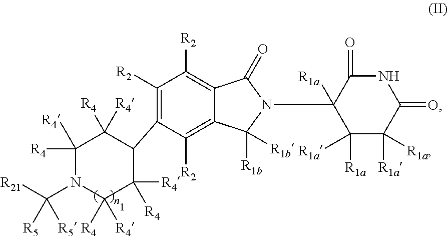
or a pharmaceutically acceptable salt, hydrate, solvate, prodrug, stereoisomer, or tautomer thereof,
wherein:
each R₁ₐ, R_{1b}, R₁ₐ′ and R_{1b}′ is independently hydrogen or (C₁-C₆)alkyl, or
R₁ₐ and R₁ₐ′, together with the same carbon atom to which they are attached, form a spiro (C₃-C₇)cycloalkyl group or a 4- to 7-membered heterocycloalkyl group, or
R₁ₐ and R₁ₐ′, when on different carbon atoms, together with the atoms to which they are attached, form a (C₃-C₇)cycloalkyl group or a 4- to 7-membered heterocycloalkyl group, or
R_{1b} and R_{1b}′ form a spiro (C₃-C₇)cycloalkyl group or a 4- to 7-membered heterocycloalkyl group; wherein said alkyl, cycloalkyl, or heterocycloalkyl is further optionally and independently substituted by one or more identical or different R₁₅ groups;
each R₂ is independently selected from the group consisting of hydrogen, hydroxy, amino, cyano, halo, (C₁-C₆)alkyl, and (C₁-C₆)haloalkyl;
each R₄ and R₄′ is independently selected from the group consisting of hydrogen, hydroxyl, amino, amido, carbonyl, cyano, halogen, (C₁-C₆)alkyl, (C₁-C₆)haloalkyl, (C₁-C₆)hydroxyalkyl, (C₃-C₇)cycloalkyl, 4- to 7-membered heterocycloalkyl, (C₆-C₁₀)aryl, monocyclic and bicyclic 5- to 10-membered heteroaryl, (C₂-C₆)alkenyl, and (C₂-C₆)alkynyl; wherein said alkyl, alkynyl, cycloalkyl, heterocycloalkyl, aryl, or heteroaryl is further optionally and independently substituted by one or more identical or different R₁₅ groups, or
R₄ and R₄′, together with the same carbon atom to which they are attached, form a spiro (C₃-C₇)cycloalkyl group or a 4- to 7-membered heterocycloalkyl group, or
R₄ and R₄′, together with the same carbon atom to which they are attached, form a C═(O), or
R₄ and R₄′, when on different carbon atoms, together with the atoms to which they are attached, form a (C₃-C₇)cycloalkyl group or a 4- to 7-membered heterocycloalkyl group, or
R₄ and R₄′, together with the carbon atoms to which they are attached, form a (C₆-C₁₀)aryl or a 5- or 6-membered heteroaryl; wherein said cycloalkyl, heterocycloalkyl, aryl or heteroaryl is further optionally and independently substituted by one or more identical or different R₁₅ groups;
R₅ and R₅′ are independently selected from the group consisting of hydrogen, (C₁-C₆)alkyl, (C₂-C₆)alkenyl, (C₂-C₆)alkynyl, (C₁-C₆)haloalkyl, (C₁-C₆)hydroxyalkyl, (C₃-C₇)cycloalkyl, 4- to 7-membered heterocycloalkyl, (C₆-C₁₀)aryl, and monocyclic and bicyclic 5- to 10-membered heteroaryl; wherein said alkyl, cycloalkyl, heterocycloalkyl, aryl or heteroaryl is further optionally and independently substituted by one or more identical or different R₁₅ groups;
each R₁₅ is independently selected from the group consisting of alkyl, alkenyl, alkynyl, halo, haloalkyl, cycloalkyl, heterocycloalkyl, hydroxy, alkoxy, cycloalkoxy, heterocycloalkoxy, haloalkoxy, aryloxy, heteroaryloxy, aralkyloxy, alkyenyloxy, alkynyloxy, amino, alkylamino, cycloalkylamino, heterocycloalkylamino, arylamino, heteroarylamino, aralkylamino, N-alkyl-N-arylamino, N-alkyl-N-heteroarylamino, N-alkyl-N-aralkylamino, hydroxyalkyl, aminoalkyl, alkylthio, haloalkylthio, alkylsulfonyl, haloalkylsulfonyl, cycloalkylsulfonyl, heterocycloalkylsulfonyl, arylsulfonyl, heteroarylsulfonyl, aminosulfonyl, alkylaminosulfonyl, cycloalkylaminosulfonyl, heterocycloalkylaminosulfonyl, arylaminosulfonyl, heteroarylaminosulfonyl, N-alkyl-N-arylaminosulfonyl, N-alkyl-N-heteroarylaminosulfonyl, formyl, alkylcarbonyl, haloalkylcarbonyl, alkenylcarbonyl, alkynylcarbonyl, carboxy, alkoxycarbonyl, alkylcarbonyloxy, amino, alkylsulfonylamino, haloalkylsulfonylamino, cycloalkylsulfonylamino, heterocycloalkylsulfonylamino, arylsulfonylamino, heteroarylsulfonylamino, aralkylsulfonylamino, alkylcarbonylamino, haloalkylcarbonylamino, cycloalkylcarbonylamino, heterocycloalkylcarbonylamino, arylcarbonylamino, heteroarylcarbonylamino, aralkylsulfonylamino, aminocarbonyl, alkylaminocarbonyl, cycloalkylaminocarbonyl, heterocycloalkylaminocarbonyl, arylaminocarbonyl, heteroarylaminocarbonyl, N-alkyl-N-arylaminocarbonyl, N-alkyl-N-heteroarylaminocarbonyl, cyano, nitro, azido, phosphinyl, phosphoryl including phosphine oxide and phosphonate, cyclic acetal, 4- to 7-membered heterocycloalkyl which contains at least one nitrogen atom and is linked via the nitrogen atom, aryl, heteroaryl, and where two adjacent R₁₅ taken together with the respective atoms to which each is bonded form aryl, heteroaryl, 5- to 8-membered cycloalkyl, or 5- to 8-membered heterocycloalkyl;
R₂₁ is a substituted C₆-aryl, provided said aryl is substituted with at least two R₁₅, and provided two of the R₁₅, when on adjacent carbon atoms, form a 5- or 6-membered heteroaryl that is substituted with at least one (C₆-C₁₀)aryl, or monocyclic or bicyclic 5- to 10-membered heteroaryl; wherein said aryl and heteroaryl are further optionally and independently substituted with one or more R₁₅, or
R₂₁ is a substituted 5- or 6-membered heteroaryl, provided said heteroaryl is substituted with at least two R₁₅, and provided two of the R₁₅, when on adjacent atoms, form a C₆-aryl, or 5- or 6-membered heteroaryl that is substituted with at least one (C₆-C₁₀)aryl, or monocyclic or bicyclic 5- to 10-membered heteroaryl; wherein said aryl and heteroaryl are further optionally and independently substituted with one or more R₁₅, or
R₂₁ is 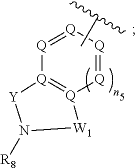
R₈ is selected from the group consisting of (C₆-C₁₀)aryl, and monocyclic and bicyclic 5- to 10-membered heteroaryl; wherein said aryl or heteroaryl is further optionally and independently substituted by one or more identical or different R₁₅ groups;
W₁ is selected from the group consisting of —O—, —S— and —NR₉—;
R₉ is independently selected from the group consisting of hydrogen, (C₁-C₆)alkyl, (C₁-C₆)haloalkyl, (C₃-C₇)cycloalkyl, 4- to 7-membered heterocycloalkyl, (C₆-C₁₀)aryl, and monocyclic and bicyclic 5- to 10-membered heteroaryl; wherein said alkyl, cycloalkyl, heterocycloalkyl, aryl or heteroaryl is further optionally and independently substituted by one or more identical or different R₁₅ groups;
Y is —SO₂— or —C═(O)—;
each Q is independently selected from C, C(R₁₆), C═(O), O, S, N, and NR₁₆;
R₁₆ is independently selected from the group consisting of hydrogen, (C₁-C₆)alkyl, (C₁-C₆)haloalkyl, (C₃-C₇)cycloalkyl, 4- to 7-membered heterocycloalkyl, (C₆-C₁₀)aryl, monocyclic and bicyclic 5- to 10-membered heteroaryl, halo, cyano, —N(R₉)₂, —OR₉, (C₁-C₆)alkoxy, (C₁-C₆)haloalkoxy, (C₂-C₆)alkenyl, (C₂-C₆)alkynyl, and a radical that participates in the formation of a single bond; wherein said alkyl, cycloalkyl, heterocycloalkyl, aryl, or heteroaryl is further optionally and independently substituted by one or more identical or different R₁₅ groups;
n₁ is 0, 1 or 2; and
n₅ is independently 0 or 1.

8. The compound of claim 7, wherein:
each R₁ₐ, R_{1b}, R₁ₐ′ and R_{1b}′ is hydrogen;
each R₂ is hydrogen;
each R₄ and R₄′ is independently hydrogen or (C₁-C₆)alkyl;
R₅ and R₅′ are each hydrogen or (C₁-C₆)alkyl;
R₂₁ is 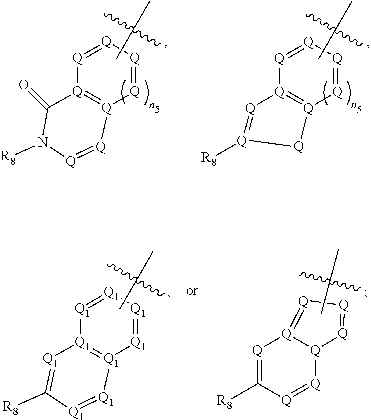
each Q₁ is independently selected from C, C(R₁₆), C═(O), O, S, N, and NR₁₆, provided that at least one Q₁ is N;
R₁₆ is independently selected from the group consisting of hydrogen, (C₁-C₆)alkyl, (C₁-C₆)haloalkyl, halo, cyano, —N(R₉)₂, —OR₉, (C₁-C₆)alkoxy, (C₁-C₆)haloalkoxy, and a radical that participates in the formation of a single bond; wherein said alkyl, cycloalkyl, heterocycloalkyl, aryl, or heteroaryl is further optionally and independently substituted by one or more identical or different R₁₅ groups; and
n₁ is 1.

9. The compound of claim 8, wherein R₂₁ is selected from: 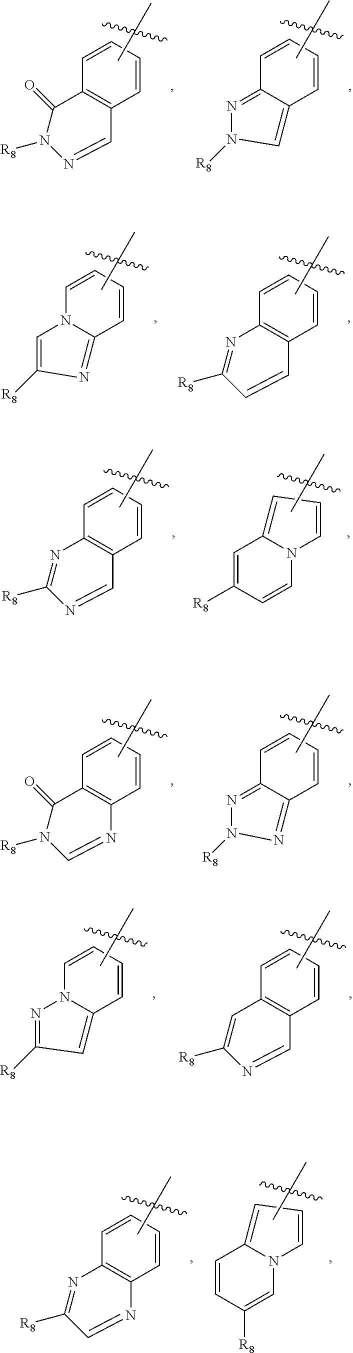 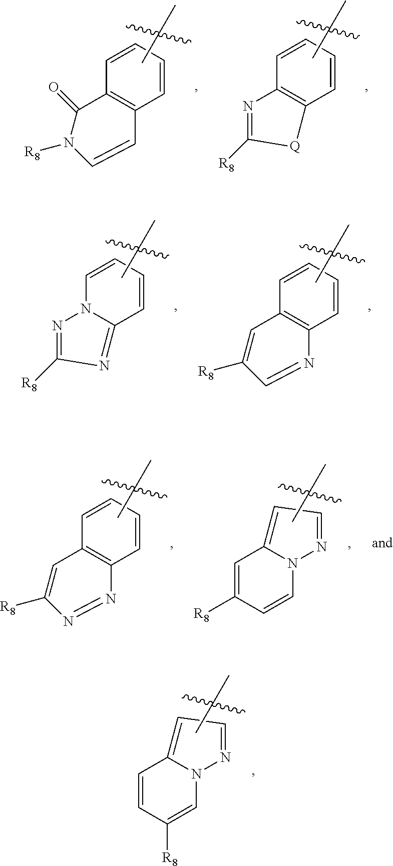
wherein R₂₁ is optionally and independently substituted with one or more (C₁-C₆)alkyl, halo, and cyano.

10. The compound of claim 8, wherein R₈ is selected from: 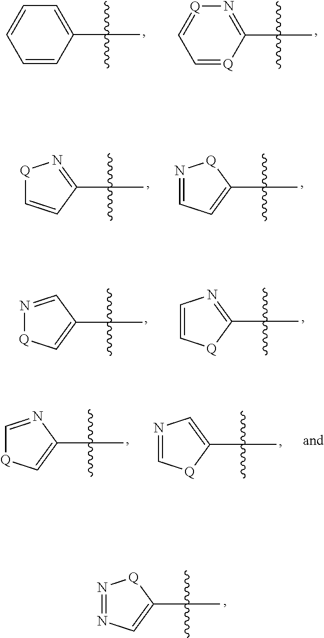
wherein R₈ is further optionally and independently substituted with one or more R₁₅.

11. The compound of claim 10, wherein R₈ is selected from: 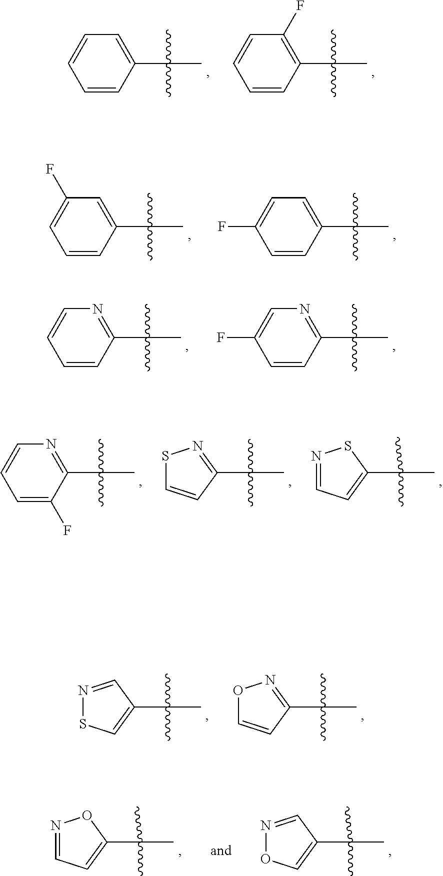
wherein R₈ is further optionally and independently substituted with one or more R₁₅.

12. (canceled)

13. The compound of claim 7, which is represented by formula IIa, IIb, IIc, IId, or IIe: 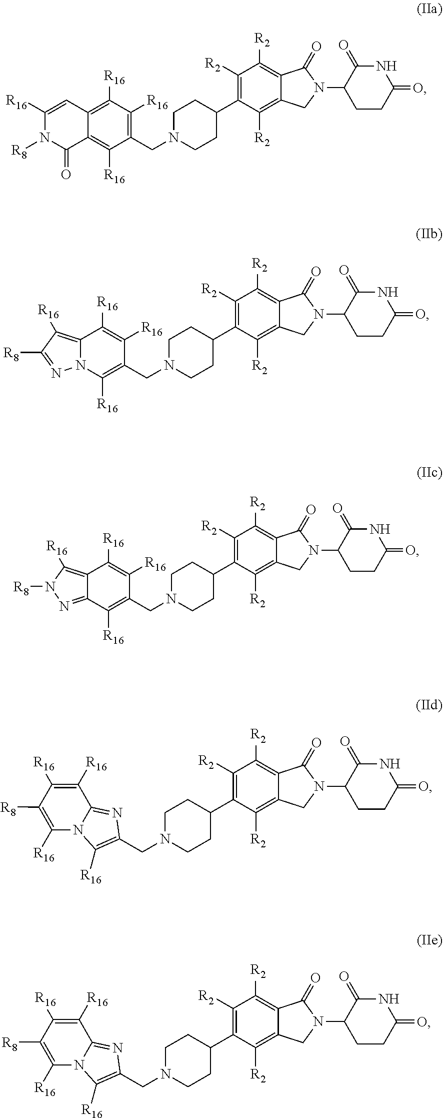
or a pharmaceutically acceptable salt, hydrate, solvate, prodrug, stereoisomer, or tautomer thereof,
wherein,
each R₂ is independently selected from the group consisting of hydrogen and halo; and
each R₁₆ is independently selected from the group consisting of hydrogen, (C₁-C₆)alkyl, (C₁-C₆)haloalkyl, and halo.

14. The compound of claim 13, wherein R₈ is selected from: 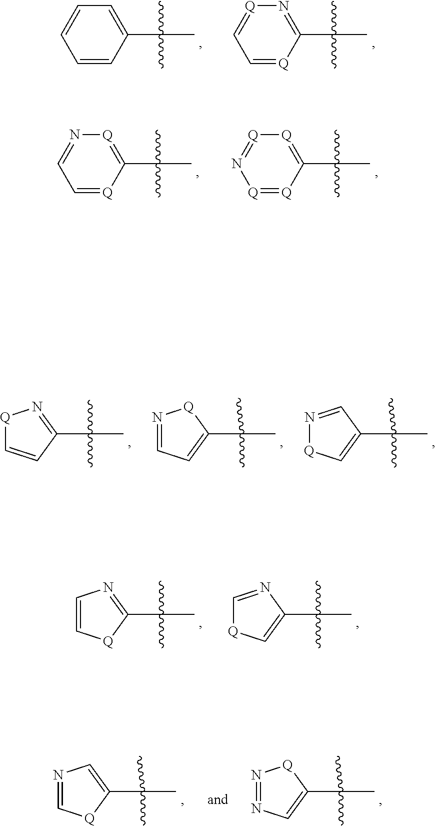
wherein R₈ is further optionally and independently substituted with one or more R₁₅.

15. The compound of claim 14, wherein R₈ is selected from: 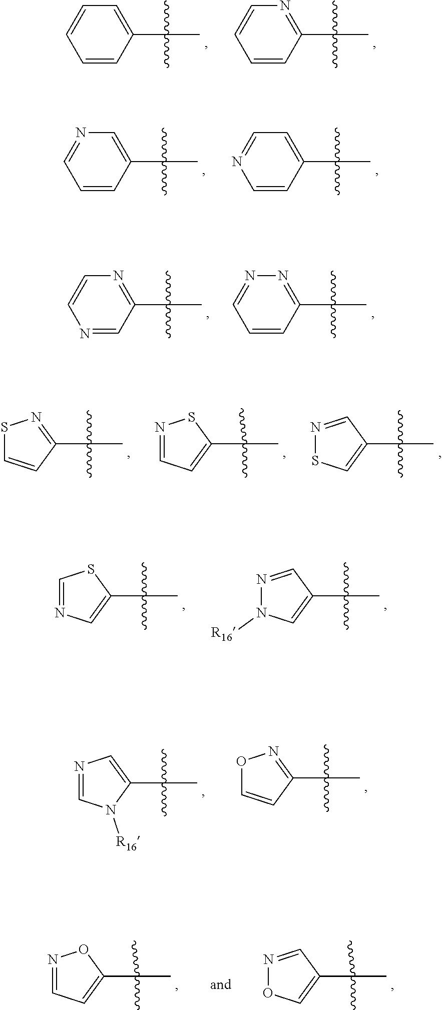
wherein
R₈ is further optionally and independently substituted with one or more (C₁-C₆)alkyl, (C₁-C₆)haloalkyl, (C₁-C₆)alkoxy, cyano, halo, and one or more groups selected from R₁₅; and
R₁₆′ is selected from the group consisting of hydrogen and (C₁-C₆)alkyl.

16. A compound which is: 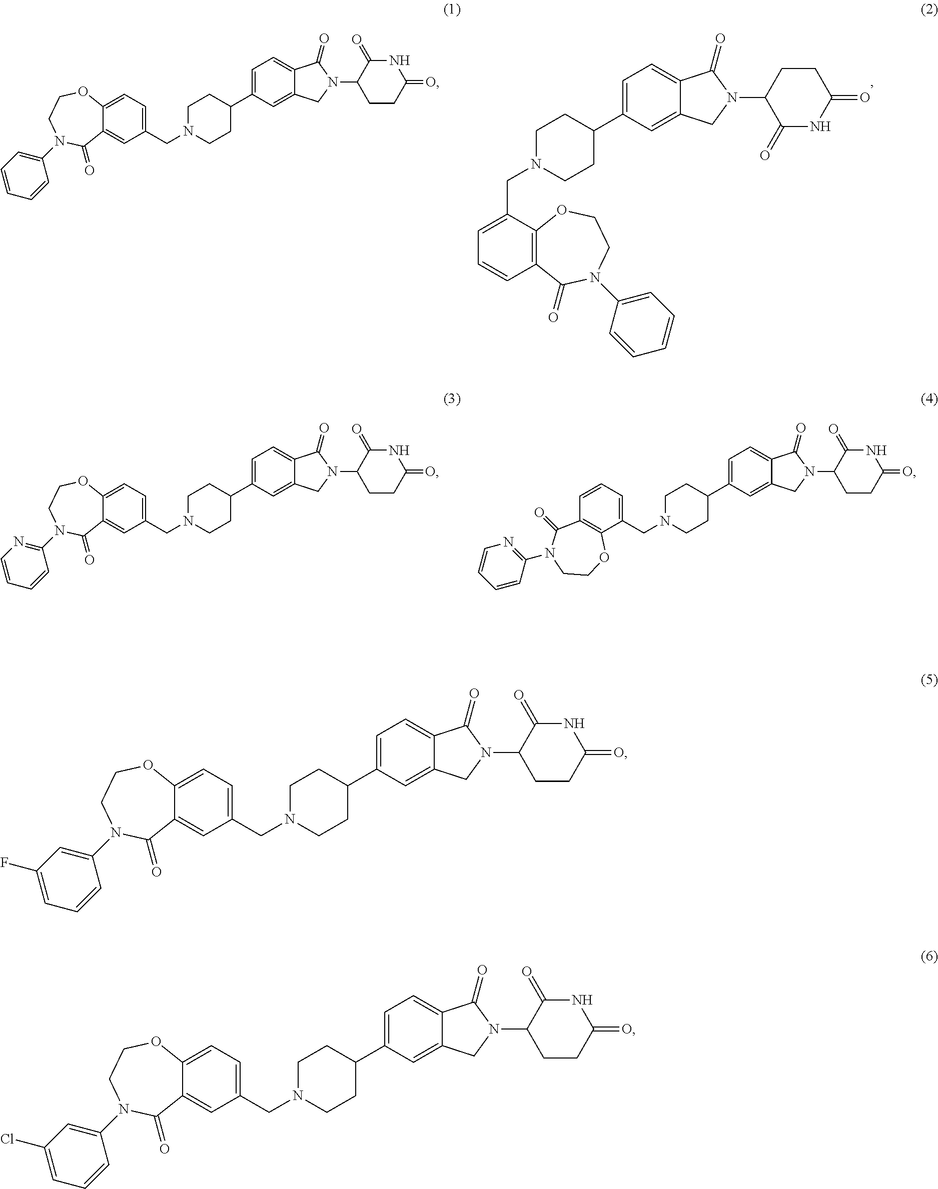 [SMILES: O=C1CCC(N2Cc3cc(C4CCN(Cc5ccc6c(c5)C(=O)N(c5ccccc5)CCO6)CC4)ccc3C2=O)C(=O)N1 ; O=C1CCC(N2Cc3cc(C4CCN(Cc5cccc6c5OCCN(c5ccccc5)C6=O)CC4)ccc3C2=O)C(=O)N1 ; O=C1CCC(N2Cc3cc(C4CCN(Cc5ccc6c(c5)C(=O)N(c5ccccn5)CCO6)CC4)ccc3C2=O)C(=O)N1 ; O=C1CCC(N2Cc3cc(C4CCN(Cc5cccc6c5OCCN(c5ccccn5)C6=O)CC4)ccc3C2=O)C(=O)N1 ; O=C1CCC(N2Cc3cc(C4CCN(Cc5ccc6c(c5)C(=O)N(c5cccc(F)c5)CCO6)CC4)ccc3C2=O)C(=O)N1 ; O=C1CCC(N2Cc3cc(C4CCN(Cc5ccc6c(c5)C(=O)N(c5cccc(Cl)c5)CCO6)CC4)ccc3C2=O)C(=O)N1] 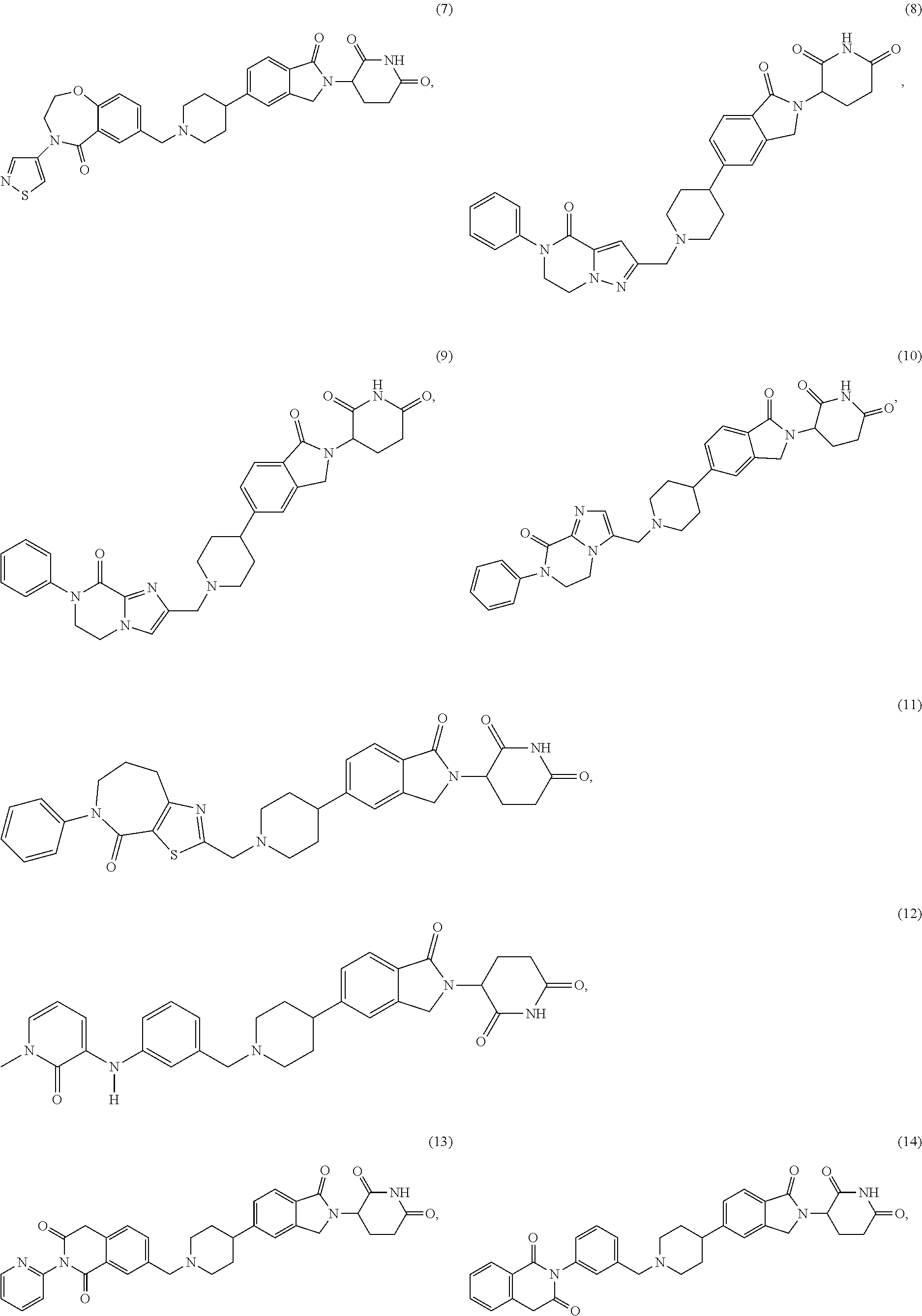 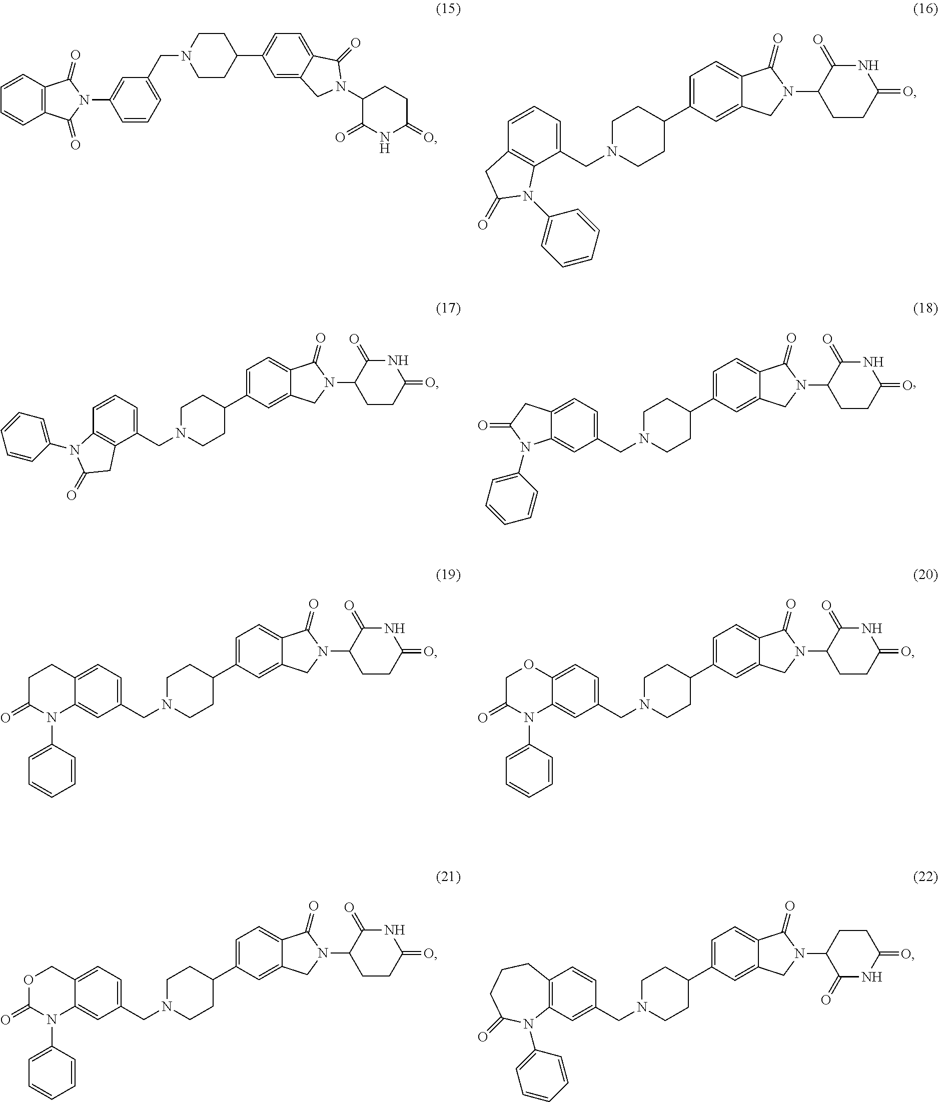 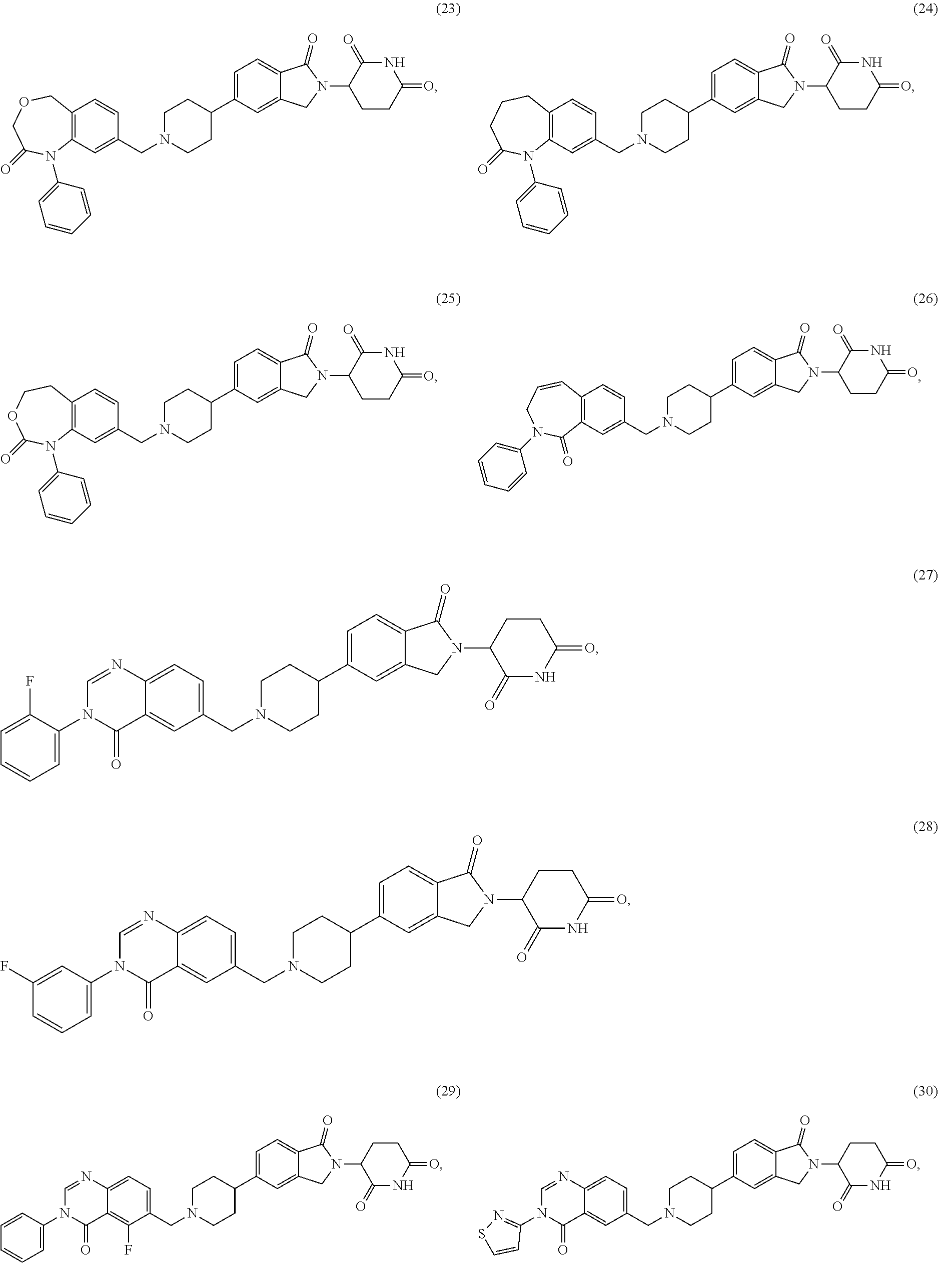 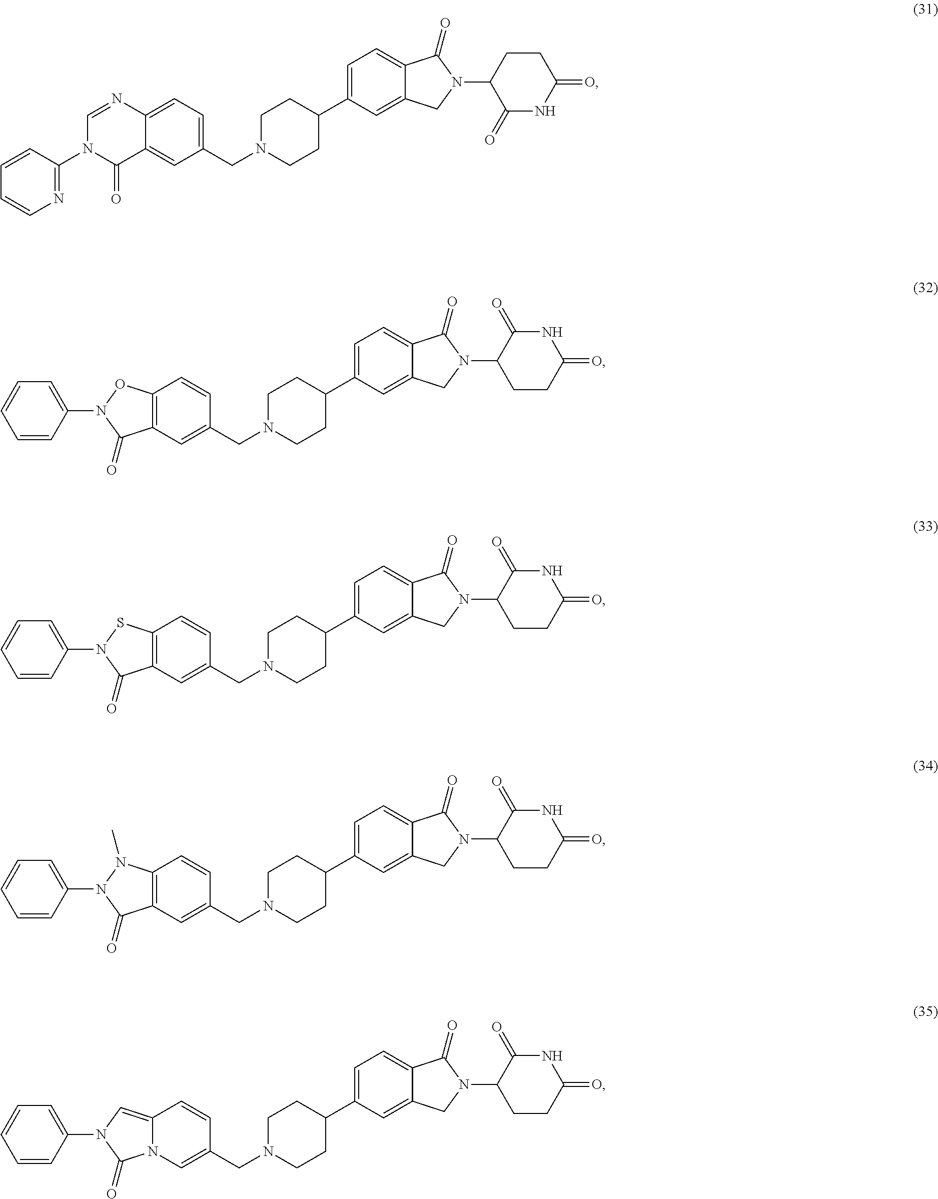 [SMILES: O=C1CCC(N2Cc3cc(C4CCN(Cc5ccc6ncn(-c7ccccn7)c(=O)c6c5)CC4)ccc3C2=O)C(=O)N1 ; O=C1CCC(N2Cc3cc(C4CCN(Cc5ccc6on(-c7ccccc7)c(=O)c6c5)CC4)ccc3C2=O)C(=O)N1 ; O=C1CCC(N2Cc3cc(C4CCN(Cc5ccc6sn(-c7ccccc7)c(=O)c6c5)CC4)ccc3C2=O)C(=O)N1 ; Cn1c2ccc(CN3CCC(c4ccc5c(c4)CN(C4CCC(=O)NC4=O)C5=O)CC3)cc2c(=O)n1-c1ccccc1 ; O=C1CCC(N2Cc3cc(C4CCN(Cc5ccc6cn(-c7ccccc7)c(=O)n6c5)CC4)ccc3C2=O)C(=O)N1] 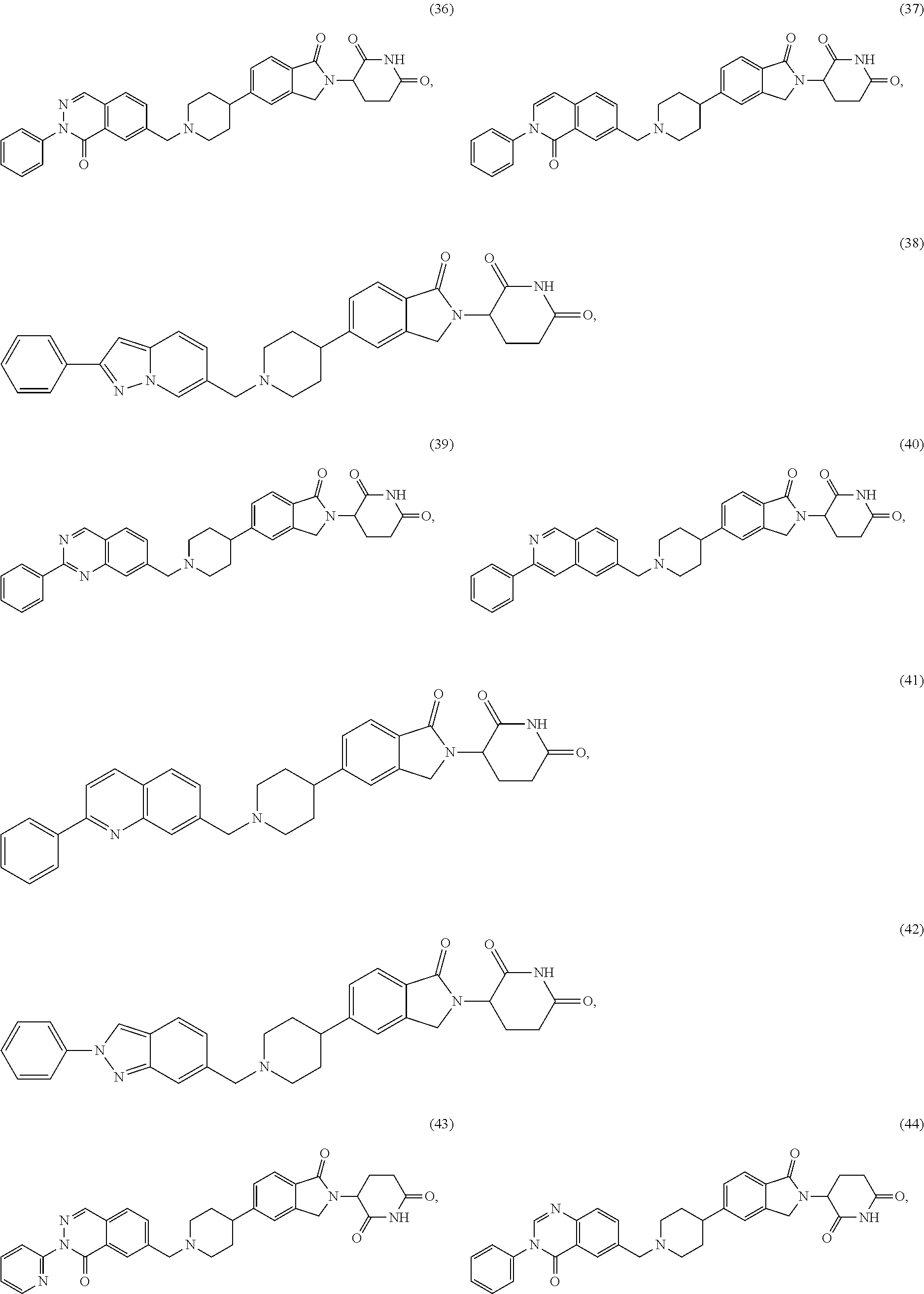 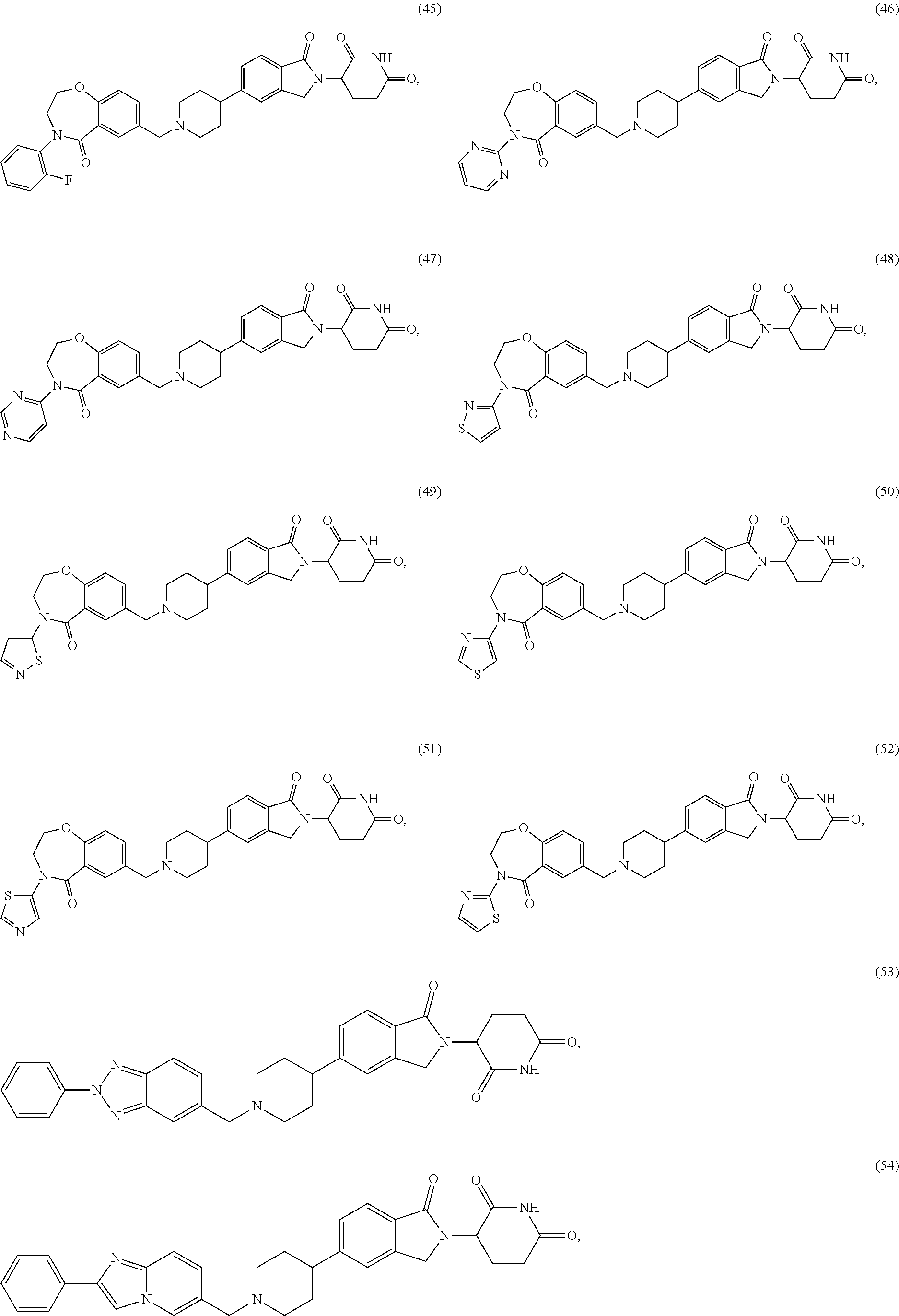 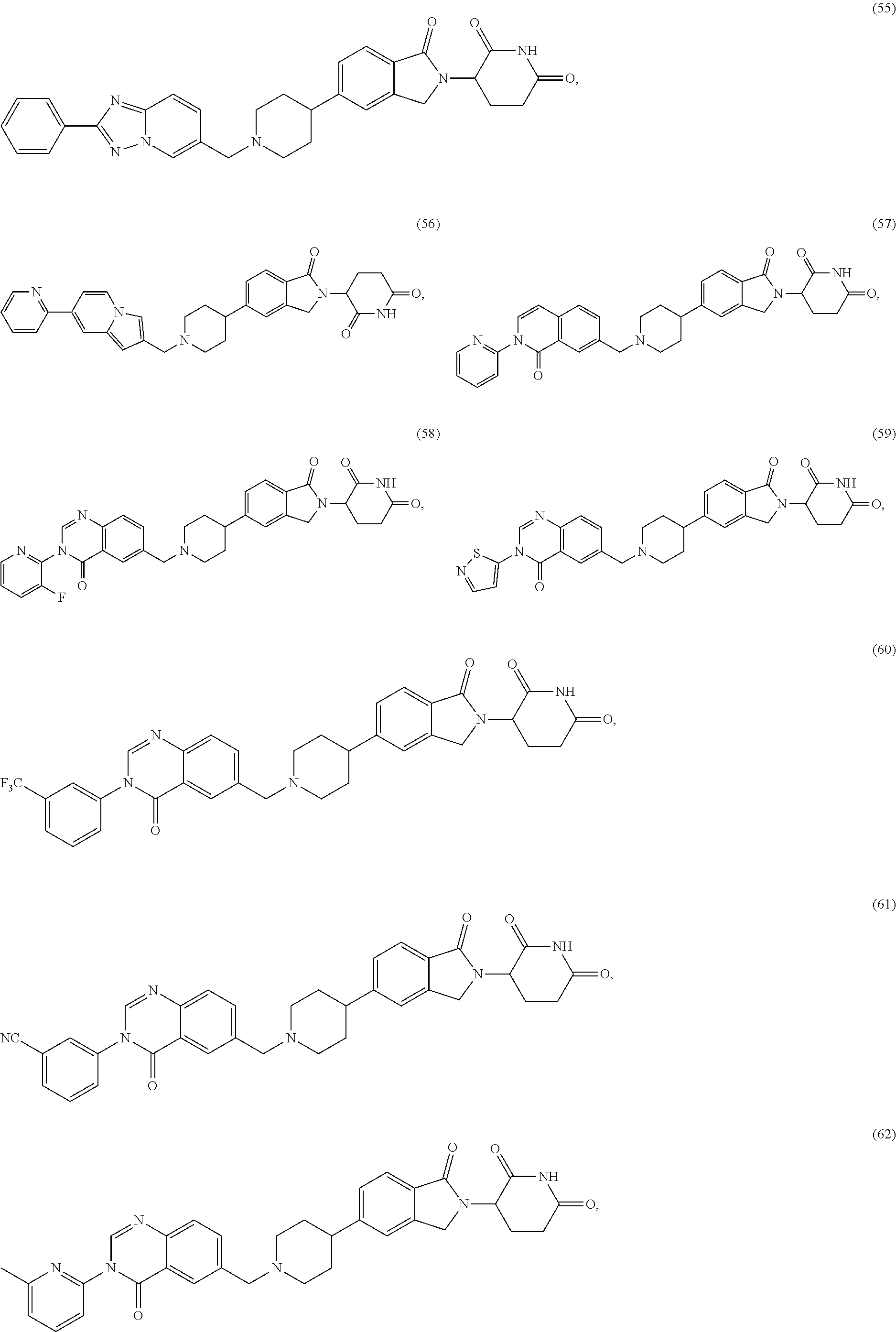 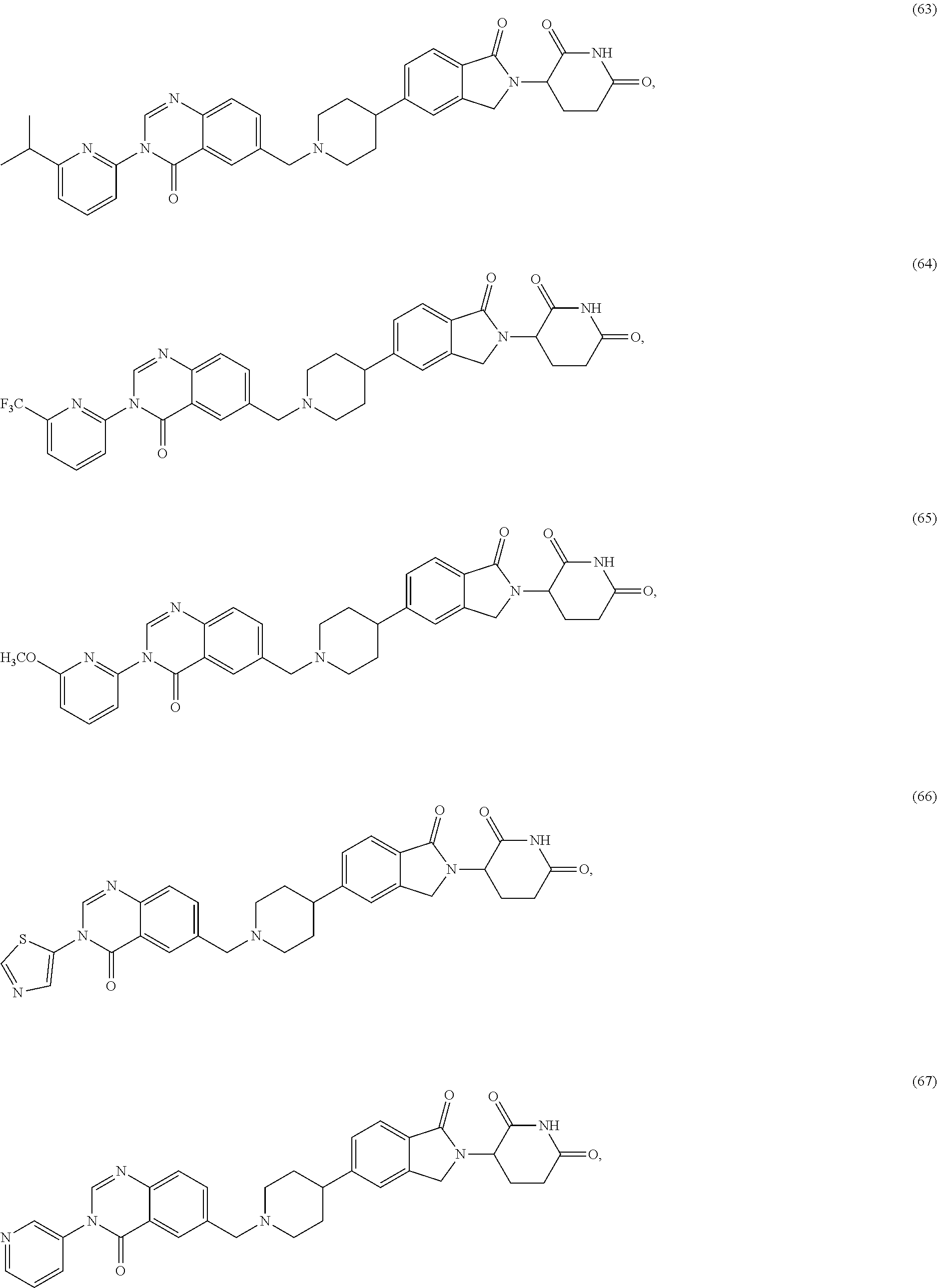 [SMILES: CC(C)c1cccc(-n2cnc3ccc(CN4CCC(c5ccc6c(c5)CN(C5CCC(=O)NC5=O)C6=O)CC4)cc3c2=O)n1 ; O=C1CCC(N2Cc3cc(C4CCN(Cc5ccc6ncn(-c7cccc(C(F)(F)F)n7)c(=O)c6c5)CC4)ccc3C2=O)C(=O)N1 ; COc1cccc(-n2cnc3ccc(CN4CCC(c5ccc6c(c5)CN(C5CCC(=O)NC5=O)C6=O)CC4)cc3c2=O)n1 ; O=C1CCC(N2Cc3cc(C4CCN(Cc5ccc6ncn(-c7cncs7)c(=O)c6c5)CC4)ccc3C2=O)C(=O)N1 ; O=C1CCC(N2Cc3cc(C4CCN(Cc5ccc6ncn(-c7cccnc7)c(=O)c6c5)CC4)ccc3C2=O)C(=O)N1] 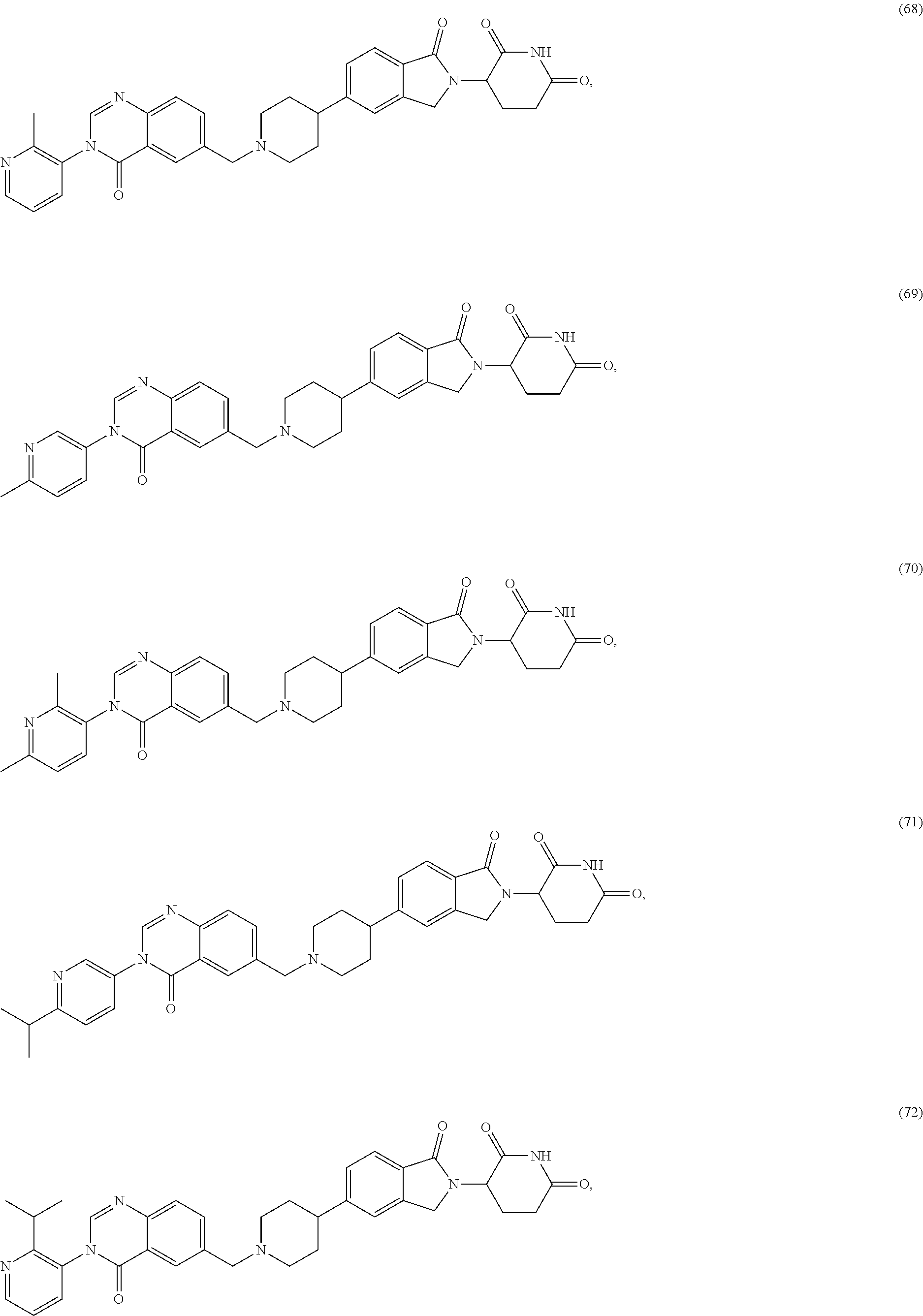 [SMILES: Cc1ncccc1-n1cnc2ccc(CN3CCC(c4ccc5c(c4)CN(C4CCC(=O)NC4=O)C5=O)CC3)cc2c1=O ; Cc1ccc(-n2cnc3ccc(CN4CCC(c5ccc6c(c5)CN(C5CCC(=O)NC5=O)C6=O)CC4)cc3c2=O)cn1 ; Cc1ccc(-n2cnc3ccc(CN4CCC(c5ccc6c(c5)CN(C5CCC(=O)NC5=O)C6=O)CC4)cc3c2=O)c(C)n1 ; CC(C)c1ccc(-n2cnc3ccc(CN4CCC(c5ccc6c(c5)CN(C5CCC(=O)NC5=O)C6=O)CC4)cc3c2=O)cn1 ; CC(C)c1ncccc1-n1cnc2ccc(CN3CCC(c4ccc5c(c4)CN(C4CCC(=O)NC4=O)C5=O)CC3)cc2c1=O] 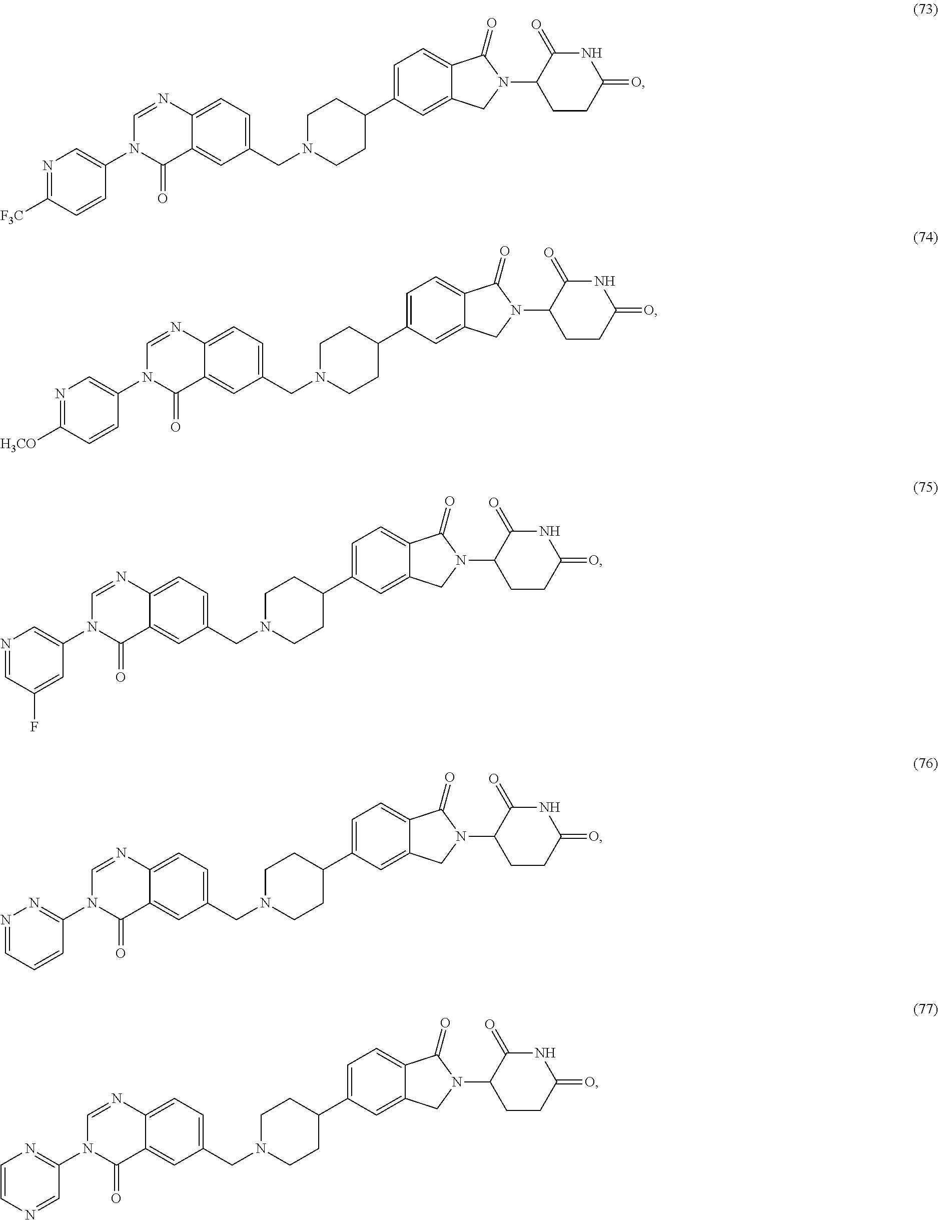 [SMILES: O=C1CCC(N2Cc3cc(C4CCN(Cc5ccc6ncn(-c7ccc(C(F)(F)F)nc7)c(=O)c6c5)CC4)ccc3C2=O)C(=O)N1 ; COc1ccc(-n2cnc3ccc(CN4CCC(c5ccc6c(c5)CN(C5CCC(=O)NC5=O)C6=O)CC4)cc3c2=O)cn1 ; O=C1CCC(N2Cc3cc(C4CCN(Cc5ccc6ncn(-c7cncc(F)c7)c(=O)c6c5)CC4)ccc3C2=O)C(=O)N1 ; O=C1CCC(N2Cc3cc(C4CCN(Cc5ccc6ncn(-c7cccnn7)c(=O)c6c5)CC4)ccc3C2=O)C(=O)N1 ; O=C1CCC(N2Cc3cc(C4CCN(Cc5ccc6ncn(-c7cnccn7)c(=O)c6c5)CC4)ccc3C2=O)C(=O)N1] 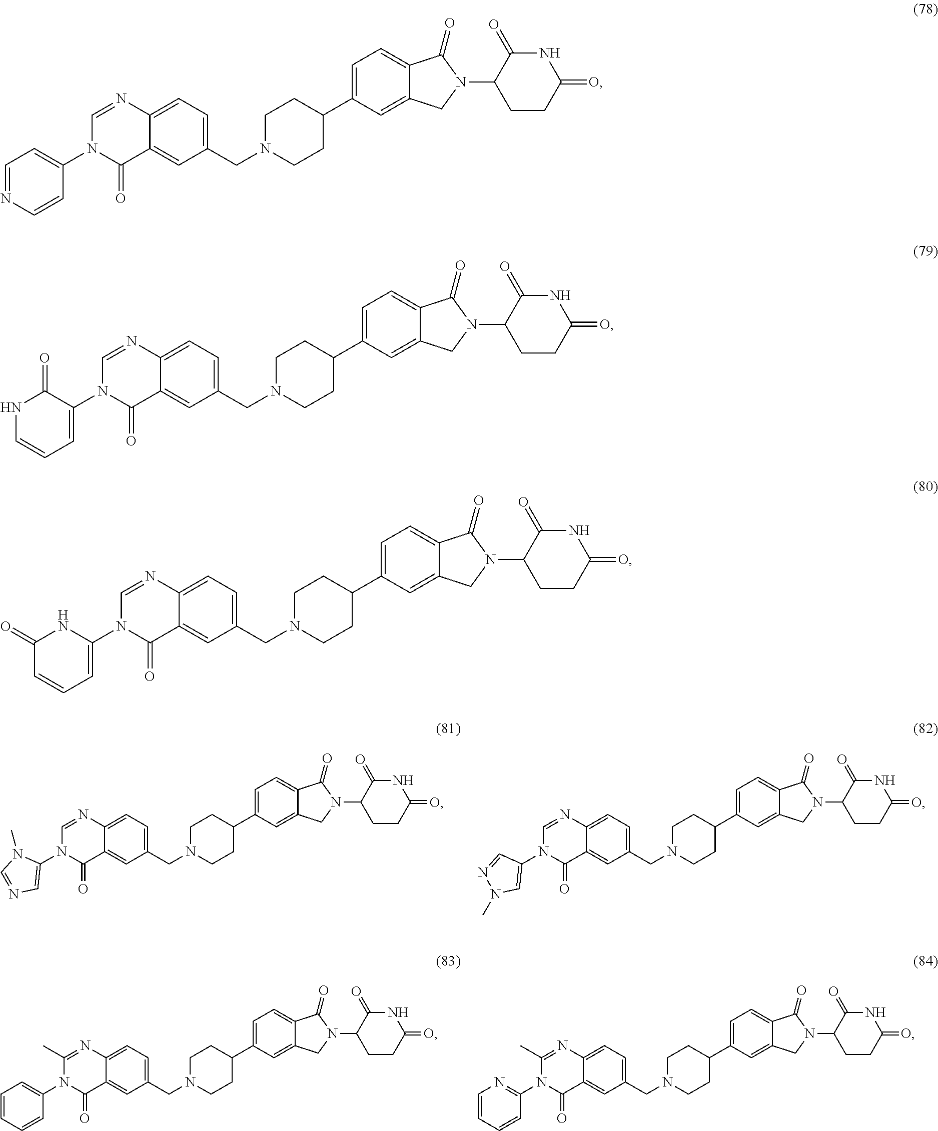 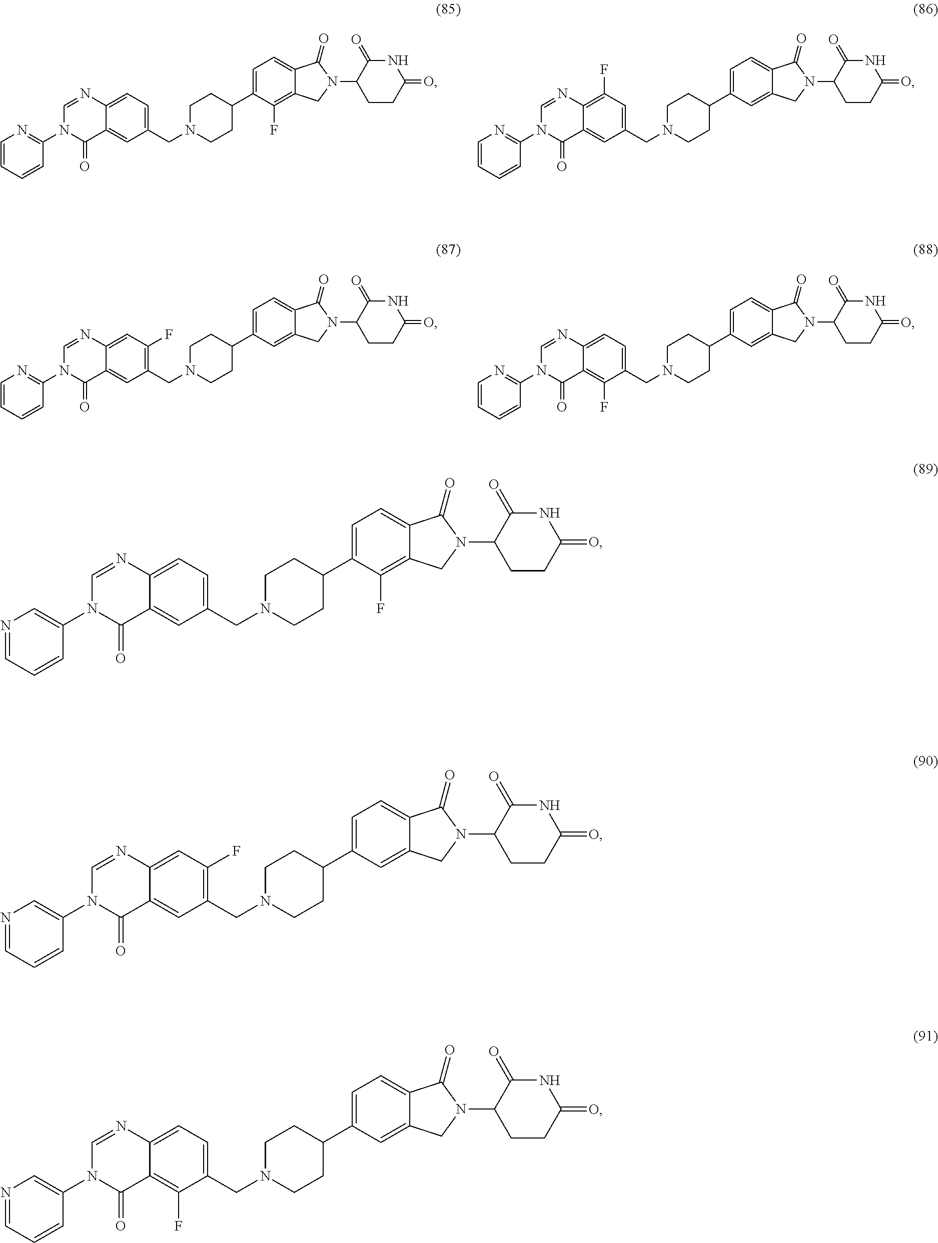 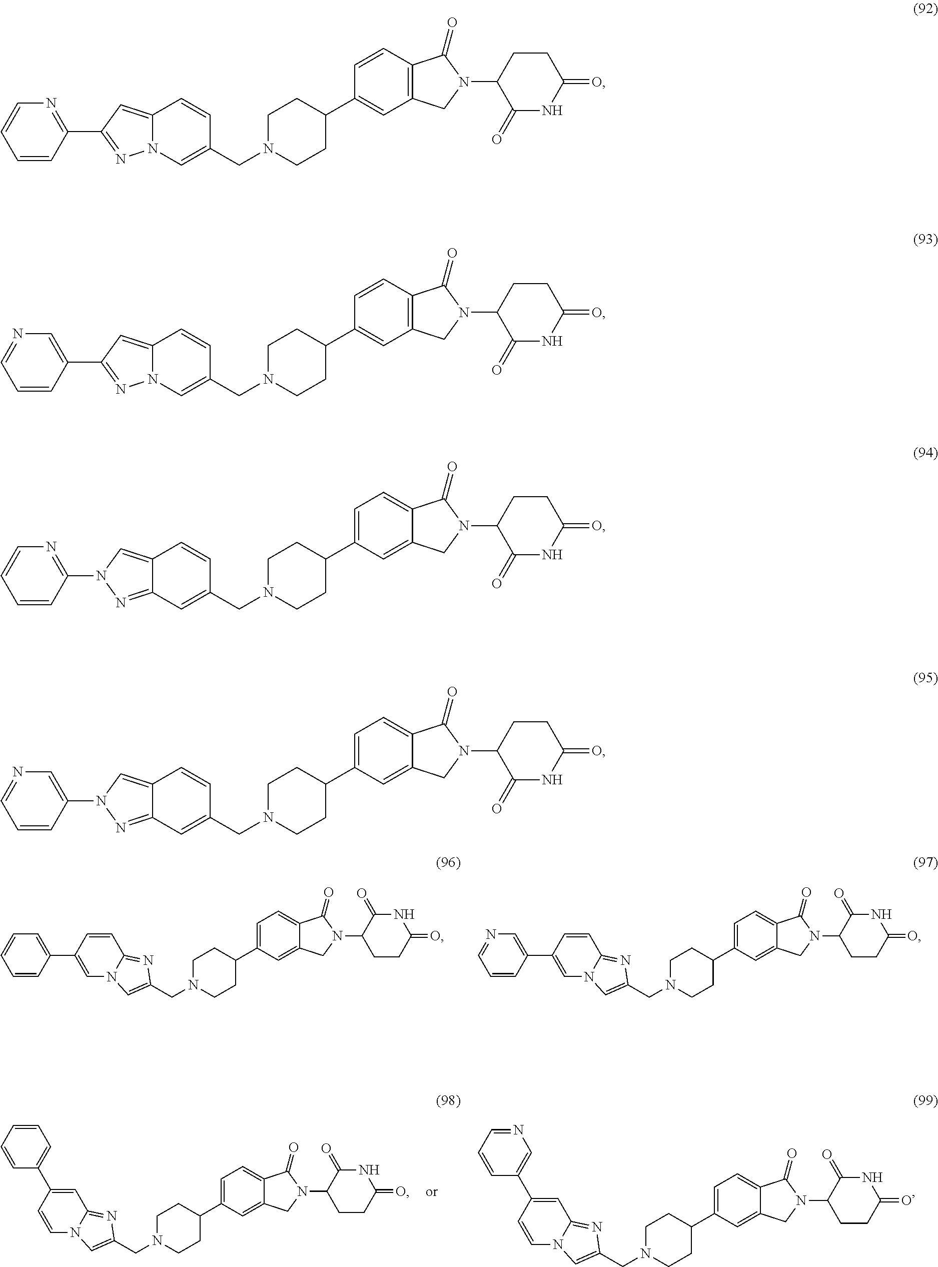
or a pharmaceutically acceptable salt, hydrate, solvate, prodrug, stereoisomer, or tautomer thereof.

17. The compound of claim 1, which is in the form of the pharmaceutically acceptable salt.

18. A pharmaceutical composition, comprising a therapeutically effective amount of the compound or pharmaceutically acceptable salt, hydrate, solvate, prodrug, stereoisomer, or tautomer thereof of claim 1, and a pharmaceutically acceptable carrier.

19. The pharmaceutical composition of claim 18, wherein the compound is in a form of a co-crystal.

20. A method of treating a disease or disorder that is associated with IKZF2 (Helios) and would benefit from IKZF2 degradation, comprising administering to a subject in need thereof a therapeutically effective amount of the compound or pharmaceutically acceptable salt, hydrate, solvate, prodrug, stereoisomer, or tautomer thereof of claim 1.

21. The method of claim 20, wherein the disease or disorder is cancer.

22. The method of claim 21, wherein the cancer is T cell leukemia, T cell lymphoma, Hodgkin's lymphoma, non-Hodgkin's lymphoma, myeloid leukemia, non-small cell lung cancer (NSCLC), melanoma, triple-negative breast cancer (TNBC), nasopharyngeal cancer (NPC), microsatellite stable colorectal cancer (mssCRC), thymoma, carcinoid, or gastrointestinal stromal tumor (GIST).

23-32. (canceled)
